SEQUENCE LISTING (3 sequences)

SEQ ID NO: 1 - DNA, 37 bp, artificial
  Read1CustSeqB Primer
gcctgtccgcggaagcagtggtatcaacgcagagtac

SEQ ID NO: 2 - DNA, 22 bp, artificial
  Cre primer
attgctgtcacttggtcgtggc

SEQ ID NO: 3 - DNA, 23 bp, artificial
  Cre primer R4
ggaaaatgcttctgtccgtttgc

(19) United States
(12) Patent Application Publication
(10) Pub. No.: US 2022/0334101 A1
(43) Pub. Date: Oct. 20, 2022

(54) MODULATING EXTRACELLULAR MATRIX MOVEMENT

(71) Applicant: Helmholtz Zentrum Muenchen - Deutsches Forschungszentrum Fuer Gesundheit Und Umwelt (GmbH), Neuherberg (DE)

(72) Inventors: Juliane Wannemacher, Mainz-Kostheim (DE); Adrian Fischer, Munich (DE); Donovan Correa-Gallegos, Munich (DE); Yuval Rinkevich, Munich (DE); Qing Yu, Wuhan (CN)

(21) Appl. No.: 17/635,334

(22) PCT Filed: Aug. 17, 2020
(86) PCT No.: PCT/EP2020/073008

§ 371 Date: Feb. 14, 2022

(30) Foreign Application Priority Data
Aug. 15, 2019 (EP) 19191998.4

(51) Int. Cl. G01N 33/50; G01N 33/68; A61K 31/496; A61P 11/00

(52) U.S. Cl. CPC G01N 33/5082; G01N 33/5044; G01N 33/5029; G01N 33/6818; A61K 31/496; A61P 11/00

(57) ABSTRACT

The present invention provides for methods for identifying modulators of extracellular matrix (ECM) movement towards a site requiring deposition of ECM. Such modulators can be applied for use in a method for the modulation of ECM movement towards a site requiring deposition of ECM, e.g. a wound, thereby allowing treatment of a condition involving ECM deposition. Since the modulator may either be an inhibitor or promoter, either excessive or insufficient ECM deposition could be dealt with by the means and methods of the present invention.

### FIELD OF THE INVENTION

The present invention provides for methods for identifying modulators of extracellular matrix (ECM) movement towards a site requiring deposition of ECM. Such modulators can be applied for use in a method for the modulation of ECM movement towards a site requiring deposition of ECM, e.g. a wound, thereby allowing treatment of a condition involving ECM deposition. Since the modulator may either be an inhibitor or promoter, either excessive or insufficient ECM deposition could be dealt with by the means and methods of the present invention.

### BACKGROUND OF THE INVENTION

In mammals, scars are formed when a specialized population of fibroblasts immigrates into wounds to locally deposit plugs of connective tissue matrix at sites of injury¹. The origin of scar-producing fibroblasts, myofibroblasts, in wounds is unclear and so, by extension, is the mechanism by which they act². Myofibroblasts are suggested to emanate from various sources, such as papillary (upper) and reticular (lower) dermal layers³, pericytes⁴, adipocytes⁵⁻⁶, and from bone-marrow derived circulating monocytes⁷.

The provenance of scar, myofibroblasts, and the mechanism by which they gain this unique capacity are thus still obscure despite scars being an extensively studied major clinical challenge. Indeed, when normal scarring fails, the result is either non-healing chronic wounds or aggravating scarring and fibrosis⁸⁻¹⁰. Impaired wounds and excessive scarring are a tremendous burden for patients and for the global healthcare system and they cost tens of billions of dollars per year, just in the US¹¹. Understanding this fundamental patching process is therefore critical to restore and preserve the normal functions of injured adult organs.

It was previously demonstrated that all scars in the back-skin come from a distinct fibroblast lineage expressing the Engrailed-1 gene in embryogenesis¹²⁻¹³. This cell lineage is present not only in the skin, but also in the strata underneath the skin, called subcutaneous fascia. The subcutaneous fascia is a gelatinous viscoelastic membranous sheet of matrix that creates a frictionless gliding interface between the skin and the body's rigid structure below. For example, in the murine back-skin, the subcutaneous fascia is a single connective sheet that is separated from the skin by the Panniculus carnosus (PC) muscle, whereas in humans there is no intervening muscle and the subcutaneous fascia is relatively thick, consisting of several membranous sheets that are continuous with the upper skin layers. In humans the facia layers incorporate fibroblasts, lymphatics, adipose tissue, neurovascular sheets and sensory neurons¹⁴⁻¹⁵.

A major component of scars is extracellular matrix (ECM). Excessive as well as insufficient deposition of ECM is undesired, since it may result, e.g. in fibroproliferative diseases or chronic wounds, respectively. Many attempted are made in the prior art to deal with medical conditions concerning excessive or insufficient ECM deposition in the scar-process, but the process is still not clearly understood which hampers the development of beneficial treatment options. Hence, there is still a need to provide further options in the treatment of excessive or insufficient scar formation.

It is therefore desired to satisfy the need to provide further options in the treatment of excessive or insufficient scar formation.

The present invention addresses this need and provides options in the treatment of conditions involving ECM deposition, e.g. excessive or insufficient scar formation. Such conditions may be either excessive deposition of ECM at a site requiring ECM deposition or insufficient deposition of ECM at a site requiring ECM deposition.

### SUMMARY OF THE INVENTION

Accordingly, in a first aspect, the present invention relates to a method for identifying modulators of extracellular matrix (ECM) movement towards a site requiring deposition of ECM, comprising (a) contacting extracellular matrix of organ tissue obtainable by biopsy from a mammalian subject with a label; (b) contacting said labelled extracellular matrix of organ tissue with a compound of interest; (c) determining whether said compound of interest modulates ECM movement towards said site requiring deposition of ECM in comparison to labelled extracellular matrix of organ tissue obtainable by biopsy from a mammalian subject which is not contacted with said compound of interest, wherein modulation of ECM movement towards said site requiring deposition of ECM is indicative for said compound of interest to be a modulator of said ECM movement.

The present invention may also comprise the method as described elsewhere herein, wherein modulation is inhibition.

Further, the present invention may also comprise the method as described elsewhere herein, wherein modulation is promotion.

Further being envisaged herein is the method as described elsewhere herein, wherein said organ tissue comprises fascia matrix, serosa and/or adventitia.

The present invention may also comprise the method as described elsewhere herein, wherein fascia matrix, serosa and/or adventitia comprises macrophages, neutrophils, mesothelial cells and/or fibroblasts.

The present invention may also encompass the method as described elsewhere herein, wherein ECM comprises proteins, polysaccharides and/or proteoglycans.

Also comprised by the present invention may be the method as described elsewhere herein, wherein the label is a dye or tag. Preferably, the dye is a fluorescent dye.

Additionally, the present invention may encompass the method as described elsewhere herein, wherein primary amine groups of extracellular matrix components are labelled.

Also envisaged herein is the method as described elsewhere herein, wherein the label is covalently coupled to extracellular matrix components.

Further, the present invention may also comprise the method as described elsewhere herein, wherein contacting extracellular matrix of organ tissue obtainable by biopsy from said mammalian subject with a label is achieved by contacting said extracellular matrix with a paper-like material comprising the label.

The present invention may also envisage the method as defined elsewhere herein, wherein fluid of said mammalian's body cavity is present during step (a), (b) and/or (c).

The present invention may also encompass the method as described elsewhere herein, further comprising step (a′) contacting said organ tissue obtainable by biopsy from said mammalian subject with a label visualizing cells comprised in the ECM.

It may also be comprised herein the method as described elsewhere herein, wherein the organ tissue is from skin, kidney, lung, heart, liver, bone, peritoneum, intestine, diaphragm or pleura.

According to a second aspect, the present invention relates to a method for identifying a biomarker associated with extracellular matrix (ECM) movement towards a site requiring deposition of ECM, comprising (a) contacting extracellular matrix of organ tissue obtainable by biopsy from a mammalian subject with a label; (b) isolating proteins from said labelled ECM which move towards said site requiring deposition of ECM; (c) determining at least a partial amino acid sequence of said proteins, thereby identifying said proteins as a biomarker associated with ECM movement.

Additionally, according to a third aspect, the present invention refers to a compound for use in a method for the modulation of extracellular matrix (ECM) movement towards a site requiring deposition of ECM, preferably in the treatment of a condition involving ECM deposition.

The present invention may also comprise the compound for the use as described elsewhere herein, wherein ECM movement is mediated by fascia matrix.

The present invention may also encompass the compound for the use as described elsewhere herein, wherein fascia matrix, serosa and/or adventitia comprises macrophages, neutrophils, mesothelial cells, and/or fibroblasts.

Also comprised by the present invention is the compound for the use as described elsewhere herein, wherein fascia matrix, serosa and/or adventitia comprises fibroblasts.

Also envisaged herein is the compound for the use as described elsewhere herein, wherein ECM comprises proteins, polysaccharides and/or proteoglycans.

Further, the present invention may also comprise the compound for the use as described elsewhere herein, wherein the site requiring deposition of ECM is a wound.

Additionally, the present invention may also encompass the compound for the use as described elsewhere herein, wherein modulation is inhibition. Preferably, inhibition of ECM movement towards a site requiring deposition of ECM prevents excessive deposition of ECM at said site. Even more preferably, excessive deposition of ECM is associated with fibroproliferative disease.

Further, the present invention may also envisage the compound for the use as described elsewhere herein, wherein the condition involving ECM deposition is excessive deposition of ECM. Preferably, excessive deposition of ECM is associated with fibroproliferative disease.

Additionally, the present invention may also encompass the compound for the use as described elsewhere herein, wherein modulation is promotion. Preferably, promotion of ECM movement towards a site requiring deposition of ECM prevents insufficient deposition of ECM at said site. Even more preferably, insufficient deposition of ECM is associated with chronic wounds.

Also envisaged herein is the compound for the use as described elsewhere herein, wherein the condition involving ECM deposition is insufficient deposition of ECM. Preferably, insufficient deposition of ECM is associated with chronic wounds.

Additionally, the present invention may also encompass the compound for the use as described elsewhere herein, wherein said compound is obtainable by the method for identifying modulators of extracellular matrix (ECM) movement towards a site requiring deposition of ECM as described elsewhere herein.

### BRIEF DESCRIPTION OF THE FIGURES

FIG. 1: Fascia is the major cellular source for wounds. a. Schematic description of chimeric grafts to determine the cellular contribution of dermis and fascia to the wound. b. Quantification of the TdTomato⁺ or GFP⁺ cells percentage from the total labeled cells (TdTomato⁺ and GFP⁺) in the wound and wound margin. N=26 sections analyzed from 4 biological replicates. One-way ANOVA, multiple comparison Tukey test, confidence interval=95%. c. Histological section of wound showing skin-derived TdTomato⁺ cells (red) and fascia-derived GFP⁺ cells (green) at 14 dpw. d-e. Immunostaining and contribution quantifications for myofibroblasts (αSMA), nerves (TUBB3), blood vessels (PECAM1-CD31), macrophages (MOMA-2), and lymphatic vessels (LYVE1). Dotted lines delimitate the wound area. Arrowheads indicate the original injury site. Scale bars=200 microns.

FIG. 2: Fascial EPFs invasion into the wound dictates scar severity. a. Schematic description of dermal or fascial EPFs labeling using chimeric grafts. b. Histological images co-stained with DAPI (blue) showing fascial-EPFs (green, left) or dermal-EPFs (right) invading the wound bed after a deep (top) or superficial injury (bottom). c. Wound size measurements from both injury conditions. N=53 and 70 images analyzed from 5 biological replicates. Unpaired, two-tailed T-test, confidence interval=95%. d. Fascial and dermal EPFs numbers in both injury conditions. N=27, 32, 27, and 22 images analyzed from 5 biological replicates. Unpaired, two-tailed T-test, confidence interval=95%. e-f. XY plots of EPFs fraction in wounds and wound size from fascial- (d) and dermal EPFs (e). Pearson correlation, confidence interval=95%. Dotted lines delimitate the wound. Scale bars=200 microns.

FIG. 3: Fascia matrix steers into wounds. a. 3D rendered SHG (a) and SEM images (b) of adult fascia (left) and dermis (right) showing the different matrix fiber arrangements. c. Scatter plot showing the fractal dimension and lacunarity values to assess the complexity and porosity, respectively, in the fiber arrangements from SHG images. N=5 and 3 images analyzed. Unpaired two-tailed T-test, confidence interval=95%. d. XY time-lapse images of the 3D rendered C57BL6/J neonate fascia biopsy in culture. SHG (Cyan) and autofluorescence (green) signals at time 0 (left), and 30 hours (right) depicting the fascial matrix movements. Lines show the length reduction in time. e. Length versus time plot of tracked points from the SHG and autofluorescence channels showing a clear contraction of the fascial matrix in culture. f. Schematic description of in situ fascial matrix labeling. Subcutaneous injections of FITC NHS ester were performed prior injury of WT mice back-skin to label the fascia matrix. g. Left: histological sections showing fascia matrix (FITC, green) and COLLAGENI+III+VI (magenta) at the defined time points after wounding. Right: Subsampled fractal dimension maps of the FITC signal at the uninjured, 3, and 5 dpw, and from collagens signal at 14 dpw. h. FITC signaling coverage quantification of the total COLLAGEN I+III+VI signal in the wound. N=3, 4, 7, and 4 sections analyzed from 3 biological replicates. One-way ANOVA, Tukey multiple comparisons. i. Scatter plot showing the average fractal dimension and lacunarity values from the subsampled maps in g. Arrowheads indicate the original injury site. Lines delimitate fascia compartment. Scale bars=30 microns (a-b), 500 microns (d), and 200 microns (g).

FIG. 4: Fascial EPFs mediate scar-forming matrix steering into wounds. a. Schematic description of the ePTFE membrane implantations to block the fascial discharges into the wound. b. Wound closure plots of ePTFE-implanted or sham control wounds (left). Wound size was determined from photographs (right) taken at the specified time points after wounding. N=3 biological replicates. Unpaired two-tailed T-test, confidence interval=95%. c. Histology showing wounds from ePTFE-implanted (right) or sham controls (left) at dpw 63. Masson's trichrome staining (top) and collagens (magenta) combined immunolabeling (middle) show that fascia involvement in the wound healing process is necessary for scar formation. High magnification images (cI-II, bottom) at the wound edges showed the presence of multiclonal dermal EPFs (orange, cyan, and red) that are incapable of forming a scar tissue on top the ePTFE membrane. d. Schematic description of the fascia release experiments. e. Wound closure plots of fascia-released or control wounds (left). Wound size was determined from photographs (right) taken at the specified time points after wounding. N=8 images analyzed from 8 biological replicates. Unpaired two-tailed T-test, confidence interval=95%. f. Masson's trichrome staining images showing wounds at 3, 5, and 7 days post wounding (from top to bottom) from fascia-released (right) or control wounds (left) showing that fascia release delays the wound healing process. g. Schematic description of the partial fascial cell depletion experiments in R26^{iDTR} neonates. AAV6-Cre or AAV6-GFP control viral particles were injected in the skin between the two wounds, followed by a daily systemic exposure to DT for seven days. h. Masson's trichrome staining and fluorescent images showing wounds 7 dpw in Cre-transduced (right) or control GFP-transduced (left) mice. Arrows indicate GFP-positive cells. i. Scar-length measurements in microns for the two conditions. N=4 and 8 sections analyzed from 3 biological replicates. Unpaired two-tailed T-test, confidence interval=95%. j. Schematic description of the fascial-EPFs depletion in chimeric skin grafts with labeled-fascial-ECM. k. Immunodetection of collagens (cyan) showing the fascial matrix (magenta) in wounds of DT-treated (right) or vehicle control grafts (left). l. Alexa Fluor 647-signaling coverage quantification proves that fascial EPF ablation severely impairs the fascial matrix discharges into the wound bed. N=6 sections analyzed from 3 biological replicates. Unpaired two-tailed T-test, confidence interval=95%. Dashed lines delimit the ePTFE membrane location. Dotted lines delimitate the wound bed. Arrowheads indicate the original injury site. Arrow indicates the remaining labeled fascial matrix in DT-treated mice. Scale bars=50 microns (cI and cII), 200 microns (h), and 500 microns (c, f, h, and k). PC=Panniculus carnosus.

FIG. 5: Keloid scars originate from subcutaneous fascia. a-b. macroscopic pictures and Masson's trichrome staining of human back skin and abdominal skin showing fascia layers embedded in subcutaneous fat. Arrows indicate the fascia tissue. c. immunostaining of CD26 and CD44, NOV and a-SMA, FAP on cryosections of fascia, dermis and keloid scar of human back skin, respectively. d. Relative fluorescence intensity of CD26, CD44, FAP, and NOV signal. n=4, One-way ANOVA with Tukey's test, 95% Confidence interval. e-f. Immunostaining for NOV (CCN3, blue) on En1^{Cre}; R26^{mTmG} 14 dpw scars. g. Relative fluorescence intensity of NOV expression. n=6 images of 3 biological replicates, One-way ANOVA, Tukey's test, 95% Confidence interval. Dotted and broken lines delimitate scar and fascia respectively. Scale bars: 2 mm (a-b), 50 microns (c), and 200 microns (e-f).

FIG. 6: Model of superficial fascia role on wound healing. Superficial injuries heal by the classical fibroblast migration and de novo matrix deposition process. In response to a deep injury, the externum repono (fascia tissue) is steered into wounds by fascial EPFs. Fascia-derived fibroblasts, macrophages, endothelial and peripheral nerves rapidly clog the open wound. Fascia matrix undergoes an initial expansion followed by a progressive contraction and remodeling until curated into a mature scar.

FIG. 7: Fascial cells tracking using Oil dye. a. Schematic description of Dil labeling of fascia. b. Histology of wounds showing Dil⁺ (red) cells in uninjured controls and 14 dpw, co-stained with DAPI (blue). c. Immunolabeling (green, left) of wound beds with Dil-labeled fascial cells (red) and fractions (right) of positive cells for mesenchymal/fibroblast markers ITGB1 (CD29), ER-TR7, THY1 (CD90), and PDGFRA, d. Immunolabeling and fraction of Dil⁺ monocytes/macrophages (MOMA-2), lymphatics (LYVE1), endothelial (PECAM1/CD31), and nerves (TUBB3). N=4 to 5 images analyzed from 5 biological replicates. Dotted lines delimitate the wound bed. Scale bars=200 microns. Ep=epidermis. PC=Panniculus carnosus.

FIG. 8: Fascial EPFs traverse the PC muscle. a. Gating strategy for fibroblasts analysis. Singlets were gated with the selected gates (red lines, see “methods”). b. percentages of fibroblasts (Lin⁻) and lineage-positive cells in fascia and dermis N=4 independent experiments. Two-way ANOVA, multiple comparison Tukey test, confidence interval=95%. c. Representative scatter plots for detection of EPFs (GFP+, Lin−) and ENFs (TdTomato+, Lin−) populations in fascia and dermis. d. Quantification of the total EPFs (GFP+, Lin−) and ENFs (TdTomato+, Lin−, d), and other resident cells types (e) fractions in fascia versus dermis. N=4 independent experiments. Two-way ANOVA, multiple comparison Tukey test, confidence interval=95%. e. Quantification of endothelial (CD31+), lymphatics (Lyve1+), macrophages (F4/80+), and nervous (CD271+) cell fractions from total cells in fascia versus dermis. N=3 technical replicates from a pooled litter. Two-way ANOVA, multiple comparison Tukey test, confidence interval=95%. f. XZ view (left) and XY cross-sections (right) of a 3D rendered En1^{Cre}; R26^{mtmg} adult fascia. g. XY view of the 3D rendered En1^{Cre}; R26^{mTmG} neonate back-skin. Imaging was made with the fascia (ventral) side up, to show the topological diversity across discrete anatomical positions. h. XYZ aerial view (top) and YZ cross-section (bottom) of an anterior location at the forelimb junction showing the presence of EPF traversing the skin muscle layer (arrow). i. XY view at a muscle breach in the mid-thoracic-cage level showing EPFs positioned in both locations. j. 3D rendered XY view image of an En1^{Cre}; R26^{VT2/Gk3} neonate back-skin at a muscle opening where nerves pass through. EPFs maintain their polyclonal state through all skin layers. Brocken lines delimitate the PC muscle layer. k. XY view (top) and XZ (below) cross-section of a 3D rendered En1^{Cre}; R26^{mTmG} adult superficial wound (3 dpw). Imaging was made with the epidermal (dorsal) side up, to show the presence of fascial EPFs arising from below the PC muscle. Dotted lines delimitate the epidermis. Scale bars=1500 microns (g), 100 microns (f, i-j), and 500 microns (k). PC=Panniculus carnosus, v=vessels, nb=nerve bundles.

FIG. 9: Fascial and dermal EPFs maintain their positions in steady conditions and fascial EPF in the wound get cleared at long term. a. Schematic description of dermal versus fascial EPFs chimeras in uninjured conditions. b. Histological images of fascial- (left) or dermal-(right) EPFs-traced chimeras. c. Scars from fascial-EPFs-traced (green) chimeras immunolabeled for CD26 (red) co-stained with DAPI (blue) in response to deep injuries at 70 dpw. d. Wounds from fascial- (left) or dermal- (right) EPFs-traced (green) chimeras in response to superficial (bottom) or deep injuries (top) at 14 dpw. Sections were co-stained with DAPI (blue) and immunostained (red) for caspase 3. e. Quantification of the fascia- or dermis-derived EPFs (GFP+) fraction positive for Cas3. Values are represented as percentages from the total labeled cells (GFP+) in the wound bed, or dermis or fascia control regions. N=images analyzed from 5 biological replicates. One-way ANOVA, multiple comparison Tukey test, confidence interval=95%. Lines delimitate the border between fascia and dermis. Dotted lines delimitate the wound bed or scar. Scale bars=200 microns.

FIG. 10: Fascial EPFs express and downregulate major wound fibroblast markers upon injury. a. Schematic description of dermal- versus fascial-EPFs-traced chimeras with two injury conditions. b-f. Immunolabeling against the fibroblast markers DPP4 (CD26, b), DLK1 (c), CD24 (d), CD34 (e), and LY6A (SCA1, f). g. Areas analyzed (top) for marker-positive EPFs quantification (bottom). The fraction of marker-positive cells was higher in fascial EPFs in the fascia, but decreased in fascial EPFs in the wound, indicating that the expression decreased after migration into the wound. N=5 images analyzed from 4 biological replicates. One-way ANOVA, multiple comparison Tukey test, confidence interval=95%. Dotted lines delimitate the wound bed. Scale bars=200 microns.

FIG. 11: Flow cytometric analysis of fibroblastic markers on fascial and dermal fibroblasts. a. gating strategy for fibroblast (Lin⁻, see “Methods”) analysis. b. Histo-plots of fibroblasts markers expression in fascia- or dermis-derived fibroblasts. c. Percentages of marker-positive cells from total fibroblast population. Two-tailed T-test, confidence interval=95%. d. Top: Gating strategy for fibroblast (Lin⁻, see “Methods”) and representative scatter plots of fascial-ENF and -EPF. Sorted GFP+EPFs were sorted for subsequent antibody labeling. Below: Representative scatter plots showing the expression of Sca1 and PDGFR1, and CD26 and CD29 in fascial EPFs.

FIG. 12: Fascia but not dermis matrix steers into wounds. a. Schematic description of fascial matrix labeling in chimeric skin grafts. Fascia biopsies of R26^{VT2/GK3} back-skin samples were separated as before and incubated with Alexa Fluor 647 NHS ester to fluorescently-label the matrix. Matrix-labeled fascia was combined with back-skin fragments of R26^{mtmg} mice and superficial injuries were performed as before. b. Left: histology showing fascial cells (green), fascial matrix (magenta), and skin cells (red). Right: Cyan channel showing the combined immunolabeling against COLLAGEN I, III, and VI to depict the total collagen content in the wound 7 dpw. c. Alexa Fluor 647-signaling coverage quantification of the total COLLAGEN I+III+VI signal in the wound at the defined time points. N=4 and 9 sections analyzed from 3 biological replicates. Unpaired two-tailed T-test, confidence interval=95%. d. Wounds 14 dpw showing fascial cells (green), fascial matrix (magenta), skin cells (red), and the combined immunolabeling against COLLAGEN I, Ill, and VI (cyan). e-f. High magnification images of inserts in “d”, showing the matrix label diminishing in the wound bed (e) but not in the deeper areas of the fascia (f). g. Schematic description of double matrix labeling in deep-injured dermal-EPFs-traced chimeras. h. histology of 7 dpw wounds showing dermal-EPFs (green), fascial matrix (magenta), dermal matrix (cyan), and TdTomato (red). Fascia matrix translocated and plugs the open wound allowing migration of dermal cells into the wound. i. Schematic description of double matrix labeling in deep-injured fascial-EPFs-traced chimeras. j. histology of 14 dpw wounds showing fascial-EPFs (green), fascial matrix (magenta), dermal matrix (cyan), and TdTomato (red). Dermal matrix remains unaltered while fascia matrix gets remodeled. k. Schematic description of double matrix labeling in superficial-injured dermal-EPFs-traced chimeras. l. histology of 14 dpw wounds showing dermal-EPFs (green), fascial matrix (magenta), dermal matrix (cyan), TdTomato (red), and COLLAGENI+III+VI (white). Superficial injuries heal by de novo deposition and not translocation of dermal matrix. Dotted lines delimit the wound bed. Arrowheads mark the original injury site. Continuous lines delimitate the epidermis-dermis margin. Scale bars=500 microns (b), 100 microns (d-f), and 200 microns (h, j, and l).

FIG. 13: Coagulation cascade within fascia matrix creates the eschar. a-b. Average fractal dimension (a) and lacunarity (b) plots from the subsampled maps showing the fascia matrix changes towards a mature scar matrix. N=5, 5, 8, and 3 images analyzed from three biological replicates. One-way ANOVA, Tukey test. Confidence interval=95%. c. Left: histological sections showing fascia matrix (FITC, green), SELP (red), and DAPI (blue) at the defined time points after wounding. Right: SELP (white) signal at the defined time points. Platelets infiltrate and get activated within the fascia matrix. Coagulated platelet clusters at the surface formed the eschar together with the fascia matrix. Arrowheads indicate the original injury site. Lines delimitate fascia compartment. Scale bars=200 microns.

FIG. 14: ePTFE membrane implants do not produce a chronic inflammatory reaction. a. Immunostaining for CD45 (green) and counterstained with DAPI (magenta) in 7 dpw sham and ePTFE-implanted wounds. Showing a higher infiltrate of immune cells at earlier time points with the ePTFE membrane. b. Fraction of CD45-positive cells from the total cells in the section. N=3 and 3 sections analyzed from 3 biological replicates. Two-tailed Student T-test, confidence interval=95%. c. Immunostaining for MOMA-2 (red), TNFα (green), and counterstained with DAPI (blue) in 7 and 63 dpw epTFE-implanted wounds. Showing a similar amount of monocytes/macrophages and TNFα expression in the presence of the ePTFE membrane. d-e. Fraction of MOMA-2-positive monocytes/macrophages from the total cells in the section (d) and mean gray value of TNFα signal (e). N=3, 3, 3 and 3 sections analyzed from 3 biological replicates. One-way ANOVA, Tukey test, confidence interval=95%. f. Immunostaining for SELP (green) and counterstained with DAPI (magenta) in 7 dpw sham and ePTFE-implanted wounds. Showing coagulation occurring even in the presence of the ePTFE membrane. g. mean gray value of SELP signal. N=3 and 3 sections analyzed from 3 biological replicates. Two-tailed Student T-test, confidence interval=95%. Brocken lines delimitates the ePTFE membranes. Scale bars=200 microns and 100 microns (inserts).

FIG. 15: EPF ablation but not proliferation inhibition in fascia inhibits matrix steering in vitro. a. Immunolabeling for COLLAGEN I, Ill, and VI (green) co-stained with DAPI (magenta) of En1^{Cre}; R26^{iDTR} biopsies at day 0 and 6 after acute treatment with DT or vehicle control. b. Collagens density quantification defined as the collagens area from the total section area. N=3 images analyzed from 3 biological replicates. Two-way ANOVA, multiple comparison Tukey test, confidence interval=95%. c. Cell density quantification defined as the number of cells (DAPI) divided by the collagens area. N=images analyzed from 3 biological replicates. One-way ANOVA, multiple comparison Tukey test, confidence interval=95%. d. XY time-lapse images of a 3D-rendered En1^{Cre}; R26^{iDTR} neonate fascia biopsy in culture treated with DT for 1 h. SHG (Cyan) and autofluorescence (green) signals at time 0 (left), 15 (middle), and 25 hours (right) showing lack of ECM movement after EPF depletion. Lines show the distance between two tracked points in the SHG channel. e. Length versus time plot of tracked points from the SHG and auto-fluorescence channels from DT-treated or control samples. f. Immunostaining for Ki67 (green) and counterstained with DAPI (magenta) in fascia biopsies treated with Etoposide at 2 days after culture. g. Fraction of Ki67-positive cells from the total cells in the section. N=3, 3, 3, and 2 sections analyzed from 2-3 biological replicates. One-way ANOVA, Tukey multiple comparisons, confidence interval=95%. h. XY time-lapse images of the 3D rendered C57BL6/J neonate fascia biopsy treated with 100 μM Etoposide in culture. SHG (Cyan) and autofluorescence (green) signals at time 0 (left), and 35 hours (right) depicting the fascial matrix movements. Lines show the length reduction in time. i. Length versus time plot of tracked points from the SHG channel of control and treated samples showing a clear contraction of the fascial matrix in culture in absence of cell proliferation. j-k. Total contraction after 25 h (e) and mean matrix velocity during the first 25 h of imaging. Two-tailed Student T-test. Confident interval=95%. Scale bars=50 microns (f), 200 microns (a and h), and 500 microns (d).

FIG. 16: Cell proliferation proceeds wound clogging by fascia matrix steering. a. Schematic description of in situ fascial matrix labeling and EdU pulses. b. Left: histological sections showing fascia matrix (FITC, green), EdU (red), and DAPI (blue) at the defined time points after wounding. Right: EdU (white) signal at the defined time points. c. Fraction of EdU-positive cells in the wound of the total EdU-positive cells. N=3, 4, 6, and 4 sections analyzed from 3 biological replicates. One-way ANOVA, Tukey multiple comparisons. Arrows indicate EdU-positive nuclei. Arrowheads indicate the original injury site. Lines delimitate fascia compartment. Scale bars=200 microns.

FIG. 17: In vivo movement of ECM after triggering an injury. Brushinng was made to mimic an injury on the surface of peritoneum. ECM was labelled in accordance with the present invention. Movement of ECM was monitored after 9 hours and 72 hours after brushing. ECM moves towards the site of brushing which mimics an injury.

FIG. 18: Schematic overview of an exemplary embodiment of the methods of the present invention for identifying modulators of extracellular matrix (ECM) movement.

FIG. 19: In vivo movement of ECM after triggering an injury. ECM was labelled and monitored for its movement into the direction of an injury. Liver, lung, kidney, heart and peritoneum is shown.

FIG. 20: In vivo effects on ECM movements in the presence of modulators of ECM movements. An injury within liver tissue was generated, a potential modulator was administered and ECM movement was monitored. GM6001 (MMP inhibitor), 1400W (iNOS inhibitor), LY255283 (leukotriene B4 receptor antagonist), and Cath-G inhibitor (Cathepsin-G inhibitor) were used as modulator having anti-fibrotic phenotypes. Additionally, Elastial (Elastase inhibitor) was used as modulator having a pro-fibrotic phenotype.

FIG. 21: Screening of 1280 chemicals via SCAD assay. (a) Scheme of SCAD assay and histologic image of a SCAD tissue on Day 5; (b) Histologic images of 26 chemicals that showed aberrant scarring effect (anti-scarring or pro-scarring) with a clear trend on scarring severity, scale bar 50 μm; (c) Fractal dimension and lacunarity analysis of the 26 chemicals revealed a clear trend of high porosity and low complexity from anti-scarring chemicals to pro-scarring ones; (d) Summary of the 26 chemical hits.

FIG. 22: Characterization of fascia sprouting and webbing. (a) Dynamic changes of fascia fibroblast migration from Day 0 to Day 4, scale bar 100 μm; (b) Ki67 staining of En1^{Cre}; R26^{mTmG} fascia tissue from Day 0 to Day 4, scale bar 50 μm; (c) and (d) Cartoon illustration of sprouting and webbing properties of fascia fibroblast migration; (e) Screenshots and tracking trajectories of En1^{Cre}; R26^{mCherry} Day 2 to Day 5 fascia live imaging, scale bar 100 μm; (f) Screenshots and tracking trajectories of En1^{Cre}; R26^{mCherry} Day 7 to Day 9 fascia live imaging, scale bar 100 μm.

FIG. 23: Re-test 26 chemicals in fascia invasion assay. (a) Invasion index of 26 chemicals; (b) Single cell images of 26 chemicals in fascia invasion assay, scar bar 10 μm.

FIG. 24: Representative whole mount tissue images of the top three anti-scarring chemicals. They showed lower invasion index with reduced cell-cell connection and aberrant cell morphology, scale bar 100 μm, 20 μm.

FIG. 25: Anti-scarring effects via hedgehog pathway. (a) Decreased expression of Gli1 protein in fascia tissue with anti-scarring chemical treatments; (b) Weakened expression of αSMA in fascia tissue with anti-scarring chemical treatments; (c) and (d) Lower expression of ki67 and higher expression of caspase 3 with anti-scarring chemical treatments; scale bar 50 μm.

FIG. 26: Anti-scarring chemicals inhibited fascia mobility in vivo.

FIG. 27: Anti-scarring chemicals inhibited scar formation in vivo.

FIG. 28: Characterization of fascia invasion assay. (a) Brightfield images of fascia growth from Day 0 to Day4. Arrows indicate the distance of cell invasion; (b) Screenshots and tracking trajectories of En1^{Cre}; R26^{mCherry} Day 1 to Day 2 fascia live imaging, scale bar 100 μm; (c) and (d) Dynamic changes of invasion index and contraction index of fascia ex vivo culture from Day 0 to Day4; (e) and (f) Sprouting and webbing behavior of migrated fascia fibroblasts.

FIG. 29: Characterization of fascia invasion assay. (a) Screenshots of En1^{Cre}; R26^{mCherry} Day 2 to Day 5 fascia live imaging showed cell proliferation during migration; (b) Screenshots of En1^{Cre}; R26^{mTmG} Day 2 to Day 5 fascia live imaging revealed how two cells distant from each other connected, scale bar 50 μm.

FIG. 30: Superficial injury induces organ wide matrix mobilization.

a) NHS-FITC labelling reveals surface ECM structures of liver, peritoneum and cecum. Representative images of three biological replicates SHG: second harmonic generation. Scale bars: 15 μM. Representative immunofluorescence image of a histological section showing NHS-FITC penetration depth. Scale bars: 10 μM. b) Patch mediated fate tracing of liver surface ECM in local electroporation injury model. c) Liver surface matrix flows upon injury response. Stereomicroscopic images of mouse livers 24 hours after electroporation against undamaged control. Representative images of three biological replicates. Scale bar overview: 2000 μM; High magnification: 100 μM. d) Fluid matrix is restructured during wound healing on liver surfaces. Representative H&E histology and multiphoton images of livers 24 hours and 14 days after electroporation. Scale bar overview: 50 μM; High magnification: 15 μM. e) Patch mediated fate tracing of peritoneal surface and local injury by brushing reveals motion of surrounding fibrous elements (NHS-FITC+) into wound areas (NHS-AF568+) after 30 minutes. Representative stereomicroscope images of three biological replicates. Scale bar overview: 1000 μM; High magnification: 100 μM. f) Peritoneal surface matrix flows laparotomy closure response. Stereomicroscopic images of mouse peritoneas 1 minute and 24 hours after laparotomy closure. Representative images of three biological replicates. Scale bar overview: 2000 μM. g) Fluid matrix currents flow into wounds for three days. FITC intensity of liver and peritoneal wound lysates after the indicated time points, n=three biological replicates. One-way ANOVA, multiple comparison Tukey's test, 95% Cl. h) Fluid matrix closes peritoneal laparotomy closures with net like structures. Representative H&E histology and multiphoton images of three biological replicates. Scale bar overview: 50 μM; High magnification: 15 μM.

FIG. 31: Fluid matrix is transformed into rigid frames in wounds.

a) Overview of patch mediated in vivo crosslink assay (see methods). b) Increasing FITC intensity of Streptavidin Pulldown samples reveals growing crosslinking over time in liver wounds. n=three biological replicates. One-way ANOVA, multiple comparison Tukey's test, 95% Cl. c) Four-week old organ fusions between Peritoneum (AF568+) and Cecum (FITC+). Representative images of n>three biological replicates. d) Matrix fusions between peritoneum and cecum in four-week old adhesions. Immunolabeling shows contribution of peritoneal Collagen 1 in cecum repair. Representative images of n>three biological replicates. e) Peritoneal matrix fuses livers and flows onto surfaces. Representative images of n>three biological replicates. Scale bar: 50 μM. f) Crosslinking between organ matrix fractions starts after two weeks. Cecum: NHS-FITC. Peritoneum: NHS-EZ-LINK-Biotin. n=three biological replicates.

FIG. 32: Fluid matrix provides many raw components for tissue repair.

a) Workflow of proteomic based identification of fluid matrix systems. b) Fluid matrix originates from multiple organ depths and layers. c) Fluid matrix fractions consist mostly of Collagens and ECM glycoproteins. d) Abundance of single proteins of fluid matrix vary between organs. e) Fluid matrix of the liver inherits pro regenerative, peritoneal fluid more pro fibrotic elements. Classification was based on uniport entries. f) Liver fluid matrix proteins are linked to metabolic regulation whereas peritoneal and cercal fluid elements are linked to fibrotic reactions.

FIG. 33: Neutrophils direct matrix flows

a) Visualization of cell populations upon liver electroporation. b) Liver cell populations show distinct ECM surface receptor expression patterns upon liver electroporation. c) Fast migrating cell populations upregulate a limited number of surface receptor genes. d) Crossing scheme of Lyz2Cre; Ai14 transgenic mouse line, Lyz2+ cells express dTomato. e) Snapshots of extended video 7 showing Lyz2+ cells transport matrix elements across liver surfaces. Arrows highlight single cells. Representative image of three biological replicates. Scale bar: 50 μM. f) Swarms of Lyz2+ cells accumulate FITC+fluid matrix. Representative image of three biological replicates. Scale bar: 50 μM. g) Lyz2+ cells transport FITC+fluid elements in a no phagocytic form. Representative image of three biological replicates. Scale bar: 5 μM. h) Accumulations of FITC+fluid matrix elements are rich with Ly6g+ positive cells. Representative immunolabeling image of three biological replicates. Scale bar: 5 μM. i) Fluid matrix flows are mediated Ly6g+ positive cells and can be directed with local application of Lipoxin. Representative stereomicroscope of three biological replicates. Scale bar: 500 μM. j) Neutrophils upregulate CD11b, CD18 and NOS reactome upon injury. k) Targeted inhibition of neutrophil ECM receptors, swarming mediators and NOS stress enzymes blocks fluid matrix flows after liver electroporation. n=4 One-way ANOVA, Dunnett's multiple comparisons, 95% Cl. ***, p=0.0001.

FIG. 34: Fibrous postsurgical adhesions are derived from fluid matrix elements

a) Representative immunofluorescence images of histological sections of murine and human abdominal postsurgical adhesions. Murine peritonea were labeled with NHS-AF568 cecums with NHS-FITC, mice were sacrificed 4 weeks after surgery. Scale bar: 20 μM.

FIG. 35: Organ injury regulates CD11b and CD18 on neutrophils

a) Percentage of individual cell population compared to the total number during liver injury. b) Time dependent abundance of cell populations during liver injury. c) Visualization of cellular abundances post liver electroporation. d) Sub clustering of activated neutrophil populations. e) Activated neutrophils show a consistent higher expression of CD11b and CD18 post injury.

FIG. 36: Neutrophils orchestrate peritoneal matrix movements

a) Crossing scheme of a transgenic mouse line; Lyz+ cells express dTomato. b) Snapshots of extended video 7 showing Lyz2+ cells transport matrix elements across peritoneal surfaces. Arrows highlight single cells. Representative image of three biological replicates. Scale bar: 50 μM. c) Swarms of Lyz2+ cells accumulate FITC+fluid matrix on peritoneal surfaces. Representative image of three biological replicates. Scale bars: Overview: 500 μM; High magnifications: 50 μM. d) Majority of Lz2 positive cells carry FITC+Elements 24 hours after organ injury. One-way ANOVA, Dunnett's multiple comparisons, 95% Cl. ***, p=0.0001. e) Accumulations of FITC+fluid matrix elements on peritoneums are rich with Ly6g+positive cells. Representative immunolabeling image of three biological replicates. Scale bar: 5 μM. f) Targeted inhibition of neutrophil ECM receptors, swarming mediators and NOS stress enzymes blocks fluid matrix flows after peritoneal injury. n=4 One-way ANOVA, Dunnett's multiple comparisons, 95% Cl. ***, p=0.0001.

FIG. 37: Matrix flows underpin regeneration and scarring

a) Overview of treatment regime in the liver electroporation setup (see methods). b) Inhibition of fluid matrix influx leads to impaired wound healing in liver electroporation sites. Representative immunofluorescence images of three >=biological replicates. Scale bar: 50 μM. One-way ANOVA, Dunnett's multiple comparisons, 95% Cl. ***, p=0.0001. c) Overview of treatment regime in the peritoneal-cercal setup (see methods). d) Treatment regime inhibits matrix flows in peritoneal and cercal injury sites, n>=4 biological replicates. Representative immunofluorescence images of histological sections FITC-NHS marked livers seven days after electroporation. Scale bar: 50 μM. One-way ANOVA, Dunnett's multiple comparisons, 95% Cl. e) Inhibition of matrix flows blocks adhesion formation in vivo, n>=3 biological replicates. Representative immunofluorescence images of histological sections FITC-NHS marked peritoneas five days post injury. Scale bar: 100 μM. One-way ANOVA, Dunnett's multiple comparisons, 95% Cl. ***, p=0.0001.

FIG. 38: NHS directed labeling of wound areas

a) Scheme of experimental Setup. Livers of mice were electroporated. On day 2 an intra peritoneal injection of NHS-Rhodamine or control PBS injection was performed. 30 minutes later the organs were harvested. b) Representative stereomicroscopic images of livers.

FIG. 39: Workflow of biomarker discovery.

a) After intra pleural injection of NHS esters, bleomycin is installed. Organs and blood are taken 14 days later. b) Histological sections of NHS-FITC labelled mouse lungs 14 days after bleomycin installation. c) Extract of proteins identified in mouse lungs treated with bleomycin after 14 days. d) Extract of protein found in the blood of mice 14 days after bleomycin.

FIG. 40: Matrix motions inhibitor screening

a) Pictures of the setup. Liver and peritoneal tissues were locally labelled after injury. Organs were harvested after 24 h, wound sites lysed and FITC amounts measured. b) Quantifications of inhibitor experiments. n=4.

FIG. 41: Extracellular matrix-fate tracing reveals interstitial matrix invasion during lung injury

(A) Workflow of pleural matrix fate tracing setup. Mice were intra-pleurally injected with N-Hydroxysuccinimide-fluorescein isothiocyanate (NHS-FITC) labelling mix and two weeks later lungs were harvested. (B) Surface matrix stays stable over 2 weeks. Light sheet images of murine lungs (n=6). Scale bars: 500 μM. (C) Pleural matrix fate tracing reveals pools of extra cellular matrix. Multiphoton images of murine lung surfaces (n=6). Scale bars: 100 μM (overview) and 15 μM (high magnification). (D) Workflow of the bleomycin induced injury model. Mice were intra-pleurally injected with NHS-FITC labelling mix. The next day bleomycin was instilled and two weeks later lungs were harvested. (E) and (F) Pleural surface matrix invades deep into the interstitium upon bleomycin injury. Light sheet microscopy and histology images of murine lungs two-week post-bleomycin injury (n=6). Statistical comparison was by unpaired t-test. Scale bars: whole organ 500 μM, histology 500 μM (overview) and 15 μM (high magnification). (G) Interstitial fibrotic plaques are filled with invaded matrix. H&E and fluorescence images of murine lungs two-weeks post-bleomycin injury (n=6). Statistical comparison was by unpaired t-test. Scale bars: 100 μM. (H) Pleural matrix pools are depleted bleomycin induced injury. Multiphoton images of murine lung surfaces two-weeks post-bleomycin injury (n=3). Scale bars: 100 μM (overview) and 15 μM (high magnification).

FIG. 42: Immune cells orchestrate fluid scar motions

(A) Workflow of murine ex vivo fluid scar tracing assay. (B) Immune cells enhance loss of protein from pleural surfaces 48 hours after incubation with immune cells (n=3). Control=mouse lungs without immune cells; healthy=mouse lungs supplemented with immune cells from healthy human volunteers; IPF=mouse lungs supplemented with immune cells from humans with lung disease. Scale bars: 100 μM. Data represented are mean±SD. One-way ANOVA was used for the multiple comparison (control 48 h vs. healthy and IPF immune cells, *P<0.05; *Healthy vs. IPF, *P<0.05, **P<0.01, ***P<0.001). (C) Immune cells accelerate interstitial fluid scar invasion of murine lung biopsies 48 hours after incubation. Plots for the NHS ester labelled ECM movement in the mouse lung biopsies. Data represented are mean±SD. One-way ANOVA was used for the multiple comparison (control 48 h vs. healthy and IPF immune cells, *P<0.05; *Healthy vs. IPF, *P<0.05, **P<0.01, ***P<0.001).

FIG. 43: Human invading matrix resembles fluid scar tissue

(A) Fluid matrix invasion in human lung tissues (n=2). Scale bars: 200 μM and 100 μM (i). (B) Fluid matrix accumulates in interstitial structures (n=2). Scale bars: 100 μM and 20 μM (i). (C) Workflow of proteomic identification of human fluid matrix systems. (D) Human pleural fluid matrix fractions have abundant fibrous elements (n=5). (E) GO enrichment reveals invading matrix resembles atrophic scar tissue with abnormal elastic tissue morphology. (F) Invading matrix harbors fibrous building blocks and crosslinking enzymes.

FIG. 44: Ablation of fluid matrix streams prevents pulmonary fibrosis

(A) Fluid scar tissue inherits SRC dependent tyrosine kinase signaling. GO enrichment of human proteomic signaling. (B) Workflow of Nintedanib treatment regime in the bleomycin-induced lung fibrosis model. (C) Nintedanib rescues pleural matrix pools after bleomycin-induced injury (n=5). (D) Immunofluorescence and H&E images of murine lungs two weeks after bleomycin injury (n=5). Statistical comparison was performed by unpaired t-test. Scale bars: 500 μM and 100 μM (Immunostainings).

FIG. 45: Bleomycin induces structural changes and protein loss from pleural surfaces.

(A) Bleomycin-induced pneumonia increased local thickness in murine lung surfaces after 14 days (n=3). (B) Fibrotic lungs have more complex surface structures (n=3).

FIG. 46: Murine pleural fluid matrix pools resemble fluid scar tissue

(A) Schema of the mass spectrometry experiment. (B) Fluid matrix consists mostly of collagenous elements (n=5). (C) Pleural fluid matrix fractions contain fibrillar and basement elements (n=5). (D) Fluid matrix composition resembles atrophic scar tissue. (n=5)

FIG. 47: Elements of fluid scars show distinct fluidity profiles

A) Calculation of fluidity factor. B) Fluid scar elements show distinct fluidity profiles (n=5).

FIG. 48: Part B of table 1 shows H and H stainings of the tissue patches treated with the respective compounds

### DETAILED DESCRIPTION OF THE INVENTION

In order to overcome some of the shortcomings of the means described so far in the prior art that there is still a need to provide further options in the treatment of excessive or insufficient scar formation, the inventors of the present invention surprisingly discovered that chronic and excessive skin wounds may be attributed to the mobility of the fascia matrix. Thus, the inventors provide herein promising new methods for identifying modulators of extracellular matrix (ECM) movement towards a site requiring deposition of ECM.

The present inventors have now surprisingly found that scars originate from prefabricated matrix in the fascia, e.g. subcutaneous fascia that home into sites requiring deposition of extracellular matrix (ECM), such as wounds. The identification of fascia as the source for, e.g. dermal scars allowed the present inventors to identify the mechanism of scar formation, by using matrix-tracing techniques, live-imaging, genetic lineage-tracing and anatomic fate-mapping models. Strikingly, the present inventors found that scars originate from, inter alia, fascia fibroblasts bundled with its prefabricated matrix. Upon injury, this assembly homes into open wounds as a movable sealant that not only drags plugs of matrix, but also vasculature, immune cells and nerves, upwards into the outer skin. Accordingly, the present inventors observed that fascia fibroblasts rise to the site requiring patching after wounding, thereby dragging their surrounding extracellular jelly-like matrix, including embedded blood vessels, macrophages, and peripheral nerves, to form a scar. Genetic ablation of fascia fibroblasts prevented matrix from homing into wounds and resulted in poor scars, whereas placing an impermeable film beneath the skin, to prevent fascia fibroblasts migrating upwards, led to chronic open wounds. Thus, fascia contains a specialised prefabricated kit of, inter alia, sentry fibroblasts, embedded within a movable sealant, that preassemble together all the cell types and matrix components needed to heal wounds. The findings of the present inventors suggest that chronic and excessive skin wounds may be attributed to the mobility of the fascia matrix.

While prior art focuses on end-point phenotypes regarding fibrosis or keloid, the present invention allows focusing on the starting point. Indeed, the present inventors succeeded for the first time in in vivo labelling of ECM and could thus observe in real-time its movement towards a site of injury. This allows interfering with ECM deposition at a much earlier point in time than known before and, thus, opens new avenues for treatment options as described herein. Accordingly, the present invention relates to a method for identifying modulators of extracellular matrix (ECM) movement towards a site requiring deposition of ECM, comprising (a) contacting extracellular matrix of organ tissue obtainable by biopsy from a mammalian subject with a label; (b) contacting said labelled extracellular matrix of organ tissue with a compound of interest; (c) determining whether said compound of interest modulates ECM movement towards said site requiring deposition of ECM in comparison to labelled extracellular matrix of organ tissue obtainable by biopsy from a mammalian subject which is not contacted with said compound of interest, wherein modulation of ECM movement towards said site requiring deposition of ECM is indicative for said compound of interest to be a modulator of said ECM movement.

The method for identifying modulators of ECM movement towards a site requiring deposition of ECM includes labelling of the ECM. Hence, by labelling ECM, the ECM is visualized for being observed. Observation of ECM movement allows the identification of modulators of ECM movement, since a modulator may either decrease or accelerate ECM movement. As explained, visualization of the movement of labelled ECM allows the identification of a modulator being an inhibitor of ECM movement on the basis of decreasing ECM movement, while a modulator being a promoter of ECM movement can be identified on the basis of accelerating ECM movement. Without being bound by theory, it is assumed that decreasing ECM movement will result in a decreased deposition of ECM at a site requiring ECM deposition, such as a wound, while accelerating ECM movement will result in an accelerated deposition of ECM at a site requiring ECM deposition, such as a wound.

Decreasing ECM movement when used herein is equivalent to inhibition of ECM movement. Inhibition of ECM movement towards a site requiring ECM deposition preferably prevents excessive deposition of ECM at said site.

Accelerating ECM movement when used herein is equivalent to promotion of ECM movement. Promotion of ECM movement towards a site requiring ECM deposition preferably prevents insufficient deposition of ECM at said site.

“Identifying modulators of ECM movement” or “identification of modulators of ECM movement” includes screening such modulators and, once identified or screened, isolating, i.e. providing such modulators.

Step (a)

An “extracellular matrix (ECM)” according to the present invention refers to a collection of extracellular molecules secreted by cells. The ECM of the organ tissue of the present invention may be composed of collagen fibrils, microfibrils, and elastic fibers, embedded in hyaluronan and proteoglycans. Preferably, said ECM comprises proteins, polysaccharides and/or proteoglycans. Those components may refer to ECM components according to the present invention. Such ECM components may be covalently coupled to said label which is used to contact the ECM, in particular the ECM components of organ tissue are obtainable by biopsy from a mammalian subject. Preferably, ECM may also comprise cells of fascia matrix, serosa and/or adventitia as described herein, such as macrophages, neutrophils, mesothelial cells and/or fibroblasts, with fibroblasts being preferred. ECM proteins, such as labelled ECM proteins described herein, are used herein preferably as surrogate marker for ECM movement.

The organ tissue which is used for contacting the ECM of said tissue with a label may refer to a tissue sample/piece comprising cells from an organ as defined elsewhere herein. The organ tissue may also refer to a biopsy punch, which is created with a biopsy puncher from said tissue sample/piece. In this context, a “biopsy punch” refers to a small, roundish organ tissue sample created with a tool important in medical diagnostics—also called biopsy puncher—which is able to punch out/stamp out small pieces of said organ tissue with cleanly defined diameter. Preferably, a disposable, round biopsy puncher with 2 mm in diameter may be used. It generates uniform round shape biopsies (punch biopsies) that reduce variability. However, the organ tissue which is used for contacting the ECM of said tissue with a label can also be a whole organ as defined elsewhere herein such as an organ withdrawal. Said organ tissue, when it refers to a tissue sample/piece as defined above may be obtainable/obtained by biopsy from a mammalian subject. A biopsy according to the present invention is a medical test involving extraction of organ tissue(s) from a mammalian subject for examination to identify modulators of said ECM movement according to the method of the present invention. The technique being applied when the organ tissue may be obtainable by biopsy is known to a person skilled in the art. According to the method of the present invention, said organ tissue obtainable by biopsy from a mammalian subject may be a healthy or a diseased organ tissue.

Preferably, said organ tissue obtainable/obtained by biopsy from said mammalian subject according to the method of the present invention is from skin, kidney, lung, heart, liver, bone, peritoneum, intestine, diaphragm or pleura. More preferably, said organ tissue obtainable/obtained by biopsy from said mammalian subject according to the method of the present invention is skin. Said mammalian subject may be any mammal known to a person skilled in the art. Preferably, said mammalian subject is a human. Thus, in a preferred embodiment of the present invention an organ tissue may be obtainable by biopsy from a human, preferably an adult.

“Obtainable by biopsy” or “obtained by biopsy” is not limited to classical biopsy. It may even be a whole organ withdrawal. However, classical biopsy and biopsy as described herein is encompassed. When referring to “biopsy” in the context of the present invention, it is meant that during or at biopsy or after biopsy an organ tissue is injured, e.g., due to brushing or any other stimulus such that a site requiring ECM deposition is generated, unless the organ tissue may already have one or more of such sites requiring ECM deposition. The latter may be fulfilled in case of a diseased organ tissue, e.g. where ECM deposition may be excessive or insufficient.

Preferably, said organ tissue according to the method of the present invention comprises fascia matrix, serosa and/or adventitia. Even more preferably, said organ tissue according to the method of the present invention comprises fascia matrix. Fascia matrix, serosa and/or adventitia being used in the method of the present invention preferably comprise macrophages, neutrophils, mesothelial cells and/or fibroblasts.

Fascia matrix may be characterized by containing cells expressing α-SMA, CD90, ER-TR7, PDGFRα, Sca1, βIIITubulin, CD31, MOMA-2, F4/80, CD24, CD34, CD26, Dlk1, Fn1, Col14a1, Emilin2, Gsn and/or Nov. In this context, the term “expressing” refers to cells “expressing” a surface or cytoplasmic marker such as α-SMA, CD90, ER-TR7, PDGFRα, Sca1, βIIITubulin, CD31, MOMA-2, F4/80, CD24, CD34, CD26, Dlk1, Fn1, Col14a1, Emilin2, Gsn and/or Nov or said term refers to cells “having expressed” when referring to a lineage marker such as En1.

The Engrailed-1-lineage-positive fibroblasts or Engailed1-history-past fibroblasts (EPFs) are the main contributor of scar tissue development in murine back skin and cranial dermis, whereas Wnt1 lineage positive fibroblasts are the main contributor of scar tissue development in murine oral cavity. This embryonic lineage within the dorsal dermis possesses many of the functional attributes and characteristics such as the similar spindle-shaped morphology commonly associated with the term “fibroblast”. However, this lineage is not only present in the skin but also in the underlying superficial fascia. These fibroblast lineages (e.g., EPFs) responsible for scar deposition are derived from circulating fibroblast-like cells. EPFs may refer to En1-lineage-positive fibroblasts, meaning the ancestor/progenitors expressed En1 in the history during embryogenesis, but EPFs most likely do not express Engrailed-1 (En1) at stage of E18.5-P10, the developmental stages where the skin tissues may be collected from mice.

Engrailed-1 (and Wnt1) is expressed only transiently during embryonic development. En1 is a transcription factor, it turns on very early during development and regulates the expression of several downstream target genes. The En1 gene marks a lineage of cells. Once it is turned on, the cells and its progeny are EPFs, no matter whether En1 is expressed or not in the cells. Therefore, En1 is not a surface marker to mark the cells, but a lineage marker, thus defining an embryonic lineage.

In the wild type mouse system or in human, there is no direct way to mark EPFs. Therefore, surrogate markers such as CD26 or other fibroblast markers as mentioned below may be used for marking EPFs. CD26 labels a large percentage of EPFs (94%) and offers the highest-fold enrichment of EPFs over ENFs that have never expressed Engrailed in the history. ENFs do not participate in scar tissue formation. By transplanting adult ENFs & EPFs, separately, in different anatomical locations, it has been determined that the difference in the capacity of EPFs & ENFs to form a scar is cell-intrinsic, and permanent, and that these are in vivo behaviours of two distinct fibroblastic cell types (Rinkevich et al., 2015, Science 348 (6232)).

For human samples, the bellow pan markers for fibroblasts may further be used, such as N-Cadherin, alpha-smooth muscle actin (α-SMA), fibroblast specific protein 1 (FSP1), and/or platelet derived growth factor receptors alpha (PDGFRα) and beta (PDGFRβ), all important indicators and markers of scar formation. The bellow pan markers for fibroblasts as mentioned above may also be used in mice as well.

When in step a) of the method of the present invention the term “contact” or “contacting” is used, it means that said ECM of organ tissue as defined elsewhere herein is brought into contact with said label, which covalently couples to said ECM components. In a preferred embodiment, the term “contact” or “contacting” refers to “selectively contact” or “contacting”. In this context, “selectively contacting” means that not the whole ECM of the organ tissue is contacted with said label as defined elsewhere herein, but one or more portion of said ECM of said organ tissue. In other words, when the term “selectively contacting” is used herein, a confined very specific spot of the ECM of said organ tissue is contacted with said label as defined elsewhere herein, thus performing a locally ECM labelling on the organ tissue of the present invention. Preferably, proteins comprised by ECM are labelled. However, it is also envisioned that other components of ECM may be labelled, such as carbohydrates.

A “label” is a molecule or material that can produce a detectable (such as visually, electronically or otherwise) signal that indicates the presence and/or concentration of the label in a sample from an organ tissue. Thereby, e.g., the presence, location and/or concentration of a labelled molecule in a sample can be detected by detecting the signal produced by the (detectable) label. A label can be detected directly or indirectly. It will be appreciated that the label may be attached to or incorporated into a molecule, for example, a protein, polypeptide, or other entity, at any position. It will be appreciated that, in certain embodiments, a label may react with a suitable substrate (e.g., a luciferin) to generate a detectable signal. In particular, the detectable label can be a fluorophore, an enzyme (peroxidase, luciferase), a radioisotope, a fluorescent protein. Other detectable labels include chemiluminescent labels, electrochemiluminescent labels, bioluminescent labels, polymers, polymer particles, metal particles, haptens, and dyes.

A “fluorophore” (or fluorochrome) is a fluorescent chemical compound that can re-emit light upon light excitation. Examples of fluorophores include 5-(and 6)-carboxyfluorescein, 5- or 6-carboxyfluorescein, 6-(fluorescein)-5-(and 6)-carboxamido hexanoic acid, fluorescein isothiocyanate, rhodamine, tetramethylrhodamine, and dyes such as Cy2, Cy3, and Cy5, optionally substituted coumarin including AMCA, PerCP, phycobiliproteins including R-phycoerythrin (RPE) and allophycoerythrin (APC), Texas Red, Princeton Red, inorganic fluorescent labels such as particles based on semiconductor material like coated CdSe nanocrystallites.

Examples for fluorescent proteins include Exemplary fluorescent proteins include, e.g., Sirius, Azurite, EBFP, EBFP2, TagBFP, mTurquoise, ECFP, Cerulean, CyPet, TagCFP, mTFPI, mUkGI, mAGI, AcGFPI, TagGFP2, EGFP, GFP, mWasabi, EmGFP, YFP, TagYPF, Ypet, EYFP, Topaz, SYFP2, Venus, Citrine, mKO, mK02, mOrange, mOrange2, TagRFP, TagRFP-T, mStrawberry, mRuby, mCherry, mRaspberry, mKate2, mPlum, mNeptune, mKalama2, T-Sapphire, mAmetrine, mKeima, UnaG, dsRed, eqFP611, Dronpa, KFP, EosFP, Dendra, and IrisFP.

Examples of enzymes used as enzymatic labels include horseradish peroxidase (HRP), alkaline phosphatase (ALP or AP), β-galactosidase (GAL), glucose-6-phosphate dehydrogenase, β-N-acetylglucosamimidase, β-glucuronidase, invertase, Xanthine Oxidase, firefly luciferase and glucose oxidase (GO).

Examples of radioactive labels include radioactive isotopes of hydrogen, iodide, cobalt, selenium, tritium, carbon, sulfur and phosphorous. ²H, ³H, ¹³C, ¹⁴C, ¹⁵N, ¹⁸F, ³¹P, ³²P, ³⁵S, ⁶⁷Ga, ⁷⁶Br, ^{99m}Tc (Tc-99m), ^{m}In, ¹²³I, ¹²⁵I, ¹³¹I, ¹⁵³Gd, ¹⁶⁹Yb, and ¹⁸⁶Re.

According to the present invention, said label is preferably a dye or a tag. When said label is a dye, a fluorescent dye is preferred. A fluorescent dye refers to a reagent coupled to a fluorophore. In particular, said reagent refers to N-Hydroxysuccinimide ester or Succinimidyl esters (NHS) or sulfodichlorophenol (SDP)-esters. When used in the present invention NHS ester means N-hydroxysuccinimide ester or Succinimidyl esters. NHS or SDP-esters react with extracellular amines, like N-termini of proteins and lysines labelling ECM-components. NHS/SDP esters conjugated with fluorophores such as Alexa 488, Alexa 568, Alexa 647, Fluorescein, Fluorescein isothiocyanate (FITC), Pacific Blue, are used to visualize ECM.

As apparent from the above a NHS ester is sufficient to label extracellular amines. An essential step in untangling the phenomenon of ECM movement is the possibility to crosslink of moved material in the wound areas. Primary amines of proteins and peptides of distinct protein classes are covalently linked. Since the NHS esters also mark primary amines, the inventors asked themselves whether the restructuring in wound areas has led to an increase in free amine groups and whether they can visualize these via intraperitoneal application of NSH-Esters which was the case. The discovery the inventors have made here has many potential implications. The data shows that there is an accumulation of primary amines in abdominal wound areas which can be labelled via NHS-linked reaction (FIG. 38). This would allow to be marked any abdominal wound by a simple intra peritoneal injection. By using an NHS ester coupled to deeper wavelength reporters which would open a new dimension of wound visualization in the clinics.

In one preferred embodiment the NHS ester of the present invention may be used to label primary amines. In another embodiment the NHS ester of the present invention may be used to label amines and primary amines in a wound as defined herein. The NHS ester labeling might be used in a diagnostic approach. A diagnostic approach might to monitoring wound healing or wound progression. In this scenario it might be advantageous to combine NHS ester with a further reporter molecule as described above. In one preferred embodiment the NHS ester stain might be combined with any kind of reporter or fluorescent dye.

Preferably, such fluorescent dye include, but is not limited to, Alexa Fluor 488 NHS-ester, NHS-Fluorescein (5/6-carboxyfluorescein succinimidyl ester), Alexa Fluor 568 NHS-ester, Pacific Blue Succinimidly Ester, Alexa Fluor 647 NHS-ester (N-hydroxysuccinimide ester or Succinimidly Ester), Alexa Fluor 488 5-SDP-ester or NHS-Rhodamine (5/6-carboxy-tetramethyl-rhodamine succinimidyl ester). Each of the abovemetioned fluorescent dyes are able to label the ECM components of the ECM matrix from each organ tissue described elsewhere herein.

A method for diagnosing the healing progress of wounds wherein the method comprises administering NHS ester systemically to a patient and thereby labeling amines in the wounds. The method for diagnosing the healing of wounds wherein the NHS ester is combined with a reporter molecule.

In addition to imaging wounds, effector molecules could also be coupled to NHS esters, and thereby targeting wound areas with a single global injection. Such effector molecules might be therapeutic compounds. In case NHS ester is coupled or linked to a compound, any kind of compound might be suitable. However, preferred are therapeutic compounds for the treatment of chronic wounds. The compound coupled to NHS ester might a modulator of the extracellular matrix (ECM) movement as described herein.

Said NHS ester might be administered systemically or locally as FIG. 38 shows. In yet another embodiment the NHS ester of the present invention is injected into the blood flow to label primary amines in wounds. In this scenario the NHS ester might be used to monitor the progress of wound healing. In another scenario the NHS ester might be coupled to a compound to target wounds systemically. In case NHS ester is coupled or linked to a compound, any kind of compound might be suitable. However, preferred are therapeutic compound for the treatment of chronic wounds. The compound coupled to NHS ester might be a modulator of the extracellular matrix (ECM) movement as described herein.

A therapeutic compound comprising NHS ester administered to a patient in the need thereof wherein the patient is suffering from chronic wounds. A therapeutic compound comprising NHS ester for use in treating chronic wounds wherein the compound is administered systemically meaning it is injected into the blood stream. A NHS ester for use in diagnosing wounds wherein the NHS ester is administered systemically and thereby labels wounds. The NHS ester for use in diagnosing wounds wherein the NHS ester is further combined with a reporter molecule. Due to the NHS ester being capable of labeling primary amines of a wound when injected into the blood stream, it can be used to monitor the extend or healing process of wounds.

The herein described NHS ester labeling of wounds which can be established by injecting the NHS ester stain into the blood flow and thereby marking primary extracellular amines, which might be used during or after surgery to monitor the extend or healing progression of a surgery wound (FIG. 38). Likewise it is encompassed that a NHS ester injection is applicable for marking chronic wounds. Chronic wounds may concern the epidermis, dermis and fascia and comprise wounds which show a poor healing process meaning they are not healing in the usual amount of time or in the usual expected way. A poor healing process might be caused by any kind of trauma, surgery, disease, infection, age, drugs, poor circulation or neuropathy. All of these causes might involve the fascia and fascia protein regulation. Thus, the healing of chronic wounds might be influenced by modulators of extracellular matrix (ECM) movement and thus the ECM movement.

A therapeutic compound comprising NHS ester and a modulator of extracellular matrix (ECM) movement administered to a patient in the need thereof wherein the patient is suffering from chronic wounds. A therapeutic compound comprising NHS ester and a modulator of extracellular matrix (ECM) movement for use in treating chronic wounds wherein the compound is administered systemically as injection into the blood stream.

Also comprised herein is that the label used in the method of the present invention is a tag. A “tag” can be an affinity tag (also called purification tag), such as a Biotin tag, histidine tag, Flag-tag, streptavidin tag, strep II tag, an intein, a maltose-binding protein, an IgA or IgG Fc portion, protein A or protein G. Preferably, said tag which is used in the method of the present invention and also conjugates with NHS/SDP esters is a Biotin tag. Such tags as defined elsewhere herein can thus also be used to analyze ECM components via protein biochemistry, like western blotting or mass spectrometry.

Depending on the ester, which may be used as a reagent of the fluorescent dye, specific reaction buffers may be used. When a NHS-ester is used as a reagent of the fluorescent dye 100 mM pH 9.0 BiCarbonate buffer is preferably used. It has been surprisingly shown that ester-reaction buffer mixtures can be applied on each organ as defined elsewhere herein without detectable toxic side effects.

When contacting ECM of organ tissue, which is obtainable by biopsy from said mammalian subject, with a label as defined elsewhere herein, a paper-like material comprising the label is used. Thus, the label, in particular the labelling solution which may be comprised by the label and the reaction buffer, can be applied locally (on one or more portions/spots of the ECM) onto the ECM of organ tissue as defined elsewhere herein preferably using such small paper pieces. Such paper-like material should be a non-reactive material meaning that said material itself does not interact/react with said ECM components of said organ tissue and/or said paper-like material should comprise an alcalic pH. Said paper-like material is thus able to soak up the label, in particular the labelling solution, leading to a local ECM labelling on the organ tissue. Examples of paper-like material include, but are not limited to, Whatman filter paper. In a preferred embodiment, 2 mm Whatman filter paper is used and an amount of 0.3 μl of the labelling solution is applied/added onto said filter paper before said paper is put onto the ECM of said organ tissue.

In particular, said label as defined elsewhere herein targets primary amine groups of ECM components as defined elsewhere herein. In other words, primary amine groups of ECM components are preferably labelled when applying the method of the present invention. Amines are compounds and functional groups that contain a basic nitrogen atom with an ion pair. They can be classified according to the nature and number of substituents on nitrogen. In nature there are primary, secondary and tertiary amines. Primary amines (also called primary amine groups) arise when one of three hydrogen atoms in ammonia is replaced by an alkyl or aromatic group. Important primary alkyl amines include, methylamine, most amino acids, while primary aromatic amines include aniline. According to the method of the present invention, primary amine groups of certain amino acids of said ECM components as defined elsewhere herein are labelled by said label as described above. In a preferred embodiment, primary amine groups of lysine of said ECM components as defined elsewhere herein are labelled.

An amine staining by Succinimidyl (NHS)-ester labelling has its effect in labelling all amine-containing ECM components and is not selective like antibodies which label one specific targets. The staining was developed for dead tissue and needs an alkaline pH, thus was assumed to damage living tissue. The inventors now surprisingly found out that living ex vivo tissue can be stained without damage. Thus, currently there are no reports on NHS/SDP-ester usage on living tissue, so no methods exists to visualize all amine-containing ECM molecules on organs.

After said ECM of said organ tissue as defined elsewhere herein has been contacted with said label, preferably contacted with a paper-like material comprising the label according to the present invention, said organ tissue may further be stamped with a biopsy punch into biopsy punches as described elsewhere herein. The contacting step of the ECM of said organ tissue with said label (labelling step) as described above followed by the punching step into small biopsy punchies can also be done in the other order.

It is also envisioned that components of the ECM, in particular proteins already comprise, e.g. non-canonical amino acids which enable a reaction with a label as described herein. For example, transgenic animals are available which express proteins comprising unusual or non-canonical amino acids which enable a reaction with a label as described herein.

The method of the present invention may also be extended by further comprising step (a′) namely contacting said organ tissue obtainable by biopsy from said mammalian subject with a label visualizing cells comprised in the ECM. In this context, said label refers to a lipophilic membrane fluorescent dye that spread through lateral diffusion capturing the entire cells. The additional labelling step may be performed before or after contacting the ECM of organ tissue with the first label as described elsewhere herein. Such membrane staining may be helpful to better identify/trace the ECM movement towards a site requiring deposition of ECM.

Step (b)

When in step b) of the method of the present invention the term “contact” or “contacting” is used, it refers that said compound of interest that is tested whether it modulates ECM movement towards a site requiring deposition of ECM is added directly onto the organ tissue which is placed in medium or said compound is added into the medium where the organ tissue is placed into. By simply adding the compounds of interest of the present invention either way (into the cultivation medium or explicitly onto the organ tissue), first ECM dynamics can be observed after about one hour or overnight. However, when ever it is deemed necessary contacting may further comprise adding compound or labeled compound into the blood stream. Such an administration may be a systemic administration, namely an injection.

The term “compound of interest” refers to a compound which is tested in the method of the present invention in order to identify whether said compound is a modulator of said ECM movement. Such modulator can be an inhibitor, thus inhibiting said ECM movement towards a site requiring deposition of ECM, once the inhibitor is contacted with said labelled ECM of organ tissue. However, such modulator may also refer to a promoter/an inducer, thus promoting/inducing said ECM movement towards a site requiring deposition of ECM, once the promoter is contacted with said labelled ECM of organ tissue. Preferably, the compound of interest may be an inhibitor. Even more preferably, said compound of interest refers to a protease inhibitor. A protease in general comprises metalloprotease, elastase or cathepsin and the like. Metalloproteases (metalloproteinase) can be divided into metalloendopeptidases, such as matrix-metallopeptidases (MMP1, 2, 3, 8, and 9), and metalloexopeptidases. In the present invention it has been shown that the metalloprotease (MMP) inhibitor GM6001 reacts specifically to Collagenases (MMP 1, 8), Gelatinases (MMP 2, 9) and Stromelysins (MMP 3).

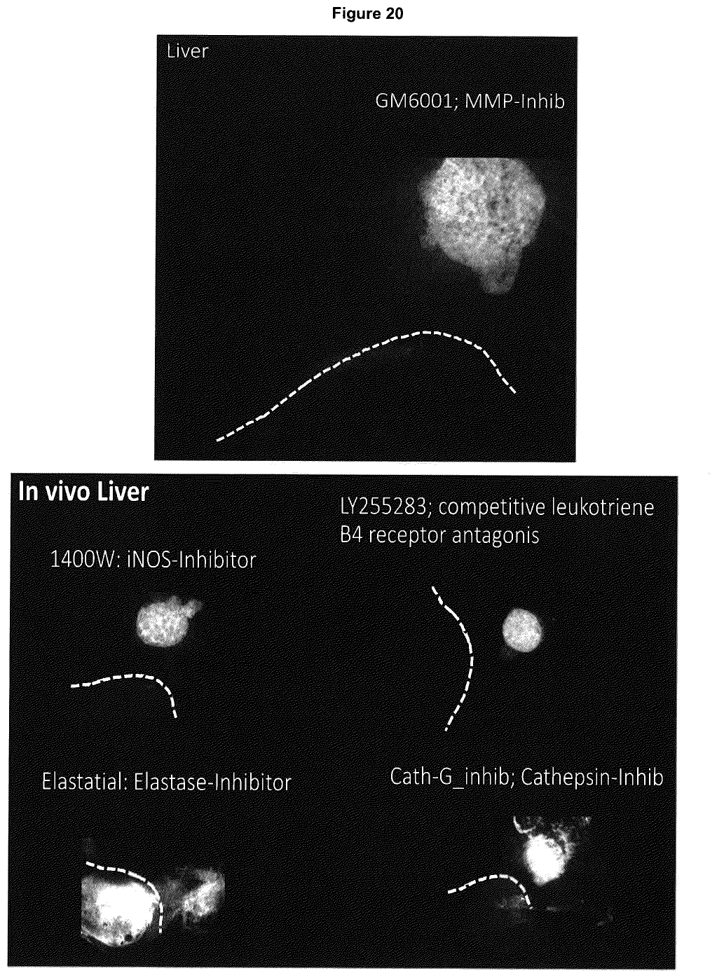

When the compound of interest is identified as an inhibitor of ECM movement on the basis of decreasing ECM movement, which results in a decreased deposition of ECM at a site requiring ECM deposition, such compound of interest may have an anti-fibrotic phenotype. Such compound of interest refers, but is not limited to GM6001, a metalloprotease (MMP) inhibitor; 1400W and L-Name, iNOS inhibitors; LY255283 and CP-105696, a leukotriene B4 receptor antagonists; and Cath-G inhibitor, a Cathepsin-G inhibitor (FIG. 20), the molecules of table 1 and especially Doxapram hydrochloride, Amorolfine hydrochloride, Flumethasone pivalate, Pyrvinium pamoate, Sulfaquinoxaline sodium salt, Piperacillin sodium salt, Iodixanol, Methylhydantoin-5-(D), Itraconazole, Azelastine HCl, Doxorubicin hydrochloride, Betamethasone, Thiostrepton, Clofazimine, Naltrexone hydrochloride dehydrate, Repaglinide, Propoxycaine hydrochloride, Tegaserod maleate, Phenylbutazone, Fluticasone propionate, Pivampicillin, Fluocinolone acetonide, Benzathine benzylpenicillin, Halofantrine hydrochloride, Sulfamethoxypyridazine, Levonordefrin, Medrysone, Oxalamine citrate salt, Ketorolac tromethamine, Bephenium hydroxynaphthoate, Fluvastatin sodium salt, Etidronic acid, disodium salt, Methotrimeprazine maleat salt, Haloprogin, Mevastatin, Domperidone, Alfacalcidol, Pyrazinamide, Eburnamonine (−), Minoxidil, Sulfaphenazole, Norethynodrel, Famotidine, Disopyramide, Amyleine hydrochloride, and Nefopam hydrochloride.

When the compound of interest is identified as a promoter of ECM movement on the basis of accelerating ECM movement, which results in an accelerated deposition of ECM at a site requiring ECM deposition, such compound of interest may have a pro-fibrotic phenotype. Such compound of interest refers, but is not limited to Elastial, an Elastase inhibitor, having a pro-fibrotic phenotype (FIG. 20).

The term “modulate” or “modulating” as used herein and described elsewhere herein in more detail means “inhibit” or “inhibiting”, if the compound may be an inhibitor of said ECM movement towards a site requiring deposition of ECM or “promote/induce”, if the compound may be a promoter/an inducer of said ECM movement towards a site requiring deposition of ECM

Step (c)

When the term “to determine” or “determining” in step c) of the method of the present invention is used herein, it may be done or achieved by visual inspection or protein biochemistry methods. The term “visual inspection” refers to the visualization whether said compound of interest indeed modulates ECM movement as defined elsewhere herein by using a microscope, preferably by using a fluorescence stereomicroscope. This determination/examination by visual inspection or even by any protein biochemistry methods known to a person skilled in the art is performed in comparison to an ECM of organ tissue, also obtainable by biopsy as defined elsewhere herein from a mammalian subject (or from the same mammalian subject as already used for taking the organ tissue for step a) of the method of the present invention), which has been labeled according to the present invention, however which has not been contacted with said compound of interest as described elsewhere herein.

During step (a), (b) and/or (c) of the method of the present invention as defined above fluid of said mammalian's body cavity may be present. For step (a) said fluid may be present in said labelling solution as described above when the ECM of organ tissue is contacted with said label comprised in said solution. When said fluid is present in step (b) of the method of the present invention, it may be added to the medium, where the organ tissue already having a labelled ECM is placed into for culturing, which may also comprise the compound of interest.

A body cavity is a space created in an organism which houses organs. It is lined with a layer of cells and is filled with the fluid being preferably used in the method of the present invention, to protect the organs from damage as the organism moves around. Said fluid of said mammalian's body cavity may enhance the labelling or culturing effect in the method of the present invention.

According to a second aspect, the present invention relates to a method for identifying a biomarker associated with extracellular matrix (ECM) movement towards a site requiring deposition of ECM, comprising (a) contacting extracellular matrix of organ tissue obtainable by biopsy from a mammalian subject with a label; (b) isolating proteins from said labelled ECM which move towards said site requiring deposition of ECM; (c) determining at least a partial amino acid sequence of said proteins, thereby identifying said proteins as a biomarker associated with ECM movement.

Examples of biomarkers for the ECM of different organs can be found in table 2 to 4.

Step (a) is carried out as described herein in the context of the methods for identifying modulators of ECM movement towards a site requiring deposition of ECM.

Step (b) is carried out by applying means and methods generally known to isolate proteins from, e.g. surfaces of membranes, paper-like material, etc. Indeed, since in step (a) preferably proteins comprised by ECM are labelled, it is possible to visualize such proteins moving towards a site requiring ECM deposition. Such ECM proteins are used as surrogate for movement of ECM towards a site requiring ECM deposition. Hence, any of such ECM proteins which move within ECM towards a site requiring deposition of ECM may be a suitable biomarker associated with ECM movement towards a site requiring deposition of ECM. Put differently, such an ECM protein identified as described herein may be indicative of ECM movement. In case of a pathological medical condition, such as fibrosis or chronic wounds, the presence, the amount, or absence of such a biomarker may be indicative of the degree or extent of the pathological medical condition.

Step (c) is carried out by applying means and methods generally known to determine at least a partial amino acid sequence of one or more proteins isolated in step (b), such as MALDI-TOF, HPLC, etc.

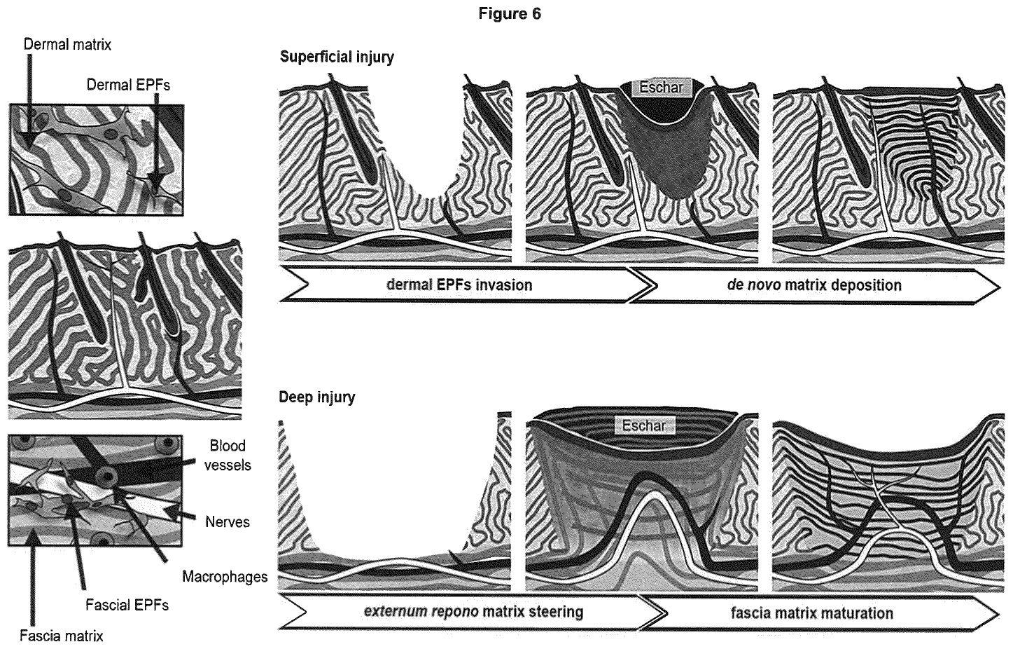

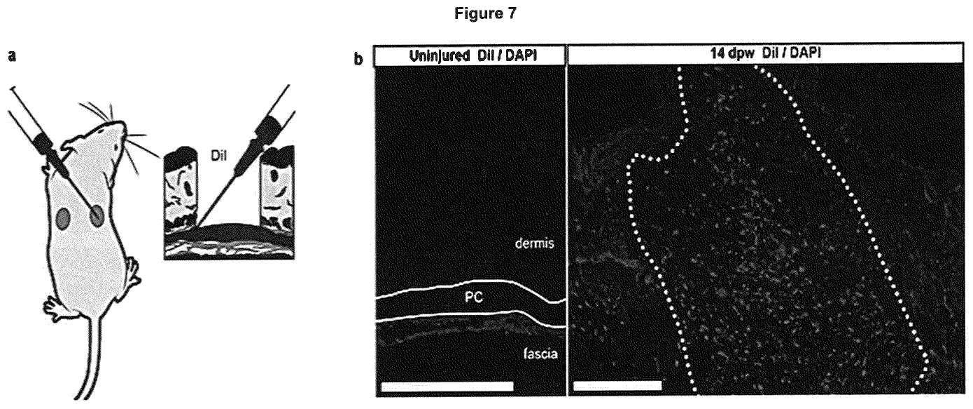

Thus far, the understanding in the prior art is that mammals form scars to quickly patch up wounds and ensure survival by an incompletely understood mechanism. Indeed, current wound healing models propose that fibroblasts migrate into sites of wounds where they locally initiate matrix deposition that is then remodeled into a mature scar. Based on their finding, the present inventors propose a revised model (see FIG. 6) where fascia fibroblasts pilot their local composite matrix into wounds where it is locally remodeled in deep injuries. Thus, instead of, e.g. dermal fibroblasts depositing matrix, scar primordium is steered by, inter alia, fascial fibroblasts, which represent a much efficient mechanism to quickly seal open wounds. Indeed, the present inventors found that the larger the wound, the more abundant is the fascia contribution. This implies that matrix steering by fascial fibroblasts is a mechanism that evolved to patch large and deep open wounds, whereas smaller more superficial wounds seem to be healed by the classical dermal fibroblast de novo deposition mechanisms. However, healing of more superficial wounds does not exclude the mechanism revealed by the present inventors. Healing of superficial wounds may thus include both mechanisms, the one by a de novo deposition and the one which involves ECM movement found by the present inventors.

The prevailing scientific view is that the body's connective tissues serves merely as a passive support framework for cells and organs and that this connective fibrous acellular network known as the ECM is stationary. The inventors disprove this idea by uncovering a fluid matrix system that radiates across internal organs. They show that injury induces gushes of fluid matrix across visceral and parietal organs. This immature fluid matrix is then cross linked, on site, to establish rigid frames thereby regenerating breaches in the structural continuums of organs and preserving organ integrity and function.

These findings challenge several widely held notions. The first dogma the inventors can dispense with is the idea that ECM is static. Secondly, they have now seen that new anatomies do not only emerge from de novo deposition of that rigid matrix, but rather from a mixture of new and shuttled fluid matrix. Finally, the data demonstrates that fibroblasts are no longer the major contributors for tissue reconstruction, but it is rather the job of the relative underappreciated immune-competent cells, the neutrophils. To recapitulate, the inventors findings indicate that rigid anatomies emerge from reservoirs of fluid matrix that are maneuvered into tissue construction sites by organ wide invasion of neutrophils. Fluid matrix then serves in injured organs as building blocks for new rigid anatomies and tissue repair.

Thus, in one preferred embodiment the presence of neutrophils might be determined or targeted when monitoring wound healing progression. In another embodiment it might be advantageous to stimulate neutrophils to move into a wound to increase the ECM movement toward a site requiring ECM deposition. Such stimulation might be established by the use of a compound or cytokines.

Fluid matrix is a pool of raw ingredients for fibrotic scars and regeneration and the inventors speculate that specific protein composition of the fluid matrix determines the diverging rigid anatomies that develop during adult tissue repair. Indeed, the composition of fluid elements varies from organ to organ. While in the liver many enzymes and pro-regenerative proteins are part of the fluid fraction, peritoneal elements consist mostly of fibrous and profibrotic elements, which are building blocks for scars.

The inventors also uncover a new exciting link between inflammation and tissue repair by showing the central role for neutrophils in piloting this fluid matrix material into wounds, and they do so in various ways. Immune cells transport cloudy matrix elements across organ surfaces within minutes. Whereas the transcriptomics analysis indicates that neutrophils are transcriptionally primed for this endeavor by activating multiple pathways. One pathway involves upregulation of the collagen binding integrins CD11b and CD18, which play an essential role in matrix movement, as blocking antibodies reduced the matrix currents. Another pathway involves LTB4 and nitric oxide synthase, and locally placing LTB4 forms new deposits of matrix, whereas inhibiting nitric oxide inhibits matrix flows. Neutrophils regulate therefore all facets of adult organ repair.

Thus, in another scenario it might be advantageous to block the ECM movement. By using the herein established reasoning an inhibition of the ECM movement might be established by using an neutrophil neutralizing antibody. A preferred neutrophil neutralizing antibody might be directed against Ly6g, CD11b or CD18.

The inventors have seen that mobilization of fluid matrix is a general principle of wound repair in adult tissues and organs, and they speculate it is in fact even more general. I.e. that flows are likely involved in development (organogenesis). They further speculate that emergence of new rigid frames during embryonic development is enacted by a similar fluid matrix process, and that ‘fluid matrix reservoirs & currents’ are newly emerged biological principles of the body plan. Their findings that matrix currents are absent in healthy adult organs suggests there are strong forces that hold-off matrix reservoirs from flowing, whereas counter-balancing forces activate matrix flows throughout adult life.

To the best of the present inventors' knowledge, the extent and magnitude of matrix movements documented in the present application; see the Examples, have never been observed during injury or regenerative settings.

The findings of the present inventors reveal an unprecedented dynamic and scale of motion for composite tissue matrix during injury that is mediated, inter alia, by specialized fibroblasts of the fascia. Thus, fascia serves as an externum repono for scar-forming matrix, and these findings indicate that matrix steering into wounds is the principle response of the fascia to large injuries.

The findings of the present inventors that fascia contributes to large scars and that its blockage leads to chronic open wounds, indicates that the ranges of chronic and excessive wound healing phenotypes of skin, such as diabetic and ulcerative wounds, as well as hypertrophic and particularly keloid scars might all be attributed to the fascia. Indeed, the superficial fascia varies widely in different species, sex, age, and anatomic skin locations²⁴. In some mammals, the superficial fascia is loose, whereas in man, dog and horse, the superficial fascia is thicker with larger connective tissue bands. The superficial fascia of human skin further varies in thickness on different regions of the body²⁵. For example, lower chest, back, thigh and arm have much thicker and multi-layered membranous sheets, and it is these anatomic sites that are prone to form large and keloid scars. Whereas other sites such as the foot have a much thinner or inexistent fascia.

Additionally, according to a third aspect, the present invention refers to a compound for use in a method for the modulation of extracellular matrix (ECM) movement towards a site requiring deposition of ECM, preferably in the treatment of a condition involving ECM deposition.

“A condition involving ECM deposition” is a medical condition which requires ECM deposition. As found by the present inventors, ECM can deliver components which, when deposited at a site requiring ECM deposition, aid in scar formation, preferably effect scar formation. Sometimes it is desired to modulate ECM movement and thereby ECM deposition and thus scar formation.

Accordingly, the present invention provides means and methods both for identifying modulators and ECM movement towards a site requiring ECM deposition and medical applications for the modulation, e.g. inhibition or promotion, of ECM movement towards a site requiring ECM deposition, preferably in the treatment of a condition involving ECM deposition.

Indeed, if scar is generated excessively, such a condition is undesired. Accordingly, the present invention provides for medical applications for the inhibition of ECM movement towards a site requiring ECM deposition, preferably in the treatment of a condition involving ECM deposition. Such a condition is, e.g., excessive deposition of ECM which may be associated with fibroproliferative disease.

Similarly, if scar is generated insufficiently, such a condition is undesired, too. Accordingly, the present invention provides for medical applications for the promotion of ECM movement towards a site requiring ECM deposition, preferably in the treatment of a condition involving ECM deposition. Such a condition is, e.g., insufficient deposition of ECM which may be associated with chronic wounds.

“A site requiring deposition of ECM”, or “a site requiring ECM deposition” as also used herein, is a site within organ tissue which signals a mammal's body the requirement for ECM deposition. The signal is triggered by, e.g. an injury caused, e.g. by a wound. Usually, ECM deposition is required for patching a wound. Thus, a site requiring ECM deposition is preferably a wound. A “wound” is a break in the continuity of any mammalian bodily tissue due to, e.g. violence, where violence is understood to encompass any action of external agency, including, for example, surgery. Said term includes open and closed wounds.

ECM movement as described herein and which can be visualized as described herein is, in accordance with the findings of the present inventors, mediated by fascia matrix. Fascia matrix, serosa and/or adventitia may comprise macrophages, neutrophils, mesothelial cells and/or fibroblasts. In particular, fascia matrix, serosa and/or adventitia may comprise fibroblasts.

A compound for use in a method for the modulation of ECM movement towards a site requiring deposition of ECM (equivalent to ECM deposition) can be any compound, such as a small molecule or the like. Such a compound includes cells or material from cells.

The effect of the compound on modulation of ECM movement may, for example, be tested in accordance with the methods of the present invention as described herein. Briefly, extracellular matrix of organ tissue obtainable by biopsy from a mammalian subject is contacted with a label; said labelled extracellular matrix of organ tissue is contacted with a compound of interest, i.e. a potential compound for use in a method for the modulation of ECM movement towards a site requiring ECM deposition; it is determined whether said compound of interest modulates ECM movement towards said site requiring deposition of ECM in comparison to labelled extracellular matrix of organ tissue obtainable by biopsy from a mammalian subject which is not contacted with said compound of interest, wherein modulation of ECM movement towards said site requiring deposition of ECM is indicative for said compound of interest to be a modulator of said ECM movement. As a modulator the compound of interest may inhibit ECM movement or may promote ECM movement.

Preferably, in accordance with the methods for identifying modulators, e.g. inhibitors or promoters, of ECM movement towards a site requiring ECM deposition as described herein, a compound for use in a method for the modulation of ECM movement towards a site requiring ECM deposition, preferably in the treatment of a condition involving ECM deposition may be identified. A thus-identified compound may then be used in a method for the modulation of ECM movement towards a site requiring ECM deposition, preferably in the treatment of a condition involving ECM deposition. Accordingly, the present invention provides for a compound which is obtainable/obtained by the methods or identifying modulators, e.g. inhibitors or promoters, of ECM movement towards a site requiring ECM deposition as described herein for the modulation of ECM movement towards a site requiring ECM deposition, preferably in the treatment of a condition involving ECM deposition.

However, although preferred, it is not necessary that a compound for use in the method for the modulation of EM movement of the present invention is tested in accordance with the methods for identifying such modulators as provided by the present inventors. Indeed, any compound can be used as long as it modulates ECM movement towards a site requiring ECM deposition. If needed, ECM movement towards a site requiring ECM deposition may be tested as described herein, e.g. as described hereinabove.

Preferably, a compound is for use in a method for the modulation of ECM movement towards a site requiring ECM deposition in the treatment of a condition involving ECM deposition. Since the present inventors found for the first time that ECM movement delivers components for scar formation to a site requiring ECM deposition, the present invention provides for an early as possible treatment of a condition involving ECM deposition. Accordingly, the treatment of a condition involving ECM deposition allows thus preferably the prevention of either excessive deposition of ECM at a site requiring ECM deposition or insufficient deposition of ECM at a site requiring ECM deposition.

Indeed, inhibition of ECM movement towards a site requiring deposition of ECM prevents excessive deposition of ECM at said site. Accordingly, a modulator of ECM movement towards a site requiring ECM deposition may preferably be an inhibitor. That said, the present invention relates to a compound for use in a method for the inhibition of extracellular matrix (ECM) movement towards a site requiring deposition of ECM, preferably in the treatment of a condition involving ECM deposition. It is preferred that inhibition of ECM movement towards a site requiring deposition of ECM prevents excessive ECM deposition at said site. An example of excessive deposition of ECM is associated with fibroproliferative diseases.

A “fibrotic” disease or a “fibroproliferative” disease refers to a disease characterized by scar formation and/or the over production of extracellular matrix by connective tissue. Fibrotic disease may occur as a result of tissue damage. It can occur in virtually every organ of the mammalian body. Examples of fibrotic or fibroproliferative diseases include, but are not limited to, idiopathic pulmonary fibrosis, fibrotic interstitial lung disease, interstitial pneumonia, fibrotic variant of non-specific interstitial pneumonia, cystic fibrosis, lung fibrosis, silicosis, asbestosis, asthma, chronic obstructive pulmonary lung disease (COPD), pulmonary arterial hypertension, liver fibrosis, liver cirrhosis, renal fibrosis, glomerulosclerosis, x kidney fibrosis, diabetic nephropathy, heart disease, fibrotic valvular heart disease, systemic fibrosis, rheumatoid arthritis, excessive scarring resulting from surgery, e.g., surgery to fix hernia, chemotherapeutic drug-induced fibrosis, radiation induced fibrosis, macular degeneration, retinal and vitreal retinopathy, atherosclerosis, and restenosis. Fibrotic disease or disorder, fibroproliferative disease or disorder and, as sometimes used herein, fibrosis, are used interchangeably herein.

For livers and in the case of peritoneas the laparotomy section as local injury (FIG. 40a) the inventors could show that the inhibition of lysyl oxidases and elastases resulted in increased ECM movement. The inhibition of motor proteins showed inhibitory effects on ECM currents only in peritoneas. Heat shock factor inhibition blocked ECM currents in both organs. Among the protease inhibitors, the broad-spectrum MMP inhibitor GM6001 proved to be the most potent.

Using the inventors signaling pathway analysis, they identified multiple molecules that inhibited or amplified matrix flows. Interestingly, some effector molecules like Blebbistatin and ciliobrevin effected matrix currents of only one organ.

For the peritoneum the lysyl oxidase BAPN and the proteases elastase inhibitor II and Elastatinal increased the ECM movement of the surrounding tissue. Thus, in a preferred embodiment BAPN and Elastatinal might be used to increase the ECM movement of the tissue surrounding the peritoneum.

A lysyl oxidase inhibitor for use in increasing the ECM movement of the tissue surrounding the peritoneum. The lysyl oxidase inhibitor for use in increasing the ECM movement wherein the lysyl oxidase inhibitor is BAPN. A lysyl oxidase inhibitor for use in increasing the wound healing capacity of the peritoneum tissue. The lysyl oxidase inhibitor for use in increasing the wound healing capacity of the peritoneum tissue.

The motor protein Ciliobrevin D and (S)-nitro-Blebbistatin, the heat shock factor Quercetin, KNK437 and the proteases cathepsin B inhibitor, GM6001 and BESTATIN decreased the ECM movement around the peritoneum.

In a preferred embodiment Ciliobrevin D, (S)-nitro-Blebbistatin, Quercetin, KNK437, cathepsin B inhibitor, GM6001 and BESTATIN might be used to decrease the ECM movement around the peritoneum.

An inhibitor of motor protein, a heat shock factor, or a protease for use in decreasing fibrosis in the peritoneum tissue.

For the liver the lysyl oxidase BAPN and the proteases elastase inhibitor II and Elastatinal increased the ECM movement. Thus, in a preferred embodiment BAPN and Elastatinal might be used to increase the ECM movement of the tissue surrounding the liver.

A lysyl oxidase inhibitor for use in increasing the ECM movement of the tissue surrounding the liver. The lysyl oxidase inhibitor for use in increasing the ECM movement of the tissue surrounding the liver wherein the lysyl oxidase inhibitor is BAPN. A lysyl oxidase inhibitor for use in increasing the wound healing capacity of the liver tissue. The lysyl oxidase inhibitor for use in increasing the wound healing capacity of the liver tissue.

The heat shock protein Qercetin and KNK437, the proteases cathepsin B inhibitor, MMP12 and Cathepsin G inhibitor and GM6001 decreased the matrix movement. In yet another embodiment Qercetin, KNK437, cathepsin B inhibitor, MMP12, Cathepsin G inhibitor and GM6001 might be used to decrease the ECM movement around the liver.

An inhibitor of motor protein, a protease for use in decreasing fibrosis in the liver tissue, wherein the motor protein inhibitor is Quercetin or KNK437, wherein the protease inhibitor is cathepsin B inhibitor, MMP12, Cathepsin G inhibitor or GM6001. Quercetin for use in decreasing fibrosis in the liver tissue. KNK437 for use in decreasing fibrosis in the liver tissue. Cathepsin B inhibitor for use in decreasing fibrosis in the liver tissue. MMP12 for use in decreasing fibrosis in the liver tissue. Cathepsin G inhibitor for use in decreasing fibrosis in the liver tissue. GM6001 for use in decreasing fibrosis in the liver tissue.

Since the composition of the fluid matrix differs from organ to organ, organ-specific modulators of the matrix currents could be applied after identification of appropriate biomarkers.

In summary, the Inventors show a new method to attach molecules to wounds, new potential markers for pulmonary fibrosis and signaling pathways to modulate matrix movements.

Bleomycin-induced pulmonary fibrosis has different degrees of severity. Robust biomarkers should therefore show different abundancies depending on the severity of pulmonary fibrosis.

Until now it was assumed that lungs scar from newly synthesized connective tissue in response to injury. The inventors data presented herein paint a new picture by revealing a system of fluid scar tissue that, after activation, migrates from the pleura into the interstitium. This new fluid scar brings fibrous building blocks as well as the corresponding enzymes to mature the tissue into fixed scar tissue on site. Thus, lungs scar primarily by restructuring pre-existing connective tissue.

It still remains possible that fibroblasts deposit matrix to contribute to scarring, as a secondary response to irrigation. Indeed, the experiments show that in the absence of matrix irrigation, fibroblasts remain dormant and remain inactive. Thus, it still remains possible that matrix irrigation stiffens the connective tissues surrounding the fibroblasts, which in turn activates them to further secrete or remodel the new matrix surrounding them.

The proteomics data of the fluid matrix from mouse and humans indicates that irrigation of human lungs leads to a much greater reduction of surface elasticity. These findings also uncover the link between inflammation and fibrosis. Monocytes and lymphocytes, not only trigger the invasion of fluid scar tissue they can accelerate the inward movement and accretion of new connective tissue. The fact that immune cells of patients with chronic lung diseases are ‘primed’ for matrix maneuvering and further enhance this effect even more has exciting therapeutic implications. This indicates that the rate of fluid movements and fibrosis depends on the ‘priming’ state of individual immune cells and that a deeper understanding of this ‘priming’ mechanism could open new therapeutic and diagnostic opportunities to combat fibrosis onset.

Taken together, the inventors findings presented herein on lungs, and the previous findings on skin (REF) that show loose connective tissues serve as a source for dermal scars, imply for matrix movements as a germinal scarring mechanism and response to injury across the body.

First mass spectrometric analyses of lung tissue found varying amounts of proteins in the lungs of bleomycin versus control animals. This indicates that the primarily labelled proteins undergo changes due to the stimulus. Proteins such as fibrinogen are known to form net-like structures. It could be that fibrinogen is covalently bound to the primary labelled proteins. In fact, the inventors were also able to identify proteins of varying abundance of the initially labelled lung matrix in the blood of the animals (FIG. 39, table c).

From the abovementioned FIG. 39, table c, it is apparent that Hmgcs2, Bhmt show the highest fold change (93 and 52 respectively) in comparison to the rest of the tested proteins in mouse lungs. Thus, in one preferred embodiment Myo1e and Hnmpa3 expression may be indicative for fibrosis or lung fibrosis in lung tissue.

Different markers were found in blood samples. As shown in FIG. 39 table d, Myo1e, Hnmpa3 show the highest fold change in comparison to the rest of the tested proteins in blood. Thus, in one preferred embodiment Myo1e and Hnmpa3 expression in the blood may be indicative for fibrosis or lung fibrosis. An analysis of lung biopsies may be combined with the analysis of fibrosis markers in the blood. Thus, a high expression of the lung tissue markers Hmgcs2, Bhmt and the blood makers Myo1e and Hnmpa3 is envisaged by the present invention.

In summary, the Inventors show here that fluid elements enter the blood stream during mobilization of the lung matrix during fibrotic events. These elements can be detected and could serve as biomarkers for fibrotic events.

In contrast, promotion of ECM movement towards a site requiring deposition of ECM prevents insufficient deposition of ECM at said site. Accordingly, a modulator of ECM movement towards a site requiring ECM deposition may preferably be a promoter. That said, the present invention relates to a compound for use in a method for the promotion of extracellular matrix (ECM) movement towards a site requiring deposition of ECM, preferably in the treatment of a condition involving ECM deposition. A compound promoting the ECM movement is for example the lysyl oxidase inhibitor BAPN or a chemokine attracting neutrophils to a site requiring ECM deposition, preferably the chemokine Lipoxin A4. As the inventors have found that neurtrophils are one of the first cells moving into a site requiring ECM deposition and that neutrophils are capable of recruiting ECM material for the deposition at that site, it is apparent that a chemokine attracting neutrophils may be able to initiate the ECM deposition and thus a natural healing process. This may be advantageous for chronic wounds which are not closed following the natural pathway or to fasten the closure of wounds. It may also be advantageous for other inflammatory diseases where healing of damaged tissue is wanted.

It is preferred that promotion of ECM movement towards a site requiring deposition of ECM prevents insufficient ECM deposition at said site. An example of insufficient deposition of ECM is associated with chronic wounds. A “chronic wound” is a wound (preferably as defined herein) that does not heal in an orderly set of stages and in a predictable amount of time the way most wounds do; wounds that do not heal within about two to three months are usually considered chronic. For example, chronic wounds often remain in the inflammatory stage for too long and remain as opening in the skin and sometimes the deeper tissue. Chronic wounds may never heal or may take years to do so.

While prior art focuses on end-point phenotypes regarding, e.g., fibrosis or keloid, the present invention—due to the findings of the present inventors—allows focusing on the starting point. Indeed, the present inventors succeeded for the first time in in vivo labelling of ECM and could thus observe in real-time its movement towards a site requiring ECM deposition, such as a wound, e.g. caused by an injury. This allows interfering with ECM deposition at a much earlier point in time than known before which opens up new treatment options which were not available before. Put differently, the treatment methods of the present invention which apply compounds which modulate ECM movement towards a site requiring ECM deposition allow preferably a prevention of either excessive or insufficient deposition of ECM at a site requiring ECM deposition, since the present inventors elucidated the mechanism which a mammal's body uses to patch wounds—by ECM movement.

Accordingly, the methods of the present invention relating to treatment aspects described herein are preferably for the prevention of either excessive deposition of ECM at a site requiring ECM deposition or insufficient deposition of ECM at a site requiring ECM deposition. Prior to the present invention, such an early (preventative) treatment was not possible, since the mechanism elucidated by the present inventors was neither known nor understood. However, thanks to the present invention, the mechanism of ECM movement is understood and, therefore, new treatment options are available, in particular a preventative treatment of either excessive deposition of ECM at a site requiring ECM deposition or insufficient deposition of ECM at a site requiring ECM deposition. Indeed, a modulator which is an inhibitor of ECM movement towards a site requiring ECM deposition may ideally prevent excessive deposition of ECM due to inhibiting ECM movement, while a modulator which is a promoter of ECM movement towards a site requiring ECM deposition may ideally prevent insufficient deposition of ECM due to promoting ECM movement.

By following the teachings of the present invention, the present inventors could already identify compounds for use in a method for the modulation of ECM movement towards a site requiring ECM deposition, preferably in the treatment of a condition involving ECM deposition as described herein. In particular, matrix metalloprotease inhibitors, such as GM6001; serine protease inhibitors, such as cathepsin G inhibitors; iNOS inhibitors, such as W1400; or leukotriene B4 receptor antagonists, such as LY255283 were identified and applied in vivo. As is shown in FIG. 20, matrix metalloprotease inhibitors, serine protease inhibitors, iNOS inhibitors or leukotriene B4 receptor antagonists are able to inhibit ECM movement towards a site requiring deposition of ECM, such as a wound. Indeed, an injury was generated at each of the organs described in FIG. 20 which required ECM deposition. However, matrix metalloprotease inhibitors, serine protease inhibitors, iNOS inhibitors or leukotriene B4 receptor antagonists inhibited ECM movement towards the site of injury.

Accordingly, matrix metalloprotease inhibitors, serine protease inhibitors, iNOS inhibitors or leukotriene B4 receptor antagonists, heat shock inhibitors, inhibitors of motor proteins and neutrophil neutralizing antibodies are preferred compounds for use in a method for the modulation of ECM movement towards a site requiring ECM deposition, preferably in the treatment of a condition involving ECM deposition as described herein. Matrix metalloprotease inhibitors, serine protease inhibitors, iNOS inhibitors or leukotriene B4 receptor antagonists, heat shock inhibitors, inhibitors of motor proteins and neutrophil neutralizing antibodies are in the sense of the present invention inhibitors of ECM movement. As such they have an anti-fibroproliferative effect.

In particular, elastase inhibitors, such as elastial was identified and applied in vivo. As is shown in FIG. 20, elastase inhibitors are able to promote ECM movement towards a site requiring deposition of ECM, such as a wound. Indeed, an injury was generated at each of the organs described in FIG. 20 which required ECM deposition. As can be seen, elastase inhibitors promoted ECM movement towards the site of injury.

Accordingly, elastase inhibitors are preferred compounds for use in a method for the modulation of ECM movement towards a site requiring ECM deposition, preferably in the treatment of a condition involving ECM deposition as described herein. Elastase inhibitors are in the sense of the present invention promoters of ECM movement. As such they have a pro-fibroproliferative effect.

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. The term “at least one” refers to one or more such as two, three, four, five, six, seven, eight, nine, ten or more. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “less than” or in turn “more than” does not include the concrete number.

For example, less than 20 means less than the number indicated. Similarly, more than or greater than means more than or greater than the indicated number, e.g. more than 80% means more than or greater than the indicated number of 80%.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”. When used herein “consisting of” excludes any element, step, or ingredient not specified.

The term “including” means “including but not limited to”. “Including” and “including but not limited to” are used interchangeably.

The term “about” means plus or minus 10%, preferably plus or minus 5%, more preferably plus or minus 2%, most preferably plus or minus 1%.

Throughout the description and claims of this specification, the singular encompasses the plural unless the context otherwise requires. In particular, where the indefinite article is used, the specification is to be understood as contemplating plurality as well as singularity, unless the context requires otherwise.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications cited throughout the text of this specification (including all patents, patent application, scientific publications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The content of all documents and patent documents cited herein is incorporated by reference in their entirety.

A better understanding of the present invention and of its advantages will be gained from the following examples, offered for illustrative purposes only. The examples are not intended to limit the scope of the present invention in any way.

The present invention may also be characterized by the following items:
1. A method for identifying modulators of extracellular matrix (ECM) movement towards a site requiring deposition of ECM, comprising
   (a) contacting extracellular matrix of organ tissue obtainable by biopsy from a mammalian subject with a label;
   (b) contacting said labelled extracellular matrix of organ tissue with a compound of interest;
   (c) determining whether said compound of interest modulates ECM movement towards said site requiring deposition of ECM in comparison to labelled extracellular matrix of organ tissue obtainable by biopsy from a mammalian subject which is not contacted with said compound of interest,
   wherein modulation of ECM movement towards said site requiring deposition of ECM is indicative for said compound of interest to be a modulator of said ECM movement.
2. The method of item 1, wherein modulation is inhibition.
3. The method of item 1, wherein modulation is promotion.
4. The method of any one of the preceding items, wherein said organ tissue comprises fascia matrix, serosa and/or adventitia.
5. The method of any one of the preceding items, wherein fascia matrix, serosa and/or adventitia comprises macrophages, neutrophils, mesothelial cells and/or fibroblasts.
6. The method of any one of the preceding items, wherein ECM comprises proteins, polysaccharides and/or proteoglycans.
7. The method of any one of the preceding items, wherein the label is a dye or tag.
8. The method of item 7, wherein the dye is a fluorescent dye.
9. The method of any one of the preceding items, wherein amine groups of extracellular matrix components are labelled.
10. The method of item 9, wherein the amine groups of extracellular matrix components are labelled by NHS ester.
11. The method of item 9 and 10, wherein the amines are primary amines.
12. The method of any one of the preceding items, wherein the label is covalently coupled to extracellular matrix components.
13. The method of any one of the preceding items, wherein contacting extracellular matrix of organ tissue obtainable by biopsy from said mammalian subject with a label is achieved by contacting said extracellular matrix with a paper-like material comprising the label.
14. The method of any one of the preceding items, wherein fluid of said mammalian's body cavity is present during the step (a), (b) and/or (c).
15. The method of any one of the preceding items, further comprising step (a′) contacting said organ tissue obtainable by biopsy from said mammalian subject with a label visualizing cells comprised in the ECM.
16. The method of any one of the preceding items, wherein the organ tissue is from skin, kidney, lung, heart, liver, bone, peritoneum, intestine, diaphragm or pleura.
17. A method for identifying a biomarker associated with extracellular matrix (ECM) movement towards a site requiring deposition of ECM, comprising:
   (a) contacting extracellular matrix of organ tissue obtainable by biopsy from a mammalian subject with a label;
   (b) isolating proteins from said labelled ECM which move towards said site requiring deposition of ECM;
   (c) determining at least a partial amino acid sequence of said proteins, thereby identifying said proteins as a biomarker associated with ECM movement.
18. A compound for use in a method for the modulation of extracellular matrix (ECM) movement towards a site requiring deposition of ECM, preferably in the treatment of a condition involving ECM deposition.
19. The compound for the use of item 18, wherein ECM movement is mediated by fascia matrix.
20. The compound for the use of item 18 or 19, wherein fascia matrix, serosa and/or adventitia comprises macrophages, neutrophils, mesothelial cells, and/or fibroblasts.
21. The compound for the use of any one of items 18 to 20, wherein fascia matrix, serosa and/or adventitia comprises fibroblasts.
22. The compound for the use of any one of items 18 to 21, wherein ECM comprises proteins, polysaccharides and/or proteoglycans.
23. The compound for the use of any one of items 18 to 22, wherein the site requiring deposition of ECM is a wound.
24. The compound for the use of any one of items 18 to 23, wherein modulation is inhibition.
25. The compound for the use of item 24, wherein the inhibitor is any one of the molecules of table 1.
26. The compound for the use of item 24 and 25, wherein the inhibitor of the ECM movement is selected from the group consisting of Doxapram hydrochloride, Amorolfine hydrochloride, Flumethasone pivalate, Pyrvinium pamoate, Sulfaquinoxaline sodium salt, Piperacillin sodium salt, Iodixanol, Methylhydantoin-5-(D), Itraconazole, Azelastine HCl, Doxorubicin hydrochloride, Betamethasone, Thiostrepton, Clofazimine, Naltrexone hydrochloride dehydrate, Repaglinide, Propoxycaine hydrochloride, Tegaserod maleate, Phenylbutazone, Fluticasone propionate, Pivampicillin, Fluocinolone acetonide, Benzathine benzylpenicillin, Halofantrine hydrochloride, Sulfamethoxypyridazine, Levonordefrin, Medrysone, Oxalamine citrate salt, Ketorolac tromethamine, Bephenium hydroxynaphthoate, Fluvastatin sodium salt, Etidronic acid, disodium salt, Methotrimeprazine maleat salt, Haloprogin, Mevastatin, Domperidone, Alfacalcidol, Pyrazinamide, Eburnamonine (−), Minoxidil, Sulfaphenazole, Norethynodrel, Famotidine, Disopyramide, Amyleine hydrochloride, and Nefopam hydrochloride.
27. The compound for the use of item 24, wherein inhibition of ECM movement towards a site requiring deposition of ECM prevents excessive deposition of ECM at said site.
28. The compound for the use of item 27, wherein excessive deposition of ECM is associated with scaring and fibroproliferative disease.
29. The compound for the use of item 28, wherein the fibroproliferative disease is any one of idiopathic pulmonary fibrosis, fibrotic interstitial lung disease, interstitial pneumonia, fibrotic variant of non-specific interstitial pneumonia, cystic fibrosis, lung fibrosis, silicosis, asbestosis, asthma, chronic obstructive pulmonary lung disease (COPD), pulmonary arterial hypertension, liver fibrosis, liver cirrhosis, renal fibrosis, glomerulosclerosis, kidney fibrosis, diabetic nephropathy, heart disease, fibrotic valvular heart disease, systemic fibrosis, rheumatoid arthritis, excessive scarring resulting from surgery, e.g., surgery to fix hernia, chemotherapeutic drug-induced fibrosis, radiation induced fibrosis, macular degeneration, retinal and vitreal retinopathy, atherosclerosis, and restenosis.
30. The compound for the use of any one of items 18 to 23, wherein the condition involving ECM deposition is excessive deposition of ECM.
31. The compound for the use of item 30, wherein excessive deposition of ECM is associated with fibroproliferative disease.
32. The compound for the use of any one of items 18 to 23, wherein modulation is promotion.
33. The compound for the use of item 32, wherein promotion of ECM movement towards a site requiring deposition of ECM prevents insufficient deposition of ECM at said site.
34. The compound for the use of item 33, wherein insufficient deposition of ECM is associated with chronic wounds.
35. The compound for the use of any one of items 18 to 23 and 32 to 34, wherein the condition involving ECM deposition is insufficient deposition of ECM.
36. The compound for the use of item 35, wherein insufficient deposition of ECM is associated with chronic wounds.
37. The compound for the use of any one of items 18-36, wherein said compound is obtainable by the method of any one of items 1 to 16.
38. A compound for use in a method for the inhibition of extracellular matrix (ECM) movement towards a site requiring deposition of ECM, preferably in the treatment of a condition involving excessive ECM deposition.
39. The compound for the use of item 38, wherein the site requiring a deposition of ECM is a wound.
40. The compound for the use of item 38 and 39, wherein inhibition of ECM movement prevents excessive deposition of ECM at said site which is associated with scaring.
41. The compound for the use of items 38 to 40, wherein the compound is an inhibitor of the ECM movement.
42. The compound for the use of items 38 to 41, wherein the inhibitor is any one of the molecules of table 1.
43. The compound for the use of item 38 and 42, wherein the inhibitor is selected from the group consisting of Doxapram hydrochloride, Amorolfine hydrochloride, Flumethasone pivalate, Pyrvinium pamoate, Sulfaquinoxaline sodium salt, Piperacillin sodium salt, Iodixanol, Methylhydantoin-5-(D), Itraconazole, Azelastine HCl, Doxorubicin hydrochloride, Betamethasone, Thiostrepton, Clofazimine, Naltrexone hydrochloride dehydrate, Repaglinide, Propoxycaine hydrochloride, Tegaserod maleate, Phenylbutazone, Fluticasone propionate, Pivampicillin, Fluocinolone acetonide, Benzathine benzylpenicillin, Halofantrine hydrochloride, Sulfamethoxypyridazine, Levonordefrin, Medrysone, Oxalamine citrate salt, Ketorolac tromethamine, Bephenium hydroxynaphthoate, Fluvastatin sodium salt, Etidronic acid, disodium salt, Methotrimeprazine maleat salt, Haloprogin, Mevastatin, Domperidone, Alfacalcidol, Pyrazinamide, Eburnamonine (−), Minoxidil, Sulfaphenazole, Norethynodrel, Famotidine, Disopyramide, Amyleine hydrochloride, and Nefopam hydrochloride.
44. The compound for the use of items 43, wherein the inhibitor is preferably any one of the anti-fibrotic agents Itraconazole, Thiostrepton, or Fluvastatin sodium salt.
45. A compound for the use in treating chronic wounds in the liver, wherein the compound is a lysyl oxidase inhibitor.
46. The compound for the use item 45, wherein the lysyl oxidase inhibitor is capable of modulating the movement of the ECM tissue surrounding the liver.
47. The compound for the use of item 46, wherein modulation is promotion.
48. The compound for the use of item 47, wherein promotion of ECM movement towards a site requiring deposition of ECM prevents insufficient deposition of ECM at said site.
49. The compound for the use of item 48, wherein insufficient deposition of ECM is associated with chronic wounds.
50. The compound for use of item 45 to 49, wherein the lysyl oxidase inhibitor is BAPN.
51. A compound for use in treating chronic wounds in the peritoneum, wherein the compound is a lysyl oxidase inhibitor.
52. The compound for the use of item 51, wherein the lysyl oxidase inhibitor is capable of modulating the movement of the ECM tissue surrounding peritoneum tissue.
53. The compound for the use of item 52, wherein modulation is promotion.
54. The compound for the use of item 53, wherein promotion of ECM movement towards a site requiring deposition of ECM prevents insufficient deposition of ECM at said site.
55. The compound for the use of item 54, wherein insufficient deposition of ECM is associated with chronic wounds.
56. The compound for the use of item 51 to 55, wherein the lysyl oxidase inhibitor is BAPN.
57. BAPN for use in promoting the movement of the ECM tissue surrounding peritoneum.
58. A method for diagnosing fibroproliferative disease, comprising
   (a) obtaining a blood sample or biopsy from a risk patient for fibroproliferative disease;
   (b) determining whether fibroproliferative disease marker are detectable in a biopsy or the blood,
   wherein the detection of fibroproliferative disease marker in the blood or the biopsy tissue is indicative for a fibroproliferative disease.
59. The method of item 58, wherein the method further comprises comparing the amount of detected fibroproliferative disease marker with a control value.
60. A method for diagnosing lung fibrosis, comprising
   (a) obtaining a blood sample from a risk patient for lung fibrosis;
   (b) determining whether lung fibrosis marker are detectable in the blood;
   wherein the detection of lung fibrosis marker in the blood is indicative for lung fibrosis.
61. The method of item 60, wherein the method further comprises comparing the amount of detected lung fibrosis marker with a control value.
62. The method of item 61, wherein the determined lung fibrosis marker in the blood are preferably Myo1e or Hnmpa3.
63. A method for diagnosing lung fibrosis, comprising
   (a) obtaining a lung biopsy from a risk patient for lung fibrosis;
   (b) determining whether lung fibrosis marker are detectable in the lung biopsy tissue, wherein the detection of lung fibrosis marker in the lung biopsy tissue is indicative for lung fibrosis.
64. The method of item 63, wherein the method further comprises comparing the amount of detected lung fibrosis marker with a control value.
65. The method of item 63 and 64, wherein the determined marker in the lung biopsy tissue are preferably Hmgcs2 or Bhmt.
66. A method of treating fibrosis, comprising administering to a subject in need thereof an effective amount of neutrophil neutralizing antibody, wherein neutralizing the neutrophils blocks the ECM movement and the thereby the deposition of ECM forming fibrotic tissue, and continuing the treatment if the fibrotic tissue is reduced as compared to the pre-treatment of the fibrotic tissue.
67. A method of treating fibroproliferative disease, wherein the fibroproliferative disease is diagnosed according to any one of item 58, 60 and 63, and wherein the treatment comprises administering of at least one inhibitor of the ECM movement to a patient in the need thereof, thereby reducing the excessive ECM deposition causing fibroproliferative tissue.
68. The method of item 67, wherein the ECM inhibitor is any one of item 42 to 44.
69. The method of items 67 and 68, wherein the method further comprises monitoring of monocytes, lymphocytes and neutrophils, preferably neutrophils.
70. The method of item 69, wherein the inhibitor of the ECM movement is combined with a further inhibitor of the ECM movement, preferably a neutrophil neutralizing antibody.
71. A compound for use in treating fibrosis, wherein the compound is a neutrophil neutralizing antibody.
72. The compound for the use of item 71, wherein the neutrophil neutralizing antibody is capable of modulating the movement of the ECM tissue.
73. The compound for the use of item 72, wherein modulation is inhibition.
74. The compound for the use of item 73, wherein inhibition of ECM movement towards a site requiring deposition of ECM prevents excessive deposition of ECM at said site.
75. The compound for the use of item 74, wherein excessive deposition of ECM is associated with fibrosis.
76. The compound for the use of item 71 to 75, wherein the neutrophil neutralizing antibody is preferably directed against Ly6g, CD11 b or CD18.
77. A method of treating lung fibrosis, comprising administering at least one neutrophil neutralizing antibody to a patient in the need thereof, thereby inhibiting the ECM movement and excessive deposition into the lung tissue, wherein the excessive deposition of ECM into the lung tissue causes fibrosis.
78. The method of item 77, wherein the neutrophils express more integrins than other neutrophil populations of the same patient.
79. The method of item 78, wherein the integrins are preferably CD11b or CD18.
80. The method of items 77 to 79, wherein the neutrophil neutralizing antibody is preferably directed against Ly6g, CD11 b or CD18.
81. A neutrophil neutralizing antibody for use in a method for inhibition of extracellular matrix (ECM) movement towards a site requiring deposition of ECM, preferably in the treatment of a condition involving ECM deposition.
82. The method for the use of item 81, wherein inhibition of ECM movement towards a site requiring deposition of ECM prevents excessive deposition of ECM at said site.
83. The compound for the use of item 82, wherein excessive deposition of ECM is associated with scaring.
84. The method for the use of items 81 to 83, wherein the neutrophil neutralizing antibody is preferably directed against Ly6g, CD11b or CD18.
85. A compound for use in treating wounds, wherein the compound is a chemokine.
86. The compound for the use of item 85, wherein the chemokine is administered to a patient in the need thereof.
87. The compound for the use of items 85 and 86, wherein the chemokine is administered systemically or locally.
88. The compound for the use of items 85 to 87, wherein the chemokine is attracting neutrophils.
89. The compound for the use of items 85 to 88, wherein attracting neutrophils promotes the ECM movement into the wound and thereby the wound healing.
90. The compound for the use of items 85 to 89, wherein the wound is a chronic wound.
91. The compound for the use of items 85 to 90, wherein the chemokine is preferably Lipoxin A4.
92. A method of preventing scar formation after an injury, comprising the use of at least one ECM movement inhibitor, wherein the method comprises contacting the injury site with at least one ECM movement inhibitor, and wherein the ECM movement inhibitor is capable of reducing the ECM deposition to the injury site, and thereby reduces the scar formation.
93. The method of item 92, wherein the ECM movement inhibitor is any one of the molecules of table 1.
94. The method of item 92 and 93, wherein the ECM movement inhibitor is any one of item 43.
95. The method of items 92 to 94, wherein the inhibitor is further a neutrophil neutralizing antibody, a inhibitor of the leukotriene receptor activity, or an inhibitor of the nitric oxide synthesis.
96. The method of items 92 to 95, wherein the injury site is contacted with the ECM movement inhibitor over the complete depth of the injury site.
97. The method of items 92 to 96, wherein the fascia close to the injury site is contacted with the ECM movement inhibitor.
98. The method of items 92 to 97, wherein the injury is a surgical wound.
99. A composition for use in preventing scar formation comprising the ECM movement inhibitor of items 93 to 95.
100. A compound for use in preventing scar formation, wherein the compound is an inhibitor of the neutrophil leukotriene receptor activity.
101. The compound for the use of item 100, wherein the inhibitor of the leukotriene receptor activity is administered to a patient in the need thereof.
102. The compound for the use of items 100 and 101, wherein the inhibitor of the leukotriene receptor activity is administered systemically or locally.
103. The compound for the use of items 100 to 102, wherein the inhibitor of the leukotriene receptor activity blocks neutrophils and thereby the ECM movement and deposition leading to scar formation.
104. The compound for the use of items 100 to 103, wherein the wound is a chronic wound.
105. The compound for the use of items 100 and 104, wherein the inhibitor of the leukotriene receptor activity is preferably LY255283 or CP-105696.
106. A compound for use in preventing scar formation, wherein the compound is an inhibitor of neutrophil nitric oxide synthesis.
107. The compound for the use of item 106, wherein the inhibitor of nitric oxide synthesis is administered to a patient in the need thereof.
108. The compound for the use of items 106 and 107, wherein the inhibitor of nitric oxide synthesis is administered systemically or locally.
109. The compound for the use of items 106 to 108, wherein the wound is a chronic wound.
110. The compound for the use of items 106 to 109, wherein the inhibitor of nitric oxide synthesis blocks neutrophils and thereby the ECM movement and deposition leading to scar formation.
111. The compound for the use of items 106 and 110, wherein the inhibitor of nitric oxide synthesis preferably is W1400 or L-Name.
112. A method of treating fibrosis, comprising the use of at least one inhibitor of the extracellular matrix (ECM) movement to reduce the ECM movement towards a site requiring less deposition of ECM, wherein the method comprises contacting the extracellular matrix of organ tissue from a mammalian subject with a labeled inhibitor of the ECM movement, and wherein a reduced ECM movement towards said site requiring less deposition of ECM is indicative for said inhibitor to reduce excessive ECM deposition.
113. The method of item 112, wherein the label is NHS ester.
114. The method of item 112 and 113, wherein the NHS ester label allows the assessment whether the ECM movement toward a site requiring less ECM deposition is reduced after the treatment.
115. The method of item 112 and 114, wherein the inhibitor of the ECM movement is any one of the molecules in table 1.
116. The method of item 112 to 115, wherein the inhibitor is any one of the molecules of item 43.
117. The method of item 112 to 116, wherein the inhibitor of the ECM movement is selected from a neutrophil neutralizing antibody, an inhibitor of the leukotriene receptor activity in neutrophils, an inhibitor of the nitric oxide synthesis in neutrophils, an inhibitor of motor proteins, an inhibitor of proteases or an inhibitor of heat shock proteins.
118. The method of item 112 to 117, wherein the NHS ester-compound combination is administered systemically.
119. A compound for use in treating fibrosis, wherein the compound is an inhibitor of the neutrophil leukotriene receptor activity.
120. The compound for the use of item 119, wherein the inhibitor of the leukotriene receptor activity is administered to a patient in the need thereof.
121. The compound for the use of items 119 and 120, wherein the inhibitor of the leukotriene receptor activity is administered systemically or locally.
122. The compound for the use of items 119 to 121, wherein the inhibitor of the leukotriene receptor activity blocks neutrophils and thereby the ECM movement.
123. The compound for the use of items 119 to 122, wherein the ECM movement causes deposition of ECM and thereby the formation of fibrotic tissue.
124. The compound for the use of items 119 and 123, wherein the inhibitor of the leukotriene receptor activity is preferably LY255283 or CP-105696.
125. A compound for treating fibrosis, wherein the compound is an inhibitor of neutrophil nitric oxide synthesis.
126. The compound for the use of item 125, wherein the inhibitor of nitric oxide synthesis is administered to a patient in the need thereof.
127. The compound for the use of items 125 and 126, wherein the inhibitor of nitric oxide synthesis is administered systemically or locally.
128. The compound for the use of items 125 to 127, wherein the wound is a chronic wound.
129. The compound for the use of items 125 to 128, wherein the inhibitor of the neutrophils nitric oxide synthesis blocks the neutrophils and thereby the ECM movement.
130. The compound of for the use of item 129, wherein blocking the ECM movement blocks excessive ECM deposition and thereby the formation of fibrotic tissue.
131. The compound for the use of items 125 and 130, wherein the inhibitor of nitric oxide synthesis preferably is W1400 or L-Name.
132. A compound for use in treating fibrosis in liver tissue, wherein the compound is an inhibitor of motor proteins.
133. The compound for the use of item 132, wherein the inhibitor of motor proteins modulates the movement of the ECM tissue surrounding the liver.
134. The compound for the use of item 133, wherein modulation is inhibition.
135. The compound for the use of item 134, wherein inhibition of ECM movement towards a site requiring deposition of ECM prevents excessive deposition of ECM at said site.
136. The compound for the use of item 135, wherein excessive deposition of ECM is associated with fibrosis in the liver.
137. The compound for the use of items 132 to 136, wherein the motor protein inhibitor is Quercetin or KNK437.
138. A compound for use in treating fibrosis in the liver tissue, wherein the compound is an inhibitor of proteases.
139. The compound for the use of item 138, wherein the inhibitor of proteases is capable of modulating the movement of the ECM tissue surrounding the liver.
140. The compound for the use of item 139, wherein modulation is inhibition.
141. The compound for the use of item 140, wherein inhibition of ECM movement towards a site requiring deposition of ECM prevents excessive deposition of ECM at said site.
142. The compound for the use of item 141, wherein excessive deposition of ECM is associated with fibrosis in the liver.
143. The compound for the use of items 138 to 142, wherein the protease inhibitor is cathepsin B inhibitor, MMP12, Cathepsin G inhibitor or GM6001.
144. Cathepsin B inhibitor for use in inhibiting the movement of the ECM tissue surrounding the liver.
145. MMP12 for use in inhibiting the movement of the ECM tissue surrounding the liver.
146. Cathepsin G inhibitor for use in inhibiting the movement of the ECM tissue surrounding the liver.
147. GM6001 for use in inhibiting the movement of the ECM tissue surrounding the liver.
148. A composition for use in treating fibrosis in the liver tissue, wherein the composition comprises any one of Quercetin, KNK437, Cathepsin B inhibitor, MMP12, Cathepsin G inhibitor and GM6001.
149. A compound for use in treating fibrosis in peritoneum tissue, wherein the compound is an inhibitor of heat shock factor.
150. The compound for the use of item 149, wherein the inhibitor of heat shock factors is capable of modulating the movement of the ECM tissue surrounding the peritoneum tissue.
151. The compound for the use of item 150, wherein modulation is inhibition.
152. The compound for the use of item 151, wherein inhibition of ECM movement towards a site requiring deposition of ECM prevents excessive deposition of ECM at said site.
153. The compound for the use of item 152, wherein excessive deposition of ECM is associated with fibrosis.
154. The compound for the use of item 150 to 153, wherein the inhibitor of heat shock factor is Quercetin or KNK437.
155. Quercetin for use in inhibiting the movement of the ECM tissue surrounding the peritoneum.
156. KNK437 for use in inhibiting the movement of the ECM tissue surrounding the peritoneum.
157. A compound for use in treating fibrosis in peritoneum tissue, wherein the compound is an inhibitor of motor protein.
158. The compound for the use of item 157, wherein the inhibitor of motor protein is capable of modulating the movement of the ECM tissue surrounding the peritoneum tissue.
159. The compound for the use of item 158, wherein modulation is inhibition.
160. The compound for the use of item 159, wherein inhibition of ECM movement towards a site requiring deposition of ECM prevents excessive deposition of ECM at said site.
161. The compound for the use of item 160, wherein excessive deposition of ECM is associated with fibrosis.
162. The compound for the use of item 157 to 161, wherein the inhibitor of motor protein is Ciliobrevin D or (S)-nitro-Blebbistatin.
163. A compound for use in treating fibrosis in peritoneum tissue, wherein the compound is an inhibitor of proteases.
164. The compound for the use of item 163, wherein the inhibitor of proteases is capable of modulating the movement of the ECM tissue surrounding the peritoneum tissue.
165. The compound for the use of item 164, wherein modulation is inhibition.
166. The compound for the use of item 165, wherein inhibition of ECM movement towards a site requiring deposition of ECM prevents excessive deposition of ECM at said site.
167. The compound for the use of item 166, wherein excessive deposition of ECM is associated with fibrosis.
168. The compound for the use of item 163 to 167, wherein the inhibitor of proteases is cathepsin B inhibitor, GM6001 or BESTATIN.
169. A composition for use in treating a fibrosis in the peritoneum, wherein the composition comprises Quercetin, KNK437, BAPN, Ciliobrevin D, (S)-nitro-Blebbistatin, cathepsin B inhibitor, GM6001 or BESTATIN.
170. A compound for use as inhibitor of ECM movement, wherein the compound is Nitedamib.
171. The compound for the use of item 170, wherein the inhibition of ECM movement towards a site requiring deposition of ECM prevents excessive deposition of ECM at said site.
172. The compound for the use of item 170 and 171, wherein excessive deposition of ECM is associated with scaring and fibroproliferative disease.
173. The compound for the use of item 172, wherein the fibroproliferative disease is any one of fibrosis, pulmonary fibrosis, systemic sclerosis, liver cirrhosis, cardiovascular disease, progressive kidney disease, and macular degeneration.
174. A composition comprising a NHS ester linked to a compound.
175. The composition of item 174, wherein NHS ester is N-hydroxysuccinimide ester or Succinimidyl ester.
176. The composition of items 174 and 175, wherein NHS ester is capable of targeting primary amines.
177. The composition of item 176, wherein primary amines occur in wounds.
178. The composition of items 174 to 177, wherein the composition is administered systemically.
179. The composition of items 174 to 178, wherein the NHS ester allows targeting wounds after being applied locally or systemically.
180. The composition of items 174 to 179, wherein the compound is obtainable by the method of any one of items 1 to 16.
181. The composition of items 174 to 180, wherein the compound is selected from the group consisting of modulator of the ECM movement, chemokines, inhibitors of the leukotriene receptor activity and nitric oxide synthesis inhibitor.
182. The composition of item 181, wherein the chemokine is preferably Lipoxin A4.
183. The composition of item 181, wherein the inhibitor of the leukotriene receptor activity is preferably LY255283 or CP-105696.
184. The composition of item 181, wherein the nitric oxide synthesis inhibitor is preferably W1400 or L-Name.
185. A NHS ester for use in diagnosing wounds, wherein the NHS ester is capable of binding extracellular amines in the extracellular matrix of wounds when administered systemically and thereby labels extracellular amines in wounds.
186. The NHS esters of item 185 for the use in diagnosing wounds, wherein the NHS ester is further combined with a reporter molecule.
187. A diagnostic composition comprising NHS ester capable of binding to extracellular amines in the extracellular matrix combined with a reporter molecule, administered to a patient in the need thereof, wherein the NHS ester is capable of targeting the reporter molecule to extracellular amines of a wound, thereby labeling the wound for diagnostic proposes.
188. The diagnostic composition of item 187, wherein the patient is suffering from at least one wound.
189. A method for detecting the extend of an internal wound, wherein the method comprises administering NHS ester to a patient having an internal wound, thereby labeling extracellular amines in the wound which makes the extend of the wound detectable.
190. The method of item 189, wherein NHS ester is N-hydroxysuccinimide ester or Succinimidyl ester.
191. The method of items 189 and 190, wherein NHS ester is combined with a reporter molecule.
192. The method of items 189 to 191, wherein the NHS ester is administered systemically.
193. A therapeutic composition for use in treating wounds comprising an NHS ester combined with a compound capable of treating wounds, wherein the NHS ester is capable of binding extracellular amines of wounds and thereby targets the compound to the wound.
194. The therapeutic composition of item 193, wherein the compound is obtainable by the method of any one of items 1 to 16.
195. The therapeutic composition of item 193 and 194, wherein the compound is selected from the group consisting of modulator of the ECM movement, chemokines, inhibitors of the leukotriene receptor activity and NOS inhibitor.
196. The therapeutic composition of item 195, wherein the compound is a chemokine, preferably chemokine Lipoxin A4.
197. The therapeutic composition of item 195, wherein the compound is an inhibitor of the leukotriene receptor activity preferably LY255283 or CP-105696.
198. The therapeutic composition of item 195, wherein the compound is a NOS inhibitor, preferably W1400 or L-Name.
199. A method for treating a chronic wound, comprising:
   (a) contacting the extracellular matrix of organ tissue with a NHS ester-compound combination;
   (b) monitoring the progression of the chronic wound after performing step (b),
   (c) continuing the treatment if the chronic wound tissue is reduced, as compared to the pre-treatment of the chronic wound.
200. The method of item 199, wherein the NHS ester capable of binding to amines in the wound is combined with a compound suitable for healing wounds, thereby creating a NHS ester-compound combination, prior to step (a).
201. The method of item 199 and 200, wherein the progression of the chronic wound is monitored for one hour up to ten days after performing step (b).
202. The method of items 199 to 201, wherein the compound is obtainable by the method of any one of items 1 to 16.
203. A method of treating a chronic wound, comprising administering to a patient in need thereof an effective amount of NHS ester-compound combination, wherein the NHS ester is capable of targeting primary amines of chronic wounds and thereby targets the compound to the chronical wound, determining the healing progression of the chronic wound, and continuing the treatment if the chronic wound tissue is reduced as compared to the pre-treatment of the chronic wound.
204. A method for treating a wound, comprising:
   (a) contacting the extracellular matrix of organ tissue with a NHS ester-compound combination;
   (b) monitoring the progression of the wound after performing step (b),
   (c) continuing the treatment if the wound tissue is reduced as compared to the pre-treatment of the wound.
205. The method of item 204, wherein NHS ester capable of binding to primary amines in wounds is combined with a compound for wound healing, thereby creating a NHS ester-compound combination, prior to step (a).
206. The method of item 204 and 205, wherein the wound is a surgery wound.
207. The method of item 204 and 206, wherein the progression of the wound is monitored for one hour up to three days after performing step (b).
208. A method of treating wounds, comprising administering to a subject in need thereof an effective amount of NHS ester-compound combination, determining the wound healing progression, and continuing the treatment if the wound tissue is reduced as compared to pre-treatment of the wound.
209. The method of items 199, 203, 204 and 208, wherein NHS ester is N-hydroxysuccinimide ester or Succinimidyl ester.
210. The method of items 199, 203, 204 and 208, wherein the compound is obtainable by the method of any one of items 1 to 16.
211. The method of items 199, 203, 204 and 208, wherein the compound is selected from the group consisting of modulator of the ECM movement, chemokines, inhibitors of the leukotriene receptor activity and NOS inhibitor.
212. The method of item 211, wherein the compound is a chemokine, preferably chemokine Lipoxin A4.
213. The method of item 211, wherein the compound is an inhibitor of the leukotriene receptor activity preferably LY255283 or CP-105696.
214. The method of item 211, wherein the compound is a NOS inhibitor, preferably W1400 or L-Name.
215. A method of identifying a biomarker associated with organ specific extracellular matrix and the movement of ECM towards a site requiring deposition of ECM, comprising:
   (a) contacting the ECM of an organ obtainable by biopsy from a mammalian subject with a label;
   (b) isolating proteins from said labelled ECM which move towards said site requiring deposition of ECM;
   (c) determining at least a partial amino acid sequence of said proteins, thereby identifying said proteins as a biomarker associated with ECM movement.
216. The method of item 215, wherein the label is N-hydroxysuccinimide ester or Succinimidyl ester.
217. A biomarker obtainable by the method of item 215.
218. The biomarker of item 217, wherein the organ is liver.
219. The biomarker of item 218, wherein the biomarker is selected from the group consisting of Rps4x, Calr, Gdi1, 1 SV, Ephx1, Cct2, Cth, Foxe3, Hadh, Acsf2, Hsp90aa1, Uba1, Slc25a13, Tuba1b, Prdx6, Ywhag, Ccdc18, Krt8, Actn4, Atp5a1, Lama2, Uqcrc1, Tubb4b, Capza1, Acaa2, Nudt7, Egfr, Acsm1, Got2, Hspa9, Sdha, Otc, Npnt, Cat, Synpo, Vcp, Etfb, Hnrnpa2b1, Sod1, Itch, Krt17, Bsg, Tst, Mdh1, Eef2, Gstm1, Pebp1, Spr, Eno1, Bdh1, Cps1, Ak3, Anxa5, Aldh2, Ppia, Hspa1l, Blvrb, Eci1, Slc27a2, Cyp2e1, Pccb, Ephx2, Gpd1, Hspd1, Rps3, Glt8d2, Myo3b, Ca3, Pygl, Tpi1, Hsp90ab1, Cyp2d10, Ndufs1, Inmt, Slc35g2, Glo1, Rpsa, Csad, Hpd, Uroc1, Sardh, Actb, Agrn, Ndrg2, SucIg2, Scp2, Atp5c1, Aox3, Lyz1, Aldh1I1, Glud1, Prdx1, Hnrnpa3, Clu, Hba, Mtco2, Dars, Lonp2, Ftcd, Prdm16, Usp5, Tufm, Ushbp1, Gbe1, Hspa8, Myh9, Pfn1, Tkt, Sdhb, Ighg1, Arsj, Hnrnpk, Selenbp1, Aco2, Pzp, Aldh6a1, Sec14I2, Sord, Hsd17b4, Eefla1, Pgk1, Aldh8a1, Gnmt, Acsl1, Cyp3a11, Ldha, Aco1, Pgm1, Cbs, Serpinc1, Pbld2, Hmgcl, Tuba4a, Idh1, Hadha, Hmgcs2, Krt10, Stard10, Asl, Psmd2, Cyp2f2, Comt, Hsp90b1, Mdh2, Akr1c6, Anxa2, Atp1a1, Aldh7a1, Cltc, Cyp2d26, Hnrnpf, Abhd14b, Ces3a, Ubb, Uox, Akr1a1, Rps20, Anxa6, Hbb-b1, Arf3, Abcd3, Chdh, H3f3c, Ywhaz, Aldh4a1, Bhmt, Myl6, Acat1, Tkfc, Mccc2, Ahcy, Rnh1, Sucla2, Hgd, Isoc2a, Fdps, Dhrs1, Pkhd111, Krt18, Rgn, Cct8, Pck1, Got1, Krt72, Tinagl1, Krt42, Tgm2, Ass1, Me1, Acadvl, Gdi2, Histlh4a, Ddx5, Haao, Agxt, Krt75, Mfap4, Aldob, Pdha1, Dmgdh, Cntf, Hagh, Hist1h2bc, Fmo5, Cs, Tcp1, Aldh9a1, Dcaf8, Aldh1a1, Mug1, Gapdh, Coq8a, Adgrl1, Eif5a, Psmd1, Pipox, Ak2, Lcp1, Vtn, Krt2, Eef1b, Nid2, Hadhb, Serpina1d, Rbm20, Fabp5, Trap1, Alb, Abat, Pklr, Ndufab1, Nit2, Hint2, Pgam1, Acly, Krt5, Eif3a, Nid1, Iqgap2, Fbp1, Urad, Ckm, Fasn, Reep6, Prdx5, Krt14, Pgrmc1, Tpm3, Rps5, Gstp1, Ehhadh, Etfa, Psmd12, Cyp2c50, Kng1, Mif, H2afz, Akr7a2, Lonp1, Selenbp2, Acat2, Hspg2, Ttc38, Rps9, Cox5a, Nsdhl, Xirp2, Hnrnpm, lvd, Vim, Apoe, Copg2, Ssr1, Amdhd1, KIb, Atxn2, Adh5, Acox1, Hacl1, Gpt, Mthfd1, Mipep, Rpn2, Ttpa, Etfdh, Rp1p0, Psmc6, Rpl6, Krt1, Esd, Eif4a1, Khk, Copa, Dbi, Fn1, Pdcd6ip, Ugdh, Gtf3c1, Gabrb3, Rps25, Atp5o, Por, Emi Sds, Klhl1, Mycn, Prdx3, Aifm1, Ywhae, Slc27a5, Lamb2, Cyp2c54, Gsto1, Col6a6, Akr1d1, Sec13, Krt77, Col4a2, Cct3, Jup, 9913 GN, Aldoa, Ywhaq, Bsn, Col6a2, Xrra1, Hdlbp, Gstz1, Ncl, Adh1, Dld, Asap2, Acox2, Uso1, Cyp3a13, Krt76, Fip111, Park7, Cfl1, Fbln1, Kyat3, Cpne7, Hint1, Gpx1, Hoxa4, Lap3, Cyb5a, Sephs2, Gsta3, Col4a3, Cyp2u1, Rrbp1, Cpt2, Krt79, Txn, Fabp1, Idh2, Acadm, Ces1, Akr1c13, Iigp1, Lamb1, Lama4, Grhpr, Mpo, Cct4, 1 SV, Lrpprc, Rpl4, Fmol, Marc2, Rpf1, Pde8b, Sec31a, Cyp2d9, Col5a2, Krt19, Msn, Rdh7, Cbr1, Lama5, Psmb3, Preip, D1Pas1, Prdx2, Ecm1, Col7a1, Plg, Rpl3, Ncf2, Lamc1, Pah, Fbn2, Lta4h, Dhdh, Col6a4, Aass, Lmna, Acaa1b, Ddx1, Dsp, Krt16, Rp115, Cmb1, Myl12b, Rab35, Senp5, Trim33, Mmrn2, Itih1, Adk, Reg3g, Cyp2c29, F13b, Acad1, Urgcp, Agmat, Ugt2b17, Rps14, Stard9, Lrfn1, Acaca, Acad11, Snd1, Acaa1a, Prodh, Col6a5, Rp122, Cdkn2aip, Glyat, Uqcrc2, Rpip1, Lum, Dpf1, Acads, Gata3, Insrr, Dync1i1, Ccs, P4hb, Cyb5r3, Dpyd, Maob, Rack1, Ppa1, Nkapl, Fah, Cdc42bpg, Rps16, Shmt1, Dcik3, Nudt6, Cdc42, Hibadh, Rpip2, Col4a1, Serpina3m, Saa2, Mtch2, Pxdn, Atp5b, Batf3, Glu1, Ugtlal, Serpina1b, Eef1d, Pgd, Ltbp4, Lgais9, Aldh3a2, Decr1, Dbt, Mcccl, Banf1, Serpina1e, Col3a1, Acta2, Ywhab, Slc25a20, Chd7, Cyp2c40, Rad23b, Serpinf2, Col15a1, Chat, F13a1, Serpina1c, Cyb5b, Tfap2d, Fbn1, Nedd4, Rai1, Pabpc1, Mat1a, Col11a1, Dgcr8, Ddt, Acacb, Rpl9, Ptbp1, Dmbt1, Krt7, Col2a1, Col6a1, Megf6, Eef1g, Tf, Col4a4, Cox5b, Tgfbi, C1qbp, S100a11, Rps12, Gys2, Rnf43, Fam208b, FIg2, Apoa1, Pcbp1, Tnc, FbIn5, Rabepk, Ambp, Hpx, Upk1b, Vwf, Mlk4, Fh, Pnma1, Eif4g1, S100a8, Flna, Adipoq, Eif4h, Loxl1, Col18a1, Hal, Ltbp1, Anks1b, Kcnk4, Mup2, Cesld, Bcl9, Col8a2, Fam65c, Aspdh, C4b, Slc25a5, Calml3, Dpysl2, Krt35, Sod2, Sall2, Myo16, Anxa7, Pggt1b, Zc3h12a, Vdac2, Atn1, Myh11, Il15ra, Msra, Hsd11b1, Hdgf, LRWD1, Pcca, Krt71, Dpt, Fgb, Apoa4, Sfswap, Efi1, Itih4, A1bg, Tnrc6c, Lasp1, Pdlim1, Zfhx3, Mgstl, Col8a1, Pc, Prg2, Ahsg, Myh4, Sptan1, Serpina3k, Hsd17b10, Krt4, Postn, Hrg, Atp8a2, Bgn, Col14a1, Polr2d, Col1a1, Anxa1, Ltk, Col16a1, Ttn, Arg1, Dcn, Col5a1, Krt6a, Fga, Efemp1, Acsl5, Col12a1, Ces2a, Arhgap5, Hspa5, Urah, S100a9, Cycs, Ptms, Rp127a, Aqp1, Elk4, Pdia3, Fgg, Ftl1, Isoc1, Fus, and C3.
220. The biomarker of item 219, wherein the biomarker is preferably Ambp, F13a1, F13b, Itih1 or PZP.
221. The biomarker of item 217, wherein the organ is peritoneum.
222. The biomarker of items 221, wherein the biomarker is selected from the group consisting of Lamc1, Nid1, Lama2, Syne2, Tpm2, Col6a2, Lamb2, Mmp20, Eno3, Col6a1, Krt86, Col14a1, Atp5b, Art3, Tinagl1, Lama4, 1 SV, Mb, Hspg2, Anxa1, Acta1, Cc2d1a, Pkm, Pygm, Apoc1, Myh3, Mdh2, Nid2, Aldoa, Plg, Apoe, Ca3, Col6a6, Tnnt3, Pgam2, Srl, Col16a1, Krt77, Vwf, Rad54I2, Pvalb, Myh7, Gsn, Dcn, Lamb1, Col15a1, Pgm1, Pgk1, Fam160b2, Aco2, Col4a2, Myh4, Mpo, Hspa8, Prelp, Ralgapa1, Prdx6, Prg2, Tnnc2, Atp5a1, Vdac1, Ldha, Efl1, Fga, Myh8, Reg3b, Fgg, Lum, Cps1, Magi3, Pfkm, Omd, Cpe, Tpi1, Ak1, Fam208b, Meltf, Krt33a, Serpina1b, Serpinf2, Col4a1, Ttn, Des, Sypl2, Hspd1, Tgfbi, Klhdc9, Mgp, HapIn1, Akap13, C3, Atp2a1, Bhmt, Fbn2, Kcng4, Mylpf, Bgn, Myot, Rev3I, Csf2rb, Mfap4, Cacna2d1, Filip1I, Fgb, Myom1, OsbpI8, Camsap1, Col9a2, Col5a1, Actn3, Egfbp2, Tpm1, Hrg, Matn1, Fbn1, Myoz1, Tmem207, Krt19, Ltbp4, Fhdc1, S100a11, Ubb, Tpm3, Ogn, Cdkn2aip, Ppa1, Emilin1, Serpina3m, Cpa3, Dpt, Lgals1, Tef, Ckm, Ampd1, Efhc1, Mmp9, S100b, Fasn, Cxcr1, Rgn, Tuba4a, Plch2, Hnrnpa2b1, Hpx, Cilp2, Kmt2a, Prdx1, Capza1, Chrnd, Casq1, S100a1, Pc, Apoa1, Col10a1, Spn, Actn2, Npepps, Apoa4, Arhgap5, Fnbp4, Bcl9, Serpina3k, Mvb12a, Sord, Ecm1, Ushbp1, Insrr, Aspn, Gapdh, Alb, Plec, Tprn, Krt10, Eef1a1, Comp, Clu, Lama5, Ppip5k1, Col5a2, Dsp, Postn, Ppfia3, Actc1, Tf, Wipf1, Itih4, Col11a1, Sepp1, Cycs, Asb3, Tgm2, Serpina1d, Fn1, Map2k4, Abcc3, Krtap3-1, Tmem184c, Vim, Fbln5, Cma1, SIc25a37, Myo3b, Med1, Col2a1, Amy2, Ube3b, Apobec2, Tnni2, Eno1, Lef1, Ryr1, Lox11, Tuba1b, Bsg, Nup98, Ass1, Acan, Sgf29, Rps15, Krt42, Col1a2, AIdh2, Dock8, Cys1, Obscn, Fbxo48, Col9a1, Kiaa1109, Wdfy3, Taco1, Lyz1, GMCL1P1, Krtap15-1, Hsp90ab1, Krt6a, Hbb-b1, Ddx25, Foxe3, Mnt, Ppia, Gabrb3, Spp1, Pou2f3, Eef1a2, Dopey2, Pcca, Fmod, Pank1, Met, Prdx2, Hba, Chad, Tmem45a, F13a1, Krt16, Irf4, Cdh13, Aldob, K1h141, Dgcr2, Ogdh, Hmcn2, Peli3, Sparc, Ywhab, Thbs4, Hist1h2bc, Hist1h4a, Matn3, S100a9, Adipoq, Col1a1, Asap2, Krt14, Krt75, Anxa6, Tcap, Cpb2, Atf4, Ldb3, Calml3, Ighg1, Trim33, Chat, Acaa1b, Brd3, Tubb4b, Hoxa4, Myl1, Spata61, S100a8, Riok3, Alms1, Jup, Krt35, Isoc2a, Myh1, Krt76, Grem1, Gnmt, Elavl2, Hcls1, Krt34, Pdha1, Krt17, Hivep2, Mrps2, Ica1, Col11a2, H2afz, Flnc, Mybpc2, Mtco2, Vtn, Tmprss6, Rtn1, Krt8, Col3a1, Col12a1, Mknk2, Anxa5, Timmdc1, Acat1, Eif3a, Bmp2k, Krt5, Ccdc18, Gsk3a, Pde8b, Krt2, Actb, Krt1, Krt72, Cd22, Krt79, Krt24, Krtap19-5, Anxa2, Serpinc1, 9913 GN, Bcl2I14, Banp, Med19, Arg1, Zmym3, Upf1, Slc25a4 and Ndrg2.
223. The biomarker of item 222, wherein the biomarker is preferably Grem1, Ogn, Chad or MMP9 in the ECM of the peritoneum.
224. The biomarker of item 217, wherein the organ is the cercum.
225. The biomarker of items 224, wherein biomarker is selected from the group consisting of Lamb1, Ubac1, Col4a2, Serpina1c, Col19a1, Tln1, Lamb2, Col8a1, Cdc37, Clu, Col4a1, Krt31, Postn, Lama5, Epg5, Ino80d, Pcca, Lamc1, Golga4, Nid2, Ptrf, Lum, Hrg, Vtn, Hspg2, Krt6a, C1qbp, Banf1, Trerf1, Col16a1, Hnrnpa3, Lama4, Col4a4, Tgfbi, Myh11, 1 SV, Serpinf2, Col15a1, Acta2, Gp2, Dpt, Cep295nl, Insrr, Fn1, Tgm2, Tpm1, Als2, Anxa6, Myl6, Plg, Lama2, Pigr, Selp, Serpina1d, Muc2, Nid1, Ecm1, Tpm2, Col3a1, Runx1, Fgb, Flna, Pc, Fga, Ltbp1, Col6a4, Col5a1, Jchain, Synm, Serpina3k, Tubb5, Fgg, Usp18, Serpinc1, Flnc, Dscaml1, Phkb, Npnt, Col5a2, Ahsg, Krt16, Itln1b, Apoa4, Mfap5, Smtn, Col6a2, Fbln2, Synpo2, Fbn2, Col1a1, Fbn1, IgIc2, Col6a1, Ltbp4, Colla2, Dmbtl, Palld, Prelp, Col6a5, CoMal, Reg3g, Reg3b, Lox11, Pou3f3, Col2a1, Hap1, Smchd1, Trpv6, Svs4, AdamtsI4, Exd2, Clca1, Ace, Krt85, Hspb1, Des, Hspd1, Ckmt1, Ivl, Zg16, Tinagl1, Abca3, Got2, Emilin1, Fndc7, Hnrnpa2b1, Slc25a4, Actn1, Myl9, Cnn1, Tubb4b, Ptma, Vim, Ca1, Epx, Col12a1, Prg2, Tagln, S100a8, Alb, Tuba1b, 9913 GN, Hnrnpk, Colec12, D1Pas1, Pglyrp1, Ezr, S100a9, Tpm3, Nlrp3, Cdsn, Psmb7, Hist2h2aa1, Mbl2, Efemp1, Krt5, Diras2, Krt79, Vcl, Lyz1, Krt42, Itih4, S100a11, Krt1, Krt77, Krt14, Krt76, Arg1, Cyfip2, Pkd1I3, Calml3, Nup214, Atp5a1, Hist1h2bc, Prdx1, Krt2, Ppia, Ykt6, Hist1h1c, Nes, Gapdh, Krt17, Dsp, Spag5, Pde8b, Lipc, Krt75, Txn, S100a6, Jup, Actb, Krt10, Mpo, H3f3c, Krt71, Dsg1b, Hspa2, Apoa1, Pkm, Hba, Plec, Hbb-b1, Eef1a1, Anxa2, Tmprss13, Anxa1, Krt25, Tnc, Rgs8, Ubb, Psmb4, Krt80, Pkp1, Cfl1, Myh9, Tgm1, Ywhaz, Hbb-b2, Poli, Hsp90ab1, Eef2, Brap, Krt24, Lmna, Prdx2, Srcin1, Rps3, Nccrp1, Mylk, Tgm3, Krt23, Cpa4, Psmb2, Eno1, Pgk1, Blmh, Eif6, Aldh9a1, Krt19, Fabp5, Krt35, Psmb5, Serpina1e, Vdac2, Hspa5, Gsdma3, Aldoa, Tpi1, Hal, Krt73, Set, Krt13, Eif4a1, Pof1b, Krt12, Psma1, Ankrd17, Capn1, Ccdc6, Psma3, Krt4, Psma6, Psma7, Ide, Ahcy, Mast1, Rpl22 and Vdac1.

### EXAMPLES OF THE INVENTION

Material and Methods being Used in the Present Invention

Mice and Genotyping.

All mouse strains (C57BL/6J, En1^{Cre}, R26^{VT2/GK3}, R26^{mtmg}, R26^{iDTR}, Rag2^{-/-}, and Fox Chase SCID) were either obtained from Jackson laboratories, Charles River, or generated at the Stanford University Research Animal Facility as described previouslyl². Animals were housed at the Helmholtz Center Animal Facility rooms were maintained at constant temperature and humidity with a 12-h light cycle. Animals were supplied with food and water ad libitum. All animal experiments were reviewed and approved by the Government of Upper Bavaria and registered under the projects 55.2-1-54-2532-61-2016 and 55.2-2532-02-19-23 and conducted under strict governmental and international guidelines. This study is compliant with all relevant ethical regulations regarding animal research. Cre-positive (Cre⁺) animals from double-transgenic reporter mice were identified by detection of relevant fluorescence in the dorsal dermis. Genotyping was performed to distinguish mouse lines containing a 200-base pair (bp) Cre fragment (Cre^{+/-}) from the wild-type (Cre^{-/-}). Genomic DNA from the ear-clips was extracted using QuickExtract DNA extraction solution (Epicenter) following the manufacturer's guidelines. DNA extract (1 μl) was added to each 24 μl PCR. The reaction mixture was set up using Taq PCR core kit (Qiagen) containing 1× coral buffer, 10 mM dNTPs, 0.625units Taq polymerase, 0.5 μM forward primer “Cre_genotype_4F”-5′ ATT GCT GTC ACT TGG TCG TGG C-3′″ (SEQ ID NO: 2, Sigma) and 0.5 μM reverse primer “Cre_genotype_4R”-5′ GGA AAA TGC TTC TGT CCG TTT GC-3′ (SEQ ID NO: 3, Sigma). PCRs were performed with initial denaturation for 10 min at 94° C., amplification for 30 cycles (denaturation for 30 s at 94° C., hybridization for 30 s at 56° C., and elongation for 30 s at 72° C.) and final elongation for 8 min at 72° C., and then cooled to 4° C. In every experiment, negative controls (non-template and extraction) and positive controls were included. The reactions were carried out in an Eppendorf master cycler. Reactions were analyzed by gel electrophoresis.

Viral Particle Production.

Adeno-associated virus serotype 6 (AAV6) expressing GFP or Cre recombinase were produced by transfecting the AAVpro® 293T Cell Line (Takara Bio, 632273) with pAAV-U6-sgRNA-CMV-GFP (Addgene, 8545142) or pAAV-CRE Recombinase vector (Takara Bio, 6654), pRC6 and pHelper plasmids procured from AAVpro Helper Free System (Takara Bio, 6651). Transfection was performed with PEI transfection reagent and vires were harvested 72 h later. AAV6 viruses were extracted and purified with an AAVpro® purification kit (Takara Bio, 6666) and titer was calculated using real-time PCR.

Human Skin Samples.

Fresh human skin and scar biopsies, from various anatomic locations, were collected from donors between 18-65 years of age, through the Section of Plastic and Aesthetic Surgery, Red Cross Hospital Munich (reference number 2018-157), and by the Department of Dermatology and Allergology, Klinikum rechts der Isar Technical University Munich (reference number 85/18S). Informed consent was obtained from all subjects prior to skin biopsies. Upon collection, these samples were directly processed for tissue culture or fixed with PFA and then processed for cryosection or paraffin section followed by histological or immunofluorescent analyses.

Fascia In Vitro Culture.

Two in vitro systems were used. To visualize the changes in matrix architecture in real time, 2 mm-diameter biopsies were excised from P0 C57BL/6J neonates and processed for live imaging (SCAD assay, Patent Application No. PLA17A13). To determine the effectiveness of the DT treatment, muscle+fascia was manually separated from the rest of the skin in the chimeric grafts experiments and incubated with DT at different concentrations for 1 h at ambient temperature. Next, samples were washed with PBS and incubated in DMEM/F12 (Thermo Fisher) supplemented with 10% Serum (Thermo Fisher), 1% penicillin/streptavidin (Thermo Fisher), 1% GlutaMAX (Thermo Fisher) and 1% non-essential amino acids solution (Thermo Fisher) in a 37° C., 5% CO₂ incubator. Medium was routinely exchanged every other day. Samples were fixed at day 6 of culture with 2% paraformaldehyde and processed for histology.

Histology.

Tissue samples were fixed overnight with 2% paraformaldehyde in PBS at 4° C. Samples were rinsed three times with PBS, embedded in optimal cutting temperature (OCT, Sakura Finetek) and flash-frozen on dry ice. 6-micron sections were made in a Cryostar™ NX70 cryostat (Thermo fisher). Masson's trichrome staining was performed with a Sigma-Aldrich Trichrome stain kit, according to the manufacturer's guidelines. For immuno-labeling, sections were air-dried for 5 min and fixed with −20° C.-chilled acetone for 20 min. Sections were rinsed three times with PBS and blocked for 1h at room temperature with 10% serum in PBS. Then, the sections were incubated with primary antibody in blocking solution for 3h at ambient temperature. Sections were then rinsed three times with PBS and incubated with secondary antibody in blocking solution for 60 min at ambient temperature. Finally, sections were rinsed three times in PBS and mounted with fluorescent mounting media with 4,6-diamidino-2-phenylindole (DAPI). Primary antibodies used: goat-anti-αSMA (1:50, Abcam), rabbit-anti-TUBB3 (1:100, Abcam), rat-anti-THY1(CD90) (1:100, Abcam), rat-anti-CD24 (1:50, BD biosciences), rabbit-anti-DPP4(CD26) (1:150, Abcam), rabbit-anti-PECAM1(CD31) (1:10, Abcam), rat-anti-CD34 (1:100, Abcam), rabbit-anti-COLLAGEN I (1:150, Rockland), rabbit-anti-COLLAGEN III (1:150, Abcam), rabbit-anti-COLLAGEN VI (1:150, Abcam), rabbit-anti-DLK1 (1:200, Abcam), rat-anti-ERTR7 (1:200, Abcam), rat-anti-F4/80 (1:400, Abcam), rabbit-anti-LYVE1 (1:100, Abcam), rat-anti-MOMA2 (1:100, Abcam), goat-anti-PDGFRA (1:50, R & D systems), rat-anti-LY6A(Sca1) (1:150, Biolegend), rat-anti-CD44 (1:100, Abcam), rabbit-anti-NOV/CCN3 (1:20, Elabscience), sheep-anti-FAP (1:100, R&D systems). PacificBlue-, AlexaFluor488-, AlexaFluor568, or AlexaFluor647-conjugated antibodies (1:500, Life technologies) against suitable species were used as secondary antibodies.

Microscopy.

Histological sections were imaged using a using a ZEISS AxioImager.Z2m (Carl Zeiss). For whole-mount 3D imaging of wounds, fixed samples were embedded in 35-mm glass bottom dishes (Ibidi) with low-melting point Agarose (Biozym) and left to solidify for 30 min. Imaging was performed using a Leica SP8 multi photon microscope (Leica, Germany). For live imaging of fascia cultures, samples were embedded as just above. Attention was paid to mount the samples with the fascia facing up towards the objective. Imaging medium (DMEM/F-12; SiR-DNA 1:1,000) was then added. Time-lapse imaging was performed over twenty hours under the multi photon microscope. A modified incubation system, with heating and gas control (ibidi 10915 & 11922), was used to guarantee physiologic and stable conditions during imaging. Temperature control was set to 35° C. with 5% CO₂-supplemented air. Second harmonic generation signal and green auto-fluorescence as a reference were recorded every hour. 3D and 4D data was processed with Imaris 9.1.0 (Bitplane) and ImageJ (1.52i). Contrast and brightness were adjusted for better visibility.

Image Analysis.

Histological images were analyzed using ImageJ. For quantification of labeled cells in the fate mapping experiments, the Inventors manually defined the wound, surrounding dermis, and adjacent fascia areas. The Inventors defined the wound as the area flanked by the near hair follicles on both sides, extending from the base of the epidermis down to the level of the hair follicles bulges. Surrounding dermis area was defined as the 200 microns immediately adjacent to the wound bed on both sides. Fascia area was defined as the tissue immediately below the wound. The number of labeled cells in each area was determined by quantifying the particles that were double-positive for DAPI and for the desired label (eg. Dil, GFP, etc) channels. The coverage of the labeled matrix in the wound area was determined by quantifying the area that was double-positive for the labeled matrix and the COLLAGEN I+III+VI staining signal. Cell density of En1^{Cre}; R26^{iDTR} cultures treated with DT was quantified by dividing the total cells (DAPI) by the matrix area (COLLAGEN I+III+VI), Collagens density was calculated as the collagens area coverage of the entire section area. Matrix movements in live imaged cultures were determined by tracking the length of the two furthest points along the sample in both the second harmonic generation (SHG) and auto-fluorescence channels. Length measurements were normalized to the original length at time 0. Wound size was normalized for each time point using the original area at day 0. Scar length was quantified from randomly selected sections taken from the middle of the scar using as a reference the two flanking hair follicles. Relative fluorescence intensity (RFI) was calculated by measuring the mean gray value and normalizes to the dermis images. Fractal analysis was performed using the ImageJ plug-in ‘FracLac’29 (FracLac2015Sep090313a9390) using the same settings and preprocessing as previously described.

Dil Labeling of Fascia in Animals.

Two 5 mm-diameter full-thickness excisional wounds were created on the back of 8-10 weeks old C57BL6/J mice with a biopsy punch. 10-20 μl of the lipophilic “Vybrant™ Dil” dye (Life technologies, V22885) were injected into the exposed fascia directly above the dorsal muscles. Wounded tissue was harvested on day 9 and day 14 post-wounding and processed for histology and imaging by fluorescence microscopy.

Chimeric Skin Transplantations.

Full-thickness 6 mm-diameter biopsies were collected from the back-skin of either R26^{mtmg}, R26^{VT2/GK3}, En1^{Cre}; R26^{mtmg}, En1^{Cre}; R26^{iDTR}, or C57BL6/J adult mice. Using the Panniculus carnosus muscle layer as an anatomical reference, the fascia together with the muscle layer were carefully separated from the dermis and epidermis using Dumont #5 forceps (Fine Science Tools) and a 26G needle under the fluorescent stereomicroscope (M205 FA, Leica). EPFs from fascia+muscle samples of En1^{Cre}; R26^{iDTR} mice were ablated by incubation with 20 pg/ml of diphteria toxin (Sigma-Aldrich, D0564) or only DMEM/F12 as vehicle for 1 h at ambient temperature followed by 3 washing steps with PBS. At this point, the matrix samples were labeled by incubation with 100 μM Alexa Fluor™ NHS Ester (Life technologies, A20006) or Pacific Blue Succinimidyl Ester (Thermo Fisher, P10163) in PBS for 1 h at ambient temperature followed by 3 washing steps with PBS. Chimeras were made by placing the epidermis+dermis portion of a mouse strain on top of the muscle+fascia of another strain and left to rest for 20 min at 4° C. inside a 35 mm culture dish with 2 ml of DMEM/F12. Special attention was paid on preserving the original order of the different layers (Top to bottom: Epidermis->Dermis->Muscle->Fascia). Then, a 2 mm “deep” full-thickness was excised from the chimeric graft using a biopsy punch in the middle of the biopsy. To create “superficial” wounds, the 2 mm excision was done only in the epidermis+dermis half, prior to reconstitution with the bottom part. “Wounded” chimeric grafts were then transplanted into freshly-made 4 mm-diameter full-thickness excisional wounds in the back of either RAG2−/− or Fox Chase SCID immunodeficient 8-10 weeks-old mice. Precautions were taken to clean out the host blood from the fresh wound before the transplant and to leave the graft to dry for at least 20 min before ending the anesthesia, to increase the transplantation success. To prevent mice from removing the graft, a transparent dressing (Tegaderm, 3M) was placed on top of the grafts.

In Situ Matrix Tracing and EdU Pulses.

C57BL6/J mice received subcutaneous 20 microliter injections of 10 mg/mi FITC NHS ester in physiological saline with 0.1M sodium bicarbonate pH9 (46409, Life technologies) four and two days before wounding. At 2, 6, or 13 days post-wounding, mice received 200 μl i.p. injections of 1 mg/ml EdU in PBS. Samples were collected 24 hours after the EdU pulse and processed for cryosection and imaging by fluorescence microscopy.

Flow Cytometry.

Fascia and dermis were physically separated from the back-skin of C57BL6/J or En1^{Cre}; R26^{mTmG} mice under the fluorescence stereomicroscope as before. Harvested tissue was minced with surgical scissors and digested with an enzymatic cocktail containing 1 mg/ml Collagenase IV, 0.5 mg/ml Hyaluronidase, and 25 U/ml DNase I (Sigma-Aldrich) at 37° C. for 30 min. The resulted single cell suspension was filtered and incubated with conjugated/unconjugated primary antibodies (dilution 1:200) at 4° C. for 30 min, followed by an incubation with a suitable secondary antibody when needed at 4° C. for 30 min. Cells were washed and stained with Sytox blue dye (dilution 1:1000. Life technologies, S34857) for dead cell exclusion. Cells were subjected to flow cytometric analysis using a FACSAria III (BD Bioscience). Primary antibodies used: anti-DLK1 (Abcam), anti-CD9 (Santa Cruz), anti-CD271(LNGFR) (Miltenyi), anti-F4/80 (Abcam), AlexaFluor790-anti-NG2 (Santa Cruz), FITC-anti-DPP4(CD26) (eBioscience), PerCP-eFluor710-anti-ITGB1(CD29) (eBioscience), anti-CD34 (Abcam), PerCP-Cy5.5-anti-CD24 (eBioscience), APC-Fire750-anti-CD34(Biolegend), APC-anti-ITGA7 (R&D systems), PerCP-Cy5.5-anti-LY6A(Sca1) (eBioscience), PE-Vio770-anti-PDGFRA (Miltenyi), PerCP-Vio700-anti-CD146 (Miltenyi), APC-anti-PECAM1(CD31) (eBioscience), eFluor660-anti-LYVE1 (Thermo fisher), APC-LY76(TER119), APC-anti-EPCAM(CD326), and APC-anti-PTPRC(CD45). Secondary antibodies used: AlexaFluor488 Goat anti-Rabbit (Life technologies), AlexaFluor568 Goat anti-Rat (Life technologies).

Scanning Electron Microscopy.

Skin biopsies of adult C57BL6/J mice were collected, and the fascia was manually separated as before. Samples were then fixed overnight with paraformaldehyde and glutaraldehyde, 3% each, in 0.1% sodium cacodylate buffer pH 7.4 (Electron Microscopy Sciences, Germany). Samples were dehydrated in gradual ethanol and dried by the critical-point method, using CO2 as the transitional fluid (Polaron Critical Point Dryer CPC E3000; Quorum Technologies) and observed by scanning electron microscopy (JSM 6300F; JEOL, Germany).

ePTFE Membrane Implants.

Two 6 mm-diameter full-thickness excisional wounds were created with a biopsy punch on the back of 8-week old En1^{Cre}; R26^{VT2/GK3} or C57BL6/J mice. Sterile 8 mm-diameter ePTFE impermeable membranes (Dualmesh®, GORE®) were implanted between the surrounding skin and the dorsal skeletal muscle underneath, to cover the open wound on the right side. For this, the surrounding skin was loosen using Dumont #5 forceps and spatula (10090-13, Fine Science Tools). The dual-surface membrane was implanted with the attaching face facing out, so to promote dermal cell attachment, while the smooth surface was in direct contact with the fascia. The left sham control wound underwent the same procedure without implanting any membrane. Each wound was photographed at indicated time points, and wound areas were measured using ImageJ. Wound sizes at any given time point after wounding were expressed as percentage of initial (day 0) wound area. At 7 or 63 days post-wounding, samples were collected and processed for histology.

Released Fascia Injury in Adult Mice.

Two 5 mm-diameter full-thickness excisional wounds were created with a biopsy punch on the back of 8-week old male C57BL6/J mice. The skin around the wound on the left side was separated from the underneath skeletal muscle using a sterilized gold-plated 3×5 mm genepaddles (Harvard Apparatus, #45-0122) to release the fascial layer. The right wound served as a control. Each wound was digitally photographed at indicated time points, and wound areas were measured using Photoshop (Adobe Systems, San Jose, Calif.). Wound sizes at any given time point after wounding were expressed as percentage of initial (day 0) wound area. The harvested tissue at the indicated time points was processed for cryosection and Masson's trichrome staining for histology.

Fascial Cells Ablation with AAV6-Cre Viral Particles and DT Treatment in Pups.

Two 3 mm-diameter full-thickness excisional wounds were created with a biopsy punch on the back of postnatal day 11 (P11) R26^{iDTR} mice. 20 μl of Cre-expressing adeno-associated virus type 6 (AAV6-Cre) or control eGFP-expressing AAV6 (AAV6-EGFP) at viral titre of 5×10¹¹/ml were injected subcutaneously at the area between the two wounds. Diphtheria toxin (DT) solution at 1 ng/μl in PBS was intraperitoneally injected to each mouse once per day for 7 days at the dosage of 5 ng/g. Tissue was harvested on 7 days after wounding.

Statistics.

All plots depict mean value and error bars represent SEM. Statistical analyses were performed using GraphPad Prism software (version 6.0, GraphPad). Statistical test and p values are specified in the figure legends and in the corresponding plots. For ease, p values below 0.0001 were stated equal as 0.0001.

### Example 1: Wound Fibroblasts, Vasculature, Macrophages and Nerves are Derived from Subcutaneous Fascia

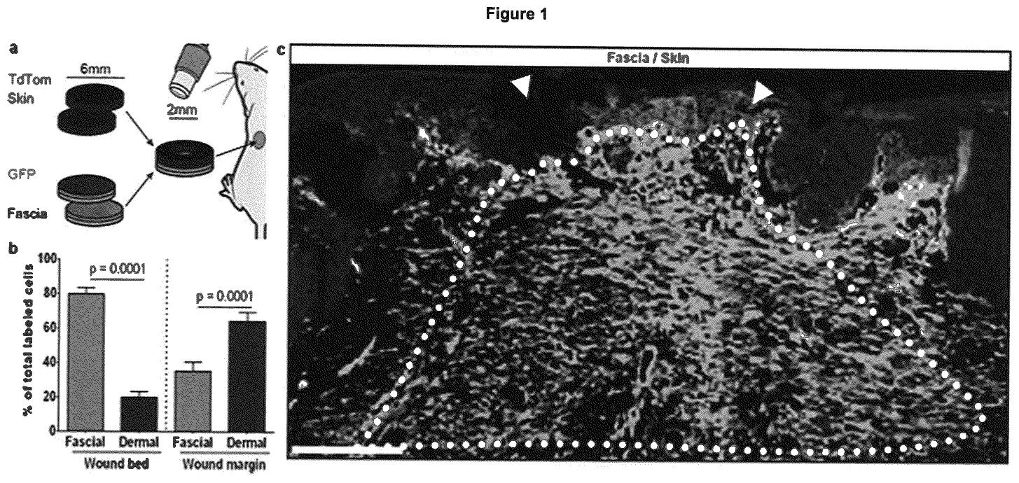

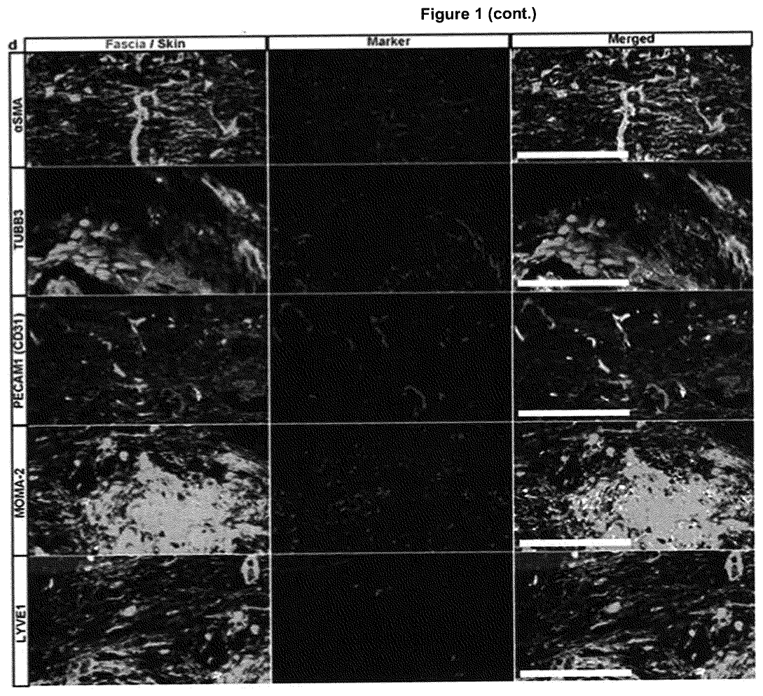

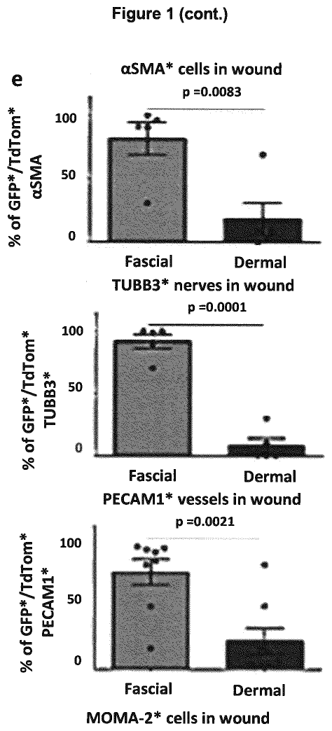

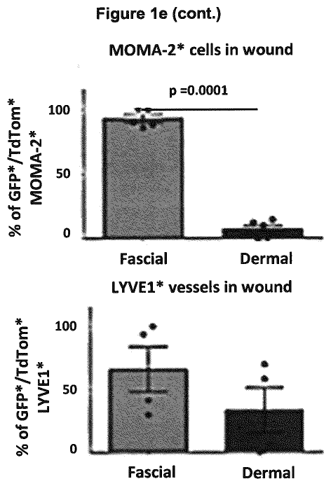

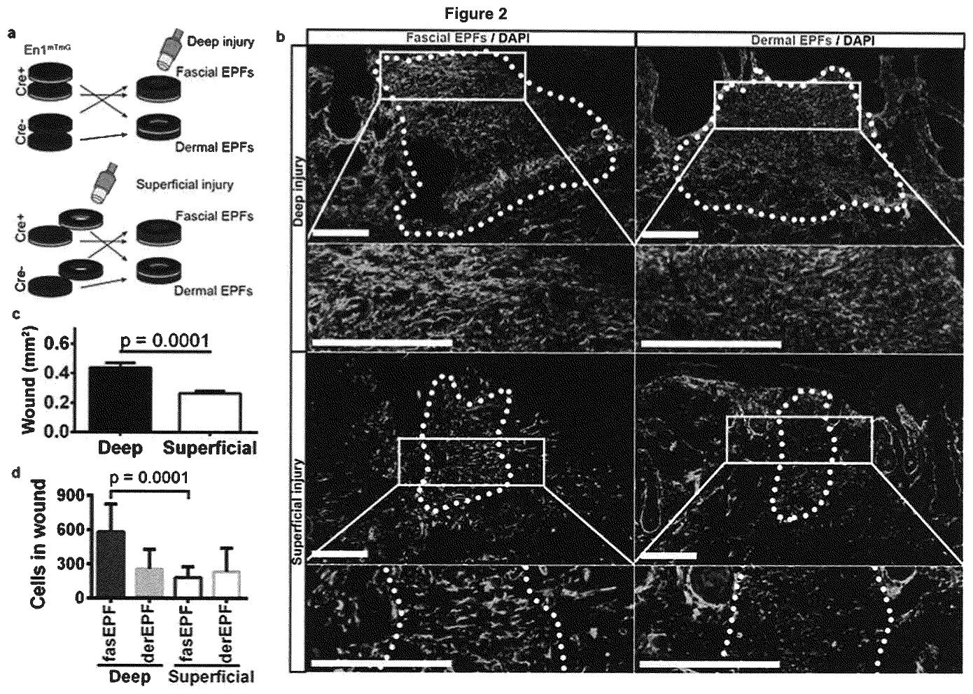

To map the origins of all cells that contribute to wounds the Inventors developed a fate mapping technique by transplanting chimeric, skin and fascia, grafts into living animals (FIG. 1a and see ‘Methods’). The Inventors harvested fascia from mice that constitutively express GFP in all their cells and separately harvested skin from mice that constitutively express TdTomato and reassembled the TdTomato⁺ skin over the GFP⁺ fascia. The Inventors made a full thickness wound in the middle of the graft, then transplanted the entire chimeric tissue into backs of adult mice. In these transplantation experiments, the entire cellular contribution in host wounds could be observed from donor fascia or dermis, by analyzing the relative presence of GFP⁺ or TdTomato⁺ cells respectively.

At 14 days post wounding (dpw), 80.04±3.443% of the labeled cells in the wound bed were GFP⁺, indicating a fascia origin (FIG. 1b). GFP⁺ fascia-derived cells clogged up the entire wound and bordered the newly regenerated epidermis that covered the wound (FIG. 1c). The cells from the fascia populated the surrounding dermis as well, making up 35.46±4.938% of the total labeled cells within a 0.2 mm radius around the wound (FIG. 1b-c). 81.63±12.84% of the αSMA⁺ wound fibroblasts derived from the fascia, and more strikingly, nerve, endothelial, and macrophages within wounds were also predominantly of fascia origin (Table 1, FIG. 1d-e).

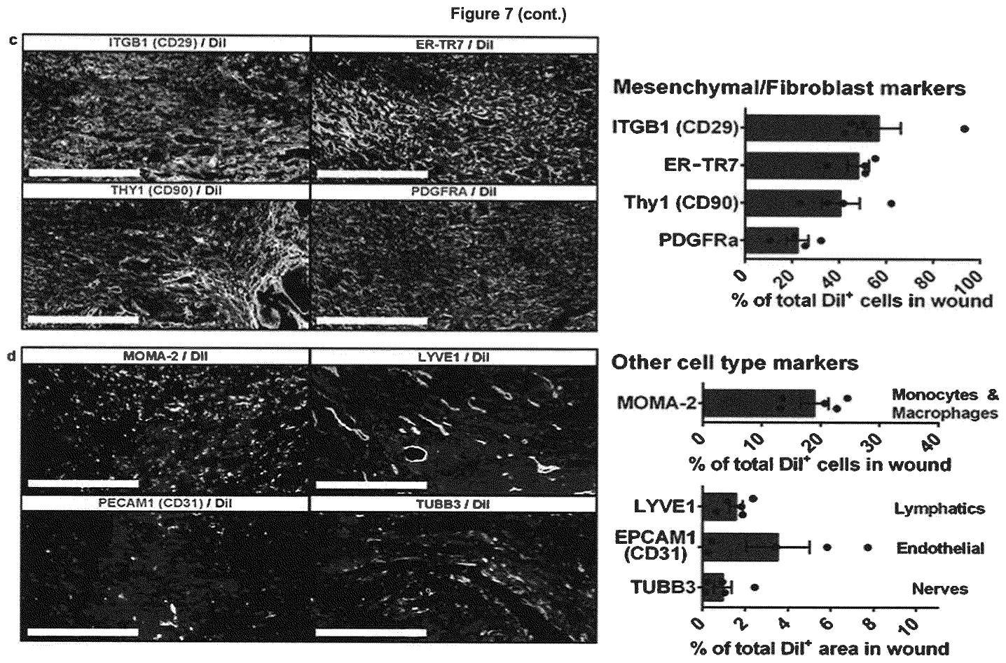

Next the Inventors took an independent in vivo labeling approach by injecting Dil-dye directly into the fascia (see ‘Methods’ and FIG. 7a). Dil-labeled cells populated the wounds and surrounding dermis at 14 dpw, similar to the chimera grafts experiments, whereas in uninjured controls, labeled cells remained in the fascia (FIG. 7b). Up to 56.71±9.319% of total fascia-derived cells in wounds were fibroblasts that expressed ITGB1, ER-TR7, THY1, or PDGFRα (FIG. 7c). Up to 18.94±2.371% of fascia-derived cells in wounds were monocytes/macrophages, with fascia additionally contributing to wound lymphatics, endothelium and nerves (FIG. 7d).

Collectively, the two-independent fate-mapping approaches demonstrate that fascia is the major reservoir for the fibroblasts, endothelial, macrophages, and peripheral nerves that populate wounds at dermal surfaces.

### Example 2: Scar Severity Depends on how Many Fibroblasts Rise from the Fascia

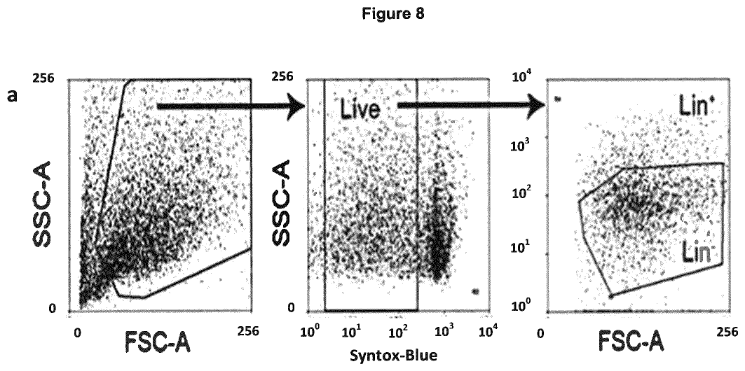

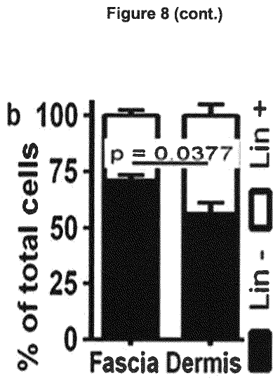

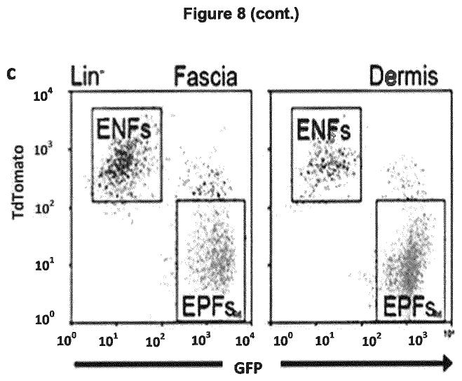

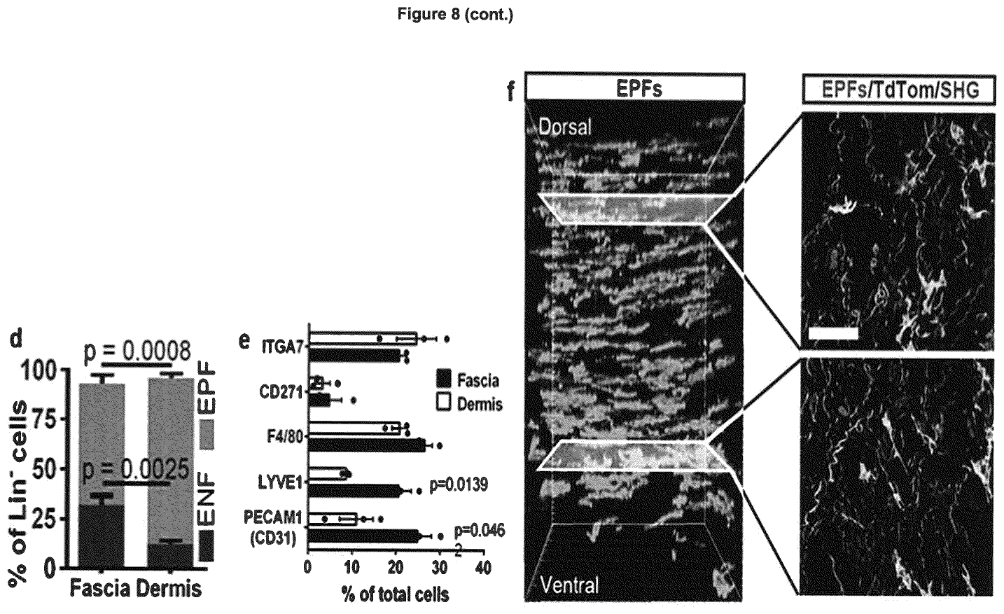

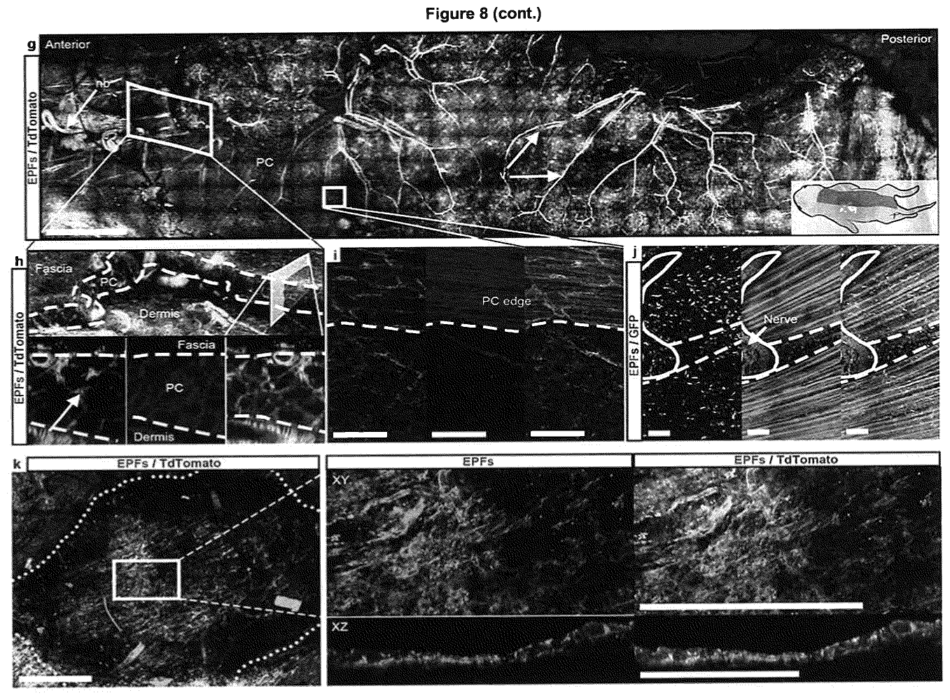

The inventors previously showed that all scar-forming fibroblasts express Engrailed-1 (En1) early on in embryogenesis and the Inventors refer to these cells as Ent-lineage positive fibroblasts or EPFs. By crossing the En1-Cre recombinase driver (En1^{Cre}) to a double-color fluorescent reporter (R26^{mtmg}) the Inventors could lineage-trace all GFP⁺ EPFs across dermal and fascial compartments¹²⁻¹³. The Inventors then analyzed the cellular makeup of the fascia compared to dermis using En1^{Cre}; R26^{mTmG} double transgenic mice. Fibroblasts were the predominant fascia cell type (71.1% of the total living cells), while dermis had a significant lower fraction of total fibroblasts (56.4%, FIG. 8a-b). From the total fibroblast fraction, there were two-times more EPFs (GFP⁺) than Engrailed1-naïve fibroblasts (ENFs, TdTomato⁺) in the fascia (61.2% and 31.8% respectively). Whereas in dermis, there was a six-fold excess of EPFs (83.13% and 12.78% for EPFs and ENFs respectively, FIG. 8c-d). Fascia was also enriched in regenerative cell types including endothelial cells (CD31) and lymphatics (Lyve-1), while macrophages (F4/80) and nerve cells (CD271) composition was similar in both fascia and dermis (FIG. 8e). Thus, a higher fibroblast, endothelial, and lymphatic cell content and a lower EPF to ENF ratio distinguishes the fascia from dermis.

The inventors then used two-photon microscopy to generate high-resolution 3D images of the whole back-fascia layer. EPFs were wedged in a specialized multilayered conformation within the fascia. EPFs were aligned in monolayers of consecutive perpendicular sheets across the dorsal-ventral axis (FIG. 8f). Fascial EPFs were present throughout the entire back (FIG. 8g). Side view 3D images showed topographic continuums of EPFs extending from the fascia and traversing the PC muscle (FIG. 8h). Regions where PC muscle layer ended, such as near the limb junctions, showed continuums of EPFs that traverses dermal and fascia layers without clear boundaries (FIG. 8i). Furthermore, similar continuums of EPFs were observed at PC openings where nerve bundles and blood vessels traversed (FIG. 8j). To see if fascial EPFs easily access upper layers, the Inventors generated excisional wounds in En1^{Cre}; R26^{mTmG} adult mice. Aggregates of EPFs surging upwards into open wounds from fissures in the underlying muscle layer were observed after only 3 dpw (FIG. 8k). Collectively, the observation suggests that fascial EPFs easily traverse upper dermal layers during wounding and are unobstructed by the PC muscle that appears porous and easily accessible.

The deeper an injury, the more severe the resulting scar. The Inventors therefore investigated if this correlation can be attributed to fascia by analyzing the extent of fibroblast contributions from the fascia and dermis in deep vs. superficial wounds.

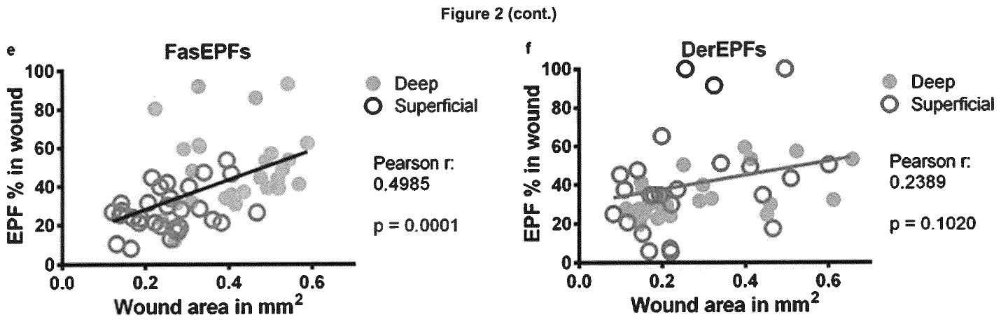

To track the fibroblast contribution even more precisely, the Inventors combined the genetic lineage-tracing approach with anatomic fate-mapping by performing chimeric skin transplants using these mice (En1^{Cre}; R26^{mtmg}). The Inventors used fascia or dermis with traceable EPFs and the untraceable complementary tissue, and then made either a superficial wound through just the dermis and not the fascia below, or a deep excision through both tissues (FIG. 2a).

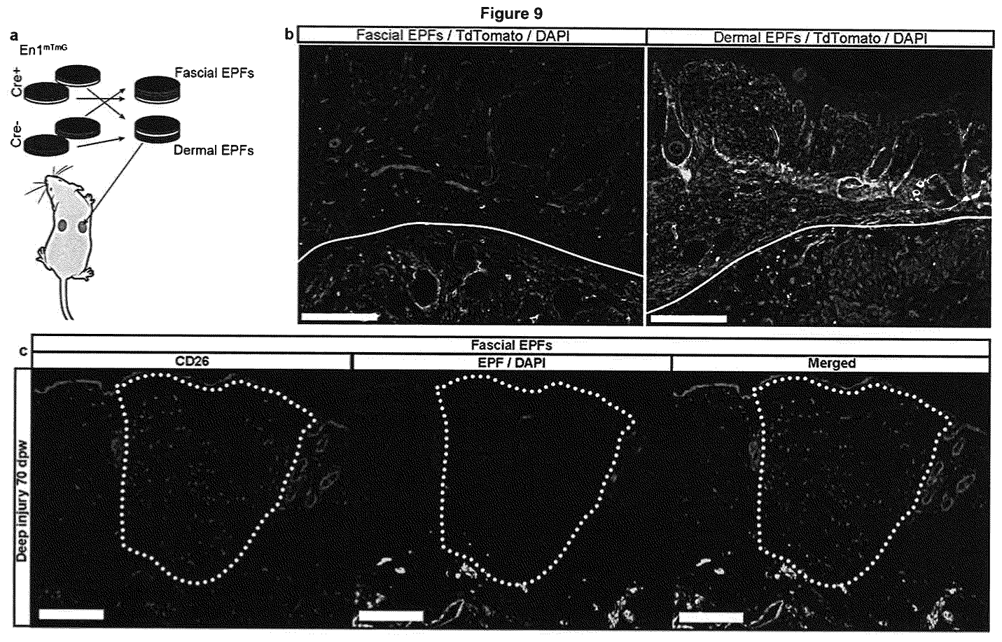

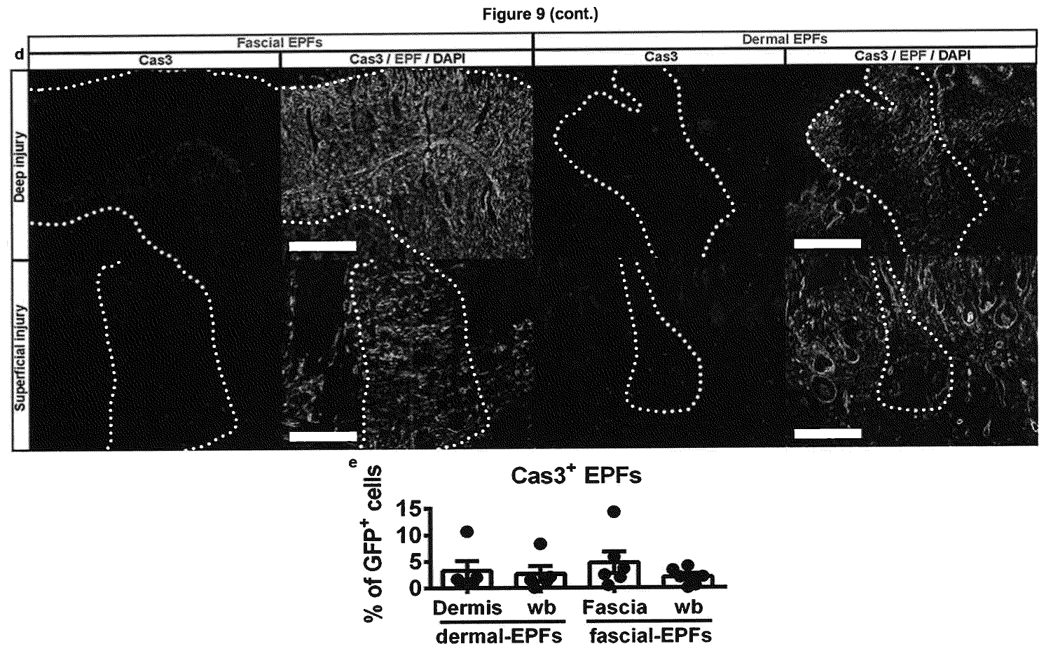

Fourteen days later, wound sizes in deep injuries were 1.7-times larger than superficial injuries (FIG. 2b-c). Fascial EPFs were two-times more numerous in deep wounds compared to superficial wounds, whereas dermal EPFs remained constant in both conditions (FIG. 2d). The abundance of fascial EPFs in the wound directly correlated with wound size and thus scar severity, whereas dermal EPFs showed no correlation (FIG. 2e-f). No crossing between these compartments was observed in uninjured control grafts, indicating that the upward influx of fascial EPFs was triggered by injury (FIG. 9a-b).

To determine the final fate of fascial fibroblasts in wounds, the Inventors performed long-term tracing of chimeric grafts with traceable fascial EPFs. After 10 weeks, fascial EPFs were completely absent from the wound bed (FIG. 9c). The Inventors found a similar and low rate of cell death (<5%) across dermal- and fascial-EPFs at earlier wound stages (FIG. 9d-e), indicating fascia-derived fibroblasts are cleared from mature scars through an apoptosis-independent mechanism.

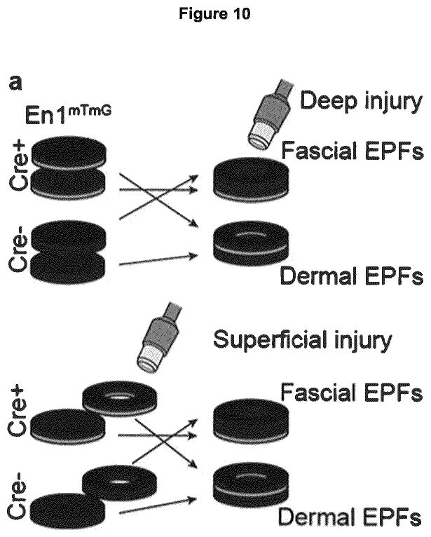

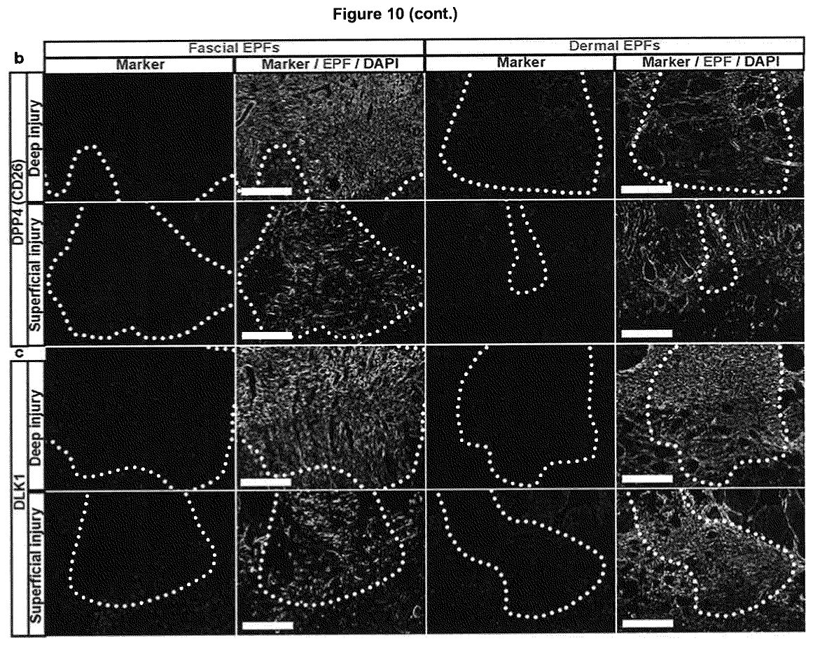

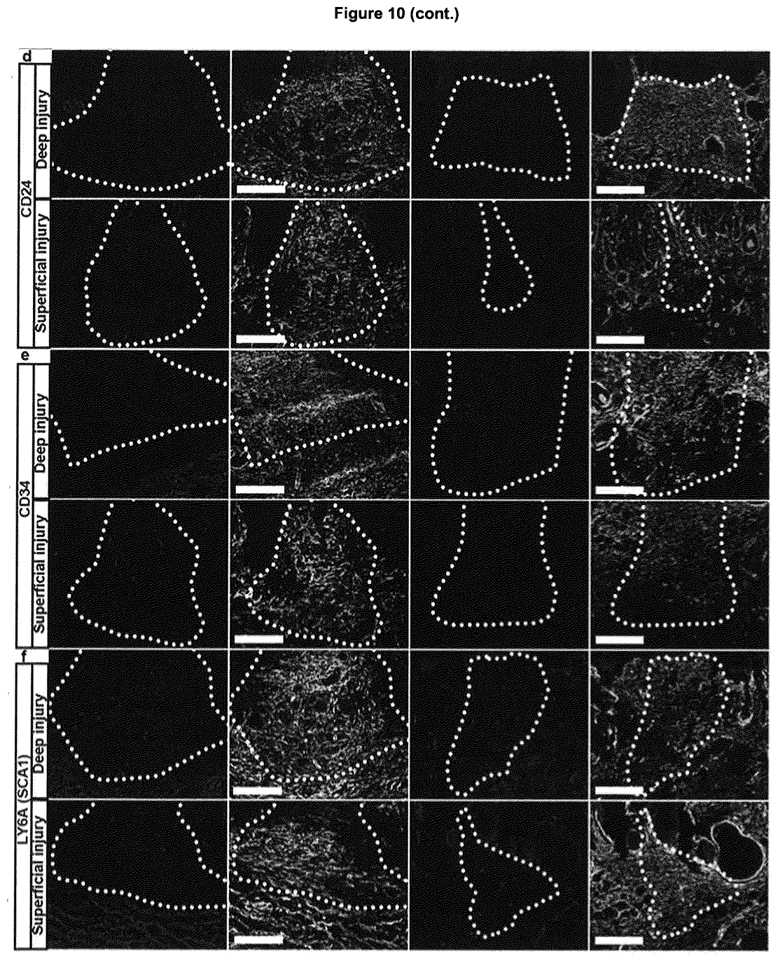

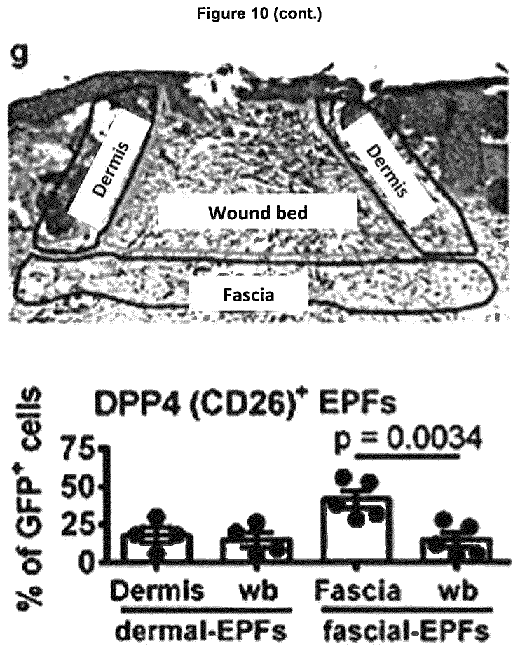

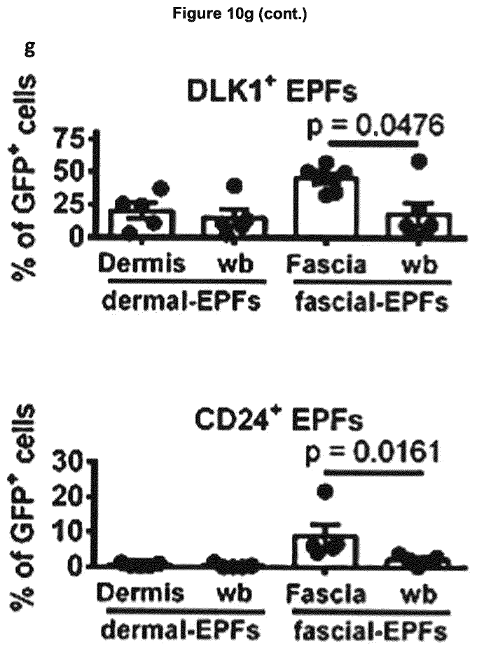

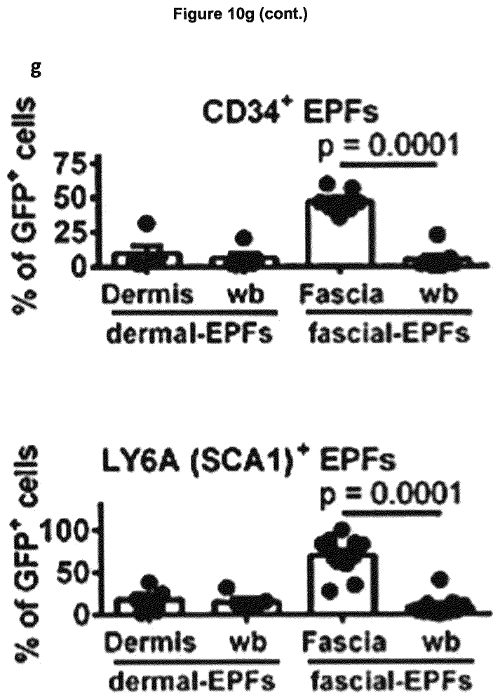

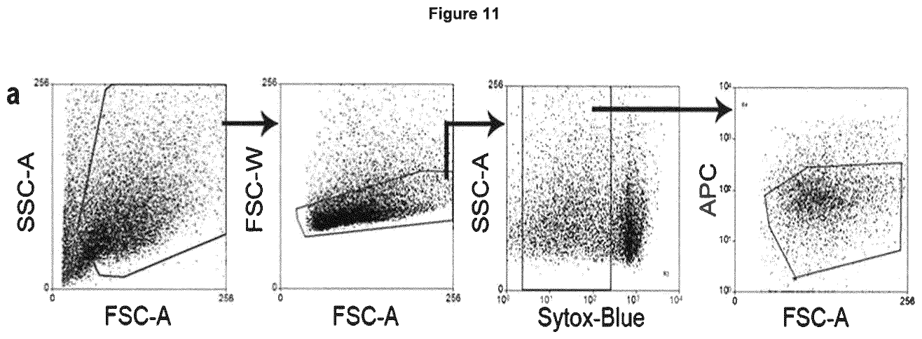

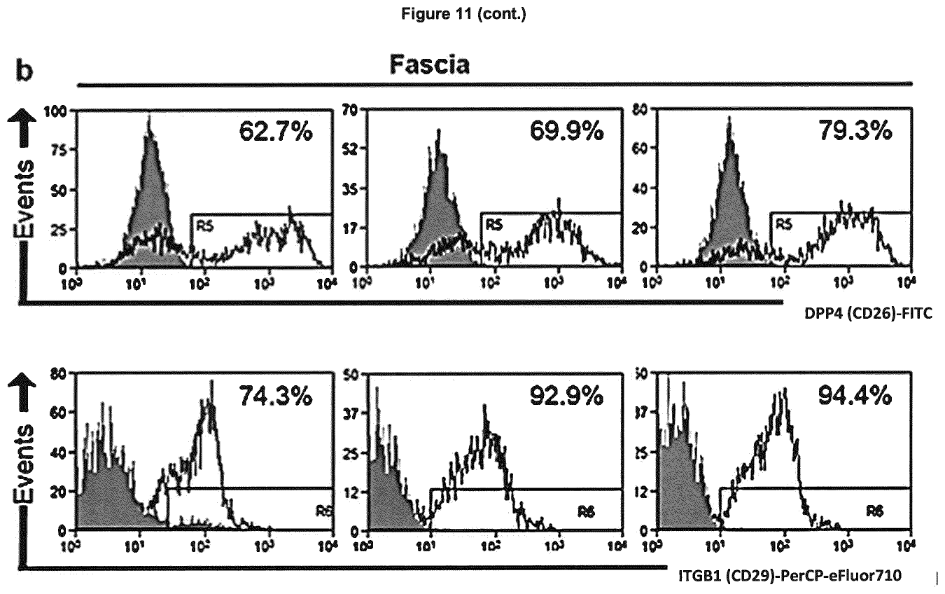

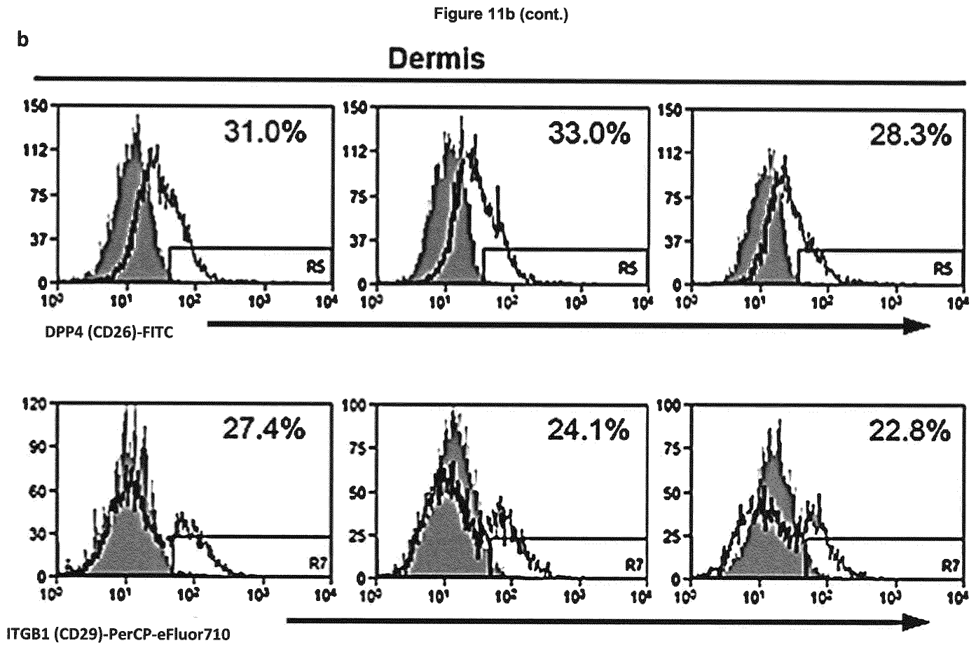

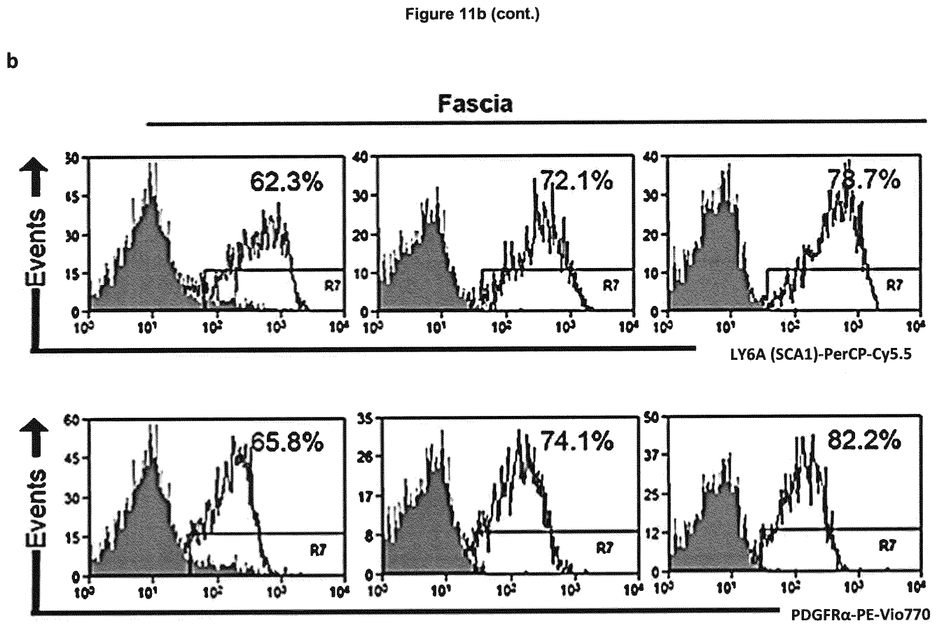

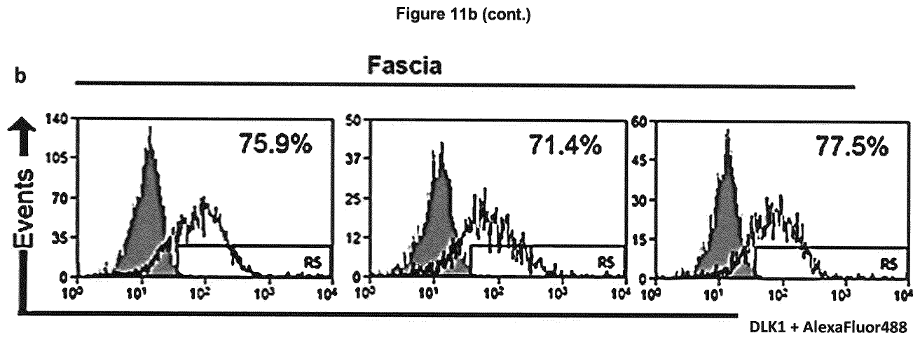

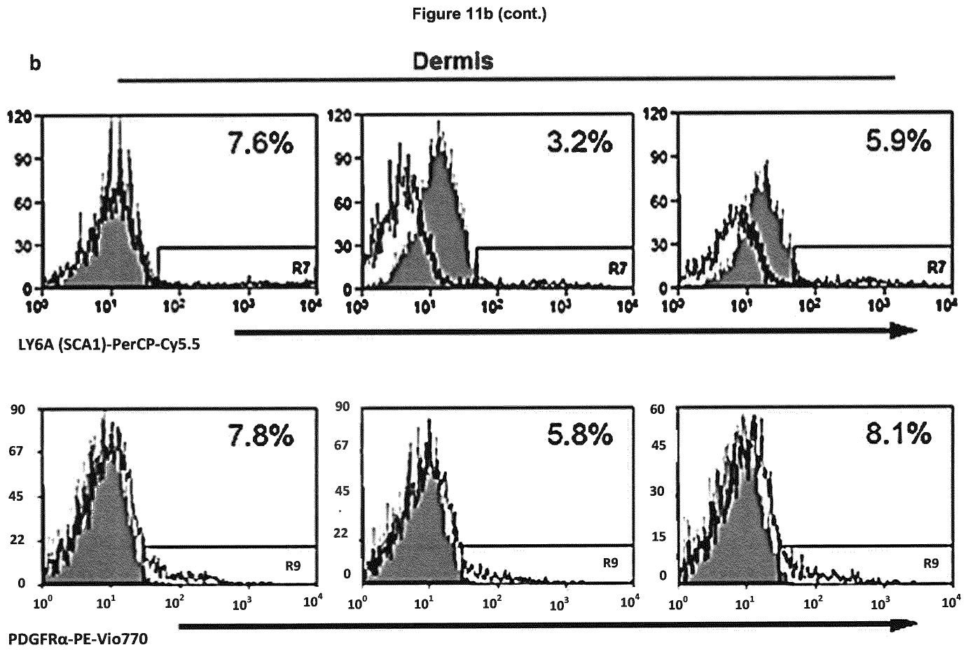

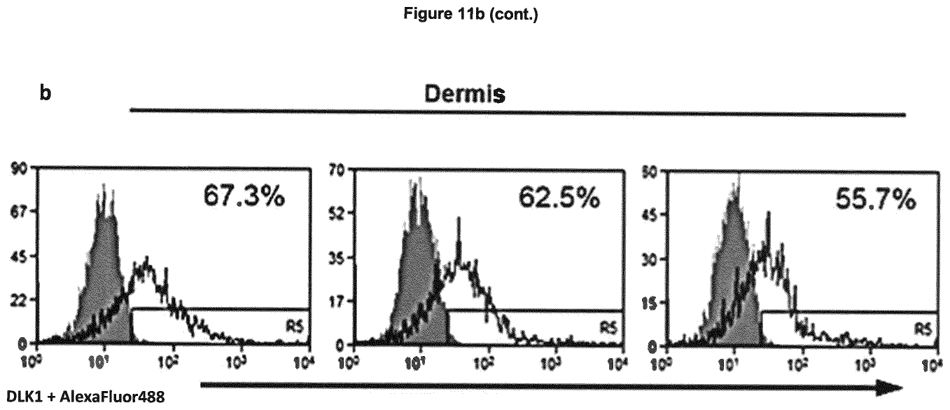

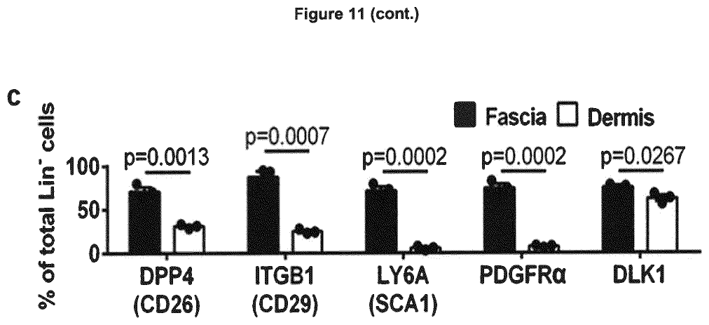

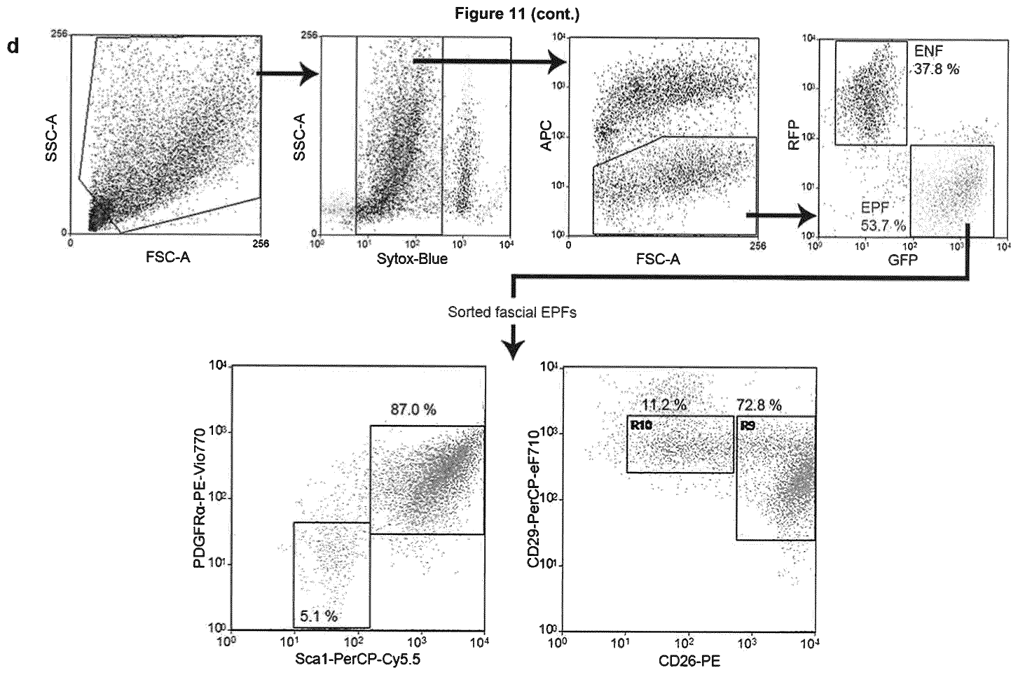

Having established that fascial EPFs are the primary cells that direct and enact wounding, the Inventors sought to place fascial EPFs in the framework of previously reported fibroblast lineage markers by co-immunostaining. Markers previously used to define other sources and lineages of wound fibroblasts CD24, CD34, DPP4 (CD26), DLK1, and LY6A (Sca1) were all more prominent in fascial-EPFs compared to dermal EPFs, and all five markers were surprisingly downregulated upon entering the wounds (FIG. 10). Flow cytometry confirmed the higher DPP4 (CD26), ITGB1 (CD29), LY6A (SCA1), and PDGFRα expression in fascial vs. dermal fibroblasts in uninjured conditions (FIG. 11a-c). Sorted fascial EPFs also revealed low cellular heterogeneity with the predominant population expressing Sca1⁺ PDGFRα⁺ (87.0%) and CD26⁺CD29⁺ (72.8%, FIG. 11d). This broad marker convergence highlights fascial-EPFs as the definitive cell source of wound fibroblasts.

### Example 3: Scars Emerge from Steering of Fascial Matrix

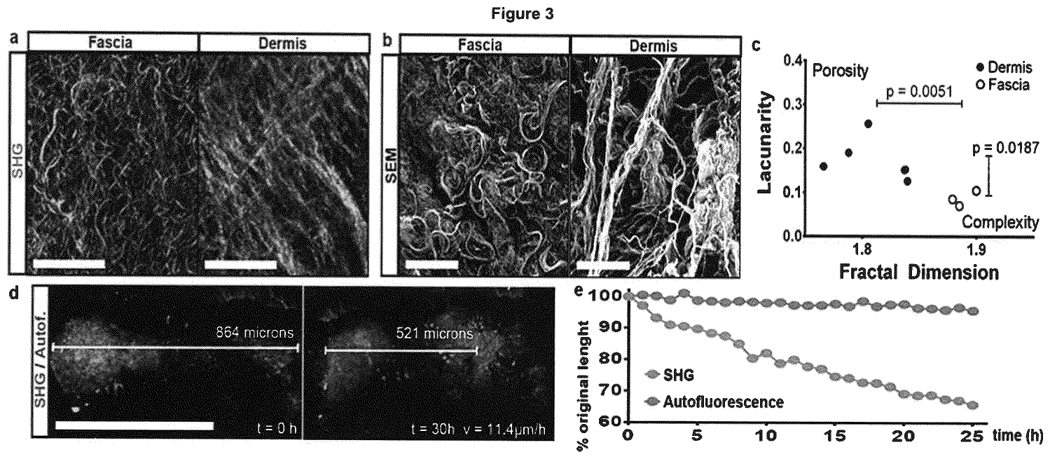

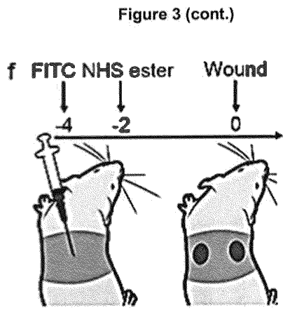

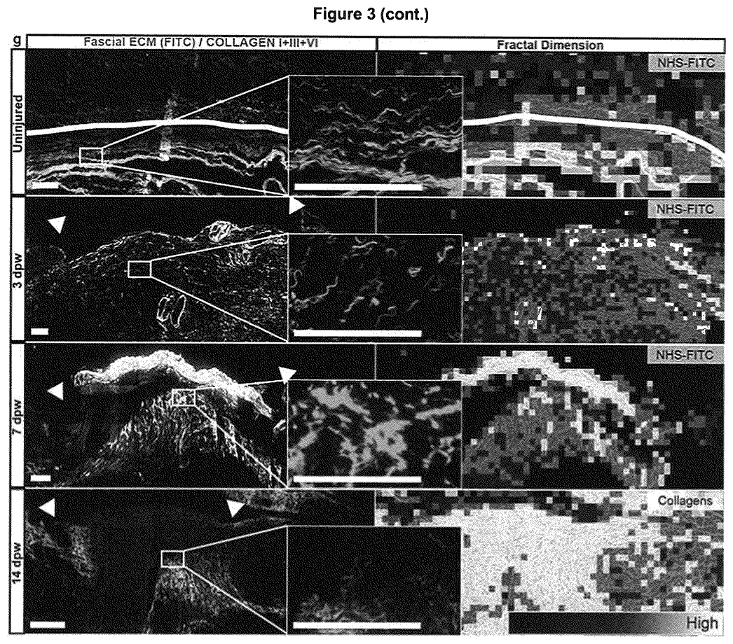

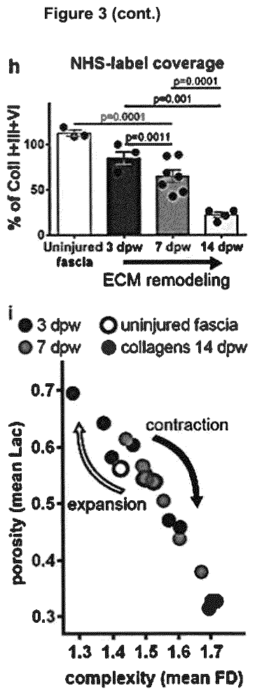

The inventors then looked at the fascia gel itself. Second harmonic generation (SHG) signal and scanning electro-micrographs both revealed profuse collagen fibrils in a coiled arrangement in the fascia, indicative of a relaxed and immature matrix reservoir (FIG. 3a-b). Fractal measurements¹³ of the fiber alignments showed that fascia exhibited a more condensed matrix configuration, with high fractal dimension and low lacunarity values. Dermal matrix on the other hand showed thick collagen fibers that were more stretched and woven (FIG. 3c).

The immaturity of the fascia matrix itself motivated us to check if it could work as a repository for provisional matrix tissue in skin wounds. To answer this question, the Inventors first developed an incubation chamber that enabled live imaging of the fascia matrix over days (see ‘Methods’). Remarkably, recording of SHG signal over 30 hours illuminated steering of the whole matrix across the fascia at a rate of 11.4 μm/hour (FIG. 3d-e). Assuming a similar rate in vivo, the fascial matrix itself could move approximately 2 mm in 7 days, and account for the dynamics of provisional matrix deposition in mammals.

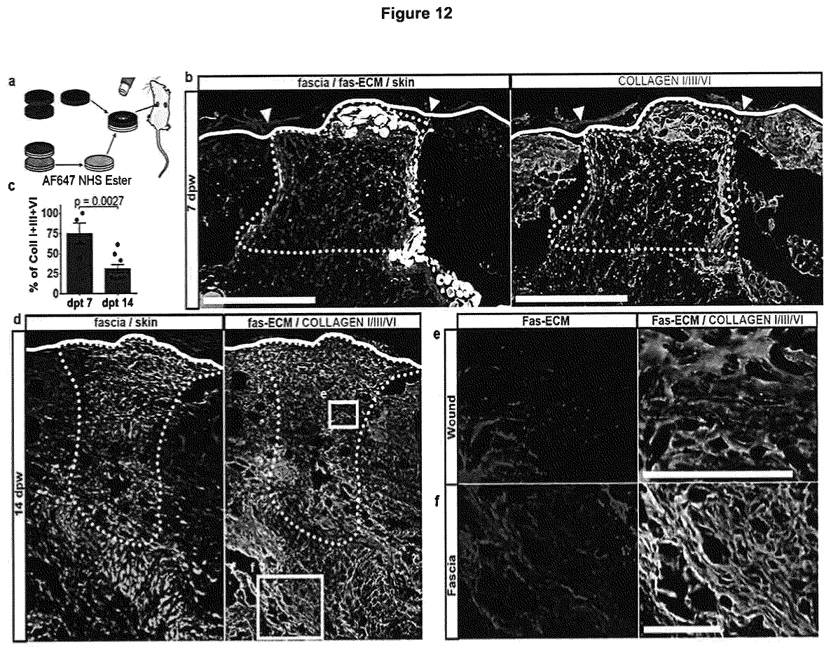

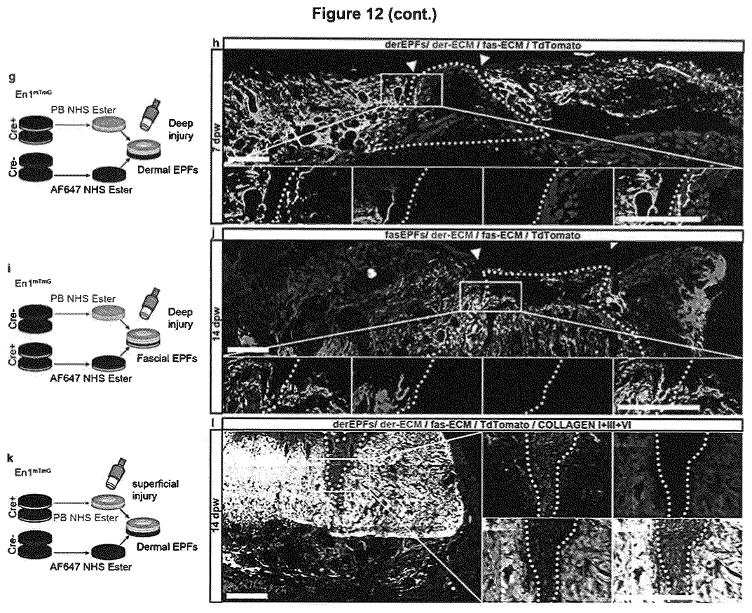

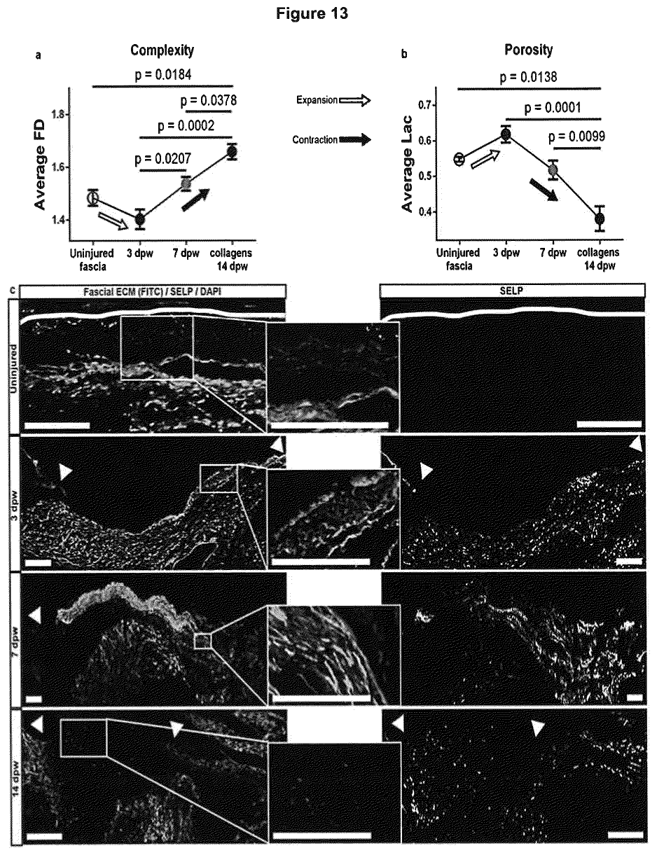

To test if fascia matrix shoots upwards into wounds in vivo, the Inventors developed a technique to trace and fate map the fascia matrix using the chimeric grafts. In this new assay, the Inventors excised the fascia and fluorescently tagged its matrix using an Alexa Fluor 647 NHS Ester. The Inventors combined the labeled fascia with unlabeled wounded dermis and transplanted the chimeric graft into the host back-skin (FIG. 12a). Seven days after wounding, streams of labeled matrix from the fascia extended upwards and plugged the open wounds (FIG. 12b). Quantifications showed that fascia derived matrix covered 74.78±12.94% of total Collagen I, Ill, and VI content in the wound (FIG. 12c). The live imaging and matrix tracing experiments indicated that fascia matrix movements were not a consequence of individual fibroblasts pulling on single fibers. Instead, these were movements of a pliable matrix gel that extended upwards to mold the wound. The Inventors followed the fluorescence label into advanced wound stages and found the fluorescence label decreasing over time in specific regions of the wound bed (FIG. 12d). High magnification images of the shuttled and labeled matrix indicated that the decrease in signal in those regions reflected active remodeling of matrix fibers within advanced wounds (FIG. 12e-f).

The inventors then asked whether dermal matrix can be steered as well. Using the chimera experiments, the Inventors labeled both dermis and fascia with different fluorescent NHS esters. Only the fascia matrix was able to plug open wounds (FIG. 12g-j), whereas dermal matrix was completely immobile. Dermal matrix remained immobile in superficial injuries as well, which healed with de novo matrix deposition (FIG. 12k-l).

To definitively prove that fascia matrix is steered into and clog open wounds, the Inventors labeled the fascia matrix in situ with FITC NHS ester prior to injury (FIG. 3f). Similar to the chimera experiments, the primary matrix within wounds was labeled and thus originated from fascia. Fractal measurements showed that while fascia fibers are normally arranged in parallel sheets, in wound borders, these sheets expand by 3 dpw, forming a highly porous plug with disorganized conformations of matrix fibers (FIG. 3g, i and FIG. 13a-b). Surprisingly, fascia matrix was also present in the eschar seven days after wounding (FIG. 3g). SELP⁺ activated platelets infiltrated and clustered in between fascia matrix fibers at 3 and 7 dpw (FIG. 13c), indicating that platelet activation and coagulation that ends up in the eschar, occurs in parallel with fascia matrix movements. At 7 dpw, fascia labeled fibers within wounds underwent compaction into thicker and more complex fiber arrangement until generating a mature scar matrix architecture (FIG. 3i and FIG. 13a-b). The in situ labeled fascia matrix that covered the wound underwent matrix remodeling at later time points (FIG. 3g-h), similarly to those seen in chimera experiments.

### Example 4: EPFs Steer Fascia Matrix into Wounds

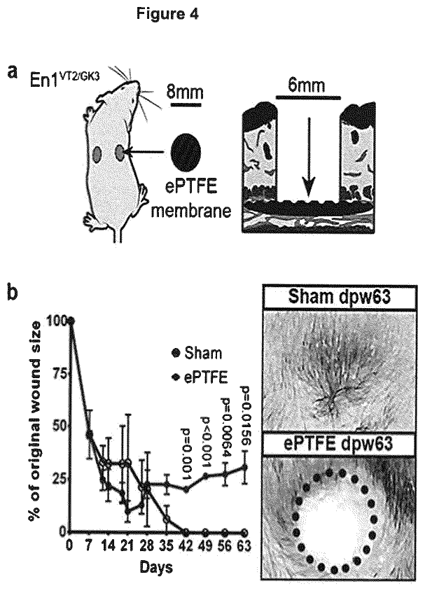

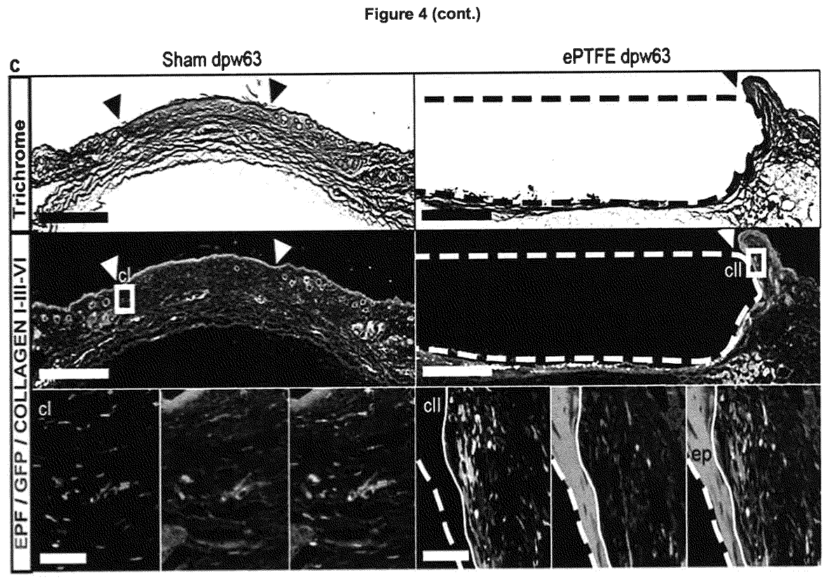

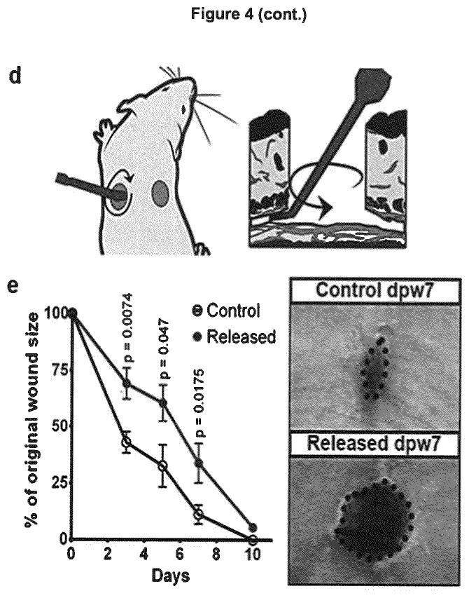

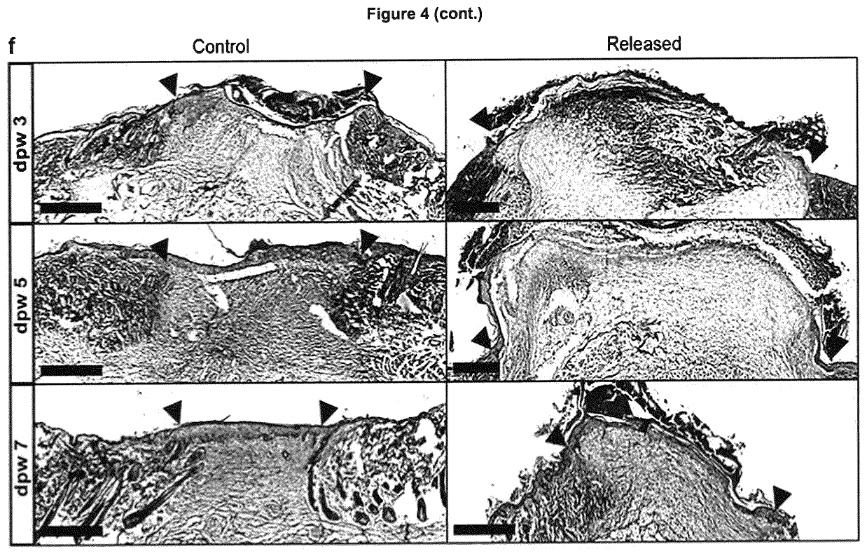

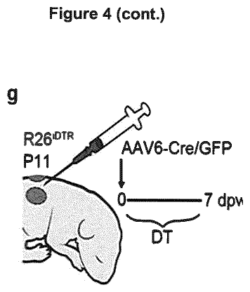

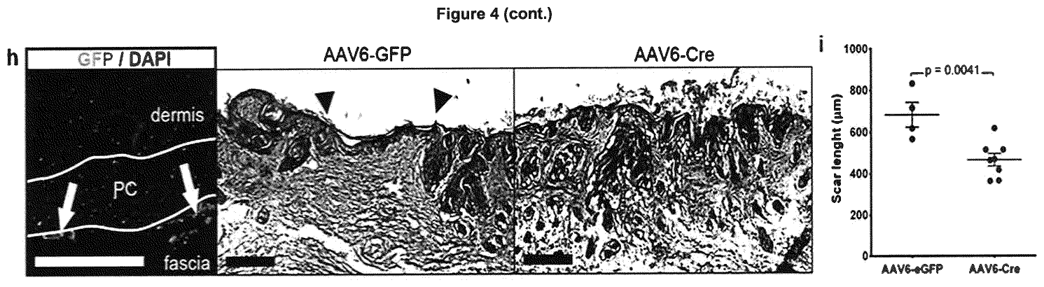

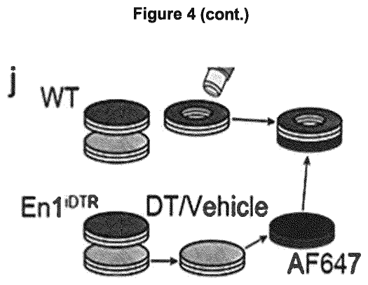

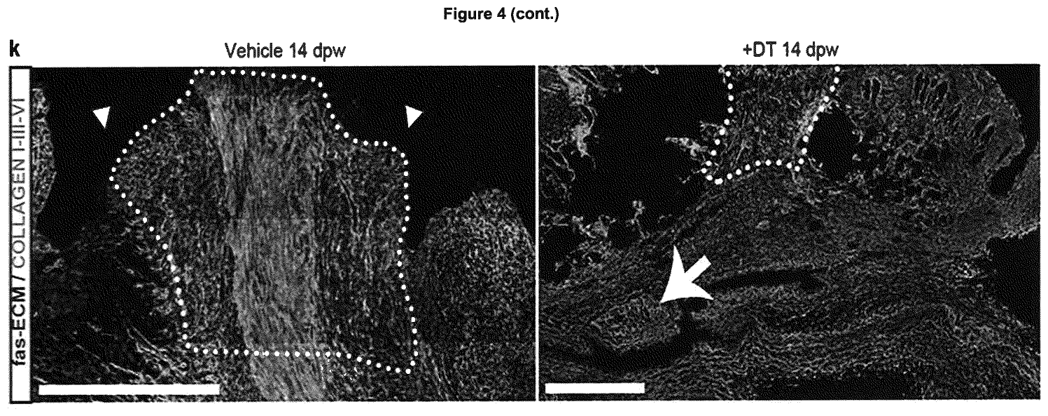

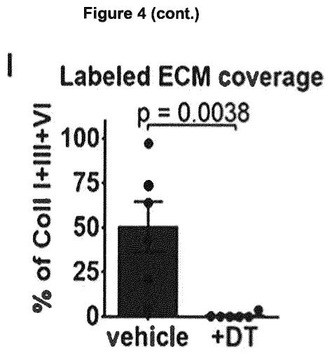

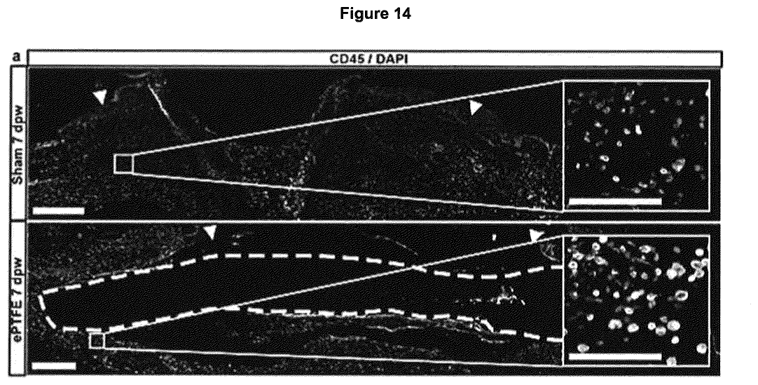

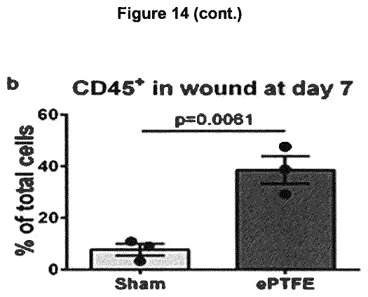

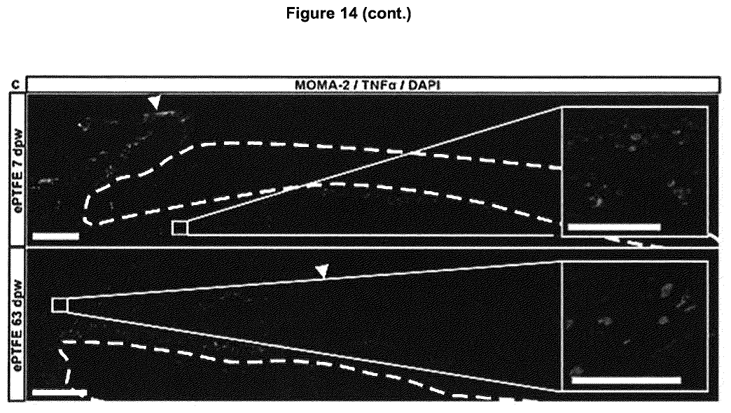

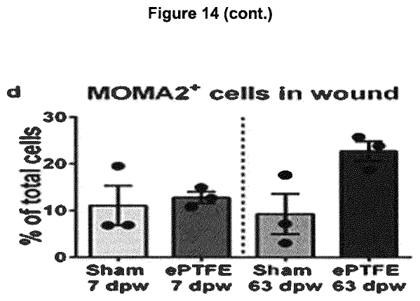

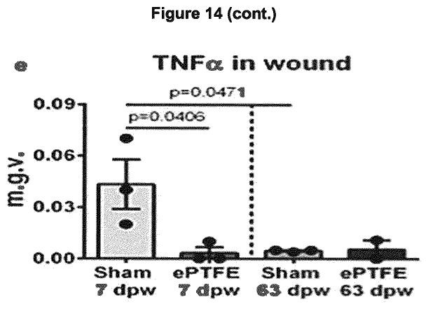

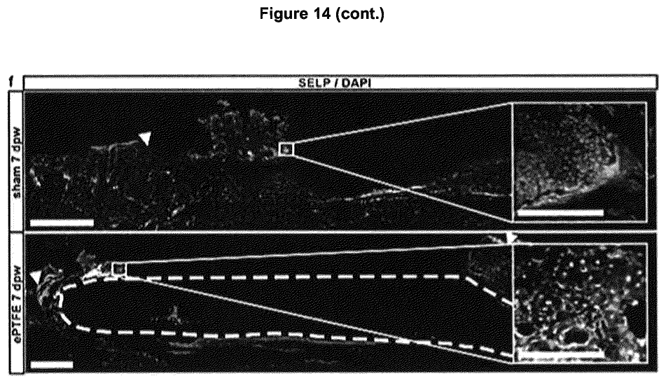

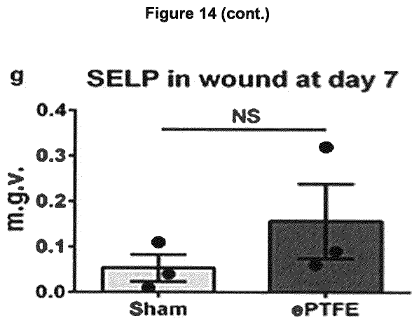

To test if matrix steering from fascia is caused by EPFs, the Inventors generated deep excisional wounds, and physically separated fascia from upper skin by implanting an impermeable dual surface ePTFE membrane between the fascia and the PC muscle layer (FIG. 4a). Due to their non-adhesive nature, ePTFE membranes are routinely used in the clinic to circumvent post-operative adhesions after laparoscopic ventral incisional hernia repairs. Therefore, the dual surface ePTFE membrane was placed with its smooth interface facing downwards thereby preventing fascia cell attachment. Membranes were implanted into wounds of back-skin made in En1^{Cre}; R26^{VT2/GK3} mice (aka. Rainbow mice), in order to document the relative contributions of EPFs from either fascia or dermis. Surprisingly, wounds that were subjected to ePTFE membrane implants remained completely open throughout all time courses of the experiment. Whereas sham controls completely sealed within 21 days, wounds with membrane implants failed to close or contract even two months after wounding (FIG. 4b). Histological sections of harvested wounds showed EPFs trailing from the wound margins under the ePTFE membrane without generating scars, whereas, control wounds developed normal scars (FIG. 4c). The ePTFE membranes did not inhibit immune cell influx (7 dpw, FIG. 14a-b) and exhibited a similar monocytic and macrophage influx to that of control wounds, with low expression of TNFα (FIG. 14c-e). The ePTFE membrane did not affect nor inhibit the coagulation cascade at the border between the dermis and the membrane (FIG. 14f-g). These results indicate that the poor wound healing seen with ePTFE membranes does not reflect chronic inflammation or poor clotting, but a fascia steering blockade that is mediated by the fascia fibroblasts. These findings further support the notion that scars are fascia-derived, since in the absence of fascia movements, dermal-EPFs or dermal matrix are unable to repair wounds with scars.

The inventors then asked whether mechanical separation between dermis and fascia alone, without barrier implants, would affect matrix steering and scar formation. To address this question, the Inventors performed full excisional wounds in wild type mice and physically released the fascia below the PC muscle surrounding the fresh wound (FIG. 4d). Wound closure and scar formation from released-fascia wounds was significantly delayed, and wounds remained open early on similarly to those documented following membrane implantations. Fascia-released wounds eventually closed but with a significant delay (FIG. 4e-f).

To definitively link fascial EPFs to matrix steering into wounds, the Inventors genetically ablated fascial EPFs using two separate strategies. First, the Inventors used a transgenic line that expresses the diphtheria toxin receptor (DTR) in a Cre-dependent manner (R26^{iDTR}). This line allowed us to deplete cells expressing Cre recombinase upon diphtheria toxin (DT) exposure. The Inventors thus generated Cre-expressing adeno-associated viral particles (AAV6-Cre) and injected them into the fascia of R26^{iDRT} pups underneath freshly made full excisional wounds (FIG. 4g). Scar size from AAV6-Cre transduced mice treated with DT were significantly smaller than controls (AAV6-eGFP, FIG. 4h-i).

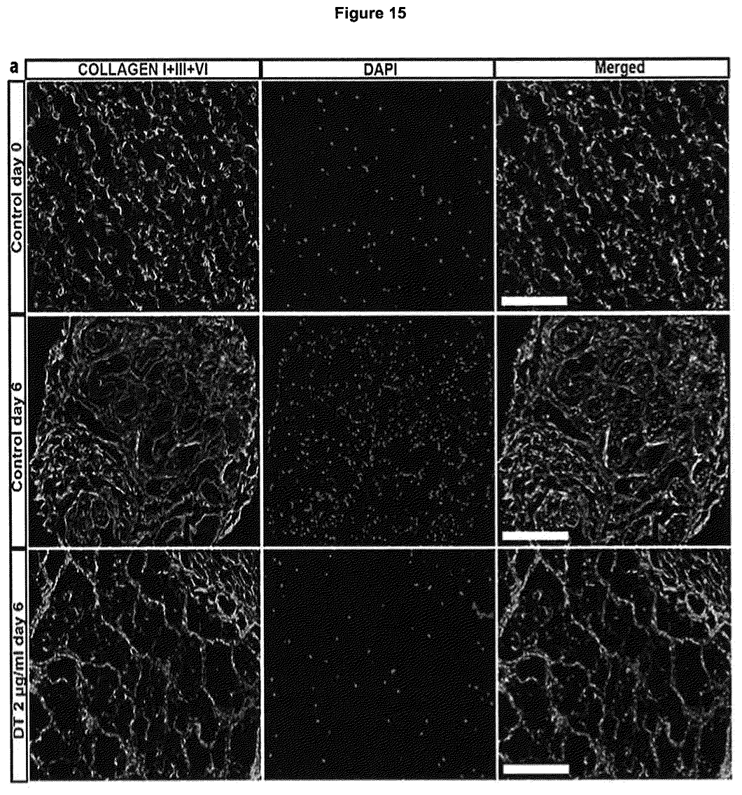

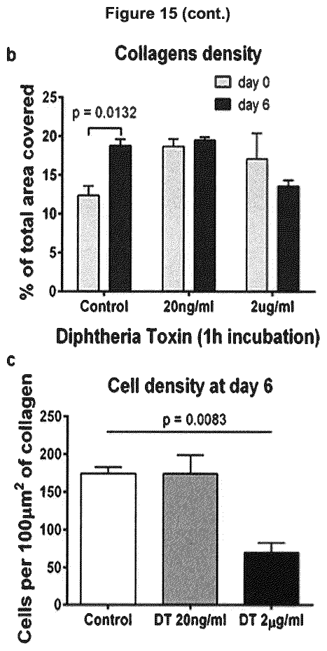

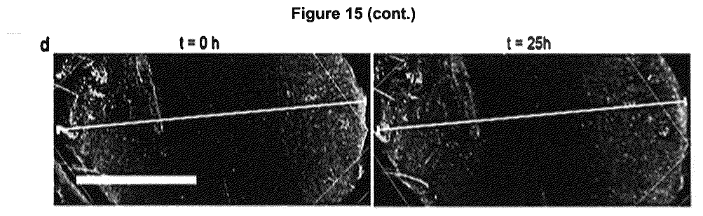

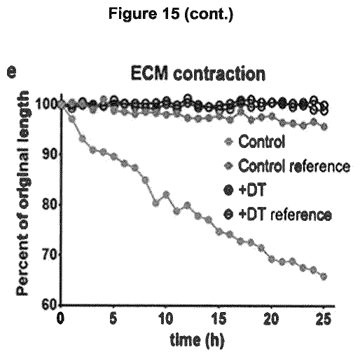

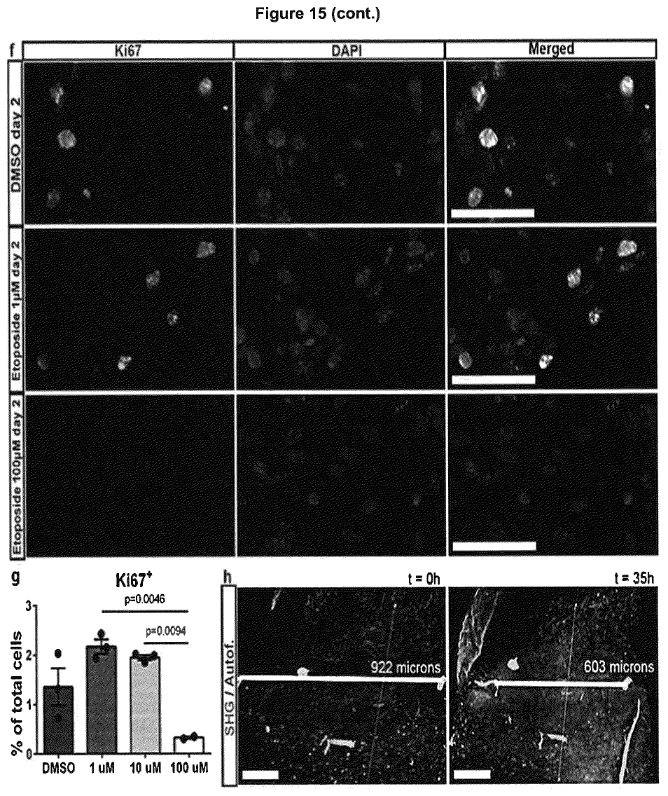

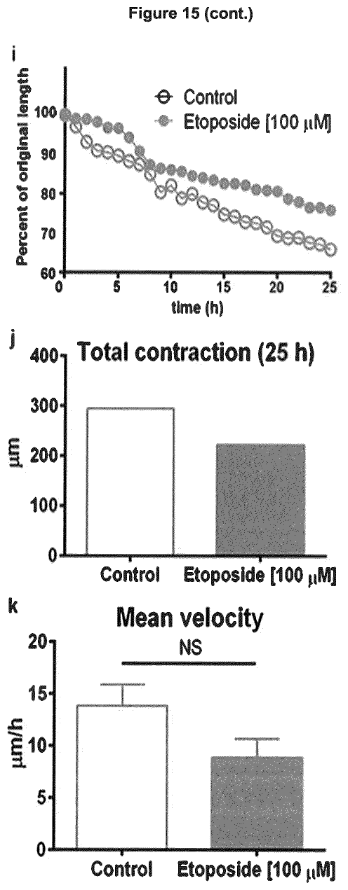

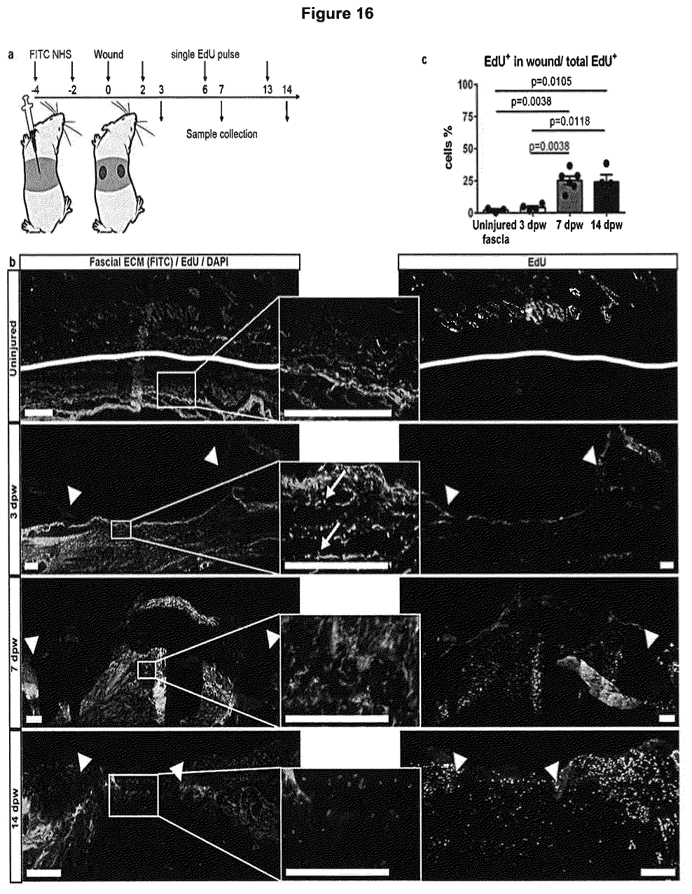

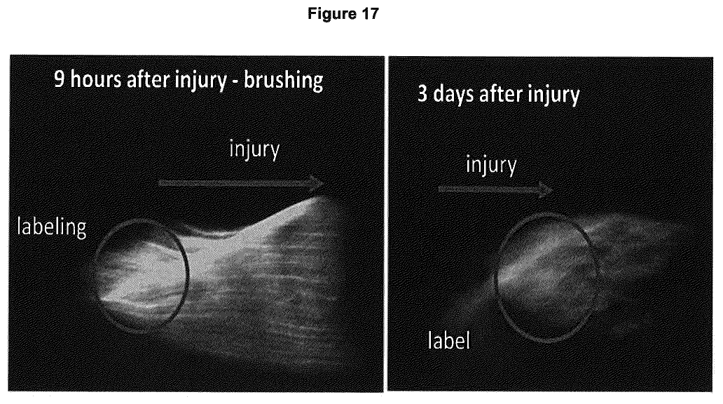

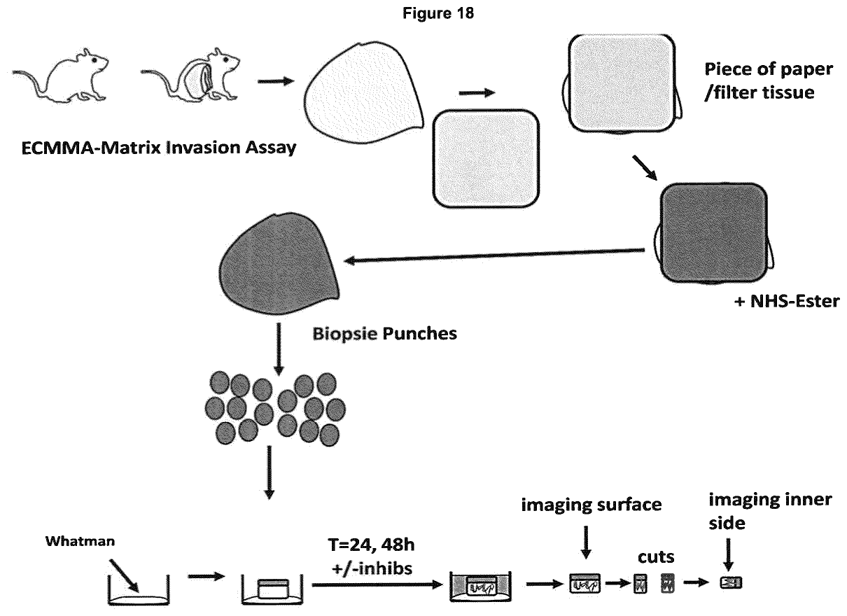

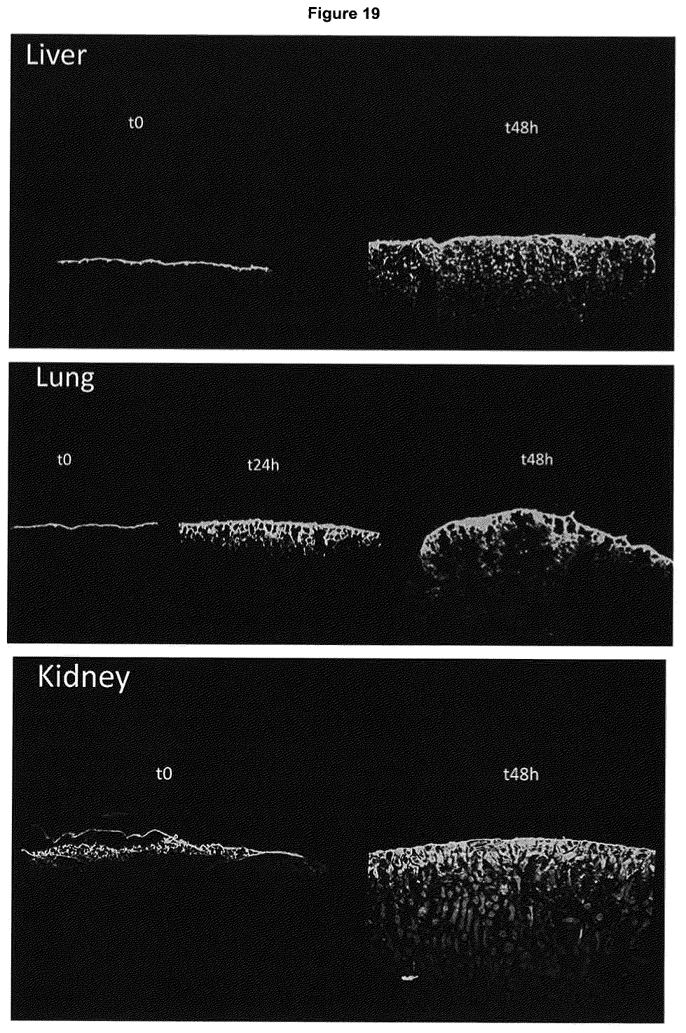

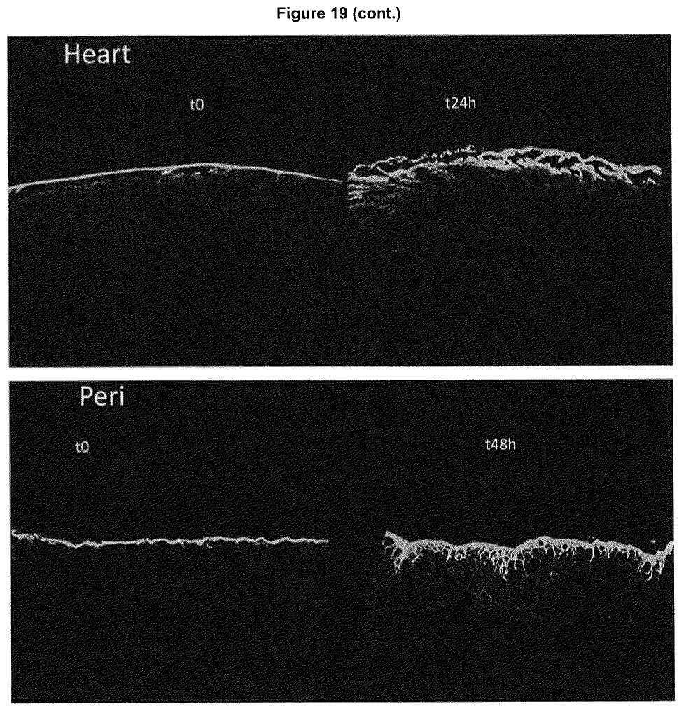

In a second independent approach, the Inventors used En1^{Cre}; R26^{iDTR} double transgenic mice in which DTR expression is restricted to EPFs, making them susceptible to DT-mediated ablation. To corroborate the ablation of EPFs in the fascia, the Inventors cultured fascia biopsies from En1^{Cre}; R26^{iDTR} for 6 days after an acute exposure to DT for 1 h ex vivo. A single exposure of 2 μg/ml DT prevented the normal increase in collagen fiber density observed in control samples and in wounds (fiber contraction and deposition, FIG. 15a-b) and led to a 2.5-times decrease in cell density within the fascia (FIG. 15c). Live imaging of fascia grafts treated with DT showed absence of any matrix steering over 25 hours (FIG. 15d-e), confirming fascial-EPFs are the cell protagonists of matrix movements.

Next, the Inventors created chimeric grafts using dermis from wild-type mice and fascia from En1^{Cre}; R26^{iDTR} mice. The Inventors ablated fascial EPFs using DT as before, then fluorescently labeled the matrix, and transplanted the skin grafts into the back-skin (FIG. 4j). Wounds after 14 days were completely absent of fascia-derived matrix (FIG. 4k-l). Instead, labeled matrix remained in the fascia layer below the wound bed. The in vitro imaging and the in vivo tracing experiments both conclusively demonstrate that fascia-resident EPFs actively steer matrix to seal open wounds, and that matrix steering is a mechanism unique to the fascia.

To check if fibroblastic proliferations preceded and was needed for matrix steering (FIG. 16a), the Inventors analyzed the proliferation rate in the matrix-tracking experiments. Expansion of the fascia gel beneath the wound occurred during the first days after injury, whereas cell proliferation peaked from day 7 post wounding and onwards (FIG. 16b-c), indicating that proliferation is not required for matrix steering. Furthermore, treatment with the proliferation inhibitor Etoposide had no effect on fascia matrix movements (FIG. 15f-k). Taken together, the results prove that fascia matrix works as an expanding sealant that quickly clogs deep wounds independently of cell proliferation.

### Example 5: Human Fascia and Keloid Scars Share a Fibroblastic Signature

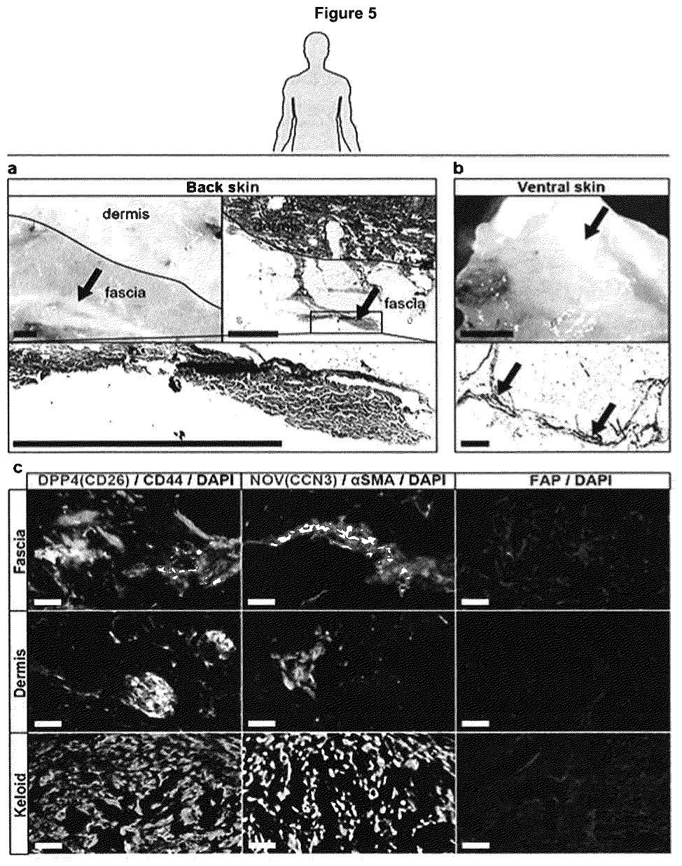

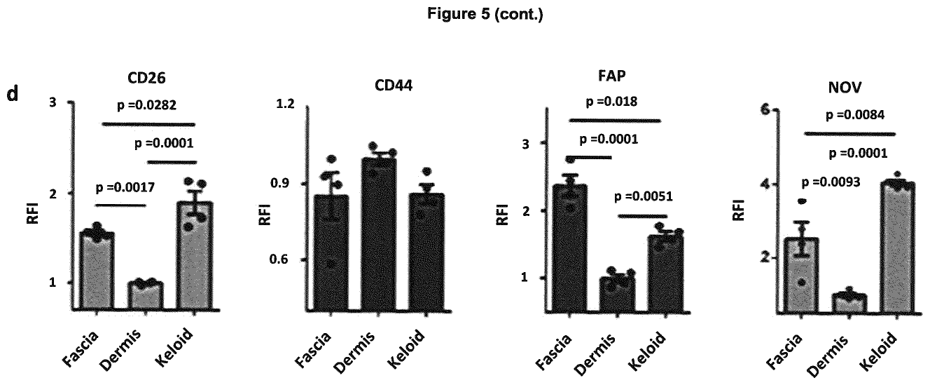

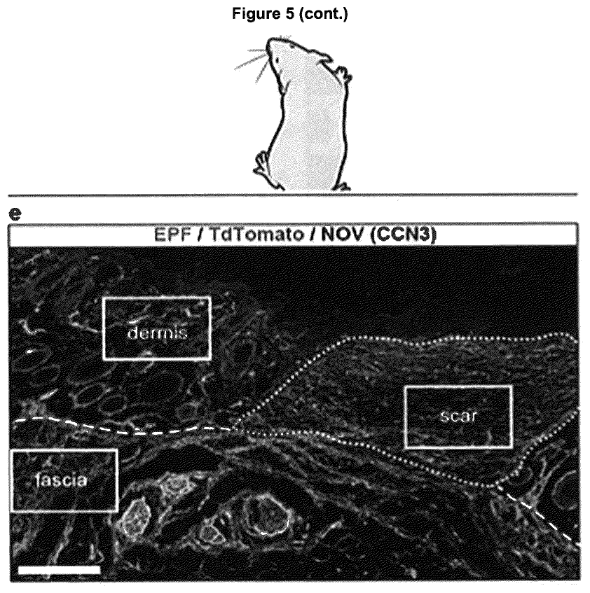

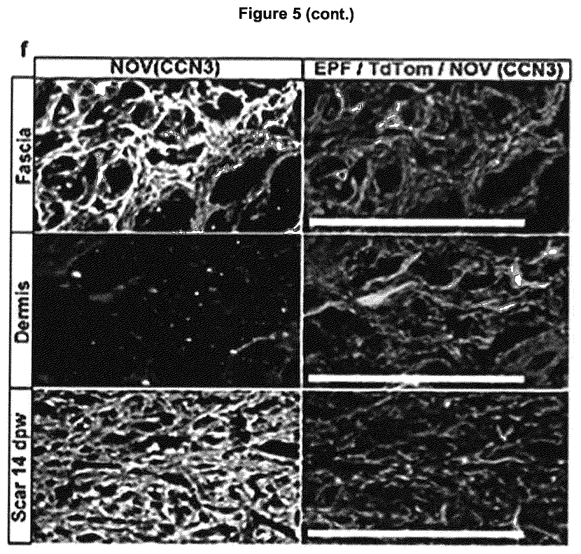

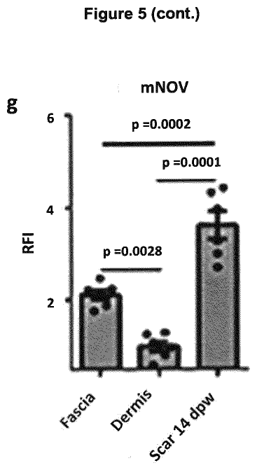

Human keloids are abnormal scars with clinical features of early and unresolved wounds (e.g. itchiness, inflammation, and pain) that progressively grow beyond the injury site²⁷. These unresolved features in human scars motivated us to investigate the presence of fascia and fascia fibroblasts in human skin and keloid tissue. The Inventors found bands of connective tissue in the subcutaneous space of human skin across multiple anatomic skin locations (FIG. 5a-b). Furthermore, markers of mouse fascia fibroblasts such as FAP and CD26 were highly expressed in the human subcutaneous fascia and in human keloid scars with low expression in healthy human dermis. The fascia-restricted cell-adhesion protein NOV/CCN3 was prominently expressed in both human and mouse fascia, as well as in human keloids and mouse scars (FIG. 5c-g). This preservation of fascia markers across mouse and human fascia and keloid scars suggests a common fascia origin for human excessive cutaneous scars.

### Example 6: Inhibitors of Connective Tissue Mobilization Induce Scarless Repair

Scars form by mobilizing fascia to sites of injury. The mechanism of this patch-repair is still obscure despite wounds being an extensively studied major clinical challenge. Here, the Inventors reveal a unique cellular mechanism of fibroblast sprouting and webbing that enact fascia mobilization and scarring. The Inventors screen live fascia explants with a library of 1280 small molecules and unearth a phenotypic class of chemicals with negligible effects on matrix biogenesis, yet completely inhibit scar formation by halting fascia mobilization, termed matrix motion inhibitors. The Inventors show that matrix motion inhibitors alter fibroblast sprouting and webbing. Inhibiting sprouting and webbing by either Thiostrepton, Fluvastatin sodium salt and Itraconazole reduced fascia jelly movements and led to a reduced scaring of wounds in animals. The findings place sprouting and webbing as a germinal mechanism of fibrotic scar formation, and a novel therapeutic space where matrix motion inhibitors provide a novel class of therapeutic treatments for the range of human fibrotic conditions.

Methods

Mouse Lines and Animal Experiments

En1^{Cre}; R26^{mTmG}, En1^{Cre}; R26^{mcherry}, C57BL/6J were purchased from Charles River or Jackson laboratories or generated in Research Animal Facility at the Stanford University as previously described (Rinkevich Y et al., 2015). Animals were housed in Animal Facility at Helmholtz Zentrum München at constant temperature and humidity with a 12-hour light cycle. Food and water were provided ad libitum. All animal experiments were reviewed and approved by the Government of Upper Bavaria and registered under the project 55.2-1-54-2532-61-16 and conducted under strict government and international guidelines. This study is compliant with all relevant ethical regulations regarding animal research.

For the chemical treatment study in live mice, splinted wounds were made in wildtype mouse back skin. Splinting rings were prepared from a 0.5-mm silicone sheet (CWS-S-0.5, Grace Bio-Labs) by cutting rings with an outer diameter of 12 mm and an inner diameter of 6 mm. After washing with detergent and rinsing with water, the splints were sterilized with 70% ethanol for 30 min and air dried in a cell culture hood and kept in a sterile bottle. Mice were anaesthetized with 100 μl MMF (medetomidine, midazolam and fentanyl). Dorsal hair was removed by a hair clipper, followed by hair removal cream for 3-5 min. Two full-thickness excisional wounds were created with a 5-mm diameter biopsy punch (Stiefel). One side of a splint was applied with silicone elastomer super glue (Kwik-Sil Adhesive, World Precision Instruments) and placed around the wound. The splint was secured with 5 sutures of 6.0 nylon, 75 μl per wound saline diluted chemical solutions with a final concentration of 250 μM were injected intra-dermally immediately after suture. Mice were recovered from anesthesia with an MMF antagonist and were supplied with metamizole (500 mg metamizole/250 ml drinking water) as postoperative analgesia. Scar samples were collected on D21.

For the fascia labelling study, 2 mg/ml NHS-fluorescein dye was subcutaneously injected into dorsal skin of wildtype mice at 4 days and 2 days prior to the surgery. After labelling, excisional wounds were made in mouse back-skin and treated with 75 μl saline diluted chemical solutions with a final concentration of 250 μM three times a week from the surgery day onwards. Scar samples were collected on D7.

Ex Vivo Skin Explant Assay

Post born Day 0 (P0) neonates of C57BL/6J wild type mouse were first sacrificed by decapitation. Then dorsal back skin was isolated to make 2 mm full thickness biopsies (0 2 mm, Stiefel) that included the epidermis, dermis and deep subcutaneous fascia layers. The whole skin tissue explant system is termed as scar-in-a-dish (SCAD) assay (Patent Application no. PLA17A13). The SCAD tissue was maintained in DMEM/F12 cell culture medium (Life Technologies) supplemented with 10% FBS (Life Technologies), 1% non-essential amino acid (Thermo Fisher), 1% Glutamax (Thermo Fisher), 1% penicillin and streptomycin (Thermo Fisher) in a 37° C. incubator supplied with 95% O₂ and 5% CO₂. Medium was changed every other day until day 5 when SCADs were collected and fixed for histology analysis. To be noticed, the excised skin was submerged dermis-side up in culture media, which confined the scar-prone fibroblasts to the explant and discouraged their adherence to the tissue culture plate.

Prestwick Chemical Library® (PCL) and Automatic Screening of Chemicals

The Prestwick library contains 1280 approved (by FDA, European Medicines Agency (EMA) or other agencies) small molecules covering a range of major anatomical therapeutic classes including central nervous system (19%), cardiovascular system (11%), metabolism (24%) and infectious diseases (16%). The purity of the compounds was >90% as reported by the provider of the compounds. The PCL provides an additional advantage as all chemicals are of stable physicochemical properties, show a high range of chemical diversity, and are with known bioavailability and safety data in humans. All these information helps to reduce the probability of screening low-quality hits and save the costs of preliminary screening process.

In order to accommodate to medium scale screening approach, the Inventors adapted the SCAD explant system into 96-well plate (Falcon) formats, with each well contained one biopsy. The novel 96-well SCAD pipeline was then combined with the 1280 approved small molecules from the Prestwick chemical library. Plate and liquid handling were performed using a high-throughput screening platform system composed of a Sciclone G3 Liquid Handler from PerkinElmer (Waltham, Mass., USA). On Day 0 tissues were treated either with the respective compound (1 mM stock solution) dissolved in 100% dimethyl sulfoxide (DMSO, Carl Roth) or DMSO alone. 0.5 μl of compounds/DMSO were transferred with a 96-array head to 200 μl DMEM/F12 medium per well to keep the final DMSO concentrations at 2.5 μM. Tissues were then incubated (37° C.; 5% CO₂) for 72 h prior to a second round of compound treatment, which was performed by exchanging cell culture medium per well and transferring 2.5 μM of compounds/DMSO into the fresh medium. After an incubation time of another 48 h (37° C.; 5% CO₂) the tissues were harvested and fixed for histological processing and imaging.

Ex Vivo Fascia Three-Dimensional (3D) Culture-Fascia Invasion Assay

To create a 3D environment that mimics the physiological environments in vivo, the Inventors established a fascia Matrigel (Corning) system. In order to observe the dynamic changes of fascia fibroblasts, the inventors isolated dorsal skin from P4 to P6 neonates of En1^{Cre}; R26^{mTmG} mouse lines. Cre positive neonates from this double transgenic mouse line were detected by green fluorescent signal in dorsal skin with a Leica M205 FA stereo microscope. Matrigel was prepared by diluting the stock aliquots with DMEM/F12 medium to a concentration of 6 mg/ml. Then 150 μl prepared gel was added in the center of a 35 mm cell culture dish (Ibidi). 4 mm biopsies were made from the dorsal skin and fascia tissues were then isolated from the dorsal skin tissues. Isolated fascia tissues were then embedded into the gel and were allowed to be solidified for one hour at 37° C. Then the tissue-gel system was maintained in DMEM/F12 medium (Life Technologies) supplemented with 10% FBS (Life Technologies), 1% non-essential amino acid (Thermo Fisher), 1% Glutamax (Thermo Fisher), 1% penicillin and streptomycin (Thermo Fisher) in a 37° C. incubator supplied with 95% O₂ and 5% CO₂. Medium was changed every other day until day 4 when tissues were collected and fixed. For fixation with Matrigel system, tissues were fixed in 2% paraformaldehyde (VWR) with 0.1% glutaraldehyde (Sigma) for 1 hour and then washed three times with phosphate buffered saline (PBS, Life Technologies) and stored in PBS at 4° C.

Invasion Index and Contraction Index Measurement

Fascia tissues were recorded everyday by a brightfield microscope to check the invasion and contraction state of the tissues. The invasion index was calculated with the following formula: Invasion index=(S_{D4}−S_{D0})/S_{D0}

S_{D4} and S_{D0} represent tissue size (including the migrated area) on Day 4 and Day 0, respectively. The contraction index was calculated with the following formula: Contraction index=(T_{D0}−T_{D4})/T_{D0}

T_{D4} and T_{D0} represent original tissue size (excluding the migrated area) on Day 4 and Day 0 respectively.

Histology

Except otherwise stated, all the samples were fixed overnight in 2% paraformaldehyde (VWR) in PBS at 4° C. and washed three times with PBS. Samples were then embedded in optimal cutting temperature compound (OCT, Sakura Finetek) and snap frozen on dry ice. 6 μm frozen sections were made by a cryostat (Cryostar NX70, Thermo fisher) and frozen section slides were stored at −20° C. Masson's trichrome staining was applied using a trichrome stain kit (Sigma-Aldrich) according to the manufacturer's instructions. Images were recorded by a ZEISS AxioImager. Z2m (Carl Zeiss) with brightfield channel. In Masson's trichrome staining, muscle fibers and keratin are stained as red color, collagen is stained as blue, cytoplasm is stained as light red and cell nuclei is stained as black.

3D Staining and Whole Mount Imaging

In order to characterize the properties of fascia samples cultured in Matrigel, the Inventors fixed the whole gel (with fascia tissue embedded inside) and conducted 3D staining. Samples were immersed overnight in PBSGT (lx PBS implemented with 0.2% gelatin (Sigma), 0.5% Triton X-100 (Sigma) and 0.01% thimerosal (Sigma)) at room temperature and incubated with primary antibodies diluted in PBSGT for three days at room temperature. The tissues were then washed three times with PBSGT for at least 30 min each time and incubated with secondary antibodies diluted in PBSGT for one day. Finally, tissues were rinsed three times with PBSGT and stored in PBS at 4° C. until imaging. 3D whole mount imaging was conducted with a Leica SP8 multi-photon microscope.

Primary and secondary antibodies applied in 3D staining: αSMA (ab21027, Abcam), Ki 67 (ab16667, Abcam), Cleaved Caspase-3 (9661S, Cell signalling), Gli1 (ab49314, Abcam), Donkey anti rabbit AF647 (A-31573, Life Technologies), Donkey anti goat AF647 (A-21447, Life Technologies).

Live Imaging

Fascia tissue cultured in Matrigel was fixed in 2% low-melting agarose (Biozym) and left at room temperature to be solidified. DMEM/F12 medium without phenol red was then added to keep the tissues alive during imaging. Four-dimension (4D) time-lapse images were performed by a Zeiss AxioObserver Z1 microscope for tissues obtained from En1^{Cre}; R26^{mCherry} mouse line or a Leica SP8 multi-photon microscope for tissues obtained from En1^{Cre}; R26^{mTmG} mouse line. Samples were placed in a qualified incubator with heating and gas control (Ibidi). The incubator temperature was adjusted to 35° C. and was supported with 5% CO₂ during imaging. Brightfield and mCherry signals were recorded for tissues from En1^{Cre}; R26 ^{mCherry}; green fluorescent protein (GFP) and tdTomato signals were recorded for tissues from En1^{Cre}; R26^{mTmG}.

Cell Tracking

4D time-lapsed imaging was subjected to maximum intensity projection in Imaris 9.3.1 (Bitplane) software. The projected data sets were proceeded to cell migration and cell-tracking analysis using Trackmate function of ImageJ. Variables, such as blob diameter, threshold, and segmentation detector were adjusted to suit the nature of the data and the samples. For the fourth dimension of the tracks, color ramp was applied to the individual tracks as a function of time (blue, first time point of the track; orange, last time point of the track).

Data Analysis

3D images and time series videos were processed with Imaris 9.3.1 (Bitplane). Brightness and contrast were modified to exclude false positive signal and to obtain better visibility. Fractal analysis was conducted using the ImageJ plug in ‘FracLac’29 (FracLac 2015Sep090313a9390) (Karperien A, 1999-2003). Fractal dimension (D_{F}) values and Lacunarity (Lac) values were calculated using the box counting approach (slipping and tighten grids were set at default sample sizes, threshold of minimum pixel density was set as 0.40).

Statistics and Reproducibility

Statistical analysis was performed using GraphPad Prism software (Version 7.0, GraphPad). Statistical significance was determined using analysis of variance (ANOVA) with Tukey's or Dunnett's multiple comparison test, as indicated in the corresponding figure legends. Until otherwise stated, all results were repeated with at least three independent experiments or three biological samples with consistent results. Cell tracking were derived with three single movies. 3D staining was performed on two samples and images were recorded at three different sites of the samples.

### Results

Anti-Fibrotic and Pro-Fibrotic Agents Identified Through Compound Screening

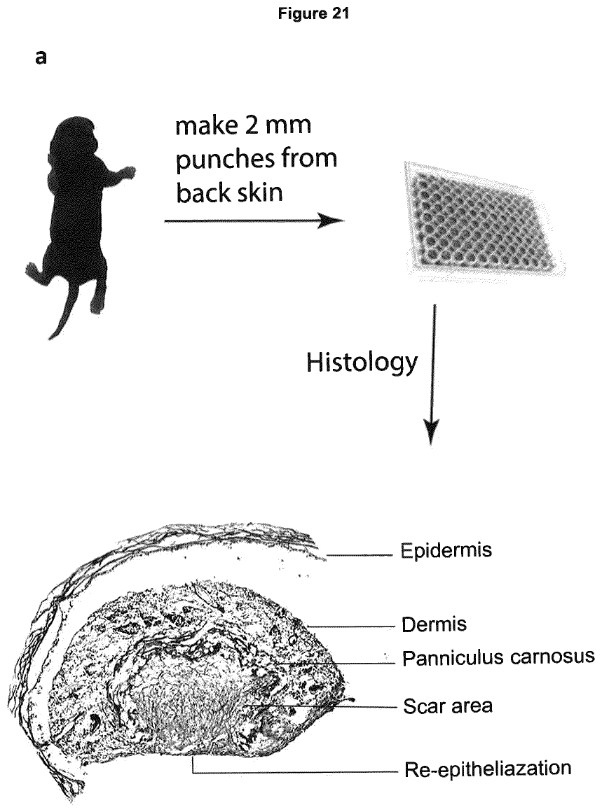

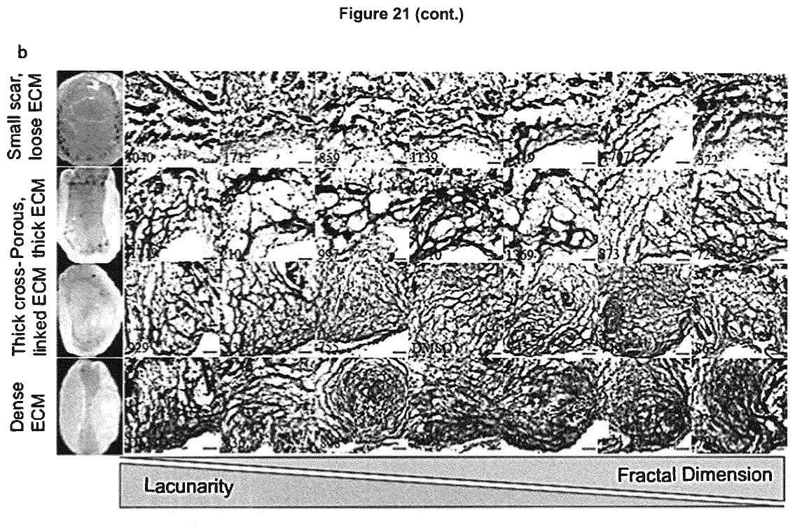

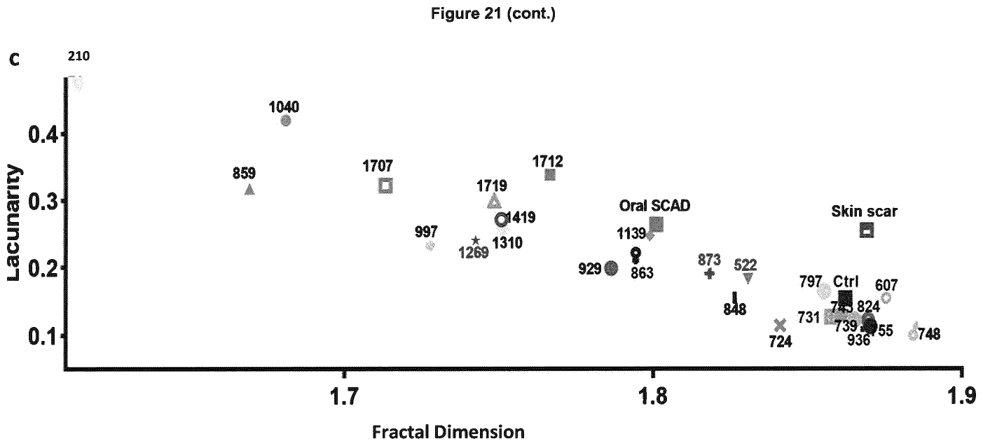

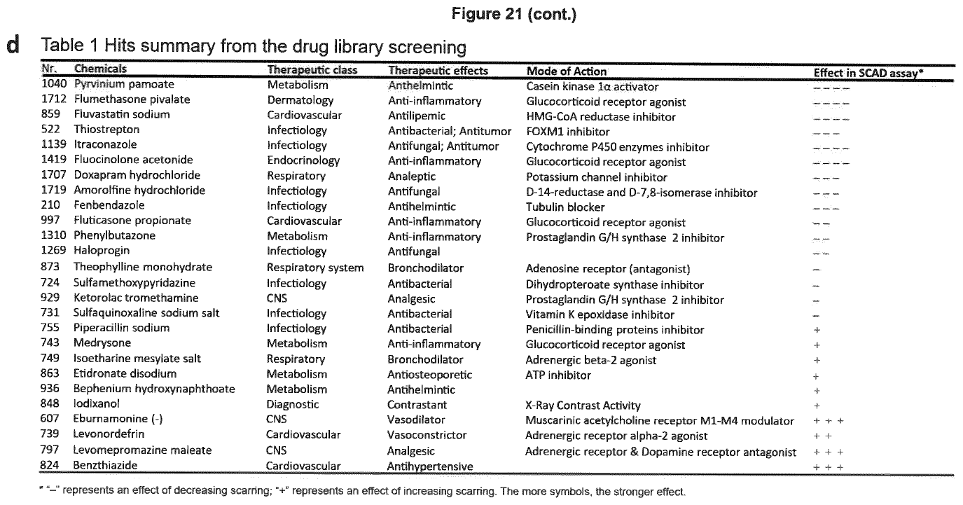

To identify the mechanism of how fascia is mobilized/centralized in wounds to form scars, the Inventors took advantage of whole skin-fascia explants, in which uniform centralized scars develop ex vivo (Correa-Gallegos et al., 2019). Briefly, the Inventors excised 2-mm full thickness skin biopsies that included epidermis, dermis, and deep subcutaneous fascia layers from mouse backs (see Methods). Skin-fascia explants were submerged fascia-side up in culture media, to confine scar-prone fibroblasts to the explant and discourage their adherence to the tissue culture plate. Under these conditions, explants develop uniform scars over a course of 5 days that contract and fold the skin (FIG. 21a). The explant technique mimics the fascia wound response by mobilizing/centralizing connective tissue to form scars as in animals, including dense opaque plugs of extracellular matrix at wound centers and skin contraction.

In order to discover novel inhibitors of fascia mobilization and scar formation, the Inventors combined the skin-fascia explant system with 1280 FDA-approved small molecules (Prestwick library) via a high-throughput screening platform. The Prestwick library has selected a diverse array of chemicals with strong physicochemical properties, with known bioavailability and clinical safety data. The library is an ideal place to start screening as it covers all major therapeutic chemical classes such as central nervous system, cardiovascular system, metabolism, and infectious diseases.

Our initial phenotypic screening of the 1280 small molecules identified 122 chemicals (9.53% ‘hits’) that consistently changed the extent of mobilization/centralization of the fascia connective tissue and of scar size and severity (part A as table 1 and part B as FIG. 48). To further validate the efficacy of the scarring phenotypes, the Inventors manually repeated the chemical screening for the 122 ‘hit’ chemicals with 6 biological replicates per chemical.

TABLE 1
(part A) 122 ‘hit’ chemicals

| | | | Mol | |
|---|---|---|---|---|
| Prestw | | | weight | CAS |
| number | Chemical name | fmla structure | structure | number |
| Prestw- | Doxapram | C24H31ClN2O2 | 414.98 | 7081-53-0 |
| 1707 | hydrochloride | | | |
| Prestw- | Amorolfine | C21H36ClNO | 353.98 | 78613-38- |
| 1719 | hydrochloride | | | 4 |
| Prestw- | Flumethasone pivalate | C27H36F2O6 | 494.58 | 2002-29-1 |
| 1712 | | | | |
| Prestw- | Pyrvinium pamoate | C75H70N6O6 | 1151.43 | 3546-41-6 |
| 1040 | | | | |
| Prestw- | Sulfaquinoxaline | C14H11N4NaO2S | 322.32 | 967-80-6 |
| 731 | sodium salt | | | |
| Prestw- | Piperacillin sodium salt | C23H26N5NaO7S | 539.55 | 59703-84- |
| 755 | | | | 3 |
| Prestw- | lodixanol | C35H44I6N6O15 | 1550.20 | 92339-11- |
| 848 | | | | 2 |
| Prestw- | Methylhydantoin-5-(D) | C4H6N2O2 | 114.10 | 55147-68- |
| 864 | | | | 7 |
| Prestw- | Itraconazole | C35H38Cl2N8O4 | 705.65 | 84625-61- |
| 1139 | | | | 6 |
| Prestw- | Azelastine HCl | C22H25Cl2N3O | 418.37 | 79307-93- |
| 1130 | | | | 0 |
| Prestw- | Doxorubicin | C27H30ClNO11 | 579.99 | 25316-40- |
| 438 | hydrochloride | | | 9 |
| Prestw- | Betamethasone | C22H29FO5 | 392.47 | 378-44-9 |
| 362 | | | | |
| Prestw- | Thiostrepton | C72H85N19O18S5 | 1664.92 | 1393-48-2 |
| 522 | | | | |
| Prestw- | Clofazimine | C27H22Cl2N4 | 473.41 | 2030-63-9 |
| 376 | | | | |
| Prestw- | Naltrexone | C20H28ClNO6 | 413.90 | 16676-29- |
| 116 | hydrochloride dihydrate | | | 2 |
| Prestw- | Repaglinide | C27H36N2O4 | 452.60 | 135062- |
| 1046 | | | | 02-1 |
| Prestw- | Propoxycaine | C16H27ClN2O3 | 330.86 | 550-83-4 |
| 1059 | hydrochloride | | | |
| Prestw- | Tegaserod maleate | C20H27N5O5 | 417.47 | 189188- |
| 1760 | | | | 57-6 |
| Prestw- | Phenylbutazone | C19H20N2O2 | 308.38 | 50-33-9 |
| 1310 | | | | |
| Prestw- | Fluticasone propionate | C25H31F3O5S | 500.58 | 80474-14- |
| 997 | | | | 2 |
| Prestw- | Pivampicillin | C22H29N3O6S | 463.56 | 33817-20- |
| 1009 | | | | 8 |
| Prestw- | Fluocinolone acetonide | C24H30F2O6 | 452.50 | 67-73-2 |
| 1419 | | | | |
| Prestw- | Benzathine | C48H56N6O10S2 | 941.14 | 5928-84-7 |
| 1028 | benzylpenicillin | | | |
| Prestw- | Halofantrine | C26H31Cl3F3NO | 536.90 | 36167-63- |
| 1031 | hydrochloride | | | 2 |
| Prestw- | Sulfamethoxypyridazine | C11H12N4O3S | 280.31 | 80-35-3 |
| 724 | | | | |
| Prestw- | Levonordefrin | C9H13NO3 | 183.21 | 829-74-3 |
| 739 | | | | |
| Prestw- | Medrysone | C22H32O3 | 344.50 | 2668-66-8 |
| 743 | | | | |
| Prestw- | Oxalamine citrate salt | C20H27N3O8 | 437.45 | 1949-20-8 |
| 826 | | | | |
| Prestw- | Ketorolac tromethamine | C19H24N2O6 | 376.41 | 74103-07- |
| 929 | | | | 4 |
| Prestw- | Bephenium | C28H29NO4 | 443.55 | 3818-50-6 |
| 936 | hydroxynaphthoate | | | |
| Prestw- | Fluvastatin sodium salt | C24H25FNNaO4 | 433.46 | 93957-55- |
| 859 | | | | 2 |
| Prestw- | Etidronic acid, disodium | C2H6Na2O7P2 | 249.99 | 7414-83-7 |
| 863 | salt | | | |
| Prestw- | Methotrimeprazine | C23H28N2O5S | 444.55 | 7104-38-3 |
| 797 | maleat salt | | | |
| Prestw- | Haloprogin | C9H4Cl3IO | 361.40 | 777-11-7 |
| 1269 | | | | |
| Prestw- | Mevastatin | C23H34O5 | 390.52 | 73573-88- |
| 1441 | | | | 3 |
| Prestw- | Domperidone | C22H24ClN5O2 | 425.92 | 57808-66- |
| 340 | | | | 9 |
| Prestw- | Alfacalcidol | C27H44O2 | 400.65 | 41294-56- |
| 1211 | | | | 8 |
| Prestw- | Pyrazinamide | C5H5N3O | 123.12 | 98-96-4 |
| 514 | | | | |
| Prestw- | Eburnamonine (−) | C19H22N2O | 294.40 | 4880-88-0 |
| 607 | | | | |
| Prestw- | Minoxidil | C9H15N5O | 209.25 | 38304-91- |
| 20 | | | | 5 |
| Prestw- | Sulfaphenazole | C15H14N4O2S | 314.37 | 526-08-9 |
| 21 | | | | |
| Prestw- | Norethynodrel | C20H26O2 | 298.43 | 68-23-5 |
| 24 | | | | |
| Prestw- | Famotidine | C8H15N7O2S3 | 337.45 | 76824-35- |
| 104 | | | | 6 |
| Prestw- | Disopyramide | C21H29N3O | 339.48 | 3737-09-5 |
| 266 | | | | |
| Prestw- | Amyleine hydrochloride | C14H22ClNO2 | 271.79 | 532-59-2 |
| 49 | | | | |
| Prestw- | Nefopam hydrochloride | C17H20ClNO | 289.81 | 23327-57- |
| 229 | | | | 3 |
| Prestw- | Epirubicin | C27H30ClNO11 | 579.99 | 56390-09- |
| 1752 | hydrochloride | | | 1 |
| Prestw- | Isoetharine mesylate | C14H25NO6S | 335.42 | 7279-75-6 |
| 749 | salt | | | |
| Prestw- | Clidinium bromide | C22H26BrNO3 | 432.36 | 3485-62-9 |
| 822 | | | | |
| Prestw- | Benzthiazide | C15H14ClN3O4S3 | 431.94 | 91-33-8 |
| 824 | | | | |
| Prestw- | Theophylline | C7H10N4O3 | 198.18 | 5967-84-0 |
| 873 | monohydrate | | | |
| Prestw- | Daunorubicin | C27H30ClNO10 | 563.99 | 23541-50- |
| 487 | hydrochloride | | | 6 |
| Prestw- | Acarbose | C25H43NO18 | 645.62 | 56180-94- |
| 1174 | | | | 0 |
| Prestw- | Fenbendazole | C15H13N3O2S | 299.35 | 43210-67- |
| 210 | | | | 9 |
| Prestw- | Vorinostat | C14H20N2O3 | 264.33 | 149647- |
| 1362 | | | | 78-9 |
| Prestw- | Prothionamide | C9H12N2S | 180.27 | 14222-60- |
| 1321 | | | | 7 |
| Prestw- | Isradipine | C19H21N3O5 | 371.40 | 75695-93- |
| 1021 | | | | 1 |
| Prestw- | Lomerizine | C27H31ClF2N2O3 | 505.01 | 101477- |
| 1775 | hydrochloride | | | 54-7 |
| Prestw- | Ampiroxicam | C20H21N3O7S | 447.47 | 99464-64- |
| 1772 | | | | 9 |
| Prestw- | Promethazine | C17H21ClN2S | 320.89 | 58-33-3 |
| 888 | hydrochloride | | | |
| Prestw- | Nisoxetine | C17H22ClNO2 | 307.82 | 57754-86- |
| 910 | hydrochloride | | | 6 |
| Prestw- | Tremorine | C12H22Cl2N2 | 265.23 | 51-73-0 |
| 331 | dihydrochloride | | | |
| Prestw- | Terconazole | C26H31Cl2N5O3 | 532.47 | 67915-31- |
| 495 | | | | 5 |
| Prestw- | Vancomycin | C66H76Cl3N9O24 | 1485.75 | 1404-93-9 |
| 497 | hydrochloride | | | |
| Prestw- | Argatroban | C23H36N6O5S | 508.64 | 74863-84- |
| 1228 | | | | 6 |
| Prestw- | Benfluorex | C19H20F3NO2 | 351.37 | 23602-78- |
| 367 | | | | 0 |
| Prestw- | Phenacetin | C10H13NO2 | 179.22 | 62-44-2 |
| 533 | | | | |
| Prestw- | Alprenolol | C15H24ClNO2 | 285.82 | 13707-88- |
| 250 | hydrochloride | | | 5 |
| Prestw- | Diflunisal | C13H8F2O3 | 250.20 | 22494-42- |
| 39 | | | | 4 |
| Prestw- | Dipyridamole | C24H40N8O4 | 504.64 | 58-32-2 |
| 142 | | | | |
| Prestw- | Econazole nitrate | C18H16Cl3N3O4 | 444.70 | 24169-02- |
| 304 | | | | 6 |
| Prestw- | Gabexate mesilate | C17H27N3O7S | 417.48 | 56974-61- |
| 1008 | | | | 9 |
| Prestw- | Sparfloxacin | C19H22F2N4O3 | 392.41 | 110871- |
| 1343 | | | | 86-8 |
| Prestw- | Alprostadil | C20H34O5 | 354.49 | 745-65-3 |
| 1018 | | | | |
| Prestw- | Hexachlorophene | C13H6Cl6O2 | 406.91 | 70-30-4 |
| 1472 | | | | |
| Prestw- | Irinotecan | C33H45ClN4O9 | 677.20 | 136572- |
| 1494 | hydrochloride trihydrate | | | 09-3 |
| Prestw- | Cimetidine | C10H16N6S | 252.34 | 51481-61- |
| 26 | | | | 9 |
| Prestw- | Lanatoside C | C49H76O20 | 985.14 | 17575-22- |
| 656 | | | | 3 |
| Prestw- | Pramoxine | C17H28ClNO3 | 329.87 | 637-58-1 |
| 716 | hydrochloride | | | |
| Prestw- | Penbutolol sulfate | C36H60N2O8S | 680.95 | 38363-32- |
| 1043 | | | | 5 |
| Prestw- | Doxycycline | C22H25ClN2O8 | 480.91 | 10592-13- |
| 1399 | hydrochloride | | | 9 |
| Prestw- | Pantoprazole sodium | C16H14F2N3NaO4S | 405.36 | 138786- |
| 1758 | | | | 67-1 |
| Prestw- | (R)-Duloxetine | C18H20ClNOS | 333.88 | 116539- |
| 1708 | hydrochloride | | | 60-7 |
| Prestw- | Donepezil | C24H30ClNO3 | 415.96 | 120011- |
| 1706 | hydrochloride | | | 70-3 |
| Prestw- | Fenoldopam | C16H16ClNO3 | 305.76 | 67227-56- |
| 1713 | | | | 9 |
| Prestw- | Valdecoxib | C16H14N2O3S | 314.37 | 181695- |
| 1759 | | | | 72-7 |
| Prestw- | Verteporfin | C41H42N4O8 | 718.81 | 129497- |
| 1105 | | | | 78-5 |
| Prestw- | Parbendazole | C13H17N3O2 | 247.30 | 14255-87- |
| 1110 | | | | 9 |
| Prestw- | Loteprednol etabonate | C24H31ClO7 | 466.96 | 82034-46- |
| 1741 | | | | 6 |
| Prestw- | Cefepime hydrochloride | C19H25ClN6O5S2 | 517.03 | 123171- |
| 1118 | | | | 59-5 |
| Prestw- | Spectinomycin | C14H26Cl2N2O7 | 405.28 | 21736-83- |
| 804 | dihydrochloride | | | 4 |
| Prestw- | Halcinonide | C24H32ClFO5 | 454.97 | 3093-35-4 |
| 655 | | | | |
| Prestw- | Cefazolin sodium salt | C14H13N8NaO4S3 | 476.49 | 27164-46- |
| 736 | | | | 1 |
| Prestw- | Suprofen | C14H12O3S | 260.31 | 40828-46- |
| 816 | | | | 4 |
| Prestw- | Pipemidic acid | C14H17N5O3 | 303.32 | 51940-44- |
| 897 | | | | 4 |
| Prestw- | Methylatropine nitrate | C18H26N2O6 | 366.42 | 52-88-0 |
| 900 | | | | |
| Prestw- | Dydrogesterone | C21H28O2 | 312.46 | 152-62-5 |
| 671 | | | | |
| Prestw- | Butacaine | C18H30N2O2 | 306.45 | 149-16-6 |
| 831 | | | | |
| Prestw- | Sulfamerazine | C11H12N4O2S | 264.31 | 127-79-7 |
| 694 | | | | |
| Prestw- | Tolmetin sodium salt | C15H18NNaO5 | 315.30 | 64490-92- |
| 856 | dihydrate | | | 2 |
| Prestw- | Tacrine hydrochloride | C13H15ClN2 | 234.73 | 1684-40-8 |
| 329 | | | | |
| Prestw- | Bisoprolol fumarate | C40H66N2O12 | 766.98 | 104344- |
| 330 | | | | 23-2 |
| Prestw- | Furosemide | C12H11ClN2O5S | 330.75 | 54-31-9 |
| 341 | | | | |
| Prestw- | Maprotiline | C20H24ClN | 313.87 | 10347-81- |
| 346 | hydrochloride | | | 6 |
| Prestw- | Clozapine | C18H19ClN4 | 326.83 | 5786-21-0 |
| 350 | | | | |
| Prestw- | Camylofine | C19H34Cl2N2O2 | 393.40 | 54-30-8 |
| 1498 | chlorhydrate | | | |
| Prestw- | Dobutamine | C18H24ClNO3 | 337.85 | 49745-95- |
| 352 | hydrochloride | | | 1 |
| Prestw- | Celiprolol HCl | C20H34ClN3O4 | 415.96 | 57470-78- |
| 1132 | | | | 7 |
| Prestw- | Zafirlukast | C31H33N3O6S | 575.69 | 107753- |
| 1364 | | | | 78-6 |
| Prestw- | Rimantadine | C12H22ClN | 215.77 | 13392-28- |
| 1331 | Hydrochloride | | | 4 |
| Prestw- | Efavirenz | C14H9ClF3NO2 | 315.68 | 154598- |
| 1400 | | | | 52-4 |
| Prestw- | Penciclovir | C10H15N5O3 | 253.26 | 39809-25- |
| 1488 | | | | 1 |
| Prestw- | Meticrane | C10H13NO4S2 | 275.35 | 1084-65-7 |
| 11 | | | | |
| Prestw- | Khellin | C14H12O5 | 260.25 | 82-02-0 |
| 91 | | | | |
| Prestw- | Benzonatate | C30H53NO11 | 603.76 | 104-31-4 |
| 12 | | | | |
| Prestw- | Vinpocetine | C22H26N2O2 | 350.46 | 42971-09- |
| 268 | | | | 5 |
| Prestw- | Dapsone | C12H12N2O2S | 248.31 | 80-08-0 |
| 35 | | | | |
| Prestw- | Haloperidol | C21H23ClFNO2 | 375.87 | 52-86-8 |
| 115 | | | | |
| Prestw- | Dicyclomine | C19H36ClNO2 | 345.96 | 67-92-5 |
| 48 | hydrochloride | | | |
| Prestw- | Triflupromazine | C18H20ClF3N2S | 388.89 | 1098-60-8 |
| 53 | hydrochloride | | | |
| Prestw- | Minaprine | C17H24Cl2N4O | 371.31 | 25953-17- |
| 66 | dihydrochloride | | | 7 |

Part B of table 1 is depicted as FIG. 48 comprising H and E stainings of the tissue samples which were treated with the respective chemical compounds listed also as table 1.

The inventors categorised the scar-active hits into 18 distinct phenotypic groups, based on overall scar dimension and morphology. For example, certain explant groups gave exuberant scars that extended beyond the skin explant boarders, with contraction and bending similar to hypertrophic scars (FIG. 21b). Other groups of scars had dense foci of fibroblastic cells at their centers; or scars with abnormally porous and thickened matrix fibres; or brittle scars with thin and loosely linked reticular matrix. There are also groups that had dramatically reduced scarring with minimal skin contraction. Remarkably, a small percentage of the chemicals had completely abolished scar formation; explants lacked any visible appearance of scars, remained flat, failed to contract (FIG. 21b). This was especially surprising as currently there are few if any known anti-scarring drugs available.

To further classify and grade the ranges of scar phenotypes and severities, the Inventors performed fractal analysis on all sample groups, by determining the fractal dimensions (D_{F}) and lacunarity (porosity) values of the ECM lattice organisation (Jiang et al., 2018). Fractal porosity and lacunarity are measures of the general organization of the extracellular matrix, with scars having higher fractal dimensions (FD) and lower lacunarity (L) values than normal healthy skin (FIG. 21c).

The combined histo-morphometric and fractal analysis, together, allowed us to extend the phenotypic groups of chemicals into 26 distinct grades/severities, each with unique scarring phenotypes. Out of the 26, ten compounds gave more severe scars, whereas sixteen reduced scarring with measurable anti-fibrotic effects (FIG. 21b). Intriguingly, the anti-scarring compounds were from a range of therapeutic classes including anti-fungal, anti-bacterial, anti-inflammatory, anti-helminthic, anti-lipemic, analeptic, bronchodilatory, and analgesic compounds that seemingly targeted diverse modes of action (Table 1).

Fascia Fibroblasts Form Reticulations Ex-Vivo

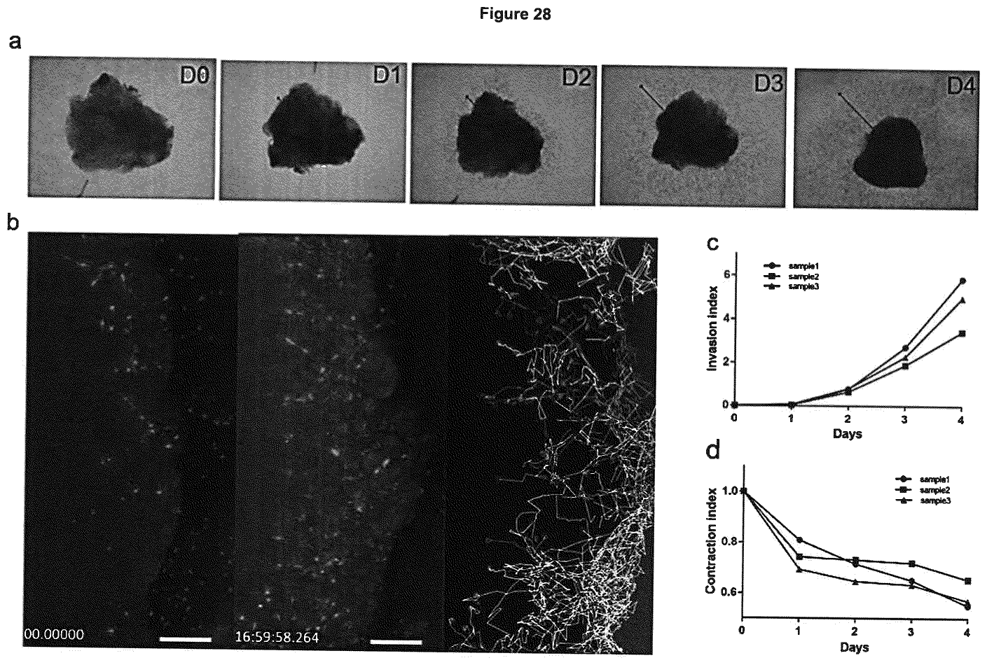

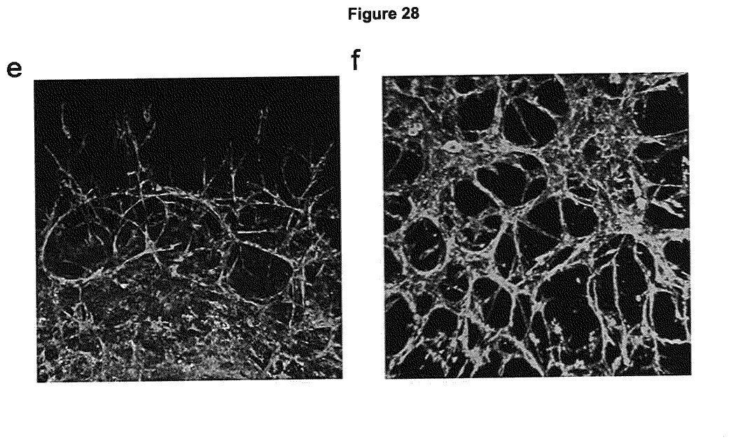

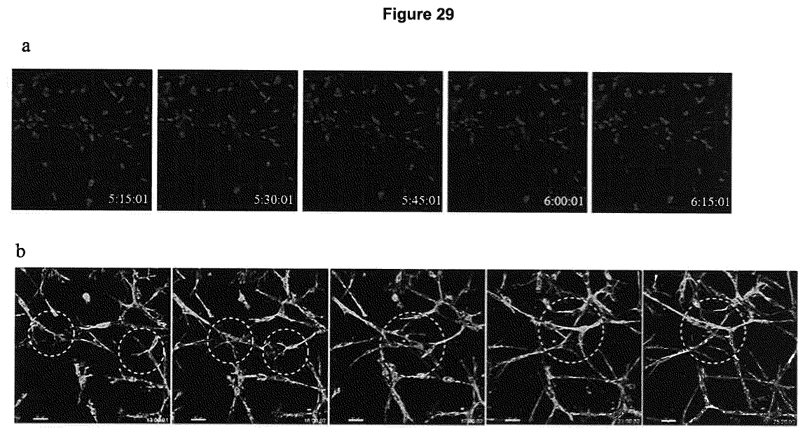

To visualize the early mechanisms that mobilize/centralize fascia connective tissue, the Inventors crossed fibroblastic lineage reporter mice (En1^{Cre}) with transgenic reporter mice (R26^{mTmG}). Double transgenic offspring (En1^{Cre}; R26^{mTmG}) express GFP under the En1 promoter, thereby genetically tagging fibrogenic lineage cells (EPFs) in the fascia with GFP. The Inventors excised fascia explants from back-skin of this double-transgenic mice and performed high-resolution live imaging to follow individual EPFs throughout scarring. EPFs within the fascia jelly became increasingly connected to one another within the jelly (FIG. 28a, FIG. 29a). This increased connectivity was mediated by forming sprouts of multiple fibroblasts trailing along leading EPFs. New sprouts quickly connected to adjacent sprouts, forming an interconnected cellular reticulum within the fascia jelly that was accompanied by a massive increase in invasion speed and change in overall fascia dimensions, extending outwards from the original borders of the tissue (FIG. 28c-d, 29b).

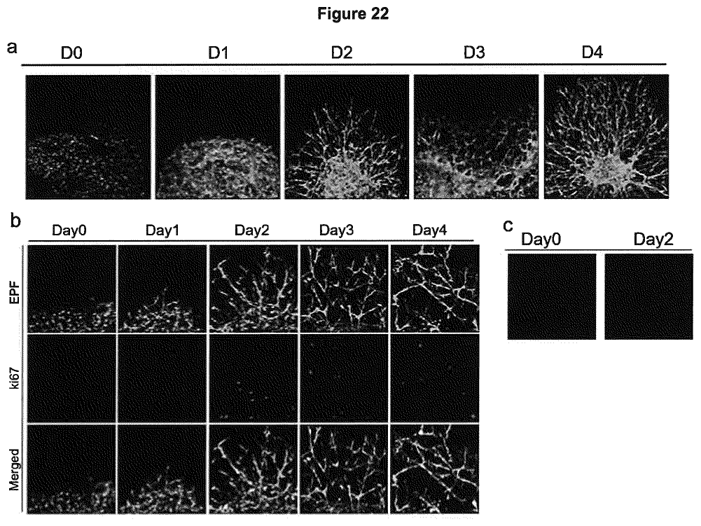

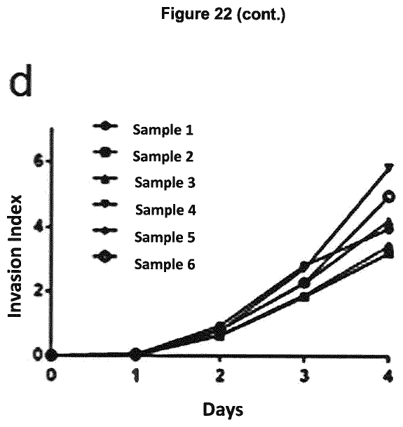

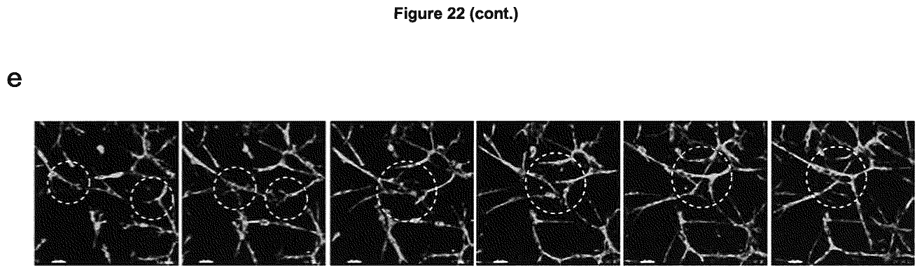

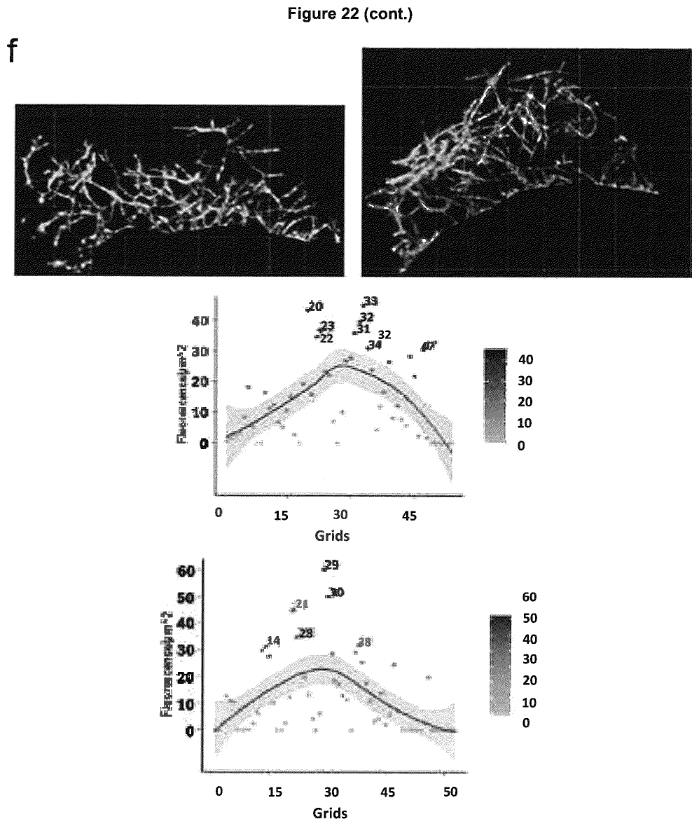

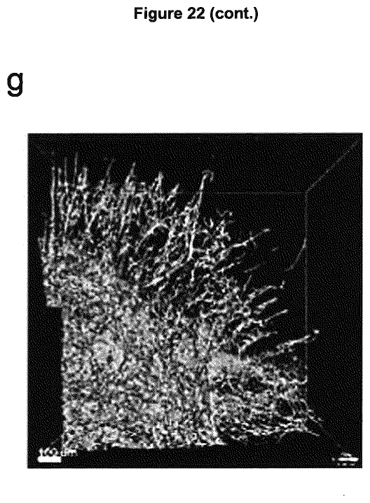

To better visualize the dynamics of sprouting and reticulation of fascia fibroblasts, the Inventors crossed the fibroblast lineage-specific promoter (En1) mice to a nuclear mCherry reporter line, allowing to computationally track and map all fibroblast dynamics. Live imaging of fascia explants from En1^{Cre}; R26^{mcherry} double transgenic mice from day 2 to day 5 revealed cell-cell connections formed a network of cell clusters. Automatic tracking data also revealed a formed network of fibroblasts that constantly sprout new filaments, which anastomose to create a web of interconnected fibroblasts (FIG. 22e. Immuno-labelling of Ki67 staining and live imaging showed that cells underwent proliferation during the enactment of migration and sprouting (FIG. 22b, FIG. 29a). Live imaging of a mass of fascia fibroblasts disconnected to the original tissue revealed the intrinsic self-contraction property of fascia, this property was proved by the tracking trajectories (FIG. 22f).

Three Top Anti-Scar Agents were Identified by Secondary Screen on Fascia

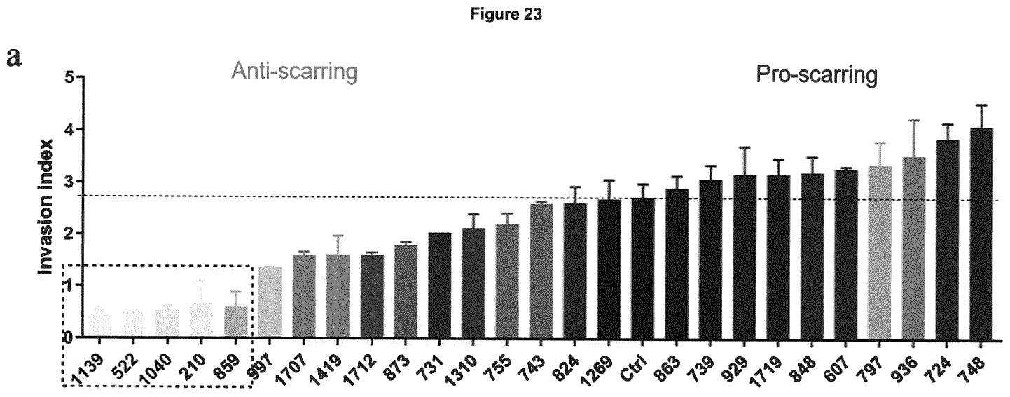

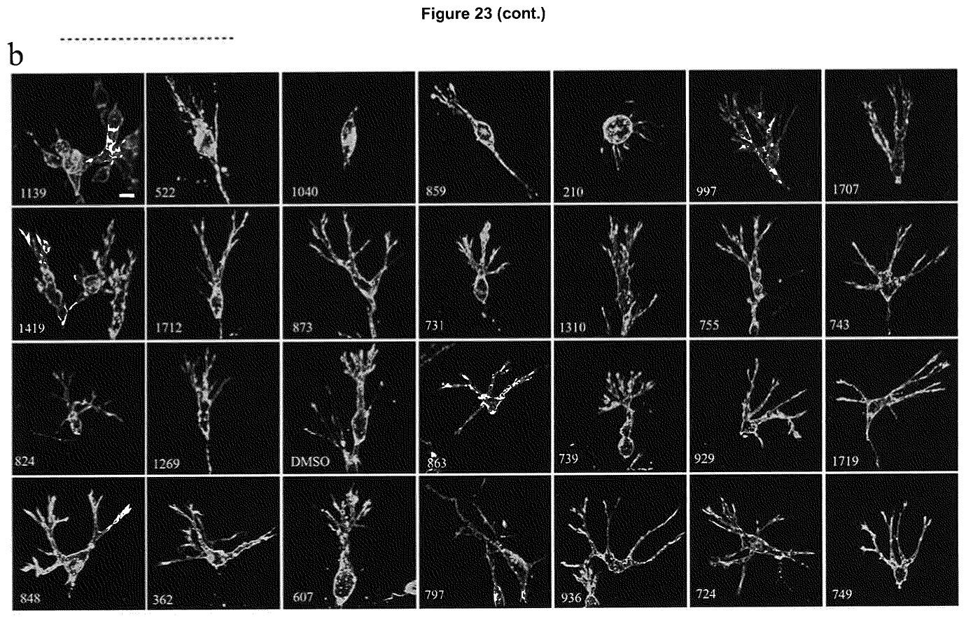

The inventors then went on to determine the anti-scarring actions of the small molecules. The Inventors isolated fascia from back-skin of En1^{Cre}; R26^{mTmG} double-transgenic mice, separately incubated whole fascia explants with 26 small molecules, and measured the invasion index (Si)=(S_{D4}−S_{D0})/S_{D0} (see Methods) as a proxy measure of sprouting and reticulation. The change of invasion index in the presence of the 26 small molecules precisely mimicked both the anti- and pro-scarring effects from the original screen (FIG. 23a). Specifically, the top five anti-scarring compounds dramatically inhibited cell migration with low invasion index (FIG. 23a). Single cell imaging of fascia showed that the small molecules inhibited reticulations by altering the type, numbers and the magnitude of cell membrane protrusions that interconnected fascia fibroblasts (FIG. 23b).

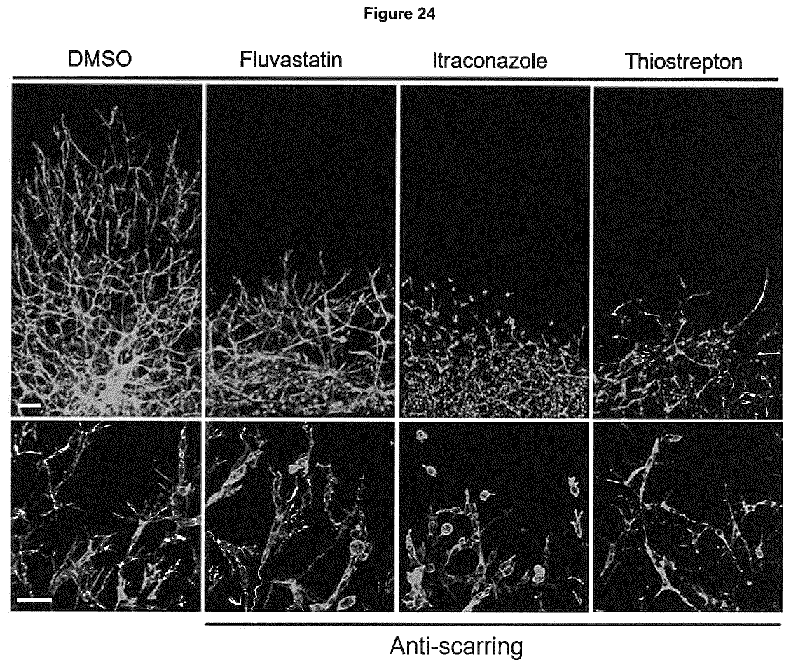

Among the five top anti-scarring chemicals, Fenbendazole (210) and Pyrvinium pamoate (1040) showed toxic effects to fascia tissues, so the Inventors focused on the other three for mechanism study. The three anti-fibrotic agents Itraconazole (1139), Thiostrepton (522), and Fluvastatin sodium salt (859) all significantly perturbed the magnitude of inter-cell connectivity and completely inhibited sprouting and reticulations within the fascia, whereas control fascia showed extensive sprouting and reticulations followed by a massive increase in invasion speed and change in overall fascia dimensions (FIG. 24).

Anti-Scar Agents Inhibit Fibroblast Reticulation Through Sonic-Hedgehog

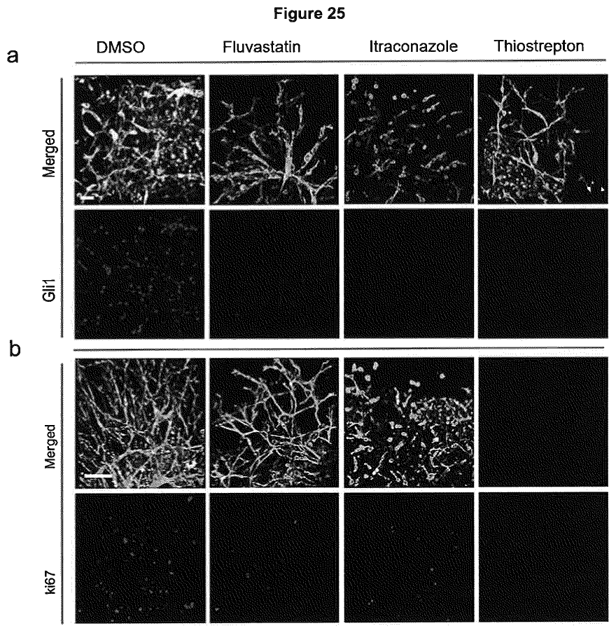

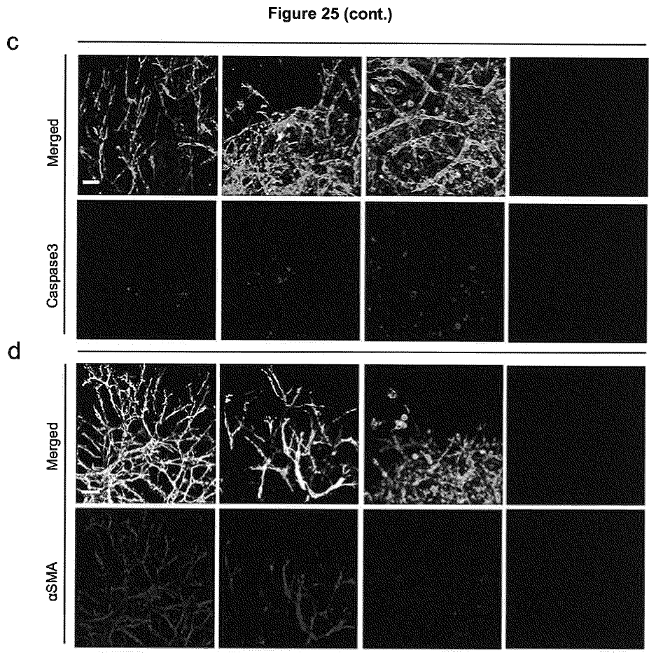

To better understand the molecular basis of reticulations that leads to scarring, the Inventors took a closer look at the molecular targets of the ‘hit’ chemicals. All three anti-scarring chemicals, including the pro-scarring chemical, all effectively targeted hedgehog pathway signaling, in multiple ways and Gli-1 nuclear protein expression was significantly decreased in the presence all three anti-scarring treatments (FIG. 25a). Thiostrepton reduces the binding activity of the transcriptional factor FoxM1; a positive regulator of Gli1 signaling (REF). Whereas Itraconazole inhibits Gli nuclear activity through stopping SMO moving to the membrane of primary cilia and thus inhibiting the pathway (James et al., 2010). Indeed, In the Itraconazole-treated sample there is no expression of Gli1. Furthermore, cells were not activated into myofibroblasts as αSMA expression was trapped in the nucleus rather than the cytoplasm. The 3^{rd} anti-scar drug Fluvastatin inhibits 3-Hydroxy-3-methylglutaryl-coenzyme A (HMG-CoA) reductase, a rate-limiting enzyme in cholesterol biogenesis that is required for hedgehog pathway activation (Huang P et al., 2016; Giovanni Luchetti et al., 2016). However, Gli1 is weakly expressed in the Fluvastatin-treated samples and there is also intact cell-cell connection and network formation. This may because that the cholesterol synthesis is not totally blocked, and cells can be activated into myofibroblast (FIG. 25b). Thus, the Inventors conclude that anti-fibrosis chemicals converge on SHH pathway signaling to modulate fibroblast sprouting and reticulations, needed to mobilize fascia jelly into sites of injury.

The hedgehog pathway was also reported to induce proliferation and to depress apoptosis. Ki67 and caspase 3 staining showed that cell proliferation activity of Thiostrepton, Itraconazole and Fluvastatin treated samples decreased (FIG. 25c), whereas cell death increased (FIG. 25d).

Anti-Scar Agents Inhibit Fascia Mobility In Vivo

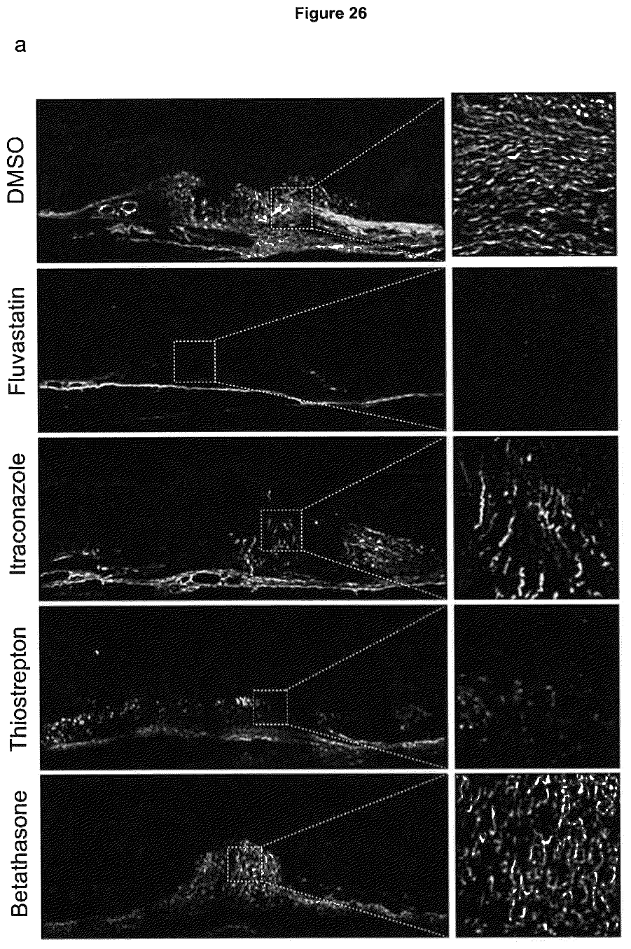

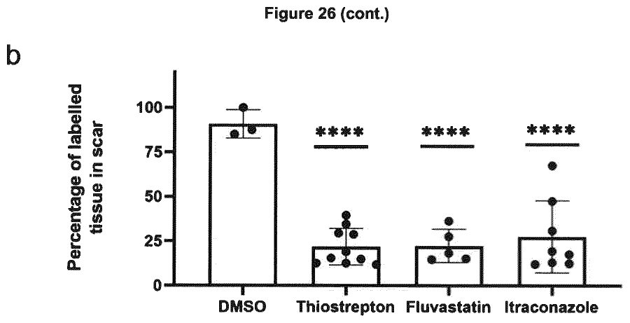

Having discovered sprouting and reticulations are altered by the compounds, the Inventors went on to check how these effect fascia mobilization in physiologic wounds in live animals. First, the Inventors labelled the subcutaneous fascia layer with a FITC-NHS ester dye in animals, then made full thickness excisional wounds on the backs of fascia-labelled mice, and followed wound healing under the presence of a weekly injection regimen using the above 3 separate compounds. Skin wounds were collected at Day 7 after wounding to determine the extent of fascia mobilization and also at 21 days post-injury to determine final wound size and scar severity. In DMSO control groups, the fascia matrix jelly was mobilized into the wound from all sides of the wound, and wounds at 7 days were completely clogged with large patches of labelled matrix jelly. All three anti-scarring drugs, on the other hand, significantly reduced fascia mobilization into wounds. Fluvastatin completely inhibited matrix jelly movements. Itraconazole and Thiostrepton treatments also inhibited fascia jelly movements, with only marginal connective tissue fragments relocating into wounds, and without the massive shift seen in controls (FIG. 26).

Anti-Scar Agents Inhibit Scar Formation In Vivo

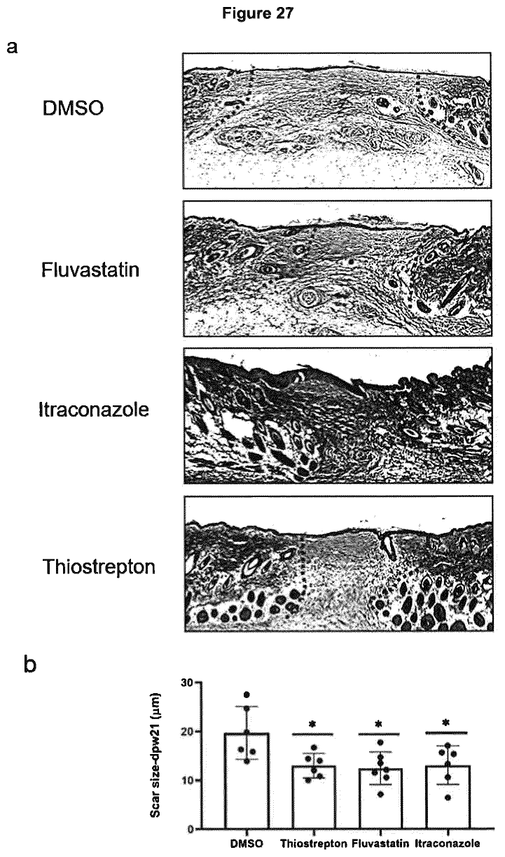

Next, the Inventors tested the three chemicals in an in vivo splinted wound model to document their overall effects on scarring. Trichrome staining showed that all the three anti-scarring chemicals gave smaller scars on day 21 after wounding (FIG. 27). This result is therefore perfectly consistent with the results of ex vivo by SCAD assay and fascia invasion assay.

### Example 7: Body-Wide Reservoirs of Fluid Matrix Fuel Organ Healing & Regeneration

Damaged organs repair injuries by forming new connective tissue, reestablishing structural and mechanical continuums that ensure survival, but it has been unclear how connective tissues repopulate and rebuild the injured site. Specifically, it was believed that local fibroblasts secrete new extracellular matrix.

Here the Inventors focus on three different internal organs to reveal the basis of damage repair. By separately tagging extracellular matrix of liver, cecum and peritoneum before injury in live mice the Inventors demonstrate that the matrix itself plays the primary role in the damage response. Thus, the Inventors identify reservoirs of fluid-like matrix in connective tissues that gush across organs to repair liver, cecal and peritoneal wounds.

Using proteomics analysis, the Inventors uncover distinct compositions of fluid matrix that lead to regeneration or scarring and fibrous adhesions. Using single cell analysis and mechanistic studies, the Inventors uncover neutrophils orchestrate matrix flows and are functionally primed for matrix transportation in multiple ways. Blocking neutrophil adherence, their chemotactic or nitric oxide signaling inhibited matrix flows, and curbed postsurgical adhesions and liver regeneration. The finding of a body-wide reservoir of fluid matrix reconfigures the traditional view of wound repair, and provides a wide potential novel therapeutic space to treat impaired wounds and excessive scarring across a range of human diseases/conditions.

Methods

### Animals

All mouse lines were obtained (C57BL/6J, B6.129P2-Lyz2tm1(cre)lfo/J (Lyz2Cre), B6; 129S6-Gt(ROSA)26Sor^{tm14(CAG-tdTomato)Hze}/J (Ai14)) from Jackson Laboratories or Charles River and bred and maintained in the Helmholtz Animal Facility in accordance with EU directive 2010/63. Animals were housed in individual ventilated cages (IVC) and animal housing rooms were maintained at constant temperature and humidity with a 12-h light cycle. Animals were supplied with water and chow ad libitum. All animal experiments were reviewed and approved by the Government of Upper Bavaria and registered under the project number ROB-55.2-2532.Vet_02-19-133 or ROB-2532.Vet_02-19-148 and conducted under strict governmental and international guidelines. This study is compliant with all relevant ethical regulations regarding animal research.

### Injury Models

Thirty minutes before surgery mice received a preemptive subcutaneous injection with Metamizole (200 mg/kg bw). Anesthesia was supplied by an intraperitoneal injection of a Medetomidin (500 μg/kg), Midazolam (5 mg/kg) and Fentanyl (50 pg/kg) cocktail, hereafter referred to as MMF. Monitoring anesthetic depth was assessed by toe reflex. Eyes were covered with Bepanthen-cream to avoid dehydration, and the abdomen was shaved and disinfected with betadine and sterile phosphate buffered saline (PBS). Animals were kept on their backs on a heating plate at 39° C. A midline laparotomy (1-1.5 cm) was performed through the skin and peritoneum. Four hooks, positioned around the incision and fixed to a retractor and magnetic base plate, allowed for clear access to the abdominal cavity and liver.

Local damage to the liver surface was induced via electroporation tweezers by applying 30V 50 ms pulses at 1s interval for 8 cycles. Before closure of the incision, Buprenorphine (0.1 mg/kg) was pipetted in the abdomen to allow for initial post-surgical analgesia. For long-term analgesia, Metamizole (Novalgin, 200 mg/kg) was provided through daily injections. The peritoneum and skin were closed with two separate 4-0 silk sutures (Ethicon). Upon closure of the incision, mice were woken up by antagonizing Medetomidin and Midazolam through a subcutaneous cocktail injection of Atipamezol (1 mg/kg) and Flumazenil (0.25 mg/kg). Mice were allowed to recover on a heating pad, after which they were single housed. Mice were sacrificed after indicated time points and liver tissue was obtained. In the peritoneal model, surgical procedure was as described above, but the peritoneal areas were marked.

To induce adhesions between liver and peritoneum, abrasion was applied to the electroporated side of the liver and to the opposite side of the peritoneum. In the peritoneal-cecal adhesion model, surfaces of cecum and peritoneum were injured with a brush, two surgical knots were placed and talcum powder was applied onto wound sides of both organs. Immune cell knockout was performed as follows: inhibitors were injected intraperitoneally 2 hours before surgery at a concentration of 10 μM in sterile PBS. Neutralizing antibodies (Bio X Cell) were applied at a concentration of 200 pg/20g body weight. Clodronate Liposomes (Liposoma), CP-105,696 and LY255283 (Sigma Aldrich), TD139 (Probechem), W1400 (Enzo) and L-NAME (Biocat) were applied at a concentration of 10 μM. Lipoxin (Merck Millipore) was applied locally by soaking the reagent in a sterile filter paper with 100 nM solution and applying the filter paper over the liver surface for 5 minutes.

### Human Tissue

All human samples were obtained from surgery at the Department of Surgery, Klinikum rechts der Isar, Technical University of Munich, following approval of the local ethics committee of the Technical University of Munich, Germany (Nr. 173/18 S). Adhesions were intraoperatively diagnosed and dissected from the respective organs and prepared for further analysis.

### Labeling of ECM on Organ Surfaces

Succinimidyl esters (NHS-esters; Thermo Fisher) were diluted in DMSO to a concentration of 25 mg/ml and stored at −80° C. To obtain ectopic labeling of matrix, the Inventors generated a labelling solution by mixing NHS-ester 1:1 with 100 mM pH 9.0 sodium bicarbonate buffer. Sterile Whatman filter paper (Sigma Aldrich) biopsy punches where soaked in NHS-labelling solution, and locally placed on the liver surface. After one minute, the labelling punch was removed. For global abdominal labelling, 20 μl of NHS-labelling solution were mixed with 100 μl sterile PBS and injected i.p. For kinetic measurements organ surfaces were marked with either a 1.0 cm (near) or 2 cm (far) 2 mm filter patch with NHS-FITC.

### Tissue Preparation

Upon organ excision, organs were fixed overnight at 4° C. in 2% formaldehyde. The next day, fixed tissues were washed three times in Dulbecco's phosphate buffered saline (DPBS, GIBCO, #14190-094), and depending on the purpose, either embedded, frozen in optimal cutting temperature compound (Sakura, #4583) and stored at −20° C., or stored at 4° C. in PBS containing 0.2% gelatin (Sigma Aldrich, #G1393), 0.5% Triton X-100 (Sigma Aldrich, #X100) and 0.01% Thimerosal (Sigma Aldrich, #T8784) (PBS-GT). Fixed tissues were embedded in optimal cutting temperature (OCT) compound and cut with a Microm HM 525 (Thermo Scientific) by the standard protocol. In short, In short, sections were fixed in ice-cold acetone for 5 min at −20° C., and then washed with PBS. Sections were then blocked for non-specific binding with 10% serum in PBS for 60 minutes at room temperature, and then incubated with primary antibody in blocking solution O/N at 4° C. The next day, following washing, sections were incubated in PBS with fluorescent secondary antibody, for 120 min at RT. Finally, sections were washed and incubated with Hoechst 33342 nucleic acid stain (Invitrogen, #H1399), washed in ddH₂O, mounted with Fluoromount-G® (Southern Biotech, #0100-01), and stored at 4° C. in the dark. Primary antibodies: rabbit-anti-collagen I (1:150, Rockland), rabbit-anti-Cytokeratin (1:100, Sigma Aldrich), rabbit-anti-Ki67 (1:100, Abcam), rabbit-anti-Fibronectin (1:100, Abcam), rabbit-anti-HSP70 (1:100, Elabscience), rabbit-anti-HSPG2 (1:100, Elabscience), rabbit-anti-Keratin9 (1:100, Elabscience), rabbit-anti-Ki67 (1:100, Abcam), rabbit-anti-cleaved Caspase 3 (1:100, Abcam), rabbit-anti-Laminin (1:100, Abcam), rabbit-anti-HSP70 (1:100, Elabscience), hamster-anti-PDPNα (1:100, Abcam), rat-anti-LY6G(Sca1) (1:100, Abcam), rabbit-anti-MMP23(1:100, Elabscience), rabbit-anti-Vitronectin (1:100, Elabscience) and rabbit-anti-WT1 (1:100, Abcam). Alexa Fluor 488-, Alexa Fluor 568- or Alexa Fluor 647-conjugated antibodies (1:500, Life technologies) against suitable species were used as secondary antibodies. H&E staining was performed according to manufacturer's protocol (Sigma).

### Microscopy

Histological sections were imaged under a M205 FCA Stereomicroscope (Leica). For whole-mount 3D imaging of tissues, fixed samples were embedded in 35-mm glass bottom dishes (Ibidi) with low-melting point agarose (Biozym) and left to solidify for 30 min. Imaging was performed with a Leica SP8 multi photon microscope (Leica, Germany). For time-lapse imaging liver and peritoneal tissues, samples were embedded as just above. Imaging medium (DMEM/F-12) was then added. Time-lapse imaging was performed under the M205 FCA Stereomicroscope. A modified incubation system, with heating and gas control (ibidi, catalogue nos. 10915 and 11922), was used to guarantee physiologic and stable conditions during imaging. Temperature control was set to 35° C. with 5% CO₂-supplemented air. 2D, 3D and 4D data was processed with Imaris 9.1.0 (Bitplane) and ImageJ (1.52i). Contrast and brightness were adjusted for better visibility.

### Protein Biochemistry

Tissues were snap frozen and grinded using a tissue lyser (Qiagen). Pulverised tissues were resuspended in lysis buffer (20 mM Tris-HCl pH 7.5, 1% Triton X-100, 2% SDS, 100 mM NaCl, 1 mM sodium orthovanandate, 9.5 mM sodium fluoride, 10 mM sodium pyruvate, 10 mM beta-glycerophosphate), and supplemented with protease inhibitors (complete protease inhibitor cocktail, Pierce) and kept 10 min on ice. Samples were sonicated and spun down for 5 minutes at 10,000g. Supernatants were stored at −80° C. Protein concentrations were determined via BCA-Assay according to the manufacturer's protocol (Pierce).

Protein pulldown was as follows. Lysates were diluted with a pulldown buffer (20 mM Tris-HCl pH 7.5, 1% Triton X-100, 100 mM NaCl, supplemented with protease and phosphatase inhibitors) and incubated overnight with dynabeads (Thermo Fisher) according to the manufacturer's instructions at 4° C. on a rotator. The next day, the samples were each diluted twice with Wash Buffer 1 (20 mM Tris-HCl pH 7.5, 1% Triton X-100, 2% SDS, 100 mM NaCl and supplemented with protease and phosphatase inhibitors) and then with Wash Buffer 2 (20 mM Tris-HCl pH 7.5, 0.5% Triton X-100, 100 mM NaCl) and supplemented with protease and phosphatase inhibitors and finally washed twice with Wash Buffer 3 (20 mM Tris-HCl pH 7.5 and 100 mM NaCl). Beads were then resuspended in Elution Buffer (20 mM Tris-HCl pH 7.5, 100 mM NaCl and 50 mM DTT) and incubated for 30 minutes at 37° C. Finally, the samples were boiled for 5 minutes at 98° C. and the supernatants were stored at −80° C. Fluorescence intensities of lysates were measured in a Fluostar optima fluorometer (BMGlabtech).

### Mass Spectrometry

Tissues were marked locally with an EZ-LINK-NHS 100:1 FITC-NHS mixture. After 24 hours the organs were removed. Tissue pieces from the original marking were separated from moved matrix fractions and snap frozen. Tissue lysis was performed as described above. Samples were digested by a modified FASP procedure²³. After reduction and alkylation using DTT and IAA, the proteins were centrifuged on Microcon® centrifugal filters (Sartorius Vivacon 500 30 kDa), washed thrice with 8 M urea in 0.1 M Tris/HCl pH 8.5 and twice with 50 mM ammoniumbicarbonate. The proteins on the filter were digested for 2 hours at room temperature using 0.5 μg Lys-C (Wako Chemicals, Neuss, Germany) and for 16 hours at 37° C. with 1 pg trypsin (Promega, Mannheim, Germany). Peptides were collected by centrifugation (10 min at 14,000 g), acidified with 0.5% TFA and stored at −20° C. until measurements. The digested peptides were loaded automatically onto an HPLC system (Thermo Fisher Scientific) equipped with a nano trap column (100 μm ID×2 cm, Acclaim PepMAP 100 C18, 5 μm, 100 Å/size, LC Packings, Thermo Fisher Scientific, Bremen, Germany) in 95% buffer A (2% ACN, 0.1% formic acid (FA) in HPLC-grade water) and 5% buffer B (98% ACN, 0.1% FA in HPLC-grade water) at 30 μl/min. After 5 min, the peptides were eluted and separated on the analytical column (nanoEase MZ HSS T3 Column, 100 Å, 1.8 μm, 75 μm×250 mm, Waters) at 250 nl/min flow rate in a 105 minute non-linear acetonitrile gradient from 3 to 40% in 0.1% formic acid. The eluting peptides were analyzed online in a Q Exactive HF mass spectrometer (Thermo Fisher Scientific, Bremen, Germany) coupled to the HPLC system with a nano spray ion source and operated in the data-dependent mode. MS spectra were recorded at a resolution of 60,000 and after each MS1 cycle, the 10 most abundant peptide ions were selected for fragmentation. Raw spectra were imported into Progenesis QIsoftware (version 4.1, Nonlinear Dynamics, Waters). After feature alignment and normalization, spectra were exported as Mascot Generic files and searched against the SwissProt mouse database (16,872 sequences) with Mascot (Matrix Science, version 2.6.2) with the following search parameters: 10 ppm peptide mass tolerance and 0.02 Da fragment mass tolerance, two missed cleavages allowed, carbamidomethylation was set as fixed modification, camthiopropanoyl, methionine and proline oxidation were allowed as variable modifications. A Mascot-integrated decoy database search calculated an average false discovery of <1% when searches were performed with a mascot percolator score cut-off of 13 and an appropriate significance threshold p.

Peptide assignments were re-imported into the Progenesis QI software and the abundances of all unique peptides allocated to each protein were summed up. The resulting normalized abundances of the individual proteins were used for calculation of protein ratios and p-values (ANOVA) between sample groups using a nested design. Gene ontology analysis was performed using the EnrichR webtool^{24,25}. Extracellular elements were identified through a database search against http://matrisomeproject.mit.edu/.

### Single Cell RNA-Seq

Three livers per experimental group were pooled for each sequencing run. For each liver, the electroporated area was punched out with a circular 4 mm biopsy punch, and subsequently minced with fine scissors into small pieces (approximately 1 mm²). The equivalent, but non-injured area was used in control livers. The resulting fragments were further processed by enzymatic digestion in 5 mL enzyme mix consisting of dispase (50 caseinolytic units/ml), collagenase (2 mg/ml), and DNase (30 pg/ml), for 30 min at 37° C. under constant agitation (180 rpm). Enzyme activity was inhibited by adding 5 ml of phosphate-buffered saline (PBS) supplemented with 10% fetal bovine serum (FBS). Dissociated cells in suspension were passed through a 70 μm strainer and centrifuged at 500×g for 5 min at 4° C. Red blood cell lysis (Thermo Fisher 00-4333-57) was performed for 2 min and stopped with 10% FBS in PBS. After another centrifugation step, the cells were counted in a Neubauer chamber and critically assessed for single-cell separation and viability. A total of 250,000 cells were aliquoted in 2.5 ml of PBS supplemented with 0.04% of bovine serum albumin and loaded for DropSeq at a final concentration of 100 cells/μL. DropSeq experiments were performed as described previously²⁶. In brief, using a microfluidic PDMS device (Nanoshift), single cells were co-encapsulated in droplets with barcoded beads (Chemgenes Corporation, Wilmington, Mass.) at a final concentration of 120 beads/uL. Droplets were collected for 15 min/sample. After droplet breakage, beads were harvested, washed, and prepared for on-bead mRNA reverse transcription (Maxima RT, Thermo Fisher). Following an exonuclease I (New England Biolabs) treatment for the removal of unused primers, beads were counted, aliquoted (2000 beads/reaction, equals ˜100 cells/reaction), and pre-amplified by 13 PCR cycles (primers, chemistry, and cycle conditions identical to those described previously²⁶). PCR products were pooled and purified twice on 0.6× clean-up beads (CleanNA). Prior to tagmentation, cDNA samples were loaded on a DNA High Sensitivity Chip on the 2100 Bioanalyzer (Agilent) to ensure transcript integrity, purity, and quantity. For each sample, 1 ng of pre-amplified cDNA from an estimated 1000 cells was tagmented by Nextera XT (Illumina) with a custom P5 primer (Integrated DNA Technologies). Single cell libraries were sequenced in a 100 bp paired-end run on the Illumina HiSeq4000 using 0.2 nM denatured sample and 5% PhiX spike-in. For priming of read 1, 0.5 μM Read1CustSeqB was used (primer sequence:

(SEQ ID NO: 1))
GCCTGTCCGCGGAAGCAGTGGTATCAACGCAGAGTAC.

Results

### Fluid Matrix Gushes Across Organs to Seed Wounds

Damaged tissues rebuild with a complex mixture of tissue and matrix, the provenance of which has remained obscure. The Inventors recently demonstrated in skin that loose connective tissue (matrix) serves a source for dermal scars. They therefore set out to test the possibility that fluid-like matrix systems might also be mobilized in response to injury in the internal organs.

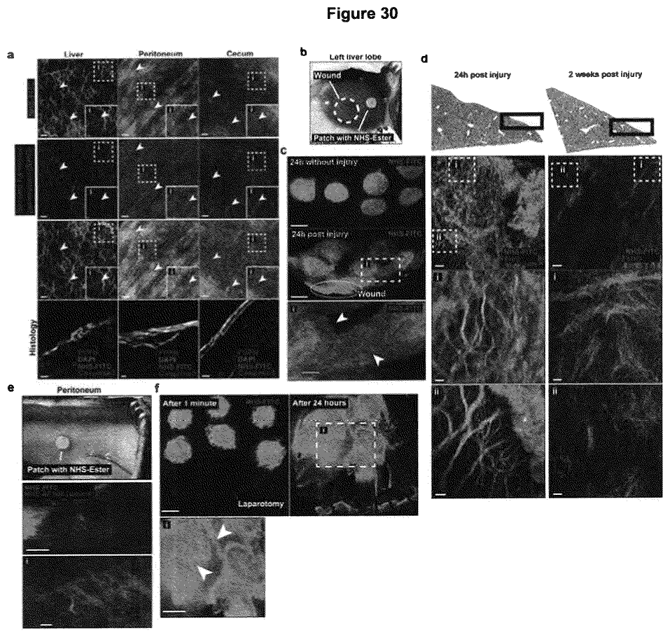

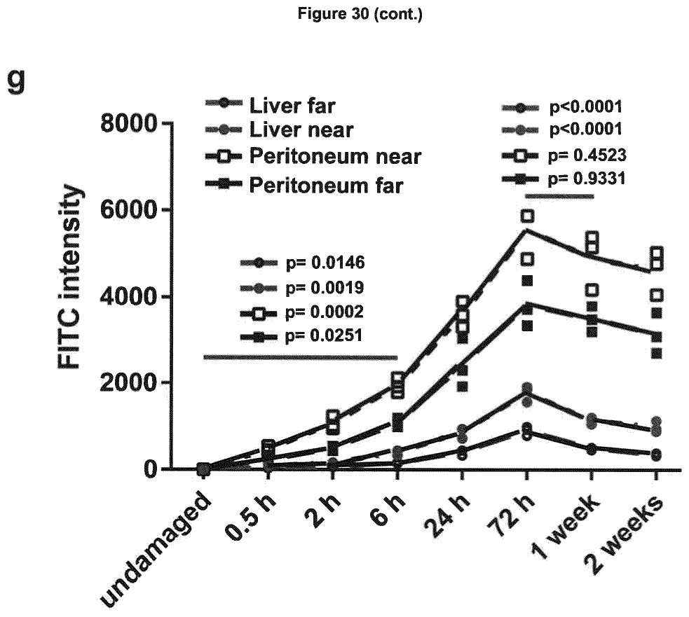

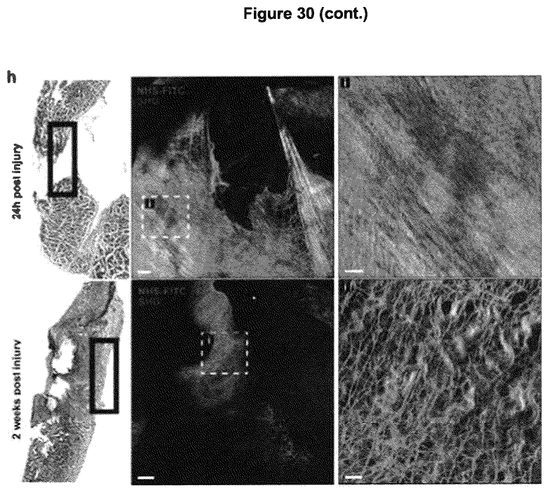

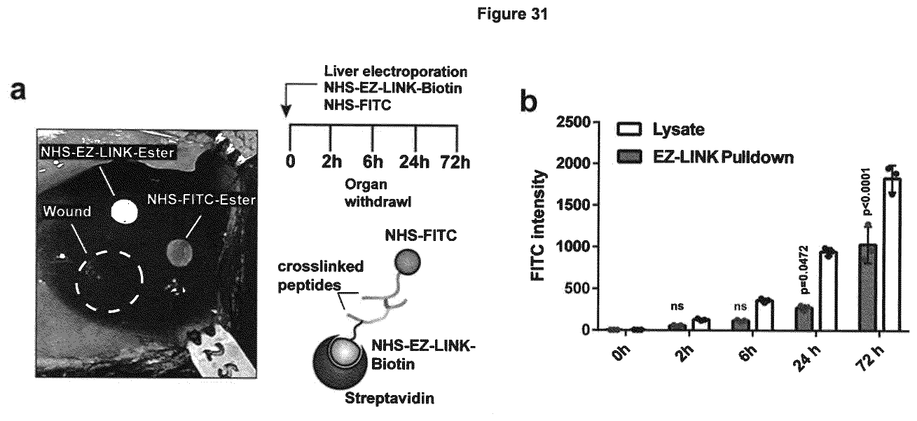

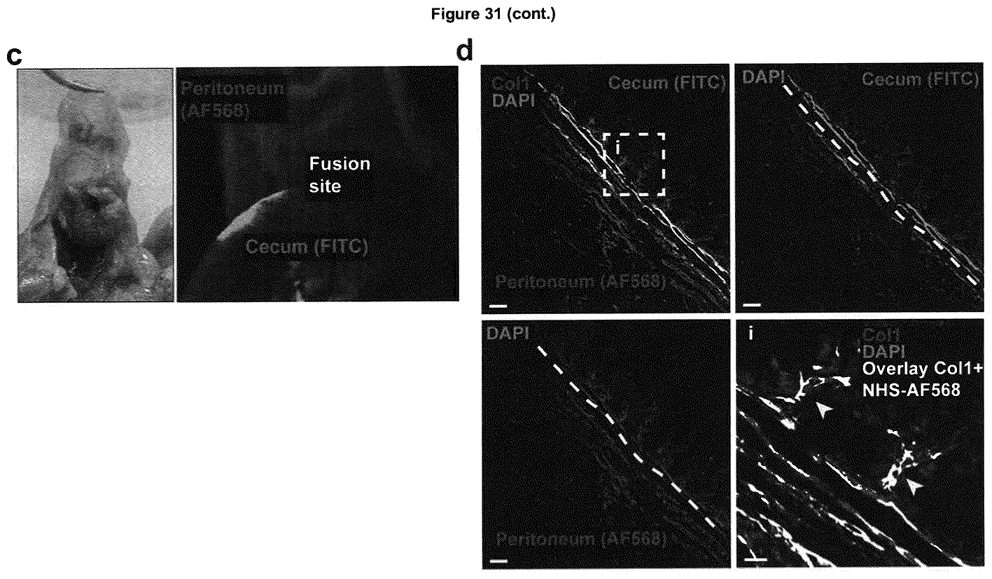

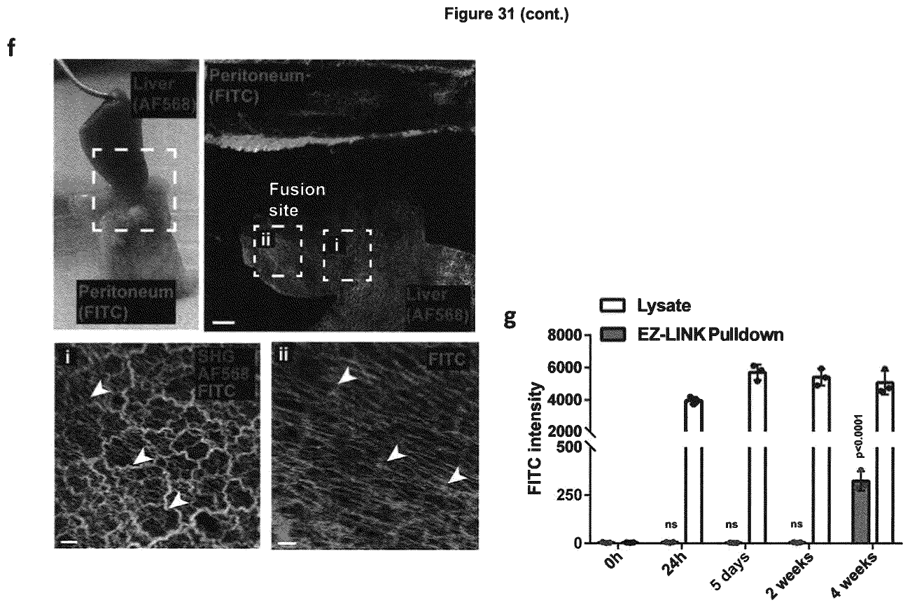

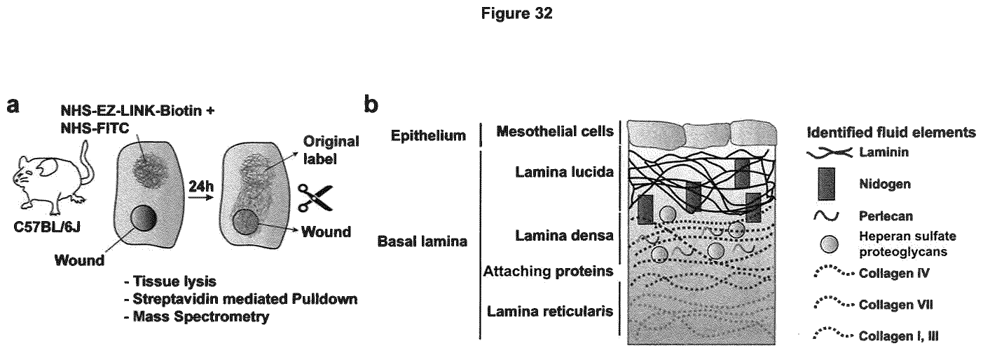

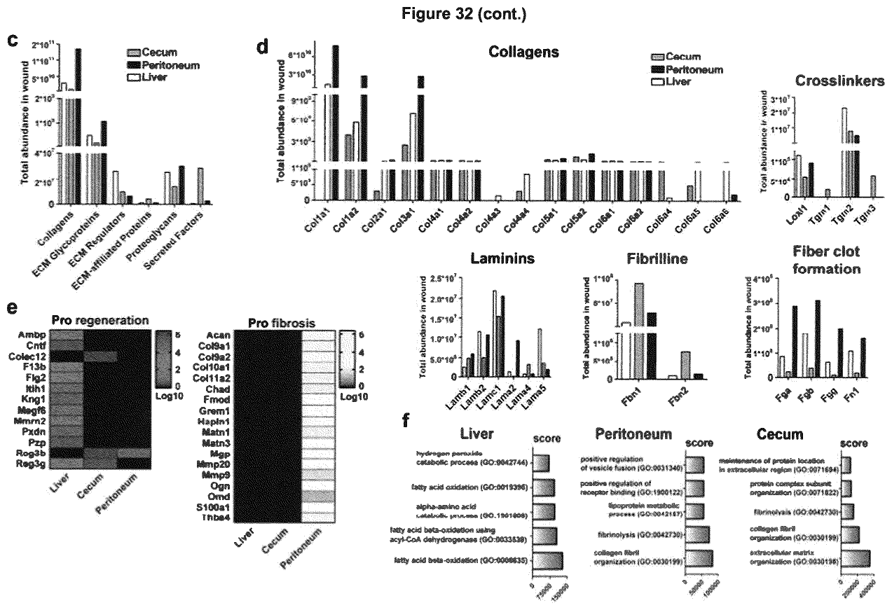

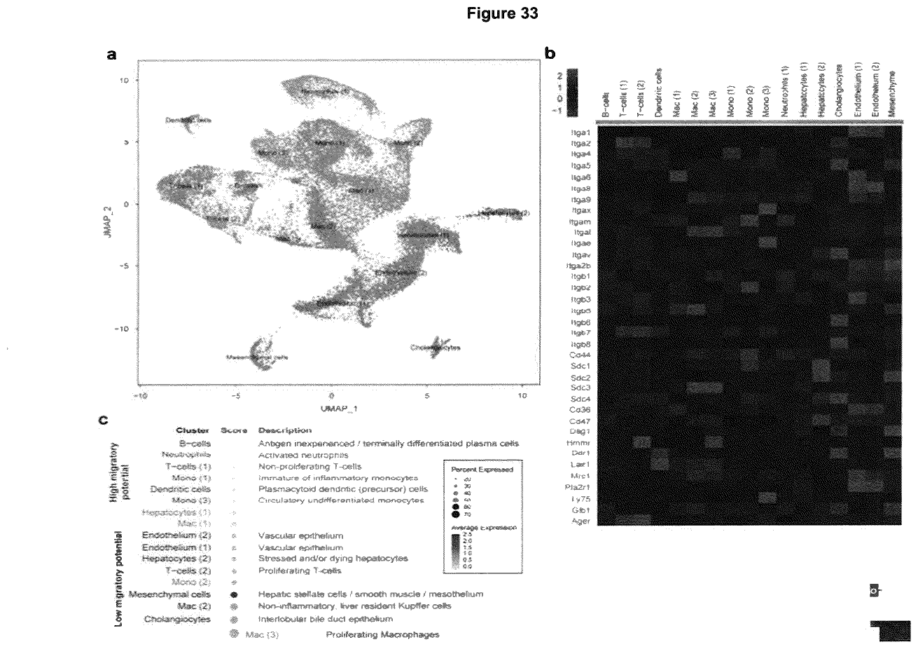

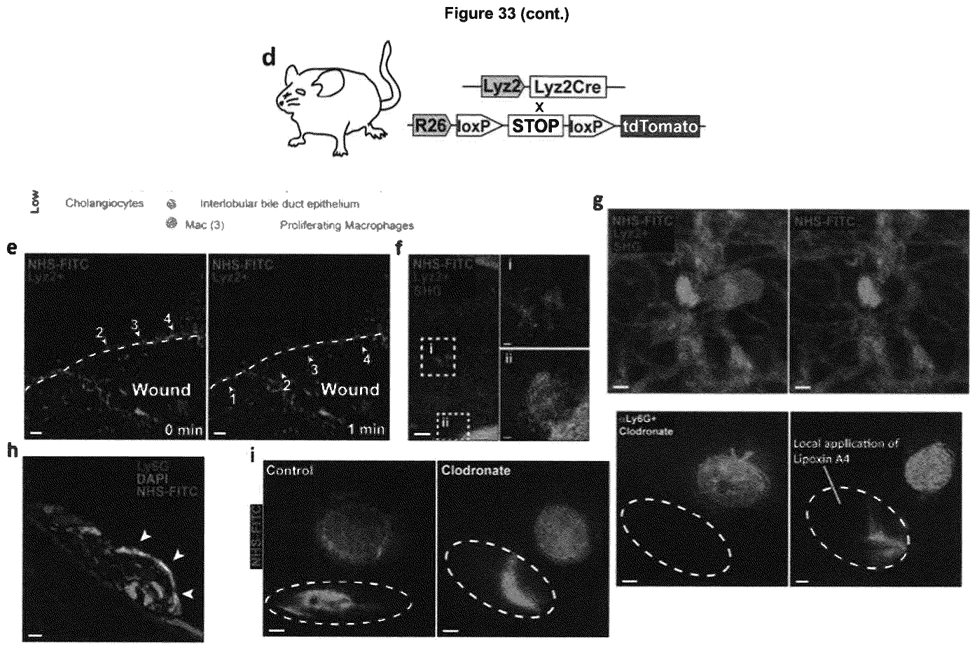

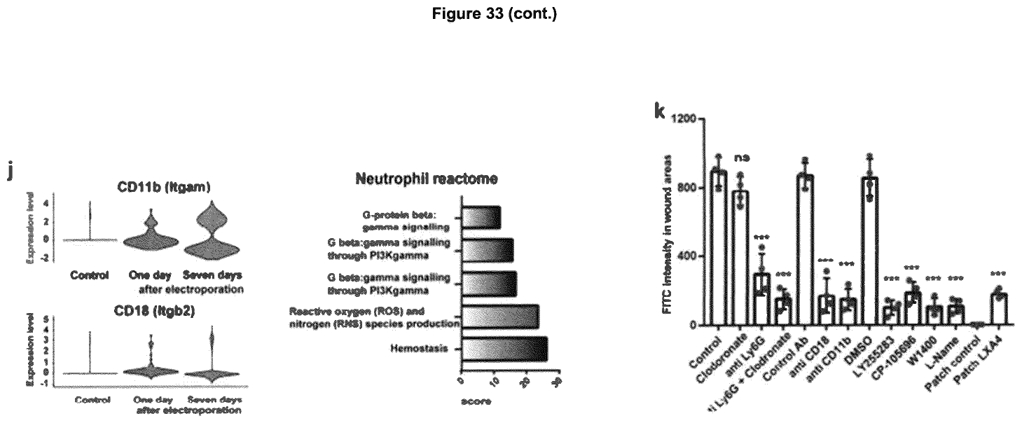

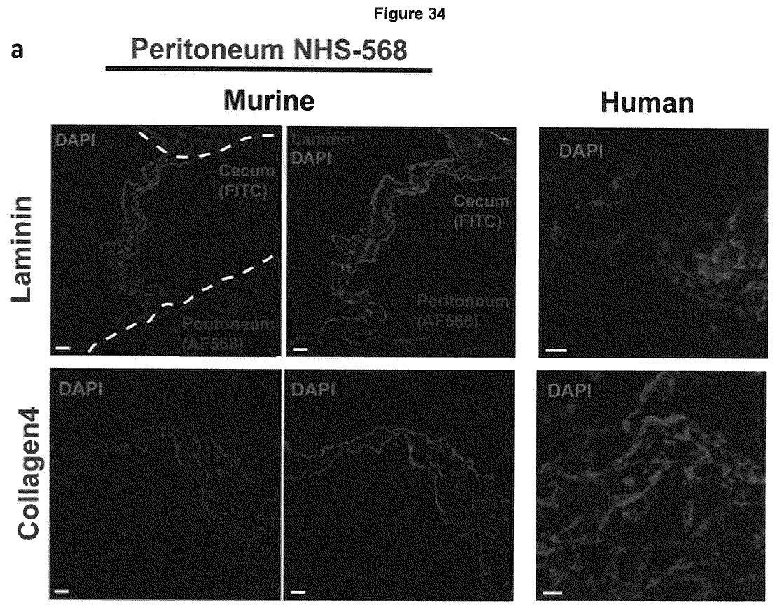

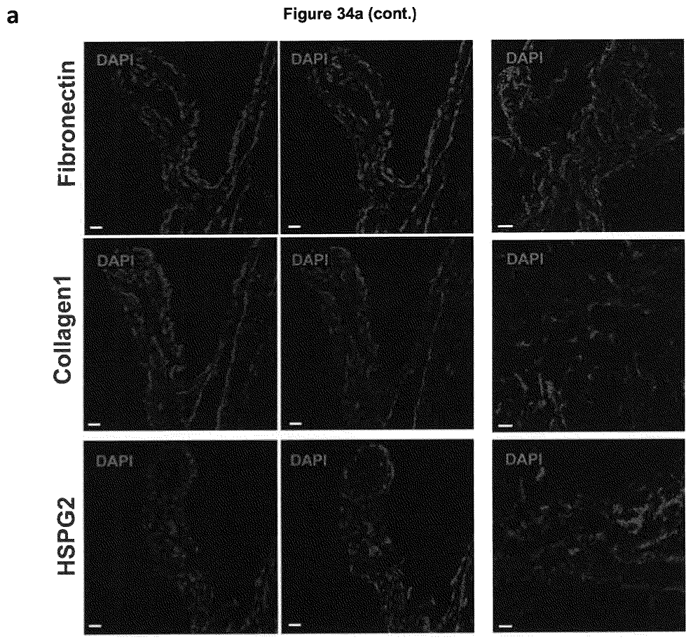

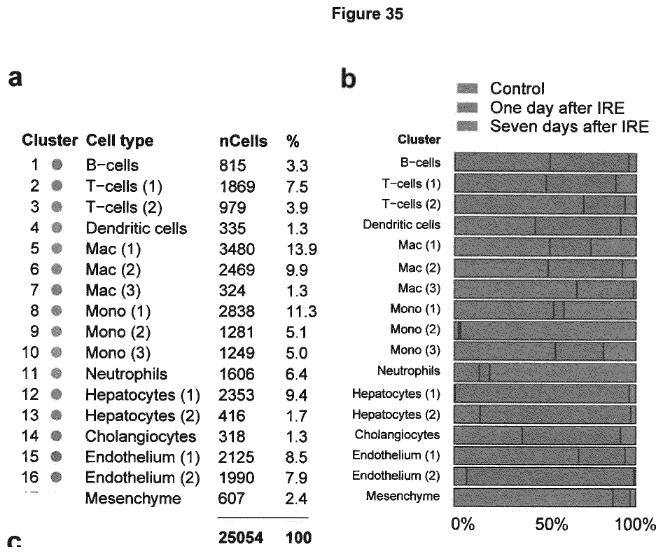

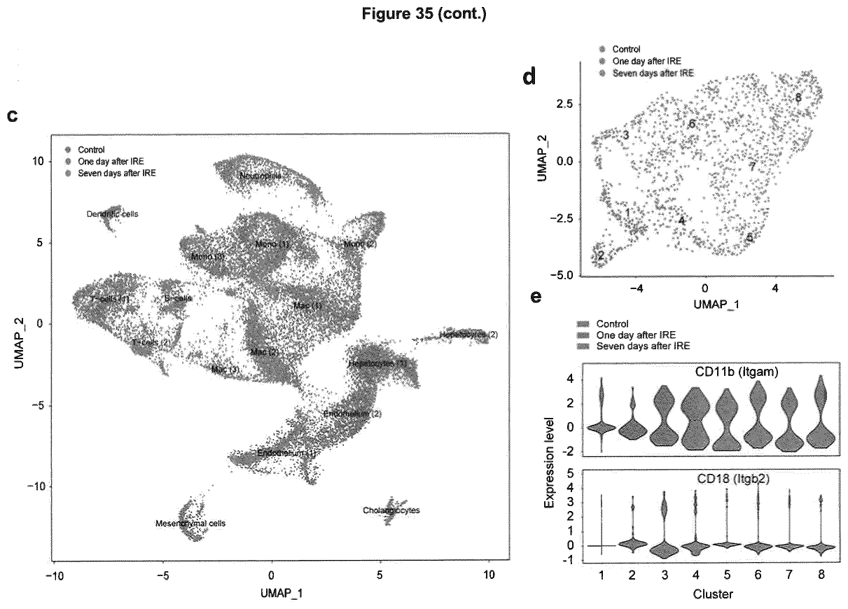

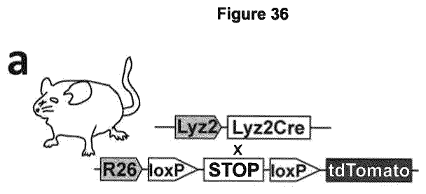

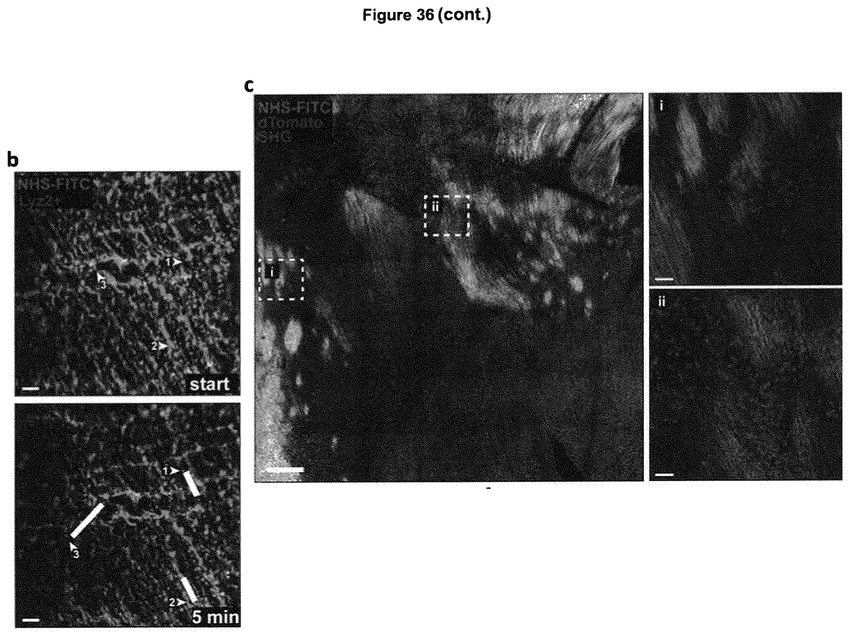

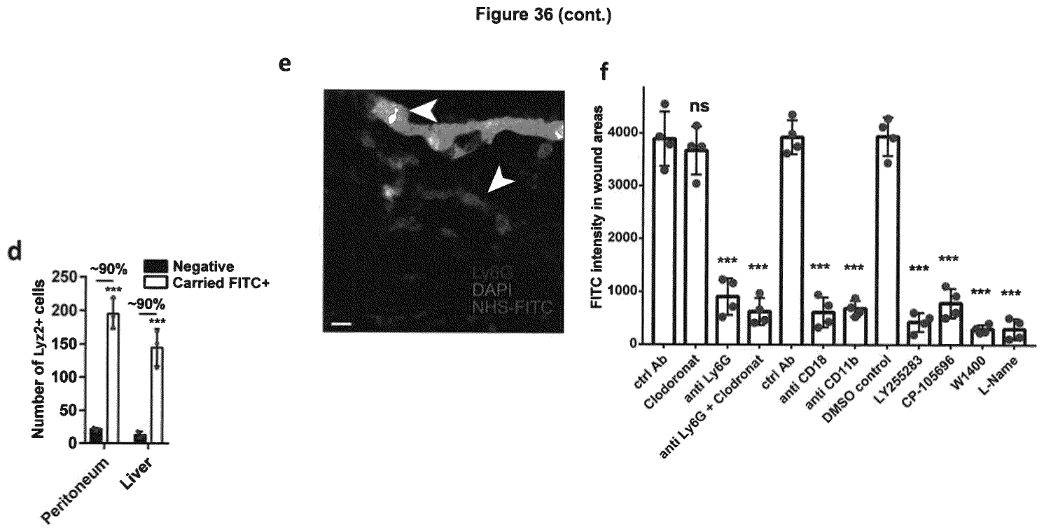

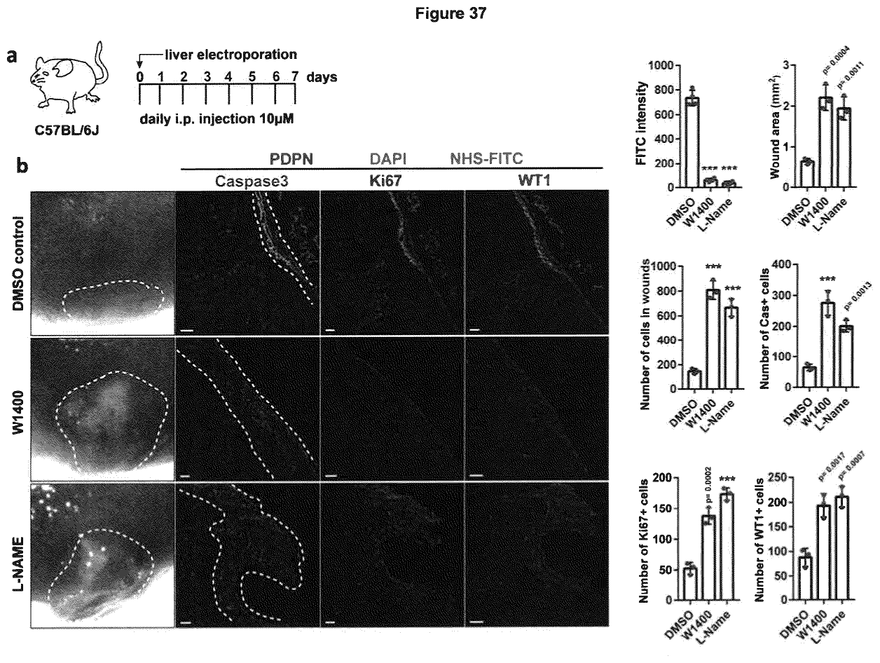

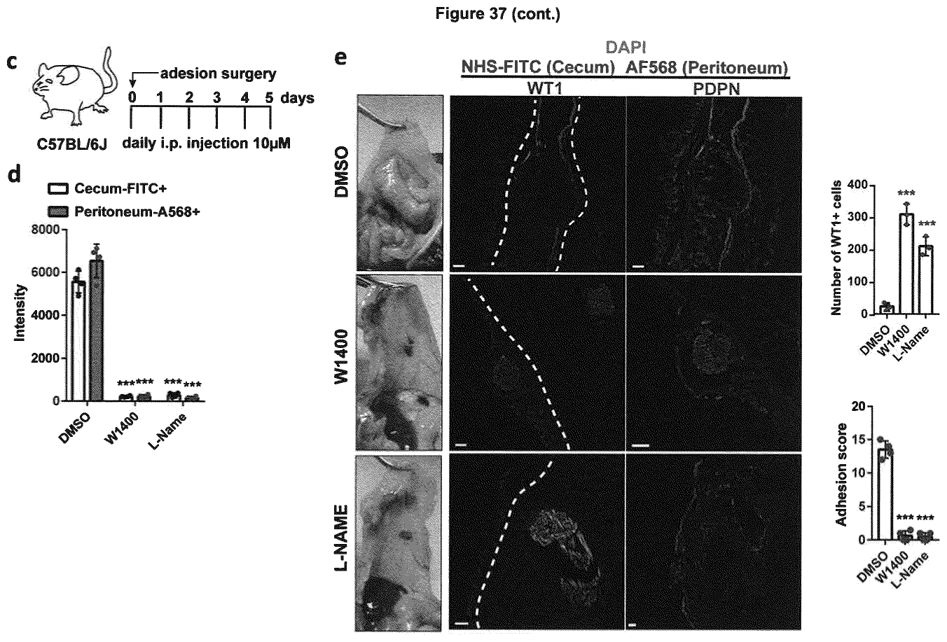

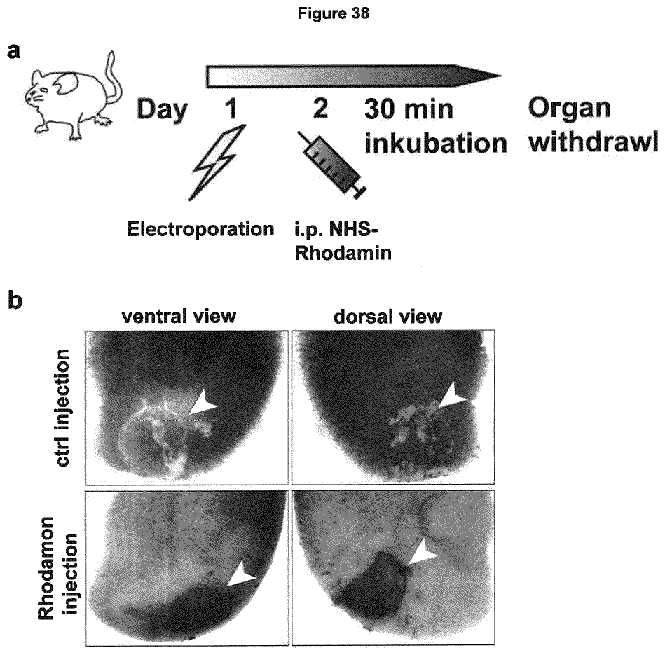

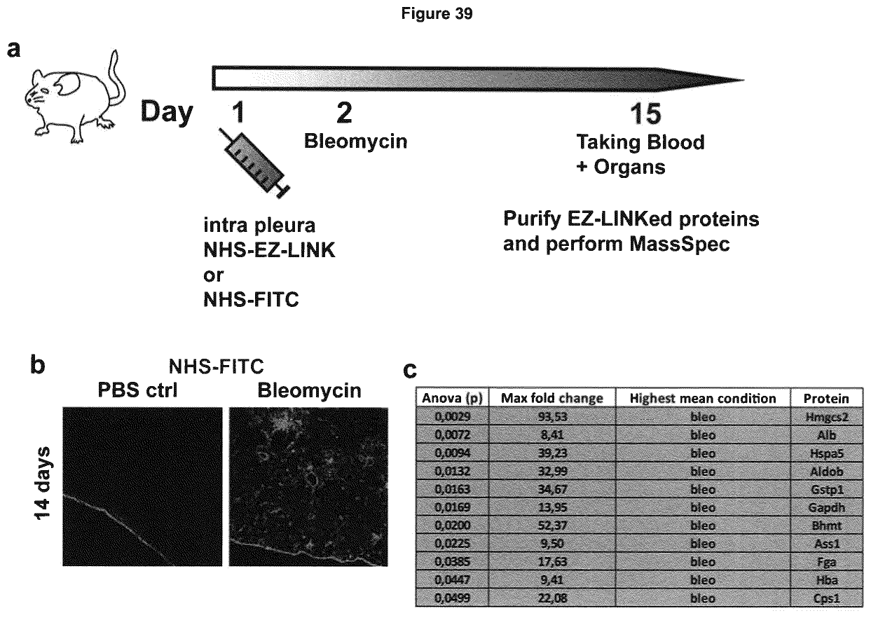

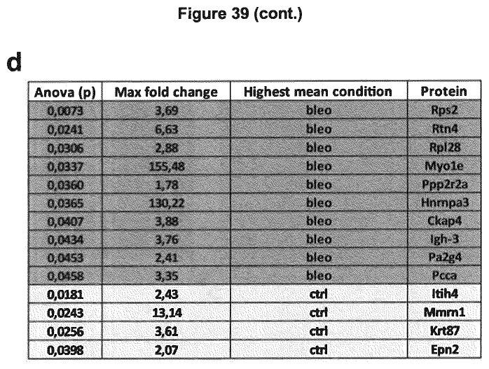

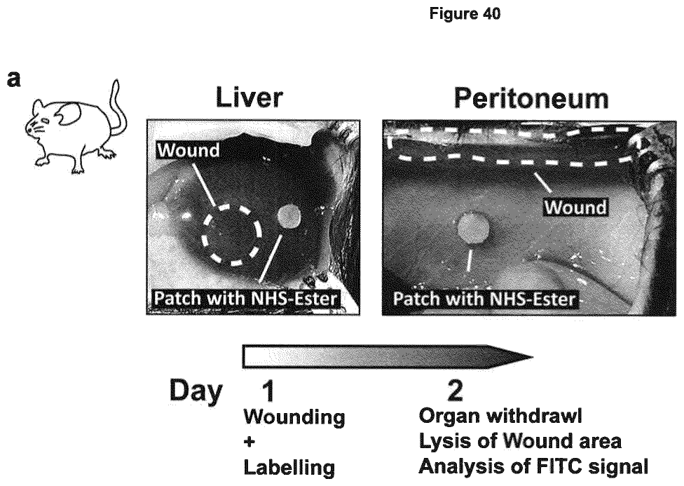

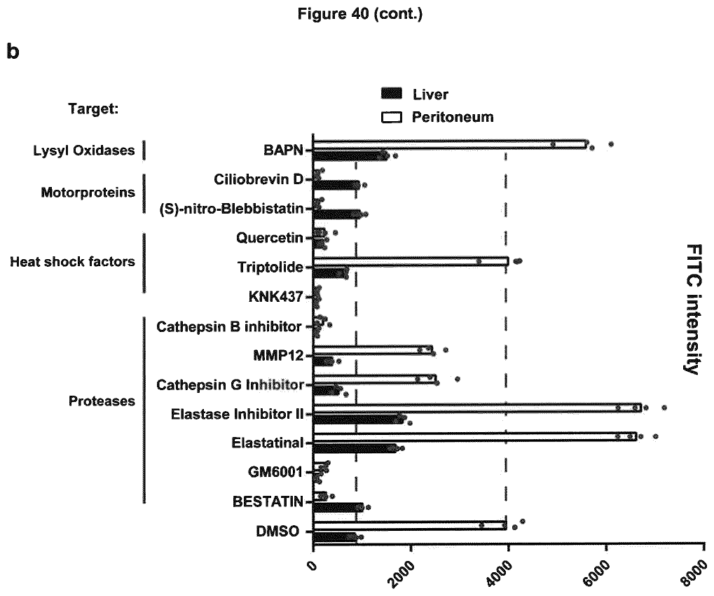

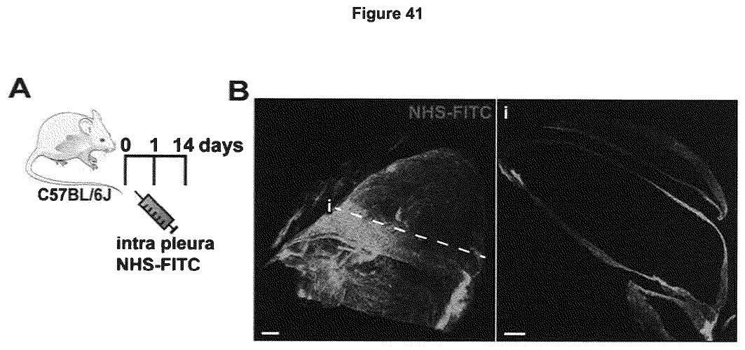

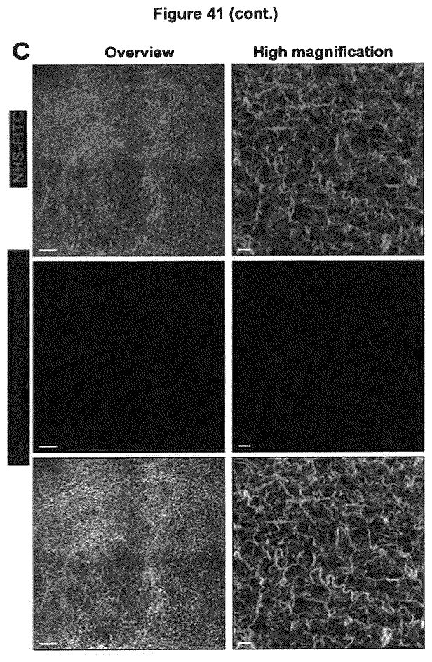

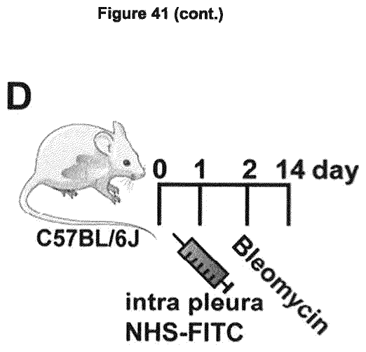

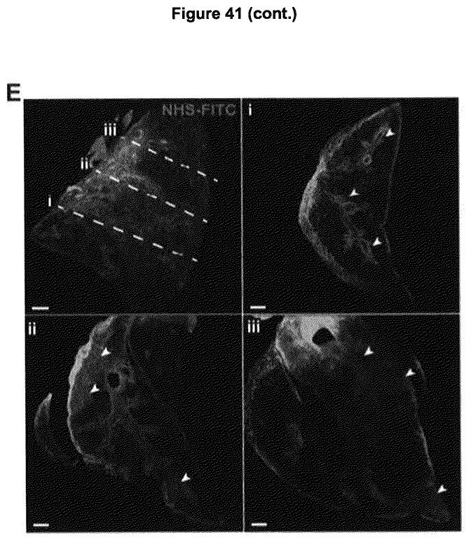

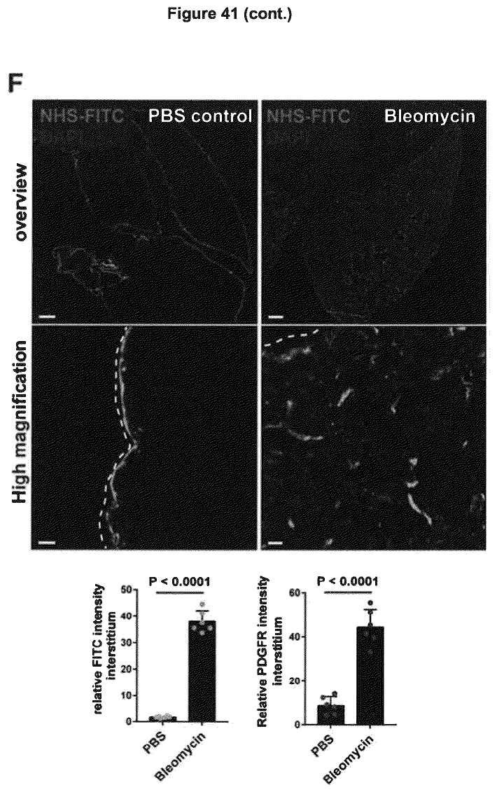

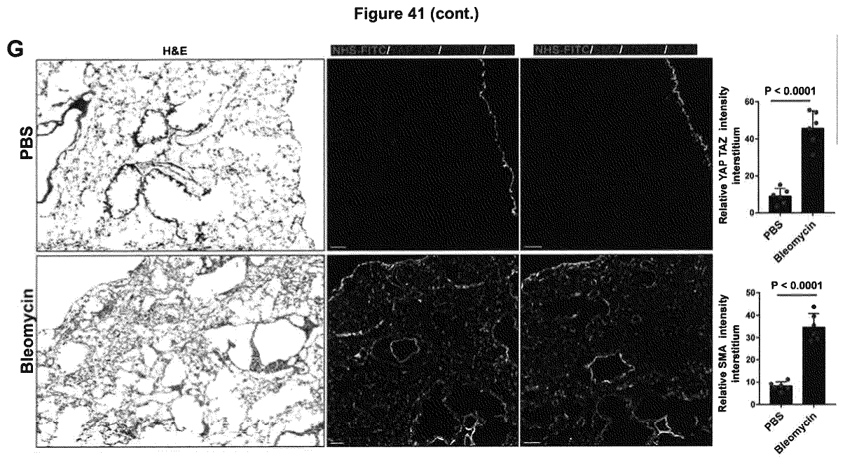

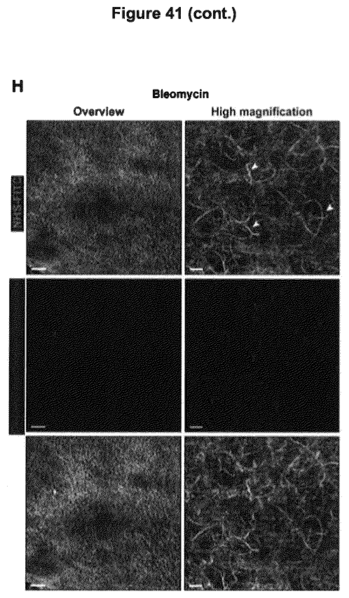

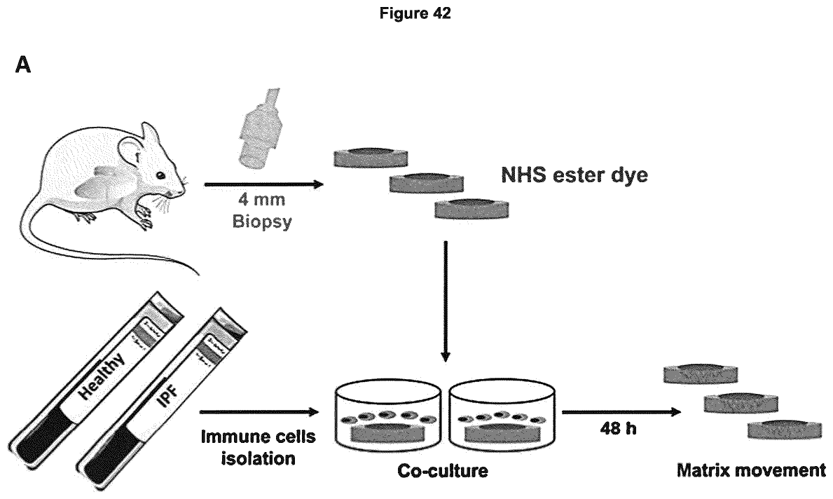

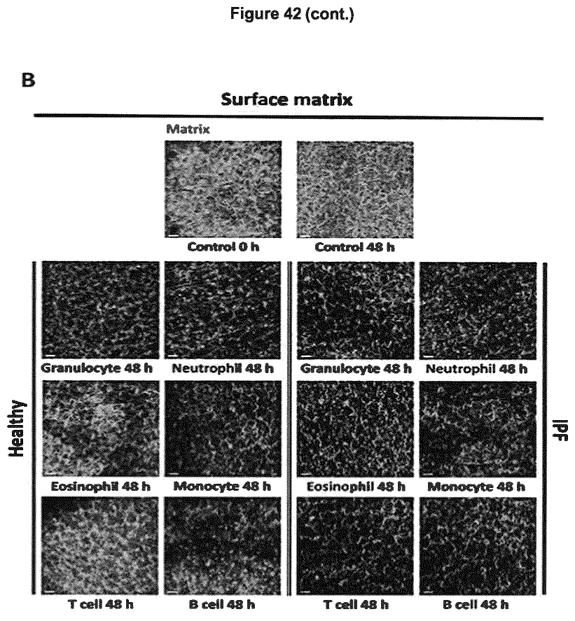

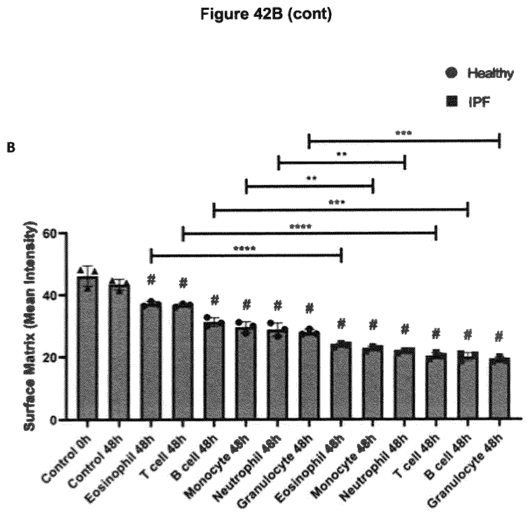

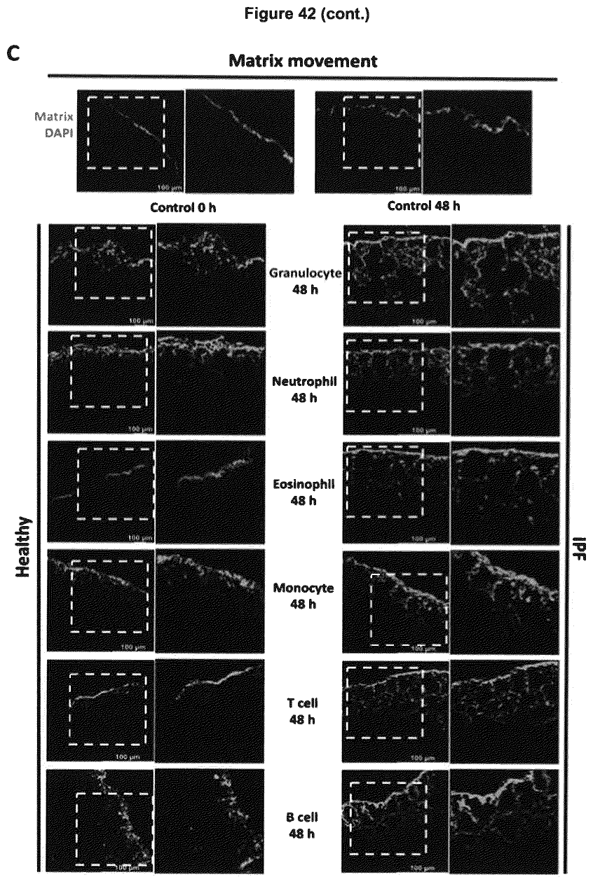

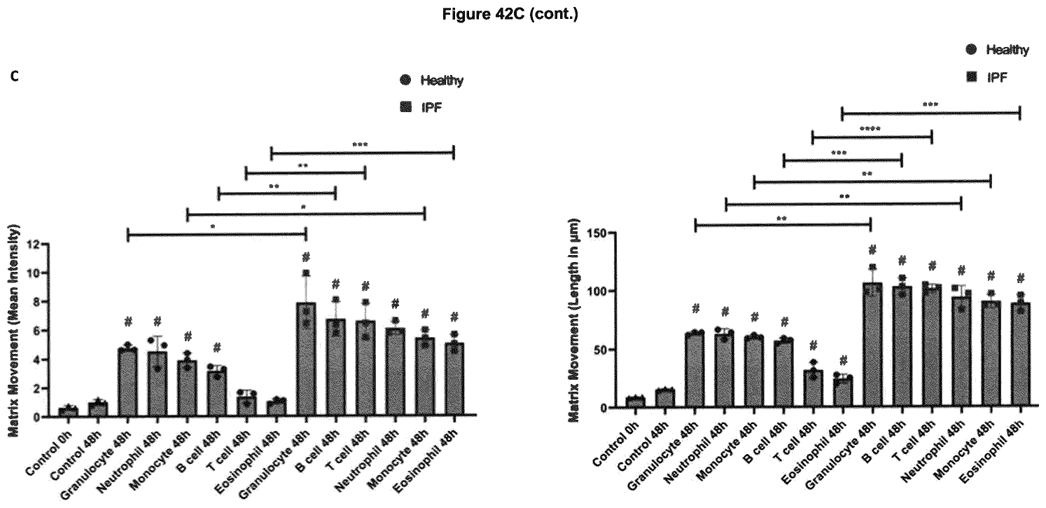

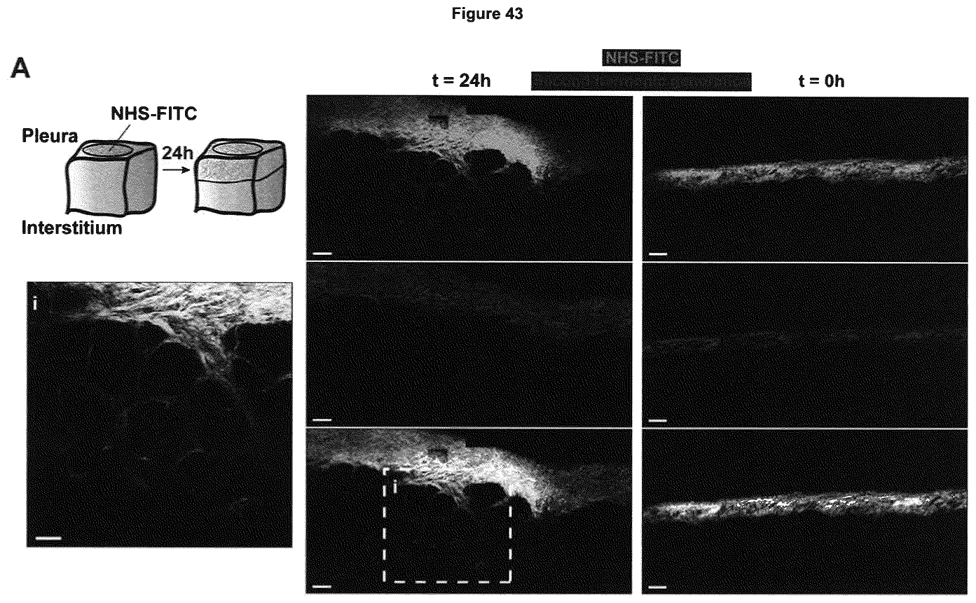

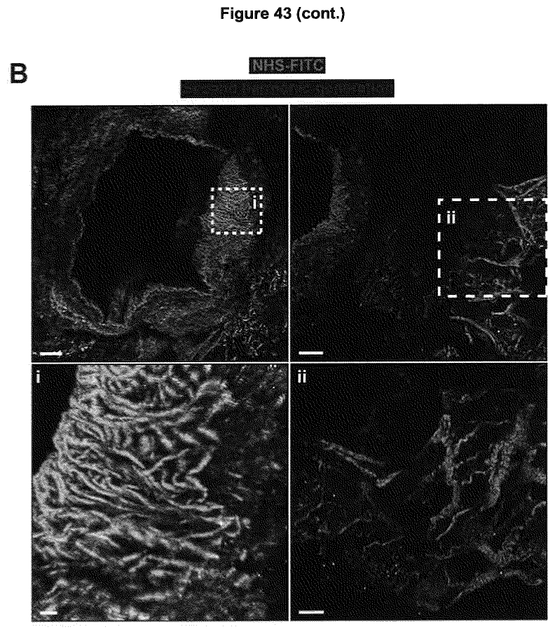

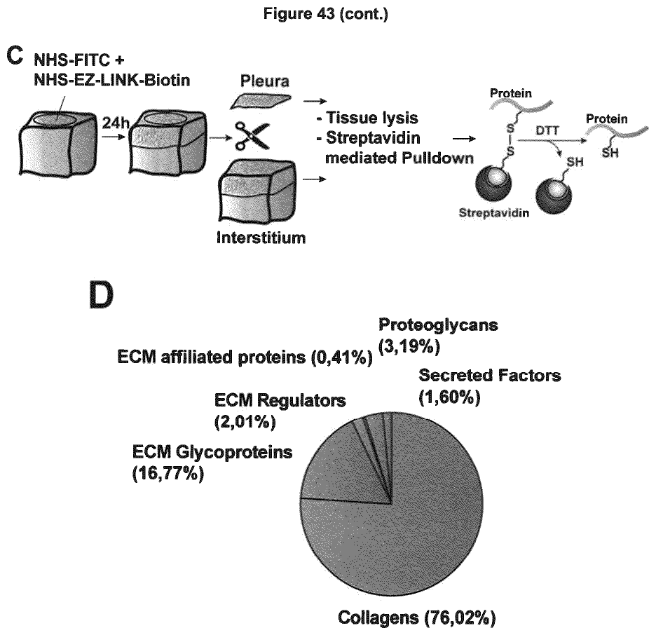

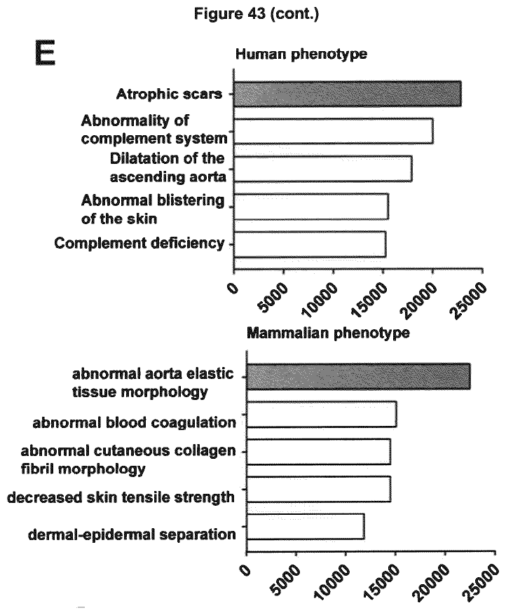

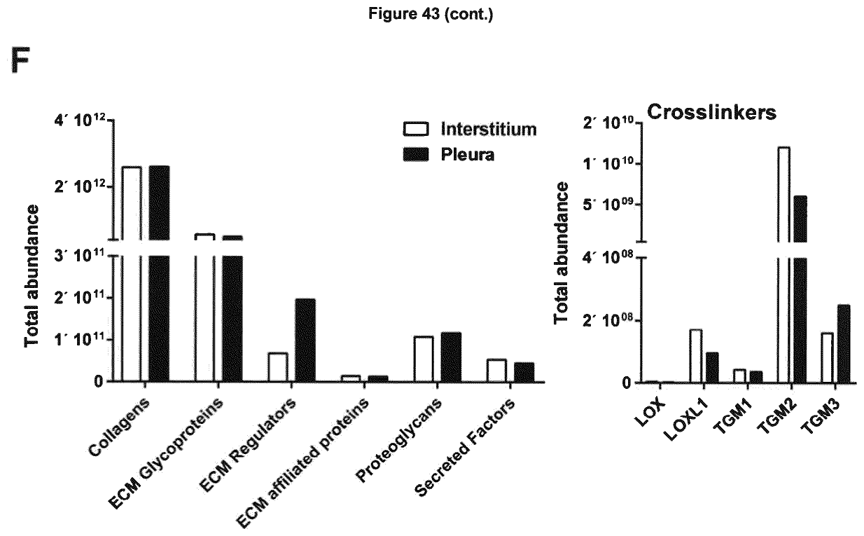

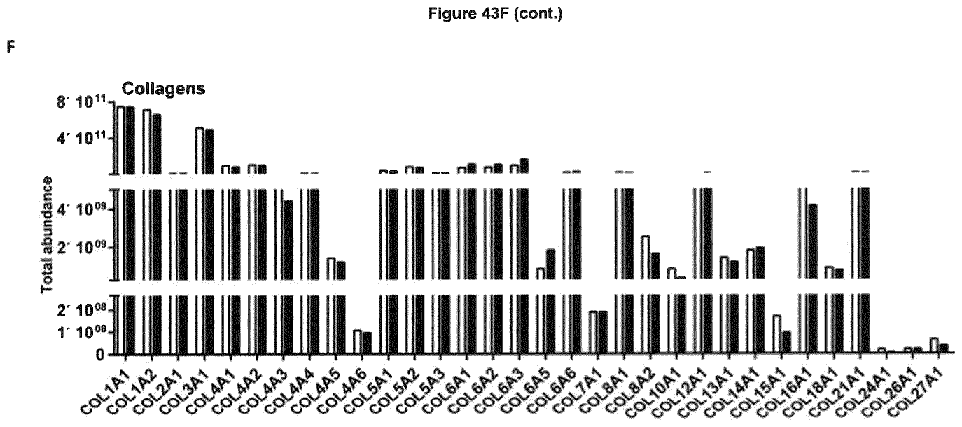

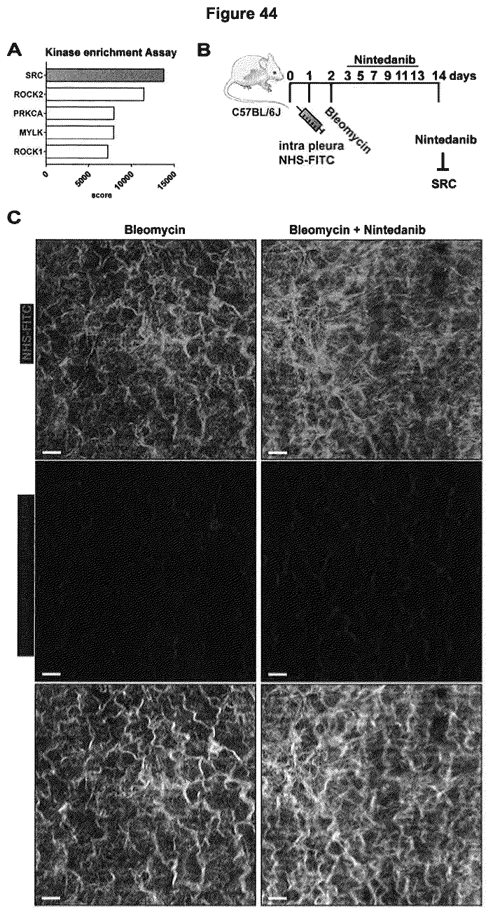

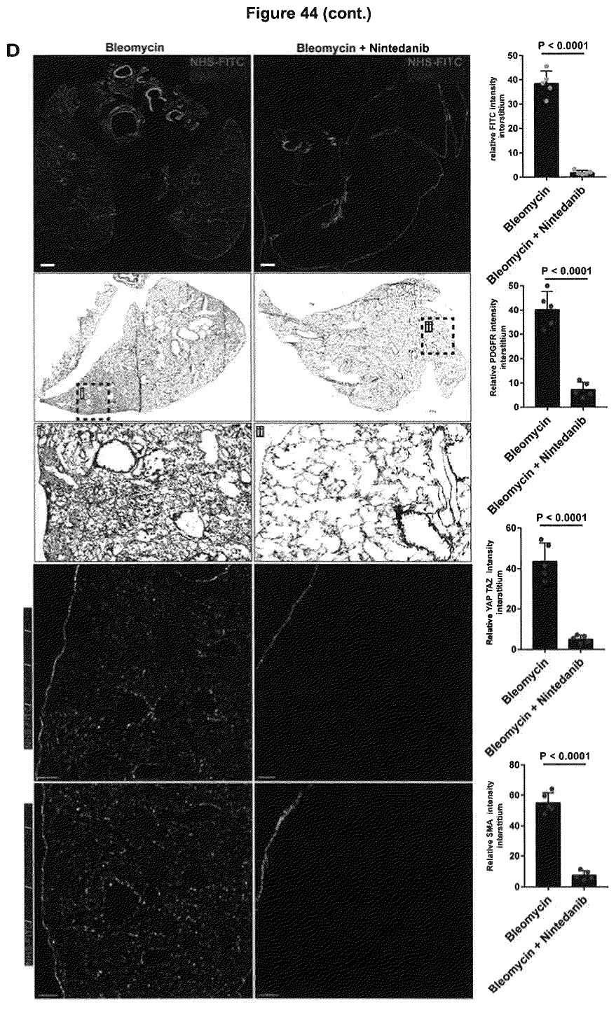

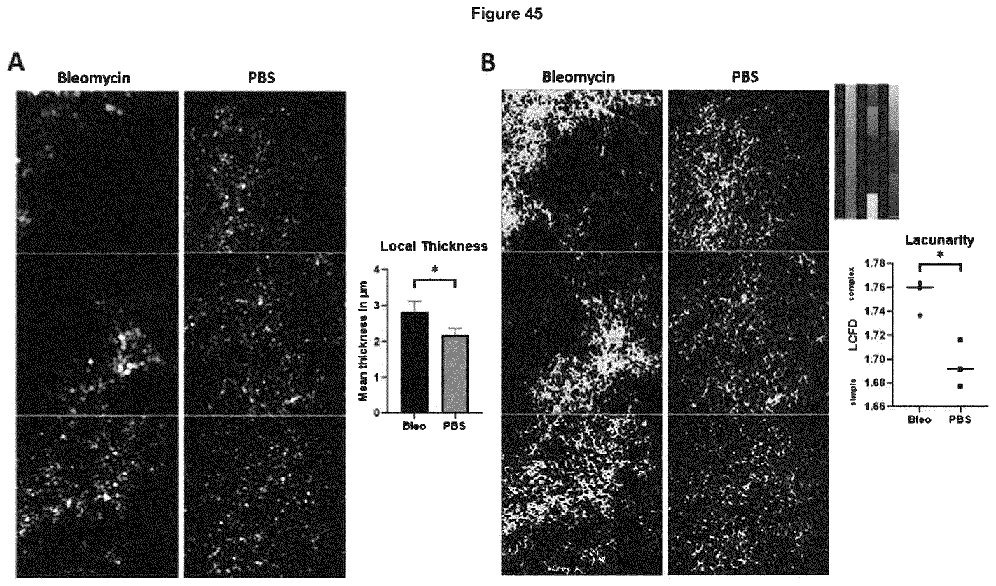

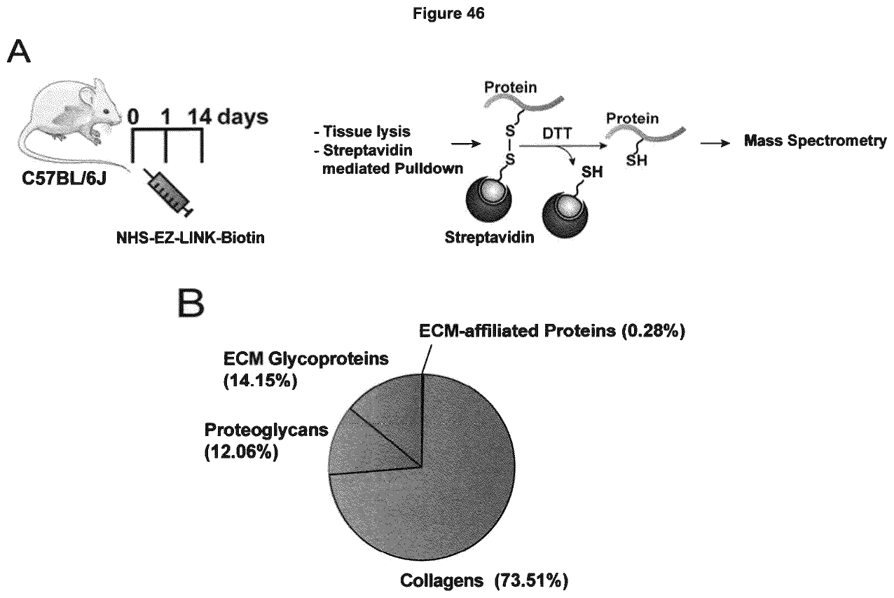

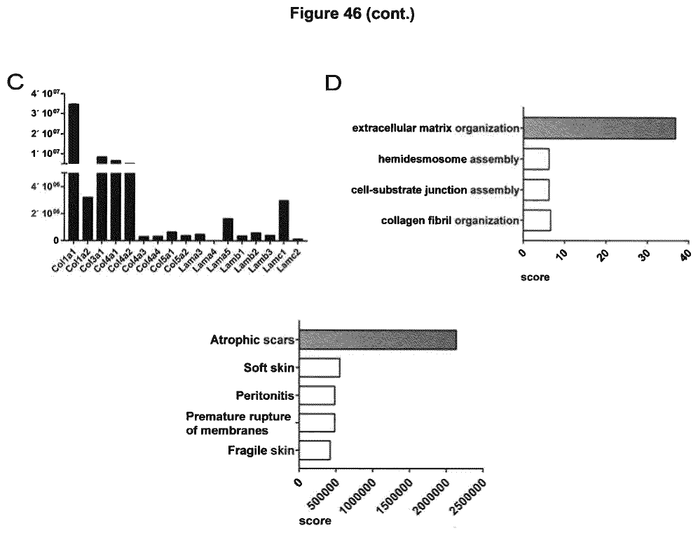

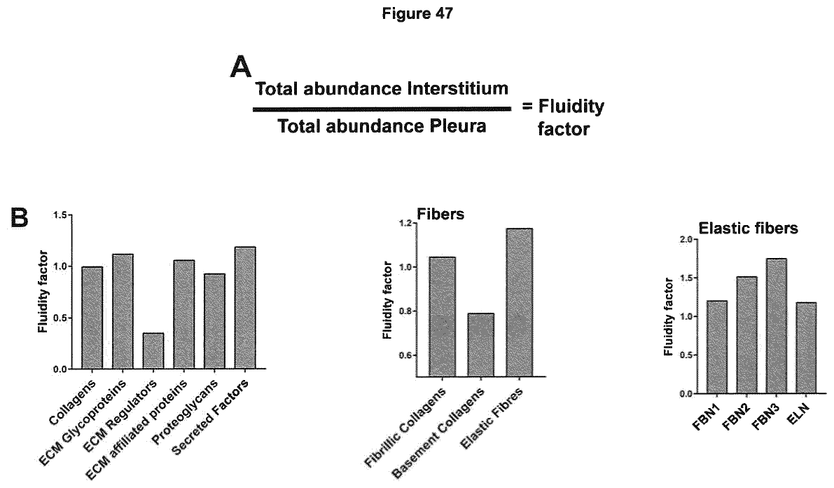

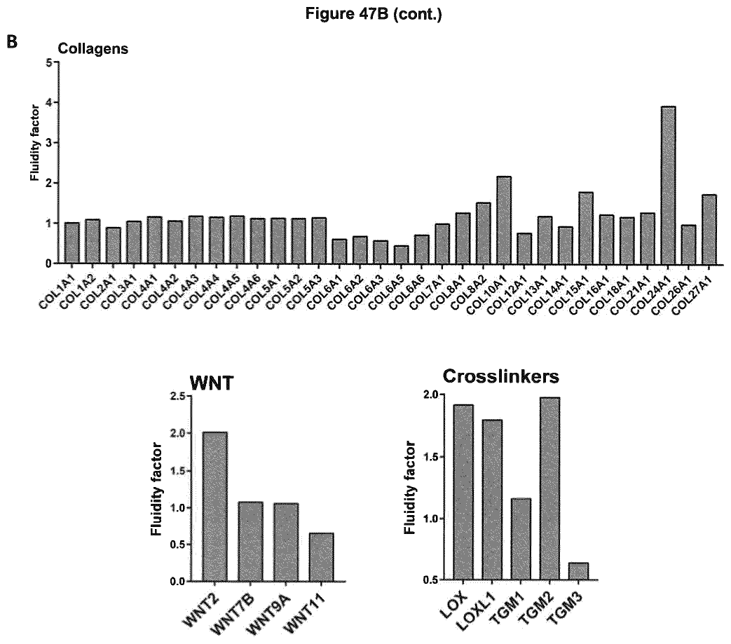

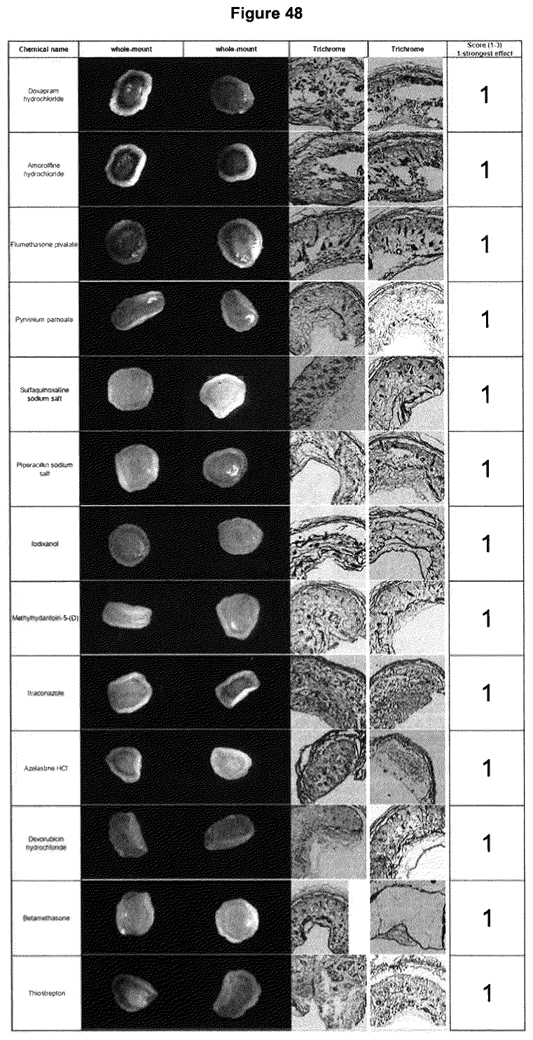

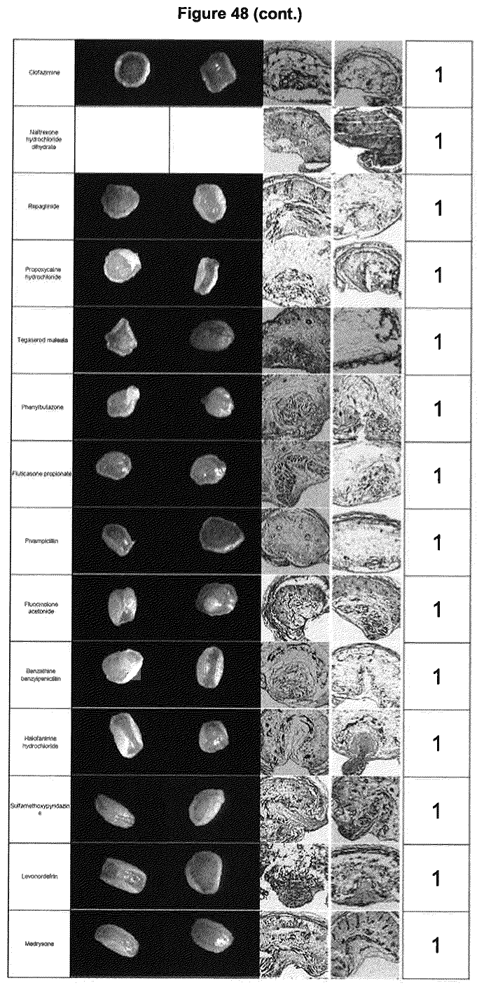

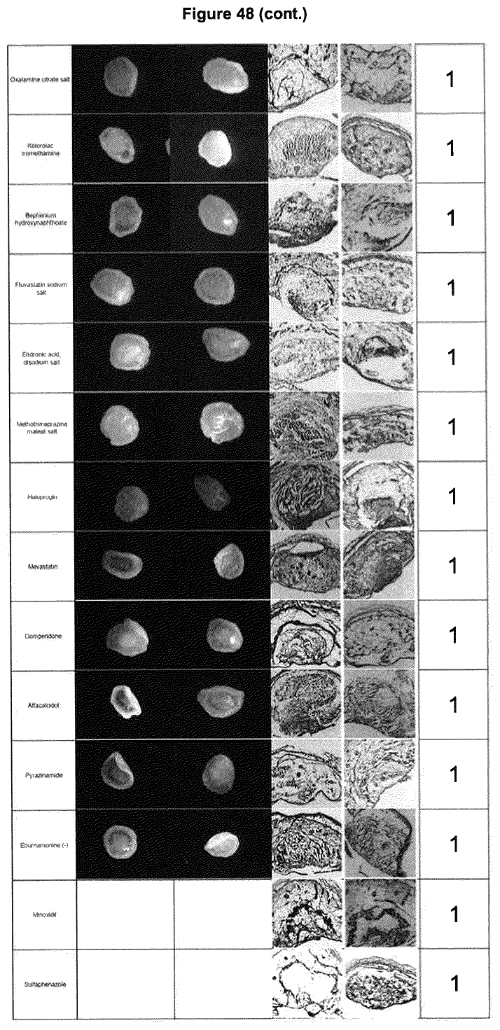

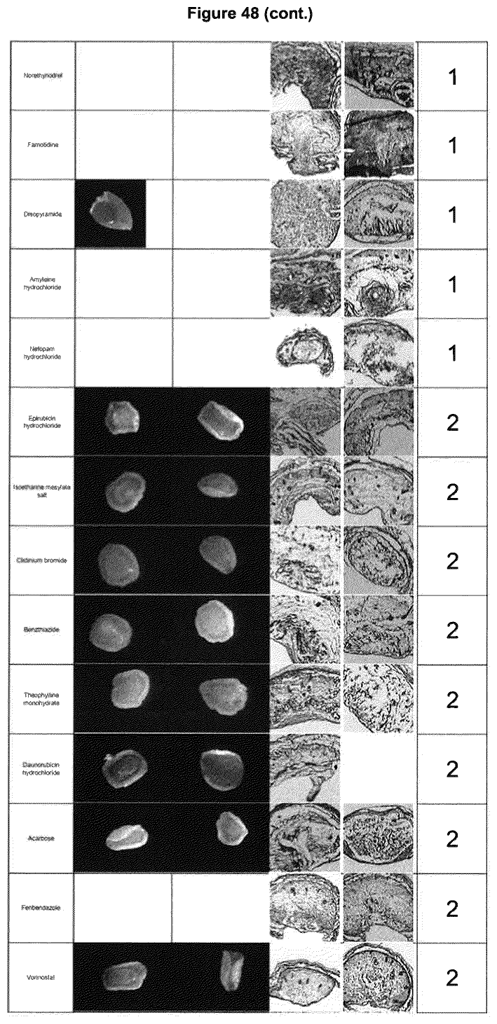

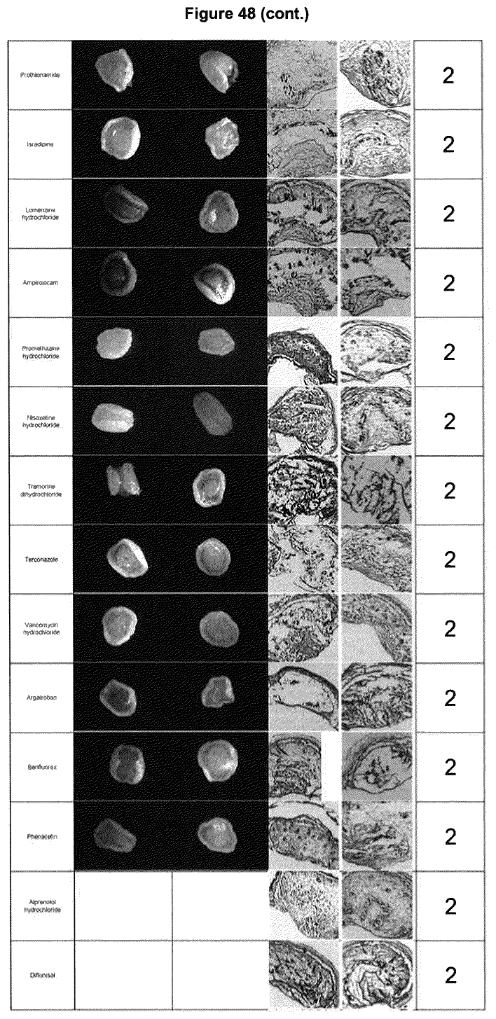

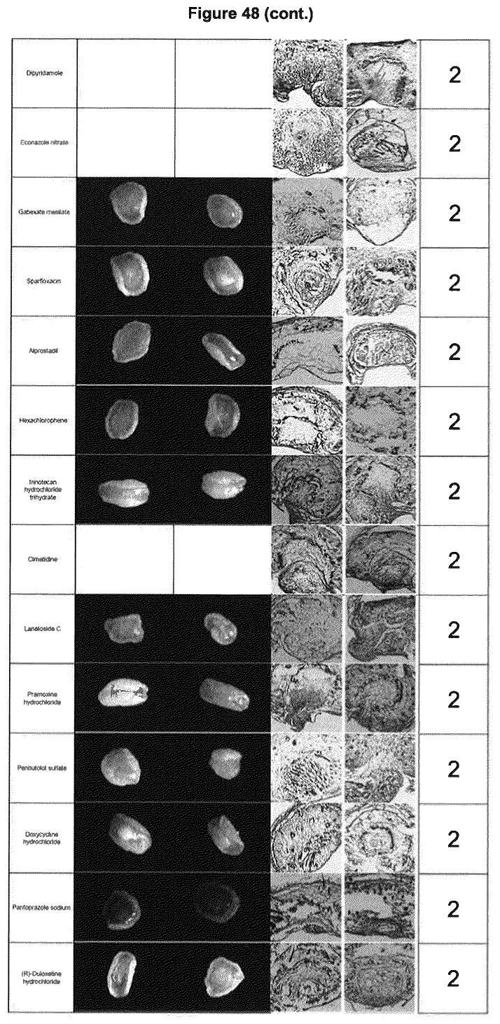

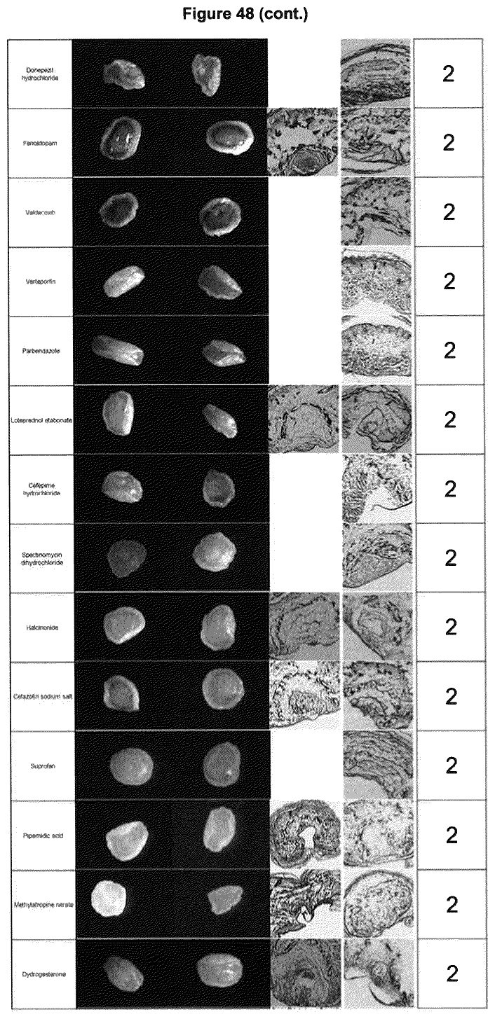

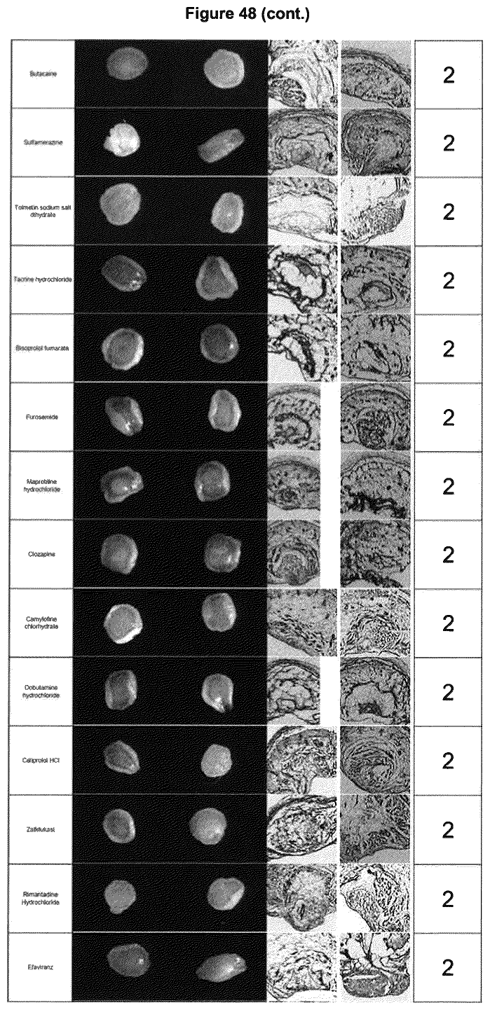

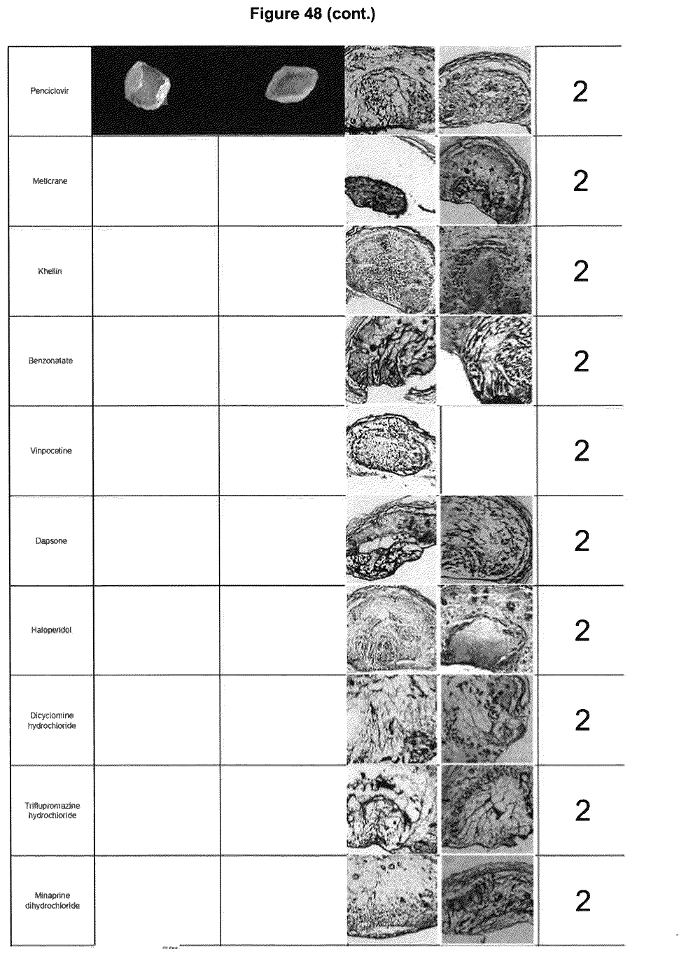

For this, the Inventors locally tagged and fate-mapped the matrix lining the visceral (serosa) and parietal (adventitia) organ surfaces of live mice using a N-hydroxysuccinimide ester fluorescein (NHS-FITC, FIG. 30a, methods). First, the Inventors concentrated on liver as a model system; foci of labeled liver matrix clearly coincided with second harmonic signal, indicating the in vivo labeling technique faithfully tags extracellular collagenous fibers. The matrix labeling approach revealed a multi-layered configuration of matrix that was vastly more detailed than that seen through second harmonic signal alone. Dye ester labeling revealed volumes of minute fibrils and multi-fibril aggregates in an immature arrangement, which filled spaces in-between larger mature collagen fibers (FIG. 30a). To see if the new arrangement was a general aspect of internal organs the Inventors examined cecum and peritoneal organ surfaces. Here the Inventors observed an identical volume of minute extracellular fibrous material that was also imperceptible by second harmonic microscopy alone. Cecum and peritoneum have mature and woven collagen fibers that were oriented along the entire organ surfaces, with volumes of micro-fibers in an immature organization that filled open spaces between the woven mature collagen fibers.

The inventors then set out to test the mobility of these volumes of immature matrix by locally applying dye ester as before and ‘fate mapping’ these local pools of matrix (FIG. 30b and methods). Tagged pools of liver matrix did not move over 24 hours in adult healthy animals (FIG. 30c). To test the mobility of the matrix under injury conditions, the Inventors focused on a clinical liver wound model based on irreversible electroporation. Irreversible electroporation approaches serve as alternative clinical approaches to ablative therapy regimens for various cancers¹⁴. The ablative conditions created by irreversible electroporation induce localized hepatocyte cell death (hence irreversible), followed by a repair response that ultimately restores liver histomorphology and function without scar tissue. The Inventors combined matrix-labeling with irreversible electroporation by locally marking pools of matrix at six distinct locations across the liver, creating circular labeled fields with clearly defined boundaries (FIG. 30c). The Inventors then damaged a discrete remote liver location by electroporation. Within twenty-four hours post-wounding, pools of matrix moved from their original confined location and intermixed extensively. Importantly, the labeled matrix pools underwent major translocations, gushing into and completely filling the wound with matrix. These findings indicate that injury induces organ-wide directed motion of fluid matrix. Extended Video 1 shows the two rigid and fluid matrix compartments in liver. Rigid frames seen through second harmonic signal (majenta) are bathed in volumes or clouds of proteins (green) that extend into the wound areas, and that are structurally distinct from the rigid frames seen through second harmonic signal (FIG. 30d). Three-dimensional imaging of the wounds after 24 hours revealed they are completely plugged with volumes of matrix clouds (FIG. 30d). At their extremities, the matrix protein clouds extended filaments that adhere to and wrap the rigid matrix frames, and interconnect with adjacent healthy connective tissue. By two weeks post injury matrix clouds had recreated new reticular connective tissue that supported liver regeneration (FIG. 30d).

To study if a similar fluid-like matrix system exists in other organs, the Inventors used a second clinically relevant model of organ injury using clinical incision (laparotomy) and local abrasion of the peritoneum. Similar to the findings in liver, peritoneal injury induced gushes of labeled matrix across the entire peritoneal surface. Foci of labeled matrix extensively intermixed, and gushed into wounds in a matter of minutes (FIG. 30e, FIG. 30f), just as the Inventors initially found in liver. Peritoneal matrix gushing occurred remotely from the injury site and across the entire cavity wall and ECM movement dynamics in peritoneum resembled that seen first in liver, initiating within minutes post injury and continuously pouring matrix into wounds over three days (FIG. 30g). In fact, peritoneal movements were even more vigorous than in liver. This was evident in the greater amount of FITC marked signal in peritoneal than liver wounds, across all experimental animals. This could also suggest that peritoneal matrix is especially rich in proteins and fibers, a point which the Inventors subsequently address. FIG. 30h shows snap shot three dimensional images of different steps of matrix fluid movement across the peritoneum. As it is propelled, fluid matrix remains coiled, and is subsequently rearranged, with fibers accumulating in wounds.

Fluid Matrix is Transformed into Rigid Frames in Wounds

To investigate whether fluid matrix matures into rigid frames the Inventors tested if transported fluid elements undergo fibrilar cross-linking in wounds. The Inventors marked live mouse liver surfaces at two distinct locations, one with NHS-EZ-LINK-Biotin and another with NHS-FITC-ester (FIG. 31a). Via Streptavidin-mediated purification of EZ-LINK-Biotin labeled proteins of wound sites, potentially cross-linked FITC labeled proteins can be obtained and measured. After wounding the Inventors pulled down EZ-link labeled proteins on streptavidin beads, washed in detergent-rich buffer and removed those with fragile interactions (see methods). There was a steady increase of FITC signal in the pull-down samples over the time course of 72 hours demonstrating that fluid matrix from distinct sites accumulate in wounds and undergo crosslinking to form mature, stably interconnected matrix (FIG. 31b). To visually prove that fluid matrix from remote, even different organs, can intermix and crosslink within wounds, the Inventors applied a surgical adhesion mouse model, where adhesions develop between peritoneum and cecum (FIG. 31c and methods). The Inventors tagged peritoneal and cecal matrix with distinct color conjugates of esters (NHS-FITC and NHS-AF568, respectively) and found matrix intermixing at the injury site where bands of fibrous adhesions developed. FIG. 31d shows the intermixing and overlap of cecal and peritoneal matrix at the adhesion site (FIG. 31d). Further, peritoneal collagen (red) contributed to cecal repair (FIG. 31d), showing that fluid matrix crosses organ boundaries where it contributes to structural repair of adjacent organs. Same types of intra-organ fiber crosslinking occurred when the Inventors tagged liver and peritoneal matrix, and induced adhesions between them (FIG. 31f).

To functionally prove crosslinking occurs between elements derived from two separate organs (cecum and peritoneum), the Inventors marked the peritoneal matrix with a distinct EZ-LINK-NHS-Biotin ester type and the cecum with another distinct FITC-NHS ester type and induced local adhesions between these two organs in a remote location by local abrasion (FIG. 31d and methods). A pull down of the wound lysate after two weeks contained abundant green (cecal) and EZ-LINK-NHS-Biotin (peritoneum) labeled proteins. Pulldown experiments detected abundant crosslinking had occurred between cecal and peritoneal elements. Collectively, the findings uncover a fluid matrix system that replenishes wounds with building blocks from remote locations, even separate organs, and that these fluid elements cross-link to form mature connective tissue in wounds.

### Fluid Matrix is an Inventory for Tissue Repair

Next, the Inventors sought to define the protein constituents of the fluid matrix in the injury models by mass-spectrometry. Briefly, the Inventors tagged pools of matrix using modified Biotin-conjugated EZ-link sulfo-NHS esters on liver, peritoneum, and cecum, and subjected them to injury models. Twenty-four hours post-injury, the Inventors collected matrix from wound sites and purified mobile matrix proteins via Streptavidin followed by proteomics of all tagged peptides (FIG. 32a). The proteomic inventory revealed hundreds of extracellular proteins are maneuvered into wounds across organs that originate from multiple organ depths and layers (FIG. 32b). Table 2-4 show the full list of proteins within the fluid matrix. Fluid matrix consisted of numerous ground substance proteins such as Glycoproteins, Proteoglycans and ECM affiliated proteins but fibrillic collagenous fibers such as Collagen type I and III were the most abundant fraction (FIGS. 32c and 32d). The Inventors also detect their crosslinking enzymes such as Lysyl oxidase and Transglutaminases involved in tissue remodeling, basement membrane formation and stability such as Collagen type IV, VI, or Laminins, elastic fiber associated proteins such as Fibrillines, and many other glycoproteins and proteoglycans that contribute to tissue remodeling, fiber clot formation, fibrinolysis, granulation and scar tissue formation (FIG. 32d). Distinctly abundant fluid elements could be assigned pro regeneration or fibrosis. For example, the fluid matrix entering liver wounds is enriched in regulators linked to tissue regeneration like Ambp, F13a1, F13b, Itih1, Kng1 and PZP, all of which support cellular growth and repair (FIG. 32e). Whereas fluid matrix entering peritoneal wounds showed pro-fibrotic and was enriched in collagenous fibers and ECM glycoproteins that induce extracellular matrix organization, maturation and scar formation. Further, peritoneal fractions included collagenous fibers such as Collagen types 9,10,11 and arbiters of fibrotic scar formation such as Grem1, Ogn, Chad, MMP9, MMP20 that were completely absent from liver or cecal fluid matrix, all of which support and enact fibrotic scars. Principle component analysis of sample distribution indicated organ-specific and distinct compositions of fluid matrix across liver, cecum and peritoneum. For example, the fluid matrix entering liver wounds is enriched in regulators of oxidative stress, metabolic enzymes and in lipid metabolism, whereas fluid matrix entering peritoneal wounds was clearly pro-fibrotic (FIG. 32f).

These analyses indicate that while fluid matrix provides building blocks for multiple steps of the repair process, its composition is organ-specific and an indicator of the ensuing repair response, to either regenerate or scar.

Next, the Inventors sought to compare if the findings translate to human wounds. The Inventors took samples from patients who have developed postoperative adhesions and determined their protein composition by immunofluorescence. Importantly the Inventors found they are composed of the same adventitial protein elements found in the mouse peritoneal fluid matrix fractions (FIG. 34a). This suggests that human wound repair develops in the same way as mouse by mobilizing fluid matrix from remote adventitial and serosa sites.

Neutrophils Pilot Fluid Matrix into Wounds

Next, the Inventors checked for a possible link between ECM movement and inflammatory onset, as both act during the early phases of the wound response. To comprehensively explore all possible cellular agents in matrix movement, the Inventors employed highly parallel single-cell transcriptomics of electroporated liver.

Single cell RNA sequencing of over 25,054 cells (see methods) across healthy liver at 1 and 7 days post electroporation revealed 17 distinct cell populations within wounds, predominantly of myeloid lineage such as monocytes, macrophages and neutrophils (FIG. 33a and FIGS. 35a, b and c). The Inventors then screened all 17 clusters for dynamic presence of membrane receptors that would imply matrix movements are facilitated by cell-ECM adhesions (FIG. 33b). Stromal and immune cells both showed high ECM binding activity with multiple ECM binding cell surface receptors. Yet, only macrophages/neutrophils exhibited the dynamic presence and migration score that overlapped with matrix relocation into wounds (FIG. 33c). To determine any possible role for macrophages and neutrophils in matrix movements, the Inventors subjected the liver electroporation and peritoneal laparotomy injury models in Lyz2^{Cre}; Ai14 transgenic mice (FIG. 33d, FIG. 36 and methods) where a red (dTomato) fluorescence reporter is expressed in all myeloid-lineage cells and allows their visualization. Live imaging of liver and peritoneal wounds in these mice revealed myeloid cells accumulate in wounds by migrating across large sweeps of organ surfaces. The Inventors found most if not all red-marked myeloid cells (n=90%) that migrated from the original labeled site into wounds, carried tack-packs' of FITC-positive matrix (FIG. 33e, f and FIGS. 36a, b, d and c). Myeloid cells back-packing FITC-positive fluid matrix had no cytoplasmic overlap of red and green signal, suggesting that the cells are not internalizing, ingesting, or phagocytosing matrix (FIG. 33g). Indeed, immunolabeling showed accumulation of neutrophils (Ly6G+) on livers and peritonea 24 hours post-injury that was clearly associated with fluid matrix on organ surfaces (FIG. 33h and FIG. 36e). The Inventors found not only individual myeloid cells piggy-backing FITC-positive fluid matrix, but also foci of myeloid aggregates, most likely neutrophil swarms that generate large local deposits of FITC-positive fluid matrix in proximity to the wound. These data imply that neutrophils are an essential part of fluid matrix mobility.

To investigate if neutrophils are responsible for matrix mobilization, the Inventors employed a chemical cell depletion strategy in combination with matrix fate mapping and organ injury. Depleting or halting neutrophils with Ly6g neutralizing antibodies completely blocked fluid matrix flows in both liver and peritoneum, whereas chemically depleting macrophages with Clodronate had no effect on matrix mobilization (FIGS. 33i and k). Next, the Inventors focused on neutrophil activation and found in the single cell analysis that neutrophils upregulate integrins CD11b and CD18 within early wounds and the expression remained throughout the entire 3-day gushing process (FIG. 33j and FIGS. 35d and e). Indeed, neutralizing antibodies against these two surface receptors led to reduction or complete cessation of matrix movements in injured animals (FIG. 33k and FIG. 36f). The Inventors then checked whether neutrophil swarming could account for matrix mobilization. Leukotrine-mediated signals in neutrophils play a key role in their accumulation into wounds²² and inhibitors of leukotriene receptor activity completely blocked matrix mobilization into wounds (FIG. 33k and FIG. 36f). Remarkably, locally harnessing neutrophils with the chemokine Lipoxin A4, led to recruitment of fluid matrix, in the absence of wounds (FIGS. 33i and k). This demonstrates that neutrophil swarms do indeed direct matrix movements and its deposition, locally. The Inventors also found that oxidative stress and nitric oxide synthesis in neutrophils are upregulated early in the injury response (FIG. 33j). Indeed, inhibitors of nitric oxide synthesis or its function, completely blocked matrix mobilization in both liver and peritoneal injury models (FIG. 33k and FIG. 36f).

In the absence of matrix mobilization wounds failed to heal. In the liver, blocking matrix mobilization into wounds led to a complete block of regeneration. Liver wounds were enlarged, failed to close and lacked structural organization (FIGS. 37a and b). Similarly blocking matrix mobilization in the peritoneum, completely eradicated fibrous adhesions from forming, with absence of any signs of adhesion formation in animals (FIG. 37c-e).

The inventors conclude that organs posses reservoirs of fluid matrix within connective tissues, and that injury triggers organ-wide mobilization of fluid matrix into new tissue construction sites where they fuel tissue repair and regeneration. Further, that neutrophils have a newly found and essential role in executing, piloting and depositing matrix into wounds.

TABLE 2
Identified proteins in liver samples

| Anova | q | Gene | |
|---|---|---|---|
| (p) | Value | symbol | Uniprot Assecion/Description |
| 0.0001 | 0.0038 | Rps4x | P62702\|RS4X_MOUSE 40S ribosomal protein S4, X isoform OS = |
| | | | Mus |
| | | | musculus GN = Rps4x PE = 1 SV = 2 |
| 0.0002 | 0.0040 | Calr | P14211\|CALR_MOUSE Calreticulin OS = Mus musculus GN = Calr |
| | | | PE = 1 SV = 1 |
| 0.0002 | 0.0040 | Gdi1 | P50396\|GDIA_MOUSE Rab GDP dissociation inhibitor alpha OS = Mus |
| | | | musculus GN = Gdi1 PE = 1 SV = 3 |
| 0.0002 | 0.0040 | 1 SV | Q9DCS2\|CP013_MOUSE UPF0585 protein C16orf13 homolog |
| | | | OS = Mus musculus PE = 1 SV = 1 |
| 0.0004 | 0.0050 | Ephx1 | Q9D379\|HYEP_MOUSE Epoxide hydrolase 1 OS = Mus musculus |
| | | | GN = Ephx1 PE = 1 SV = 2 |
| 0.0004 | 0.0050 | Cct2 | P80314\|TCPB_MOUSE T-complex protein 1 subunit beta OS = Mus |
| | | | musculus GN = Cct2 PE = 1 SV = 4 |
| 0.0004 | 0.0050 | Cth | Q8VCN5\|CGL_MOUSE Cystathionine gamma-lyase OS = Mus |
| | | | musculus GN = Cth PE = 1 SV = 1 |
| 0.0006 | 0.0056 | Foxe3 | Q9QY14\|FOXE3_MOUSE Forkhead box protein E3 OS = Mus |
| | | | musculus GN = Foxe3 PE = 1 SV = 1 |
| 0.0006 | 0.0056 | Hadh | Q61425\|HCDH_MOUSE Hydroxyacyl-coenzyme A dehydrogenase, |
| | | | mitochondrial OS = Mus musculus GN = Hadh PE = 1 SV = 2 |
| 0.0007 | 0.0056 | Acsf2 | Q8VCW8\|ACSF2_MOUSE Acyl-CoA synthetase family member 2, |
| | | | mitochondrial OS = Mus musculus GN = Acsf2 PE = 1 SV = 1 |
| 0.0008 | 0.0056 | Hsp90aa1 | P07901\|HS90A_MOUSE Heat shock protein HSP 90-alpha OS = Mus |
| | | | musculus GN = Hsp90aa1 PE = 1 SV = 4 |
| 0.0008 | 0.0056 | Uba1 | Q02053\|UBA1_MOUSE Ubiquitin-like modifier-activating enzyme 1 |
| | | | OS = Mus musculus GN = Uba1 PE = 1 SV = 1 |
| 0.0008 | 0.0056 | Slc25a13 | Q9QXX4\|CMC2_MOUSE Calcium-binding mitochondrial carrier |
| | | | protein Aralar2 OS = Mus musculus GN = Slc25a13 PE = 1 SV = 1 |
| 0.0011 | 0.0065 | Tuba1b | P05213\|TBA1B_MOUSE Tubulin alpha-1B chain OS = Mus musculus |
| | | | GN = Tuba1b PE = 1 SV = 2 |
| 0.0014 | 0.0072 | Prdx6 | O08709\|PRDX6_MOUSE Peroxiredoxin-6 OS = Mus musculus |
| | | | GN = Prdx6 PE = 1 SV = 3 |
| 0.0015 | 0.0072 | Ywhag | P61982\|1433G_MOUSE 14-3-3 protein gamma OS = Mus musculus |
| | | | GN = Ywhag PE = 1 SV = 2 |
| 0.0015 | 0.0072 | Ccdc18 | Q640L5\|CCD18_MOUSE Coiled-coil domain-containing protein 18 |
| | | | OS = Mus musculus GN = Ccdc18 PE = 1 SV = 1 |
| 0.0016 | 0.0072 | Krt8 | P11679\|K2C8_MOUSE Keratin, type II cytoskeletal 8 OS = Mus |
| | | | musculus GN = Krt8 PE = 1 SV = 4 |
| 0.0020 | 0.0072 | Actn4 | P57780\|ACTN4_MOUSE Alpha-actinin-4 OS = Mus musculus |
| | | | GN = Actn4 PE = 1 SV = 1 |
| 0.0021 | 0.0072 | Atp5a1 | Q03265\|ATPA_MOUSE ATP synthase subunit alpha, mitochondrial |
| | | | OS = Mus musculus GN = Atp5a1 PE = 1 SV = 1 |
| 0.0022 | 0.0072 | Lama2 | Q60675\|LAMA2_MOUSE Laminin subunit alpha-2 OS = Mus musculus |
| | | | GN = Lama2 PE = 1 SV = 2 |
| 0.0023 | 0.0072 | Uqcrc1 | Q9CZ13\|QCR1_MOUSE Cytochrome b-c1 complex subunit 1, |
| | | | mitochondrial OS = Mus musculus GN = Uqcrc1 PE = 1 SV = 2 |
| 0.0024 | 0.0072 | Tubb4b | P68372\|TBB4B_MOUSE Tubulin beta-4B chain OS = Mus musculus |
| | | | GN = Tubb4b PE = 1 SV = 1 |
| 0.0024 | 0.0072 | Capza1 | P47753\|CAZA1_MOUSE F-actin-capping protein subunit alpha-1 |
| | | | OS = Mus musculus GN = Capza1 PE = 1 SV = 4 |
| 0.0025 | 0.0072 | Acaa2 | Q8BWT1\|THIM_MOUSE 3-ketoacyl-CoA thiolase, mitochondrial |
| | | | OS = Mus musculus GN = Acaa2 PE = 1 SV = 3 |
| 0.0025 | 0.0072 | Nudt7 | Q99P30\|NUDT7_MOUSE Peroxisomal coenzyme A diphosphatase |
| | | | NUDT7 OS = Mus musculus GN = Nudt7 PE = 1 SV = 2 |
| 0.0025 | 0.0072 | Egfr | Q01279\|EGFR_MOUSE Epidermal growth factor receptor OS = Mus |
| | | | musculus GN = Egfr PE = 1 SV = 1 |
| 0.0026 | 0.0072 | Acsm1 | Q91VA0\|ACSM1_MOUSE Acyl-coenzyme A synthetase ACSM1, |
| | | | mitochondrial OS = Mus musculus GN = Acsm1 PE = 1 SV = 1 |
| 0.0027 | 0.0072 | Got2 | P05202\|AATM_MOUSE Aspartate aminotransferase, mitochondrial |
| | | | OS = Mus musculus GN = Got2 PE = 1 SV = 1 |
| 0.0028 | 0.0072 | Hspa9 | P38647\|GRP75_MOUSE Stress-70 protein, mitochondrial OS = Mus |
| | | | musculus GN = Hspa9 PE = 1 SV = 3 |
| 0.0028 | 0.0072 | Sdha | Q8K2B3\|SDHA_MOUSE Succinate dehydrogenase [ubiquinone] |
| | | | flavoprotein subunit, mitochondrial OS = Mus musculus GN = Sdha |
| | | | PE = 1 SV = 1 |
| 0.0028 | 0.0072 | Otc | P11725\|OTC_MOUSE Ornithine carbamoyltransferase, mitochondrial |
| | | | OS = Mus musculus GN = Otc PE = 1 SV = 1 |
| 0.0028 | 0.0072 | Npnt | Q91V88\|NPNT_MOUSE Nephronectin OS = Mus musculus GN = Npnt |
| | | | PE = 1 SV = 1 |
| 0.0029 | 0.0072 | Cat | P24270\|CATA_MOUSE Catalase OS = Mus musculus GN = Cat PE = 1 |
| | | | SV = 4 |
| 0.0029 | 0.0072 | Synpo | Q8CC35\|SYNPO_MOUSE Synaptopodin OS = Mus musculus |
| | | | GN = Synpo PE = 1 SV = 2 |
| 0.0029 | 0.0072 | Vcp | Q01853\|TERA_MOUSE Transitional endoplasmic reticulum ATPase |
| | | | OS = Mus musculus GN = Vcp PE = 1 SV = 4 |
| 0.0029 | 0.0072 | Etfb | Q9DCW4\|ETFB_MOUSE Electron transfer flavoprotein subunit beta |
| | | | OS = Mus musculus GN = Etfb PE = 1 SV = 3 |
| 0.0030 | 0.0072 | Hnrnpa2b1 | O88569\|ROA2_MOUSE Heterogeneous nuclear ribonucleoproteins |
| | | | A2/B1 OS = Mus musculus GN = Hnrnpa2b1 PE = 1 SV = 2 |
| 0.0031 | 0.0072 | Sod1 | P08228\|SODC_MOUSE Superoxide dismutase [Cu—Zn] OS = Mus |
| | | | musculus GN = Sod1 PE = 1 SV = 2 |
| 0.0031 | 0.0072 | Itch | Q8C863\|ITCH_MOUSE E3 ubiquitin-protein ligase Itchy OS = Mus |
| | | | musculus GN = Itch PE = 1 SV = 2 |
| 0.0033 | 0.0073 | Krt17 | Q9QWL7\|K1C17_MOUSE Keratin, type I cytoskeletal 17 OS = Mus |
| | | | musculus GN = Krt17 PE = 1 SV = 3 |
| 0.0034 | 0.0073 | Bsg | P18572\|BASI_MOUSE Basigin OS = Mus musculus GN = Bsg PE = 1 |
| | | | SV = 2 |
| 0.0034 | 0.0073 | Tst | P52196\|THTR_MOUSE Thiosulfate sulfurtransferase OS = Mus |
| | | | musculus GN = Tst PE = 1 SV = 3 |
| 0.0035 | 0.0073 | Mdh1 | P14152\|MDHC_MOUSE Malate dehydrogenase, cytoplasmic |
| | | | OS = Mus musculus GN = Mdh1 PE = 1 SV = 3 |
| 0.0035 | 0.0073 | Eef2 | P58252\|EF2_MOUSE Elongation factor 2 OS = Mus musculus |
| | | | GN = Eef2 PE = 1 SV = 2 |
| 0.0036 | 0.0074 | Gstm1 | P10649\|GSTM1_MOUSE Glutathione S-transferase Mu 1 OS = Mus |
| | | | musculus GN = Gstm1 PE = 1 SV = 2 |
| 0.0039 | 0.0075 | Pebp1 | P70296\|PEBP1_MOUSE Phosphatidylethanolamine-binding protein 1 |
| | | | OS = Mus musculus GN = Pebp1 PE = 1 SV = 3 |
| 0.0039 | 0.0075 | Spr | Q64105\|SPRE_MOUSE Sepiapterin reductase OS = Mus musculus |
| | | | GN = Spr PE = 1 SV = 1 |
| 0.0040 | 0.0075 | Eno1 | P17182\|ENOA_MOUSE Alpha-enolase OS = Mus musculus GN = Eno1 |
| | | | PE = 1 SV = 3 |
| 0.0040 | 0.0075 | Bdh1 | Q80XN0\|BDH_MOUSE D-beta-hydroxybutyrate dehydrogenase, |
| | | | mitochondrial OS = Mus musculus GN = Bdh1 PE = 1 SV = 2 |
| 0.0041 | 0.0075 | Cps1 | Q8C196\|CPSM_MOUSE Carbamoyl-phosphate synthase [ammonia], |
| | | | mitochondrial OS = Mus musculus GN = Cps1 PE = 1 SV = 2 |
| 0.0041 | 0.0075 | Ak3 | Q9WTP7\|KAD3_MOUSE GTP: AMP phosphotransferase AK3, |
| | | | mitochondrial OS = Mus musculus GN = Ak3 PE = 1 SV = 3 |
| 0.0043 | 0.0075 | Anxa5 | P48036\|ANXA5_MOUSE Annexin A5 OS = Mus musculus GN = Anxa5 |
| | | | PE = 1 SV = 1 |
| 0.0044 | 0.0075 | Aldh2 | P47738\|ALDH2_MOUSE Aldehyde dehydrogenase, mitochondrial |
| | | | OS = Mus musculus GN = Aldh2 PE = 1 SV = 1 |
| 0.0045 | 0.0075 | Ppia | P17742\|PPIA_MOUSE Peptidyl-prolyl cis-trans isomerase A OS = Mus |
| | | | musculus GN = Ppia PE = 1 SV = 2 |
| 0.0045 | 0.0075 | Hspa1l | P16627\|HS71L_MOUSE Heat shock 70 kDa protein 1-like OS = Mus |
| | | | musculus GN = Hspa1l PE = 1 SV = 4 |
| 0.0046 | 0.0075 | Blvrb | Q923D2\|BLVRB_MOUSE Flavin reductase (NADPH) OS = Mus |
| | | | musculus GN = Blvrb PE = 1 SV = 3 |
| 0.0046 | 0.0075 | Eci1 | P42125\|ECI1_MOUSE Enoyl-CoA delta isomerase 1, mitochondrial |
| | | | OS = Mus musculus GN = Eci1 PE = 1 SV = 2 |
| 0.0047 | 0.0076 | Slc27a2 | O35488\|S27A2_MOUSE Very long-chain acyl-CoA synthetase |
| | | | OS = Mus musculus GN = Slc27a2 PE = 1 SV = 2 |
| 0.0048 | 0.0076 | Cyp2e1 | Q05421\|CP2E1_MOUSE Cytochrome P450 2E1 OS = Mus musculus |
| | | | GN = Cyp2e1 PE = 1 SV = 1 |
| 0.0052 | 0.0078 | Pccb | Q99MN9\|PCCB_MOUSE Propionyl-CoA carboxylase beta chain, |
| | | | mitochondrial OS = Mus musculus GN = Pccb PE = 1 SV = 2 |
| 0.0052 | 0.0078 | Ephx2 | P34914\|HYES_MOUSE Bifunctional epoxide hydrolase 2 OS = Mus |
| | | | musculus GN = Ephx2 PE = 1 SV = 2 |
| 0.0052 | 0.0078 | Gpd1 | P13707\|GPDA_MOUSE Glycerol-3-phosphate dehydrogenase |
| | | | [NAD(+)], cytoplasmic OS = Mus musculus GN = Gpd1 PE = 1 SV = 3 |
| 0.0054 | 0.0080 | Hspd1 | P63038\|CH60_MOUSE 60 kDa heat shock protein, mitochondrial |
| | | | OS = Mus musculus GN = Hspd1 PE = 1 SV = 1 |
| 0.0058 | 0.0083 | Rps3 | P62908\|RS3_MOUSE 40S ribosomal protein S3 OS = Mus musculus |
| | | | GN = Rps3 PE = 1 SV = 1 |
| 0.0059 | 0.0083 | Glt8d2 | Q640P4\|GL8D2_MOUSE Glycosyltransferase 8 domain-containing |
| | | | protein 2 OS = Mus musculus GN = Glt8d2 PE = 2 SV = 1 |
| 0.0059 | 0.0083 | Myo3b | Q1EG27\|MYO3B_MOUSE Myosin-IIIb OS = Mus musculus GN = Myo3b |
| | | | PE = 1 SV = 2 |
| 0.0061 | 0.0084 | Ca3 | P16015\|CAH3_MOUSE Carbonic anhydrase 3 OS = Mus musculus |
| | | | GN = Ca3 PE = 1 SV = 3 |
| 0.0063 | 0.0084 | Pygl | Q9ET01\|PYGL_MOUSE Glycogen phosphorylase, liver form OS = Mus |
| | | | musculus GN = Pygl PE = 1 SV = 4 |
| 0.0064 | 0.0084 | Tpi1 | P17751\|TPIS_MOUSE Triosephosphate isomerase OS = Mus |
| | | | musculus GN = Tpi1 PE = 1 SV = 4 |
| 0.0065 | 0.0084 | Hsp90ab1 | P11499\|HS90B_MOUSE Heat shock protein HSP 90-beta OS = Mus |
| | | | musculus GN = Hsp90ab1 PE = 1 SV = 3 |
| 0.0066 | 0.0084 | Cyp2d10 | P24456\|CP2DA_MOUSE Cytochrome P450 2D10 OS = Mus musculus |
| | | | GN = Cyp2d10 PE = 1 SV = 2 |
| 0.0066 | 0.0084 | Ndufs1 | Q91VD9\|NDUS1_MOUSE NADH-ubiquinone oxidoreductase 75 kDa |
| | | | subunit, mitochondrial OS = Mus musculus GN = Ndufs1 PE = 1 SV = 2 |
| 0.0068 | 0.0084 | Inmt | P40936\|INMT_MOUSE Indolethylamine N-methyltransferase OS = Mus |
| | | | musculus GN = Inmt PE = 1 SV = 1 |
| 0.0068 | 0.0084 | Slc35g2 | D3YVE8\|S35G2_MOUSE Solute carrier family 35 member G2 |
| | | | OS = Mus musculus GN = Slc35g2 PE = 1 SV = 1 |
| 0.0068 | 0.0084 | Glo1 | Q9CPU0\|LGUL_MOUSE Lactoylglutathione lyase OS = Mus musculus |
| | | | GN = Glo1 PE = 1 SV = 3 |
| 0.0069 | 0.0084 | Rpsa | P14206\|RSSA_MOUSE 40S ribosomal protein SA OS = Mus musculus |
| | | | GN = Rpsa PE = 1 SV = 4 |
| 0.0070 | 0.0084 | Csad | Q9DBEO\|CSAD_MOUSE Cysteine sulfinic acid decarboxylase |
| | | | OS = Mus musculus GN = Csad PE = 1 SV = 1 |
| 0.0070 | 0.0084 | Hpd | P49429\|HPPD_MOUSE 4-hydroxyphenylpyruvate dioxygenase |
| | | | OS = Mus musculus GN = Hpd PE = 1 SV = 3 |
| 0.0071 | 0.0084 | Uroc1 | Q8VC12\|HUTU_MOUSE Urocanate hydratase OS = Mus musculus |
| | | | GN = Uroc1 PE = 1 SV = 2 |
| 0.0072 | 0.0084 | Sardh | Q99LB7\|SARDH_MOUSE Sarcosine dehydrogenase, mitochondrial |
| | | | OS = Mus musculus GN = Sardh PE = 1 SV = 1 |
| 0.0072 | 0.0084 | Actb | P60710\|ACTB_MOUSE Actin, cytoplasmic 1 OS = Mus musculus |
| | | | GN = Actb PE = 1 SV = 1 |
| 0.0073 | 0.0084 | Agrn | A2ASQ1\|AGRIN_MOUSE Agrin OS = Mus musculus GN = Agrn PE = 1 |
| | | | SV = 1 |
| 0.0073 | 0.0084 | Ndrg2 | Q9QYG0\|NDRG2_MOUSE Protein NDRG2 OS = Mus musculus |
| | | | GN = Ndrg2 PE = 1 SV = 1 |
| 0.0074 | 0.0084 | Suclg2 | Q9Z2I8\|SUCB2_MOUSE Succinate--CoA ligase [GDP-forming] |
| | | | subunit beta, mitochondrial OS = Mus musculus GN = Suclg2 PE = 1 |
| | | | SV = 3 |
| 0.0075 | 0.0084 | Scp2 | P32020\|NLTP_MOUSE Non-specific lipid-transfer protein OS = Mus |
| | | | musculus GN = Scp2 PE = 1 SV = 3 |
| 0.0078 | 0.0085 | Atp5c1 | Q91VR2\|ATPG_MOUSE ATP synthase subunit gamma, mitochondrial |
| | | | OS = Mus musculus GN = Atp5c1 PE = 1 SV = 1 |
| 0.0078 | 0.0085 | Aox3 | G3X982\|AOXC_MOUSE Aldehyde oxidase 3 OS = Mus musculus |
| | | | GN = Aox3 PE = 1 SV = 1 |
| 0.0079 | 0.0085 | Lyz1 | P17897\|LYZ1_MOUSE Lysozyme C-1 OS = Mus musculus GN = Lyz1 |
| | | | PE = 1 SV = 1 |
| 0.0079 | 0.0085 | Aldh1l1 | Q8ROY6\|AL1L1_MOUSE Cytosolic 10-formyltetrahydrofolate |
| | | | dehydrogenase OS = Mus musculus GN = Aldh1l1 PE = 1 SV = 1 |
| 0.0083 | 0.0085 | Glud1 | P26443\|DHE3_MOUSE Glutamate dehydrogenase 1, mitochondrial |
| | | | OS = Mus musculus GN = Glud1 PE = 1 SV = 1 |
| 0.0084 | 0.0085 | Prdx1 | P35700\|PRDX1_MOUSE Peroxiredoxin-1 OS = Mus musculus |
| | | | GN = Prdx1 PE = 1 SV = 1 |
| 0.0084 | 0.0085 | Hnrnpa3 | Q8BG05\|ROA3_MOUSE Heterogeneous nuclear ribonucleoprotein A3 |
| | | | OS = Mus musculus GN = Hnrnpa3 PE = 1 SV = 1 |
| 0.0085 | 0.0085 | Clu | Q06890\|CLUS_MOUSE Clusterin OS = Mus musculus GN = Clu PE = 1 |
| | | | SV = 1 |
| 0.0088 | 0.0085 | Hba | P01942\|HBA_MOUSE Hemoglobin subunit alpha OS = Mus musculus |
| | | | GN = Hba PE = 1 SV = 2 |
| 0.0090 | 0.0085 | Mtco2 | P00405\|COX2_MOUSE Cytochrome c oxidase subunit 2 OS = Mus |
| | | | musculus GN = Mtco2 PE = 1 SV = 1 |
| 0.0091 | 0.0085 | Dars | Q922B2\|SYDC_MOUSE Aspartate--tRNA ligase, cytoplasmic |
| | | | OS = Mus musculus GN = Dars PE = 1 SV = 2 |
| 0.0092 | 0.0085 | Lonp2 | Q9DBN5\|LONP2_MOUSE Lon protease homolog 2, peroxisomal |
| | | | OS = Mus musculus GN = Lonp2 PE = 1 SV = 1 |
| 0.0093 | 0.0085 | Ftcd | Q91XD4\|FTCD_MOUSE Formimidoyltransferase-cyclodeaminase |
| | | | OS = Mus musculus GN = Ftcd PE = 1 SV = 1 |
| 0.0094 | 0.0085 | Prdm16 | A2A935\|PRD16_MOUSE PR domain zinc finger protein 16 OS = Mus |
| | | | musculus GN = Prdm16 PE = 1 SV = 1 |
| 0.0095 | 0.0085 | Usp5 | P56399\|UBP5_MOUSE Ubiquitin carboxyl-terminal hydrolase 5 |
| | | | OS = Mus musculus GN = Usp5 PE = 1 SV = 1 |
| 0.0095 | 0.0085 | Tufm | Q8BFR5\|EFTU_MOUSE Elongation factor Tu, mitochondrial OS = Mus |
| | | | musculus GN = Tufm PE = 1 SV = 1 |
| 0.0096 | 0.0085 | Ushbp1 | Q8R370\|USBP1_MOUSE Usher syndrome type-1C protein-binding |
| | | | protein 1 OS = Mus musculus GN = Ushbp1 PE = 1 SV = 2 |
| 0.0098 | 0.0085 | Gbe1 | Q9D6Y9\|GLGB_MOUSE 1,4-alpha-glucan-branching enzyme |
| | | | OS = Mus musculus GN = Gbe1 PE = 1 SV = 1 |
| 0.0098 | 0.0085 | Hspa8 | P63017\|HSP7C_MOUSE Heat shock cognate 71 kDa protein |
| | | | OS = Mus musculus GN = Hspa8 PE = 1 SV = 1 |
| 0.0101 | 0.0085 | Myh9 | Q8VDD5\|MYH9_MOUSE Myosin-9 OS = Mus musculus GN = Myh9 |
| | | | PE = 1 SV = 4 |
| 0.0101 | 0.0085 | Pfn1 | P62962\|PROF1_MOUSE Profilin-1 OS = Mus musculus GN = Pfn1 |
| | | | PE = 1 SV = 2 |
| 0.0102 | 0.0085 | Tkt | P40142\|TKT_MOUSE Transketolase OS = Mus musculus GN = Tkt |
| | | | PE = 1 SV = 1 |
| 0.0103 | 0.0085 | Sdhb | Q9CQA3\|SDHB_MOUSE Succinate dehydrogenase [ubiquinone] iron- |
| | | | sulfur subunit, mitochondrial OS = Mus musculus GN = Sdhb PE = 1 |
| | | | SV = 1 |
| 0.0103 | 0.0085 | Ighg1 | P01868\|IGHG1_MOUSE Ig gamma-1 chain C region secreted form |
| | | | OS = Mus musculus GN = Ighg1 PE = 1 SV = 1 |
| 0.0105 | 0.0085 | Arsj | Q8BM89\|ARSJ_MOUSE Arylsulfatase J OS = Mus musculus GN = Arsj |
| | | | PE = 2 SV = 1 |
| 0.0105 | 0.0085 | Hnrnpk | P61979\|HNRPK_MOUSE Heterogeneous nuclear ribonucleoprotein K |
| | | | OS = Mus musculus GN = Hnrnpk PE = 1 SV = 1 |
| 0.0105 | 0.0085 | Selenbp1 | P17563\|SBP1_MOUSE Selenium-binding protein 1 OS = Mus |
| | | | musculus GN = Selenbp1 PE = 1 SV = 2 |
| 0.0106 | 0.0085 | Aco2 | Q99KIO\|ACON_MOUSE Aconitate hydratase, mitochondrial OS = Mus |
| | | | musculus GN = Aco2 PE = 1 SV = 1 |
| 0.0106 | 0.0085 | Pzp | Q61838\|PZP_MOUSE Pregnancy zone protein OS = Mus musculus |
| | | | GN = Pzp PE = 1 SV = 3 |
| 0.0106 | 0.0085 | Aldh6a1 | Q9EQ20\|MMSA_MOUSE Methylmalonate-semialdehyde |
| | | | dehydrogenase [acylating], mitochondrial OS = Mus musculus |
| | | | GN = Aldh6a1 PE = 1 SV = 1 |
| 0.0107 | 0.0085 | Sec14l2 | Q99J08\|S14L2_MOUSE SEC14-like protein 2 OS = Mus musculus |
| | | | GN = Sec14l2 PE = 1 SV = 1 |
| 0.0107 | 0.0085 | Sord | Q64442\|DHSO_MOUSE Sorbitol dehydrogenase OS = Mus musculus |
| | | | GN = Sord PE = 1 SV = 3 |
| 0.0107 | 0.0085 | Hsd17b4 | P51660\|DHB4_MOUSE Peroxisomal multifunctional enzyme type 2 |
| | | | OS = Mus musculus GN = Hsd17b4 PE = 1 SV = 3 |
| 0.0107 | 0.0085 | Eef1a1 | P10126\|EF1A1_MOUSE Elongation factor 1-alpha 1 OS = Mus |
| | | | musculus GN = Eef1a1 PE = 1 SV = 3 |
| 0.0107 | 0.0085 | Pgk1 | P09411\|PGK1_MOUSE Phosphoglycerate kinase 1 OS = Mus |
| | | | musculus GN = Pgk1 PE = 1 SV = 4 |
| 0.0109 | 0.0085 | Aldh8a1 | Q8BH00\|AL8A1_MOUSE Aldehyde dehydrogenase family 8 member |
| | | | A1 OS = Mus musculus GN = Aldh8a1 PE = 1 SV = 1 |
| 0.0110 | 0.0085 | Gnmt | Q9QXF8\|GNMT_MOUSE Glycine N-methyltransferase OS = Mus |
| | | | musculus GN = Gnmt PE = 1 SV = 3 |
| 0.0111 | 0.0085 | Acsl1 | P41216\|ACSL1_MOUSE Long-chain-fatty-acid--CoA ligase 1 OS = Mus |
| | | | musculus GN = Acsl1 PE = 1 SV = 2 |
| 0.0112 | 0.0085 | Cyp3a11 | Q64459\|CP3AB_MOUSE Cytochrome P450 3A11 OS = Mus musculus |
| | | | GN = Cyp3a11 PE = 1 SV = 1 |
| 0.0112 | 0.0085 | Ldha | P06151\|LDHA_MOUSE L-lactate dehydrogenase A chain OS = Mus |
| | | | musculus GN = Ldha PE = 1 SV = 3 |
| 0.0113 | 0.0085 | Aco1 | P28271\|ACOC_MOUSE Cytoplasmic aconitate hydratase OS = Mus |
| | | | musculus GN = Aco1 PE = 1 SV = 3 |
| 0.0114 | 0.0085 | Pgm1 | Q9D0F9\|PGM1_MOUSE Phosphoglucomutase-1 OS = Mus musculus |
| | | | GN = Pgm1 PE = 1 SV = 4 |
| 0.0114 | 0.0085 | Cbs | Q91WT9\|CBS_MOUSE Cystathionine beta-synthase OS = Mus |
| | | | musculus GN = Cbs PE = 1 SV = 3 |
| 0.0114 | 0.0085 | Serpinc1 | P32261\|ANT3_MOUSE Antithrombin-III OS = Mus musculus |
| | | | GN = Serpinc1 PE = 1 SV = 1 |
| 0.0114 | 0.0085 | Pbld2 | Q9CXN7\|PBLD2_MOUSE Phenazine biosynthesis-like domain- |
| | | | containing protein 2 OS = Mus musculus GN = Pbld2 PE = 1 SV = 1 |
| 0.0115 | 0.0085 | Hmgcl | P38060\|HMGCL_MOUSE Hydroxymethylglutaryl-CoA lyase, |
| | | | mitochondrial OS = Mus musculus GN = Hmgcl PE = 1 SV = 2 |
| 0.0116 | 0.0085 | Tuba4a | P68368\|TBA4A_MOUSE Tubulin alpha-4A chain OS = Mus musculus |
| | | | GN = Tuba4a PE = 1 SV = 1 |
| 0.0117 | 0.0085 | Idh1 | O88844\|IDHC_MOUSE Isocitrate dehydrogenase [NADP] cytoplasmic |
| | | | OS = Mus musculus GN = Idh1 PE = 1 SV = 2 |
| 0.0118 | 0.0085 | Hadha | Q8BMS1\|ECHA_MOUSE Trifunctional enzyme subunit alpha, |
| | | | mitochondrial OS = Mus musculus GN = Hadha PE = 1 SV = 1 |
| 0.0119 | 0.0085 | Hmgcs2 | P54869\|HMCS2_MOUSE Hydroxymethylglutaryl-CoA synthase, |
| | | | mitochondrial OS = Mus musculus GN = Hmgcs2 PE = 1 SV = 2 |
| 0.0119 | 0.0085 | Krt10 | P02535\|K1C10_MOUSE Keratin, type I cytoskeletal 10 OS = Mus |
| | | | musculus GN = Krt10 PE = 1 SV = 3 |
| 0.0119 | 0.0085 | Stard10 | Q9JMD3\|PCTL_MOUSE PCTP-like protein OS = Mus musculus |
| | | | GN = Stard10 PE = 1 SV = 1 |
| 0.0123 | 0.0086 | Asl | Q91YI0\|ARLY_MOUSE Argininosuccinate lyase OS = Mus musculus |
| | | | GN = Asl PE = 1 SV = 1 |
| 0.0124 | 0.0086 | Psmd2 | Q8VDM4\|PSMD2_MOUSE 26S proteasome non-ATPase regulatory |
| | | | subunit 2 OS = Mus musculus GN = Psmd2 PE = 1 SV = 1 |
| 0.0125 | 0.0086 | Cyp2f2 | P33267\|CP2F2_MOUSE Cytochrome P450 2F2 OS = Mus musculus |
| | | | GN = Cyp2f2 PE = 1 SV = 1 |
| 0.0126 | 0.0086 | Comt | O88587\|COMT_MOUSE Catechol O-methyltransferase OS = Mus |
| | | | musculus GN = Comt PE = 1 SV = 2 |
| 0.0127 | 0.0086 | Hsp90b1 | P08113\|ENPL_MOUSE Endoplasmin OS = Mus musculus |
| | | | GN = Hsp90b1 PE = 1 SV = 2 |
| 0.0127 | 0.0086 | Mdh2 | P08249\|MDHM_MOUSE Malate dehydrogenase, mitochondrial |
| | | | OS = Mus musculus GN = Mdh2 PE = 1 SV = 3 |
| 0.0130 | 0.0088 | Akr1c6 | P70694\|DHB5_MOUSE Estradiol 17 beta-dehydrogenase 5 OS = Mus |
| | | | musculus GN = Akr1c6 PE = 1 SV = 1 |
| 0.0131 | 0.0088 | Anxa2 | P07356\|ANXA2_MOUSE Annexin A2 OS = Mus musculus GN = Anxa2 |
| | | | PE = 1 SV = 2 |
| 0.0132 | 0.0088 | Atp1a1 | Q8VDN2\|AT1A1_MOUSE Sodium/potassium-transporting ATPase |
| | | | subunit alpha-1 OS = Mus musculus GN = Atp1a1 PE = 1 SV = 1 |
| 0.0133 | 0.0088 | Aldh7a1 | Q9DBF1\|AL7A1_MOUSE Alpha-aminoadipic semialdehyde |
| | | | dehydrogenase OS = Mus musculus GN = Aldh7a1 PE = 1 SV = 4 |
| 0.0135 | 0.0088 | Cltc | Q68FD5\|CLH1_MOUSE Clathrin heavy chain 1 OS = Mus musculus |
| | | | GN = Cltc PE = 1 SV = 3 |
| 0.0136 | 0.0088 | Cyp2d26 | Q8CIM7\|CP2DQ_MOUSE Cytochrome P450 2D26 OS = Mus |
| | | | musculus GN = Cyp2d26 PE = 1 SV = 1 |
| 0.0137 | 0.0088 | Hnrnpf | Q9Z2X1\|HNRPF_MOUSE Heterogeneous nuclear ribonucleoprotein F |
| | | | OS = Mus musculus GN = Hnrnpf PE = 1 SV = 3 |
| 0.0138 | 0.0088 | Abhd14b | Q8VCR7\|ABHEB_MOUSE Protein ABHD14B OS = Mus musculus |
| | | | GN = Abhd14b PE = 1 SV = 1 |
| 0.0139 | 0.0088 | Ces3a | Q63880\|EST3A_MOUSE Carboxylesterase 3A OS = Mus musculus |
| | | | GN = Ces3a PE = 1 SV = 2 |
| 0.0139 | 0.0088 | Ubb | P0CG49\|UBB_MOUSE Polyubiquitin-B OS = Mus musculus GN = Ubb |
| | | | PE = 2 SV = 1 |
| 0.0140 | 0.0088 | Uox | P25688\|URIC_MOUSE Uricase OS = Mus musculus GN = Uox PE = 1 |
| | | | SV = 2 |
| 0.0140 | 0.0088 | Akr1a1 | Q9JII6\|AK1A1_MOUSE Alcohol dehydrogenase [NADP(+)] OS = Mus |
| | | | musculus GN = Akr1a1 PE = 1 SV = 3 |
| 0.0144 | 0.0089 | Rps20 | P60867\|RS20_MOUSE 40S ribosomal protein S20 OS = Mus musculus |
| | | | GN = Rps20 PE = 1 SV = 1 |
| 0.0145 | 0.0089 | Anxa6 | P14824\|ANXA6_MOUSE Annexin A6 OS = Mus musculus GN = Anxa6 |
| | | | PE = 1 SV = 3 |
| 0.0145 | 0.0089 | Hbb-b1 | P02088\|HBB1_MOUSE Hemoglobin subunit beta-1 OS = Mus |
| | | | musculus GN = Hbb-b1 PE = 1 SV = 2 |
| 0.0145 | 0.0089 | Arf3 | P61205\|ARF3_MOUSE ADP-ribosylation factor 3 OS = Mus musculus |
| | | | GN = Arf3 PE = 2 SV = 2 |
| 0.0152 | 0.0093 | Abcd3 | P55096\|ABCD3_MOUSE ATP-binding cassette sub-family D member |
| | | | 3 OS = Mus musculus GN = Abcd3 PE = 1 SV = 2 |
| 0.0157 | 0.0095 | Chdh | Q8BJ64\|CHDH_MOUSE Choline dehydrogenase, mitochondrial |
| | | | OS = Mus musculus GN = Chdh PE = 1 SV = 1 |
| 0.0162 | 0.0097 | H3f3c | P02301\|H3C_MOUSE Histone H3.3C OS = Mus musculus GN = H3f3c |
| | | | PE = 3 SV = 3 |
| 0.0162 | 0.0097 | Ywhaz | P63101\|1433Z_MOUSE 14-3-3 protein zeta/delta OS = Mus musculus |
| | | | GN = Ywhaz PE = 1 SV = 1 |
| 0.0167 | 0.0098 | Aldh4a1 | Q8CHTO\|AL4A1_MOUSE Delta-1-pyrroline-5-carboxylate |
| | | | dehydrogenase, mitochondrial OS = Mus musculus GN = Aldh4a1 PE = 1 |
| | | | SV = 3 |
| 0.0169 | 0.0098 | Bhmt | O35490\|BHMT1_MOUSE Betaine--homocysteine S-methyltransferase |
| | | | 1 OS = Mus musculus GN = Bhmt PE = 1 SV = 1 |
| 0.0169 | 0.0098 | Myl6 | Q60605\|MYL6_MOUSE Myosin light polypeptide 6 OS = Mus musculus |
| | | | GN = Myl6 PE = 1 SV = 3 |
| 0.0170 | 0.0098 | Acat1 | Q8QZT1\|THIL_MOUSE Acetyl-CoA acetyltransferase, mitochondrial |
| | | | OS = Mus musculus GN = Acat1 PE = 1 SV = 1 |
| 0.0172 | 0.0098 | Tkfc | Q8VC30\|TKFC_MOUSE Triokinase/FMN cyclase OS = Mus musculus |
| | | | GN = Tkfc PE = 1 SV = 1 |
| 0.0172 | 0.0098 | Mccc2 | Q3ULD5\|MCCB_MOUSE Methylcrotonoyl-CoA carboxylase beta |
| | | | chain, mitochondrial OS = Mus musculus GN = Mccc2 PE = 1 SV = 1 |
| 0.0172 | 0.0098 | Ahcy | P50247\|SAHH_MOUSE Adenosylhomocysteinase OS = Mus musculus |
| | | | GN = Ahcy PE = 1 SV = 3 |
| 0.0172 | 0.0098 | Rnh1 | Q91VI7\|RINI_MOUSE Ribonuclease inhibitor OS = Mus musculus |
| | | | GN = Rnh1 PE = 1 SV = 1 |
| 0.0173 | 0.0098 | Sucla2 | Q9Z2I9\|SUCB1_MOUSE Succinate--CoA ligase [ADP-forming] |
| | | | subunit beta, mitochondrial OS = Mus musculus GN = Sucla2 PE = 1 |
| | | | SV = 2 |
| 0.0179 | 0.0100 | Hgd | O09173\|HGD_MOUSE Homogentisate 1,2-dioxygenase OS = Mus |
| | | | musculus GN = Hgd PE = 1 SV = 2 |
| 0.0180 | 0.0100 | Isoc2a | P85094\|ISC2A_MOUSE Isochorismatase domain-containing protein |
| | | | 2A OS = Mus musculus GN = Isoc2a PE = 1 SV = 1 |
| 0.0180 | 0.0100 | Fdps | Q920E5\|FPPS_MOUSE Farnesyl pyrophosphate synthase OS = Mus |
| | | | musculus GN = Fdps PE = 1 SV = 1 |
| 0.0181 | 0.0101 | Dhrs1 | Q99L04\|DHRS1_MOUSE Dehydrogenase/reductase SDR family |
| | | | member 1 OS = Mus musculus GN = Dhrs1 PE = 1 SV = 1 |
| 0.0184 | 0.0101 | Pkhd1l1 | Q80ZA4\|PKHL1_MOUSE Fibrocystin-L OS = Mus musculus |
| | | | GN = Pkhd1l1 PE = 1 SV = 1 |
| 0.0187 | 0.0102 | Krt18 | P05784\|K1C18_MOUSE Keratin, type I cytoskeletal 18 OS = Mus |
| | | | musculus GN = Krt18 PE = 1 SV = 5 |
| 0.0192 | 0.0104 | Rgn | Q64374\|RGN_MOUSE Regucalcin OS = Mus musculus GN = Rgn PE = 1 |
| | | | SV = 1 |
| 0.0193 | 0.0104 | Cct8 | P42932\|TCPQ_MOUSE T-complex protein 1 subunit theta OS = Mus |
| | | | musculus GN = Cct8 PE = 1 SV = 3 |
| 0.0193 | 0.0104 | Pck1 | Q9Z2V4\|PCKGC_MOUSE Phosphoenolpyruvate carboxykinase, |
| | | | cytosolic [GTP] OS = Mus musculus GN = Pck1 PE = 1 SV = 1 |
| 0.0193 | 0.0104 | Got1 | P05201\|AATC_MOUSE Aspartate aminotransferase, cytoplasmic |
| | | | OS = Mus musculus GN = Got1 PE = 1 SV = 3 |
| 0.0195 | 0.0104 | Krt72 | Q6IME9\|K2C72_MOUSE Keratin, type II cytoskeletal 72 OS = Mus |
| | | | musculus GN = Krt72 PE = 3 SV = 1 |
| 0.0195 | 0.0104 | Tinagl1 | Q99JR5\|TINAL_MOUSE Tubulointerstitial nephritis antigen-like |
| | | | OS = Mus musculus GN = Tinagl1 PE = 1 SV = 1 |
| 0.0197 | 0.0104 | Krt42 | Q6IFX2\|K1C42_MOUSE Keratin, type I cytoskeletal 42 OS = Mus |
| | | | musculus GN = Krt42 PE = 1 SV = 1 |
| 0.0197 | 0.0104 | Tgm2 | P21981\|TGM2_MOUSE Protein-glutamine gamma- |
| | | | glutamyltransferase 2 OS = Mus musculus GN = Tgm2 PE = 1 SV = 4 |
| 0.0200 | 0.0104 | Ass1 | P16460\|ASSY_MOUSE Argininosuccinate synthase OS = Mus |
| | | | musculus GN = Ass1 PE = 1 SV = 1 |
| 0.0204 | 0.0105 | Me1 | P06801\|MAOX_MOUSE NADP-dependent malic enzyme OS = Mus |
| | | | musculus GN = Me1 PE = 1 SV = 2 |
| 0.0204 | 0.0105 | Acadvl | P50544\|ACADV_MOUSE Very long-chain specific acyl-CoA |
| | | | dehydrogenase, mitochondrial OS = Mus musculus GN = Acadvl PE = 1 |
| | | | SV = 3 |
| 0.0206 | 0.0105 | Gdi2 | Q61598\|GDIB_MOUSE Rab GDP dissociation inhibitor beta OS = Mus |
| | | | musculus GN = Gdi2 PE = 1 SV = 1 |
| 0.0208 | 0.0105 | Hist1h4a | P62806\|H4_MOUSE Histone H4 OS = Mus musculus GN = Hist1h4a |
| | | | PE = 1 SV = 2 |
| 0.0208 | 0.0105 | Ddx5 | Q61656\|DDX5_MOUSE Probable ATP-dependent RNA helicase |
| | | | DDX5 OS = Mus musculus GN = Ddx5 PE = 1 SV = 2 |
| 0.0209 | 0.0105 | Haao | Q78JT3\|3HAO_MOUSE 3-hydroxyanthranilate 3,4-dioxygenase |
| | | | OS = Mus musculus GN = Haao PE = 1 SV = 1 |
| 0.0210 | 0.0105 | Agxt | O35423\|SPYA_MOUSE Serine--pyruvate aminotransferase, |
| | | | mitochondrial OS = Mus musculus GN = Agxt PE = 1 SV = 3 |
| 0.0210 | 0.0105 | Krt75 | Q8BGZ7\|K2C75_MOUSE Keratin, type II cytoskeletal 75 OS = Mus |
| | | | musculus GN = Krt75 PE = 1 SV = 1 |
| 0.0210 | 0.0105 | Mfap4 | Q9D1H9\|MFAP4_MOUSE Microfibril-associated glycoprotein 4 |
| | | | OS = Mus musculus GN = Mfap4 PE = 1 SV = 1 |
| 0.0210 | 0.0105 | Aldob | Q91Y97\|ALDOB_MOUSE Fructose-bisphosphate aldolase B OS = Mus |
| | | | musculus GN = Aldob PE = 1 SV = 3 |
| 0.0212 | 0.0105 | Pdha1 | P35486\|ODPA_MOUSE Pyruvate dehydrogenase E1 component |
| | | | subunit alpha, somatic form, mitochondrial OS = Mus musculus |
| | | | GN = Pdha1 PE = 1 SV = 1 |
| 0.0213 | 0.0105 | Dmgdh | Q9DBT9\|M2GD_MOUSE Dimethylglycine dehydrogenase, |
| | | | mitochondrial OS = Mus musculus GN = Dmgdh PE = 1 SV = 1 |
| 0.0217 | 0.0106 | 1 SV | P01631\|KV2A7_MOUSE Ig kappa chain V-II region 26-10 OS = Mus |
| | | | musculus PE = 1 SV = 1 |
| 0.0217 | 0.0106 | Cntf | P51642\|CNTF_MOUSE Ciliary neurotrophic factor OS = Mus musculus |
| | | | GN = Cntf PE = 2 SV = 1 |
| 0.0220 | 0.0107 | Hagh | Q99KB8\|GLO2_MOUSE Hydroxyacylglutathione hydrolase, |
| | | | mitochondrial OS = Mus musculus GN = Hagh PE = 1 SV = 2 |
| 0.0221 | 0.0107 | Hist1h2bc | Q6ZWY9\|H2B1C_MOUSE Histone H2B type 1-C/E/G OS = Mus |
| | | | musculus GN = Hist1h2bc PE = 1 SV = 3 |
| 0.0222 | 0.0107 | Fmo5 | P97872\|FMO5_MOUSE Dimethylaniline monooxygenase [N-oxide- |
| | | | forming] 5 OS = Mus musculus GN = Fmo5 PE = 1 SV = 4 |
| 0.0226 | 0.0108 | Cs | Q9CZU6\|CISY_MOUSE Citrate synthase, mitochondrial OS = Mus |
| | | | musculus GN = Cs PE = 1 SV = 1 |
| 0.0226 | 0.0108 | Tcp1 | P11983\|TCPA_MOUSE T-complex protein 1 subunit alpha OS = Mus |
| | | | musculus GN = Tcp1 PE = 1 SV = 3 |
| 0.0227 | 0.0108 | Aldh9a1 | Q9JLJ2\|AL9A1_MOUSE 4-trimethylaminobutyraldehyde |
| | | | dehydrogenase OS = Mus musculus GN = Aldh9a1 PE = 1 SV = 1 |
| 0.0228 | 0.0108 | Dcaf8 | Q8N7N5\|DCAF8_MOUSE DDB1- and CUL4-associated factor 8 |
| | | | OS = Mus musculus GN = Dcaf8 PE = 1 SV = 1 |
| 0.0235 | 0.0110 | Aldh1a1 | P24549\|AL1A1_MOUSE Retinal dehydrogenase 1 OS = Mus musculus |
| | | | GN = Aldh1a1 PE = 1 SV = 5 |
| 0.0236 | 0.0110 | Mug1 | P28665\|MUG1_MOUSE Murinoglobulin-1 OS = Mus musculus |
| | | | GN = Mug1 PE = 1 SV = 3 |
| 0.0237 | 0.0110 | Gapdh | P16858\|G3P_MOUSE Glyceraldehyde-3-phosphate dehydrogenase |
| | | | OS = Mus musculus GN = Gapdh PE = 1 SV = 2 |
| 0.0237 | 0.0110 | Coq8a | Q60936\|COQ8A_MOUSE Atypical kinase COQ8A, mitochondrial |
| | | | OS = Mus musculus GN = Coq8a PE = 1 SV = 2 |
| 0.0244 | 0.0112 | Adgrl1 | Q80TR1\|AGRL1_MOUSE Adhesion G protein-coupled receptor L1 |
| | | | OS = Mus musculus GN = Adgrl1 PE = 1 SV = 2 |
| 0.0244 | 0.0112 | Eif5a | P63242\|IF5A1_MOUSE Eukaryotic translation initiation factor 5A-1 |
| | | | OS = Mus musculus GN = Eif5a PE = 1 SV = 2 |
| 0.0246 | 0.0113 | Psmd1 | Q3TXS7\|PSMD1_MOUSE 26S proteasome non-ATPase regulatory |
| | | | subunit 1 OS = Mus musculus GN = Psmd1 PE = 1 SV = 1 |
| 0.0253 | 0.0115 | Pipox | Q9D826\|SOX_MOUSE Peroxisomal sarcosine oxidase OS = Mus |
| | | | musculus GN = Pipox PE = 1 SV = 1 |
| 0.0254 | 0.0115 | Ak2 | Q9WTP6\|KAD2_MOUSE Adenylate kinase 2, mitochondrial OS = Mus |
| | | | musculus GN = Ak2 PE = 1 SV = 5 |
| 0.0255 | 0.0115 | Lcp1 | Q61233\|PLSL_MOUSE Plastin-2 OS = Mus musculus GN = Lcp1 PE = 1 |
| | | | SV = 4 |
| 0.0257 | 0.0115 | Vtn | P29788\|VTNC_MOUSE Vitronectin OS = Mus musculus GN = Vtn PE = 1 |
| | | | SV = 2 |
| 0.0257 | 0.0115 | Krt2 | Q3TTY5\|K22E_MOUSE Keratin, type II cytoskeletal 2 epidermal |
| | | | OS = Mus musculus GN = Krt2 PE = 1 SV = 1 |
| 0.0261 | 0.0116 | Eef1b | O70251\|EF1B_MOUSE Elongation factor 1-beta OS = Mus musculus |
| | | | GN = Eef1b PE = 1 SV = 5 |
| 0.0263 | 0.0117 | Nid2 | O88322\|NID2_MOUSE Nidogen-2 OS = Mus musculus GN = Nid2 PE = 1 |
| | | | SV = 2 |
| 0.0265 | 0.0117 | Hadhb | Q99JYO\|ECHB_MOUSE Trifunctional enzyme subunit beta, |
| | | | mitochondrial OS = Mus musculus GN = Hadhb PE = 1 SV = 1 |
| 0.0274 | 0.0120 | Serpina1d | Q00897\|A1AT4_MOUSE Alpha-1-antitrypsin 1-4 OS = Mus musculus |
| | | | GN = Serpina1d PE = 1 SV = 1 |
| 0.0280 | 0.0123 | Rbm20 | Q3UQS8\|RBM20_MOUSE RNA-binding protein 20 OS = Mus musculus |
| | | | GN = Rbm20 PE = 1 SV = 3 |
| 0.0284 | 0.0123 | Fabp5 | Q05816\|FABP5_MOUSE Fatty acid-binding protein, epidermal |
| | | | OS = Mus musculus GN = Fabp5 PE = 1 SV = 3 |
| 0.0284 | 0.0123 | Trap1 | Q9CQN1\|TRAP1_MOUSE Heat shock protein 75 kDa, mitochondrial |
| | | | OS = Mus musculus GN = Trap1 PE = 1 SV = 1 |
| 0.0286 | 0.0124 | Alb | P07724\|ALBU_MOUSE Serum albumin OS = Mus musculus GN = Alb |
| | | | PE = 1 SV = 3 |
| 0.0289 | 0.0124 | Abat | P61922\|GABT_MOUSE 4-aminobutyrate aminotransferase, |
| | | | mitochondrial OS = Mus musculus GN = Abat PE = 1 SV = 1 |
| 0.0289 | 0.0124 | Pklr | P53657\|KPYR_MOUSE Pyruvate kinase PKLR OS = Mus musculus |
| | | | GN = Pklr PE = 1 SV = 1 |
| 0.0291 | 0.0124 | Ndufab1 | Q9CR21\|ACPM_MOUSE Acyl carrier protein, mitochondrial OS = Mus |
| | | | musculus GN = Ndufab1 PE = 1 SV = 1 |
| 0.0294 | 0.0125 | Nit2 | Q9JHW2\|NIT2_MOUSE Omega-amidase NIT2 OS = Mus musculus |
| | | | GN = Nit2 PE = 1 SV = 1 |
| 0.0294 | 0.0125 | Hint2 | Q9D0S9\|HINT2_MOUSE Histidine triad nucleotide-binding protein 2, |
| | | | mitochondrial OS = Mus musculus GN = Hint2 PE = 1 SV = 1 |
| 0.0298 | 0.0125 | Pgam1 | Q9DBJ1\|PGAM1_MOUSE Phosphoglycerate mutase 1 OS = Mus |
| | | | musculus GN = Pgam1 PE = 1 SV = 3 |
| 0.0298 | 0.0125 | Acly | Q91V92\|ACLY_MOUSE ATP-citrate synthase OS = Mus musculus |
| | | | GN = Acly PE = 1 SV = 1 |
| 0.0302 | 0.0125 | Krt5 | Q922U2\|K2C5_MOUSE Keratin, type II cytoskeletal 5 OS = Mus |
| | | | musculus GN = Krt5 PE = 1 SV = 1 |
| 0.0302 | 0.0125 | Eif3a | P23116\|EIF3A_MOUSE Eukaryotic translation initiation factor 3 |
| | | | subunit A OS = Mus musculus GN = Eif3a PE = 1 SV = 5 |
| 0.0303 | 0.0125 | Nid1 | P10493\|NID1_MOUSE Nidogen-1 OS = Mus musculus GN = Nid1 PE = 1 |
| | | | SV = 2 |
| 0.0305 | 0.0126 | Iqgap2 | Q3UQ44\|IQGA2_MOUSE Ras GTPase-activating-like protein IQGAP2 |
| | | | OS = Mus musculus GN = Iqgap2 PE = 1 SV = 2 |
| 0.0310 | 0.0127 | Fbp1 | Q9QXD6\|F16P1_MOUSE Fructose-1,6-bisphosphatase 1 OS = Mus |
| | | | musculus GN = Fbp1 PE = 1 SV = 3 |
| 0.0313 | 0.0128 | Urad | Q283N4\|URAD_MOUSE 2-oxo-4-hydroxy-4-carboxy-5- |
| | | | ureidoimidazoline decarboxylase OS = Mus musculus GN = Urad PE = 1 |
| | | | SV = 1 |
| 0.0315 | 0.0128 | Ckm | P07310\|KCRM_MOUSE Creatine kinase M-type OS = Mus musculus |
| | | | GN = Ckm PE = 1 SV = 1 |
| 0.0318 | 0.0128 | Fasn | P19096\|FAS_MOUSE Fatty acid synthase OS = Mus musculus |
| | | | GN = Fasn PE = 1 SV = 2 |
| 0.0319 | 0.0128 | Reep6 | Q9JM62\|REEP6_MOUSE Receptor expression-enhancing protein 6 |
| | | | OS = Mus musculus GN = Reep6 PE = 1 SV = 1 |
| 0.0323 | 0.0129 | Prdx5 | P99029\|PRDX5_MOUSE Peroxiredoxin-5, mitochondrial OS = Mus |
| | | | musculus GN = Prdx5 PE = 1 SV = 2 |
| 0.0325 | 0.0130 | Krt14 | Q61781\|K1C14_MOUSE Keratin, type I cytoskeletal 14 OS = Mus |
| | | | musculus GN = Krt14 PE = 1 SV = 2 |
| 0.0328 | 0.0130 | Pgrmc1 | O55022\|PGRC1_MOUSE Membrane-associated progesterone |
| | | | receptor component 1 OS = Mus musculus GN = Pgrmc1 PE = 1 SV = 4 |
| 0.0329 | 0.0130 | Tpm3 | P21107\|TPM3_MOUSE Tropomyosin alpha-3 chain OS = Mus |
| | | | musculus GN = Tpm3 PE = 1 SV = 3 |
| 0.0332 | 0.0131 | Rps5 | P97461\|RS5_MOUSE 40S ribosomal protein S5 OS = Mus musculus |
| | | | GN = Rps5 PE = 1 SV = 3 |
| 0.0334 | 0.0131 | Gstp1 | P19157\|GSTP1_MOUSE Glutathione S-transferase P 1 OS = Mus |
| | | | musculus GN = Gstp1 PE = 1 SV = 2 |
| 0.0334 | 0.0131 | Ehhadh | Q9DBM2\|ECHP_MOUSE Peroxisomal bifunctional enzyme OS = Mus |
| | | | musculus GN = Ehhadh PE = 1 SV = 4 |
| 0.0335 | 0.0131 | Etfa | Q99LC5\|ETFA_MOUSE Electron transfer flavoprotein subunit alpha, |
| | | | mitochondrial OS = Mus musculus GN = Etfa PE = 1 SV = 2 |
| 0.0341 | 0.0132 | Psmd12 | Q9D8W5\|PSD12_MOUSE 26S proteasome non-ATPase regulatory |
| | | | subunit 12 OS = Mus musculus GN = Psmd12 PE = 1 SV = 4 |
| 0.0343 | 0.0132 | Cyp2c50 | Q91X77\|CY250_MOUSE Cytochrome P450 2C50 OS = Mus musculus |
| | | | GN = Cyp2c50 PE = 1 SV = 2 |
| 0.0346 | 0.0132 | Kng1 | O08677\|KNG1_MOUSE Kininogen-1 OS = Mus musculus GN = Kng1 |
| | | | PE = 1 SV = 1 |
| 0.0347 | 0.0132 | Mif | P34884\|MIF_MOUSE Macrophage migration inhibitory factor OS = Mus |
| | | | musculus GN = Mif PE = 1 SV = 2 |
| 0.0348 | 0.0132 | H2afz | P0C0S6\|H2AZ_MOUSE Histone H2A.Z OS = Mus musculus GN = H2afz |
| | | | PE = 1 SV = 2 |
| 0.0349 | 0.0132 | Akr7a2 | Q8CG76\|ARK72_MOUSE Aflatoxin B1 aldehyde reductase member 2 |
| | | | OS = Mus musculus GN = Akr7a2 PE = 1 SV = 3 |
| 0.0349 | 0.0132 | Lonp1 | Q8CGK3\|LONM_MOUSE Lon protease homolog, mitochondrial |
| | | | OS = Mus musculus GN = Lonp1 PE = 1 SV = 2 |
| 0.0351 | 0.0132 | Selenbp2 | Q63836\|SBP2_MOUSE Selenium-binding protein 2 OS = Mus |
| | | | musculus GN = Selenbp2 PE = 1 SV = 2 |
| 0.0358 | 0.0134 | Acat2 | Q8CAY6\|THIC_MOUSE Acetyl-CoA acetyltransferase, cytosolic |
| | | | OS = Mus musculus GN = Acat2 PE = 1 SV = 2 |
| 0.0363 | 0.0135 | Hspg2 | Q05793\|PGBM_MOUSE Basement membrane-specific heparan |
| | | | sulfate proteoglycan core protein OS = Mus musculus GN = Hspg2 PE = 1 |
| | | | SV = 1 |
| 0.0365 | 0.0135 | Ttc38 | A3KMP2\|TTC38_MOUSE Tetratricopeptide repeat protein 38 |
| | | | OS = Mus musculus GN = Ttc38 PE = 1 SV = 2 |
| 0.0365 | 0.0135 | Rps9 | Q6ZWN5\|RS9_MOUSE 40S ribosomal protein S9 OS = Mus musculus |
| | | | GN = Rps9 PE = 1 SV = 3 |
| 0.0368 | 0.0136 | Cox5a | P12787\|COX5A_MOUSE Cytochrome c oxidase subunit 5A, |
| | | | mitochondrial OS = Mus musculus GN = Cox5a PE = 1 SV = 2 |
| 0.0368 | 0.0136 | Nsdhl | Q9R1J0\|NSDHL_MOUSE Sterol-4-alpha-carboxylate 3- |
| | | | dehydrogenase, decarboxylating OS = Mus musculus GN = Nsdhl PE = 1 |
| | | | SV = 1 |
| 0.0371 | 0.0136 | Xirp2 | Q4U4S6\|XIRP2_MOUSE Xin actin-binding repeat-containing protein 2 |
| | | | OS = Mus musculus GN = Xirp2 PE = 1 SV = 1 |
| 0.0371 | 0.0136 | Hnrnpm | Q9D0E1\|HNRPM_MOUSE Heterogeneous nuclear ribonucleoprotein |
| | | | M OS = Mus musculus GN = Hnrnpm PE = 1 SV = 3 |
| 0.0376 | 0.0137 | Ivd | Q9JHI5\|IVD_MOUSE Isovaleryl-CoA dehydrogenase, mitochondrial |
| | | | OS = Mus musculus GN = Ivd PE = 1 SV = 1 |
| 0.0378 | 0.0137 | Vim | P20152\|VIME_MOUSE Vimentin OS = Mus musculus GN = Vim PE = 1 |
| | | | SV = 3 |
| 0.0380 | 0.0137 | Apoe | P08226\|APOE_MOUSE Apolipoprotein E OS = Mus musculus |
| | | | GN = Apoe PE = 1 SV = 2 |
| 0.0381 | 0.0137 | Copg2 | Q9QXK3\|COPG2_MOUSE Coatomer subunit gamma-2 OS = Mus |
| | | | musculus GN = Copg2 PE = 1 SV = 1 |
| 0.0385 | 0.0138 | Ssr1 | Q9CY50\|SSRA_MOUSE Translocon-associated protein subunit alpha |
| | | | OS = Mus musculus GN = Ssr1 PE = 1 SV = 1 |
| 0.0387 | 0.0138 | Amdhd1 | Q9DBA8\|HUTI_MOUSE Probable imidazolonepropionase OS = Mus |
| | | | musculus GN = Amdhd1 PE = 1 SV = 1 |
| 0.0394 | 0.0140 | Klb | Q99N32\|KLOTB_MOUSE Beta-klotho OS = Mus musculus GN = Klb |
| | | | PE = 1 SV = 1 |
| 0.0394 | 0.0140 | Atxn2 | O70305\|ATX2_MOUSE Ataxin-2 OS = Mus musculus GN = Atxn2 PE = 1 |
| | | | SV = 1 |
| 0.0402 | 0.0142 | Adh5 | P28474\|ADHX_MOUSE Alcohol dehydrogenase class-3 OS = Mus |
| | | | musculus GN = Adh5 PE = 1 SV = 3 |
| 0.0405 | 0.0142 | Acox1 | Q9R0H0\|ACOX1_MOUSE Peroxisomal acyl-coenzyme A oxidase 1 |
| | | | OS = Mus musculus GN = Acox1 PE = 1 SV = 5 |
| 0.0405 | 0.0142 | Hacl1 | Q9QXE0\|HACL1_MOUSE 2-hydroxyacyl-CoA lyase 1 OS = Mus |
| | | | musculus GN = Hacl1 PE = 1 SV = 2 |
| 0.0406 | 0.0142 | Gpt | Q8QZR5\|ALAT1_MOUSE Alanine aminotransferase 1 OS = Mus |
| | | | musculus GN = Gpt PE = 1 SV = 3 |
| 0.0407 | 0.0142 | Mthfd1 | Q922D8\|C1TC_MOUSE C-1-tetrahydrofolate synthase, cytoplasmic |
| | | | OS = Mus musculus GN = Mthfd1 PE = 1 SV = 4 |
| 0.0409 | 0.0142 | Mipep | A6H611\|MIPEP_MOUSE Mitochondrial intermediate peptidase |
| | | | OS = Mus musculus GN = Mipep PE = 1 SV = 1 |
| 0.0409 | 0.0142 | Rpn2 | Q9DBG6\|RPN2_MOUSE Dolichyl-diphosphooligosaccharide--protein |
| | | | glycosyltransferase subunit 2 OS = Mus musculus GN = Rpn2 PE = 1 |
| | | | SV = 1 |
| 0.0417 | 0.0144 | Ttpa | Q8BWP5\|TTPA_MOUSE Alpha-tocopherol transfer protein OS = Mus |
| | | | musculus GN = Ttpa PE = 1 SV = 1 |
| 0.0420 | 0.0145 | Etfdh | Q921G7\|ETFD_MOUSE Electron transfer flavoprotein-ubiquinone |
| | | | oxidoreductase, mitochondrial OS = Mus musculus GN = Etfdh PE = 1 |
| | | | SV = 1 |
| 0.0425 | 0.0146 | Rplp0 | P14869\|RLA0_MOUSE 60S acidic ribosomal protein P0 OS = Mus |
| | | | musculus GN = Rplp0 PE = 1 SV = 3 |
| 0.0426 | 0.0146 | Psmc6 | P62334\|PRS10_MOUSE 26S protease regulatory subunit 10B |
| | | | OS = Mus musculus GN = Psmc6 PE = 1 SV = 1 |
| 0.0427 | 0.0146 | Rpl6 | P47911\|RL6_MOUSE 60S ribosomal protein L6 OS = Mus musculus |
| | | | GN = Rpl6 PE = 1 SV = 3 |
| 0.0436 | 0.0148 | Krt1 | P04104\|K2C1_MOUSE Keratin, type II cytoskeletal 1 OS = Mus |
| | | | musculus GN = Krt1 PE = 1 SV = 4 |
| 0.0437 | 0.0148 | Esd | Q9ROP3\|ESTD_MOUSE S-formylglutathione hydrolase OS = Mus |
| | | | musculus GN = Esd PE = 1 SV = 1 |
| 0.0437 | 0.0148 | Eif4a1 | P60843\|IF4A1_MOUSE Eukaryotic initiation factor 4A-I OS = Mus |
| | | | musculus GN = Eif4a1 PE = 1 SV = 1 |
| 0.0439 | 0.0148 | Khk | P97328\|KHK_MOUSE Ketohexokinase OS = Mus musculus GN = Khk |
| | | | PE = 1 SV = 1 |
| 0.0442 | 0.0148 | Copa | Q8CIE6\|COPA_MOUSE Coatomer subunit alpha OS = Mus musculus |
| | | | GN = Copa PE = 1 SV = 2 |
| 0.0445 | 0.0149 | Dbi | P31786\|ACBP_MOUSE Acyl-CoA-binding protein OS = Mus musculus |
| | | | GN = Dbi PE = 1 SV = 2 |
| 0.0450 | 0.0150 | Fn1 | P11276\|FINC_MOUSE Fibronectin OS = Mus musculus GN = Fn1 PE = 1 |
| | | | SV = 4 |
| 0.0456 | 0.0151 | Pdcd6ip | Q9WU78\|PDC6I_MOUSE Programmed cell death 6-interacting |
| | | | protein OS = Mus musculus GN = Pdcd6ip PE = 1 SV = 3 |
| 0.0457 | 0.0151 | Ugdh | O70475\|UGDH_MOUSE UDP-glucose 6-dehydrogenase OS = Mus |
| | | | musculus GN = Ugdh PE = 1 SV = 1 |
| 0.0457 | 0.0151 | Gtf3c1 | Q8K284\|TF3C1_MOUSE General transcription factor 3C polypeptide |
| | | | 1 OS = Mus musculus GN = Gtf3c1 PE = 1 SV = 2 |
| 0.0459 | 0.0151 | Gabrb3 | P63080\|GBRB3_MOUSE Gamma-aminobutyric acid receptor subunit |
| | | | beta-3 OS = Mus musculus GN = Gabrb3 PE = 2 SV = 1 |
| 0.0466 | 0.0153 | Rps25 | P62852\|RS25_MOUSE 40S ribosomal protein S25 OS = Mus musculus |
| | | | GN = Rps25 PE = 1 SV = 1 |
| 0.0474 | 0.0155 | Atp5o | Q9DB20\|ATPO_MOUSE ATP synthase subunit O, mitochondrial |
| | | | OS = Mus musculus GN = Atp5o PE = 1 SV = 1 |
| 0.0475 | 0.0155 | Por | P37040\|NCPR_MOUSE NADPH--cytochrome P450 reductase |
| | | | OS = Mus musculus GN = Por PE = 1 SV = 2 |
| 0.0487 | 0.0158 | Emilin1 | Q99K41\|EMIL1_MOUSE EMILIN-1 OS = Mus musculus GN = Emilin1 |
| | | | PE = 1 SV = 1 |
| 0.0491 | 0.0159 | Sds | Q8VBT2\|SDHL_MOUSE L-serine dehydratase/L-threonine deaminase |
| | | | OS = Mus musculus GN = Sds PE = 1 SV = 3 |
| 0.0496 | 0.0160 | Klhl1 | Q9JI74\|KLHL1_MOUSE Kelch-like protein 1 OS = Mus musculus |
| | | | GN = Klhl1 PE = 2 SV = 2 |
| 0.0497 | 0.0160 | Mycn | P03966\|MYCN_MOUSE N-myc proto-oncogene protein OS = Mus |
| | | | musculus GN = Mycn PE = 2 SV = 2 |
| 0.0502 | 0.0160 | Prdx3 | P20108\|PRDX3_MOUSE Thioredoxin-dependent peroxide reductase, |
| | | | mitochondrial OS = Mus musculus GN = Prdx3 PE = 1 SV = 1 |
| 0.0503 | 0.0160 | Aifm1 | Q9Z0X1\|AIFM1_MOUSE Apoptosis-inducing factor 1, mitochondrial |
| | | | OS = Mus musculus GN = Aifm1 PE = 1 SV = 1 |
| 0.0503 | 0.0160 | Ywhae | P62259\|1433E_MOUSE 14-3-3 protein epsilon OS = Mus musculus |
| | | | GN = Ywhae PE = 1 SV = 1 |
| 0.0504 | 0.0160 | Slc27a5 | Q4LDG0\|S27A5_MOUSE Bile acyl-CoA synthetase OS = Mus |
| | | | musculus GN = Slc27a5 PE = 1 SV = 2 |
| 0.0519 | 0.0164 | Lamb2 | Q61292\|LAMB2_MOUSE Laminin subunit beta-2 OS = Mus musculus |
| | | | GN = Lamb2 PE = 1 SV = 2 |
| 0.0520 | 0.0164 | Cyp2c54 | Q6XVG2\|CP254_MOUSE Cytochrome P450 2C54 OS = Mus musculus |
| | | | GN = Cyp2c54 PE = 1 SV = 1 |
| 0.0524 | 0.0164 | Gsto1 | O09131\|GSTO1_MOUSE Glutathione S-transferase omega-1 |
| | | | OS = Mus musculus GN = Gsto1 PE = 1 SV = 2 |
| 0.0524 | 0.0164 | Col6a6 | Q8C6K9\|CO6A6_MOUSE Collagen alpha-6(VI) chain OS = Mus |
| | | | musculus GN = Col6a6 PE = 1 SV = 2 |
| 0.0529 | 0.0165 | Akr1d1 | Q8VCX1\|AK1D1_MOUSE 3-oxo-5-beta-steroid 4-dehydrogenase |
| | | | OS = Mus musculus GN = Akr1d1 PE = 1 SV = 1 |
| 0.0530 | 0.0165 | Sec13 | Q9D1M0\|SEC13_MOUSE Protein SEC13 homolog OS = Mus |
| | | | musculus GN = Sec13 PE = 1 SV = 3 |
| 0.0539 | 0.0168 | Krt77 | Q6IFZ6\|K2C1B_MOUSE Keratin, type II cytoskeletal 1b OS = Mus |
| | | | musculus GN = Krt77 PE = 1 SV = 1 |
| 0.0546 | 0.0169 | Col4a2 | P08122\|CO4A2_MOUSE Collagen alpha-2(IV) chain OS = Mus |
| | | | musculus GN = Col4a2 PE = 1 SV = 4 |
| 0.0551 | 0.0170 | Cct3 | P80318\|TCPG_MOUSE T-complex protein 1 subunit gamma OS = Mus |
| | | | musculus GN = Cct3 PE = 1 SV = 1 |
| 0.0557 | 0.0172 | Jup | Q02257\|PLAK_MOUSE Junction plakoglobin OS = Mus musculus |
| | | | GN = Jup PE = 1 SV = 3 |
| 0.0564 | 0.0173 | 9913 GN | P02769\|ALBU_BOVIN Serum albumin OS = Bos taurus OX = 9913 |
| | | | GN = ALB PE = 1 SV = 4 |
| 0.0571 | 0.0175 | Aldoa | P05064\|ALDOA_MOUSE Fructose-bisphosphate aldolase A OS = Mus |
| | | | musculus GN = Aldoa PE = 1 SV = 2 |
| 0.0572 | 0.0175 | Ywhaq | P68254\|1433T_MOUSE 14-3-3 protein theta OS = Mus musculus |
| | | | GN = Ywhaq PE = 1 SV = 1 |
| 0.0575 | 0.0175 | Bsn | O88737\|BSN_MOUSE Protein bassoon OS = Mus musculus GN = Bsn |
| | | | PE = 1 SV = 4 |
| 0.0583 | 0.0177 | Col6a2 | Q02788\|CO6A2_MOUSE Collagen alpha-2(VI) chain OS = Mus |
| | | | musculus GN = Col6a2 PE = 1 SV = 3 |
| 0.0586 | 0.0177 | Xrra1 | Q3U3V8\|XRRA1_MOUSE X-ray radiation resistance-associated |
| | | | protein 1 OS = Mus musculus GN = Xrra1 PE = 2 SV = 1 |
| 0.0589 | 0.0178 | Hdlbp | Q8VDJ3\|VIGLN_MOUSE Vigilin OS = Mus musculus GN = Hdlbp PE = 1 |
| | | | SV = 1 |
| 0.0593 | 0.0179 | Gstz1 | Q9WVL0\|MAAI_MOUSE Maleylacetoacetate isomerase OS = Mus |
| | | | musculus GN = Gstz1 PE = 1 SV = 1 |
| 0.0597 | 0.0179 | Ncl | P09405\|NUCL_MOUSE Nucleolin OS = Mus musculus GN = Ncl PE = 1 |
| | | | SV = 2 |
| 0.0599 | 0.0179 | Adh1 | P00329\|ADH1_MOUSE Alcohol dehydrogenase 1 OS = Mus musculus |
| | | | GN = Adh1 PE = 1 SV = 2 |
| 0.0607 | 0.0181 | Dld | O08749\|DLDH_MOUSE Dihydrolipoyl dehydrogenase, mitochondrial |
| | | | OS = Mus musculus GN = Dld PE = 1 SV = 2 |
| 0.0608 | 0.0181 | Asap2 | Q7SIG6\|ASAP2_MOUSE Arf-GAP with SH3 domain, ANK repeat and |
| | | | PH domain-containing protein 2 OS = Mus musculus GN = Asap2 PE = 1 |
| | | | SV = 3 |
| 0.0612 | 0.0182 | Acox2 | Q9QXD1\|ACOX2_MOUSE Peroxisomal acyl-coenzyme A oxidase 2 |
| | | | OS = Mus musculus GN = Acox2 PE = 1 SV = 2 |
| 0.0615 | 0.0182 | Uso1 | Q9Z1Z0\|USO1_MOUSE General vesicular transport factor p115 |
| | | | OS = Mus musculus GN = Uso1 PE = 1 SV = 2 |
| 0.0623 | 0.0183 | Cyp3a13 | Q64464\|CP3AD_MOUSE Cytochrome P450 3A13 OS = Mus musculus |
| | | | GN = Cyp3a13 PE = 1 SV = 1 |
| 0.0624 | 0.0183 | Krt76 | Q3UV17\|K22O_MOUSE Keratin, type II cytoskeletal 2 oral OS = Mus |
| | | | musculus GN = Krt76 PE = 1 SV = 1 |
| 0.0636 | 0.0186 | Fip1l1 | Q9D824\|FIP1_MOUSE Pre-mRNA 3′-end-processing factor FIP1 |
| | | | OS = Mus musculus GN = Fip1l1 PE = 1 SV = 1 |
| 0.0644 | 0.0188 | Park7 | Q99LX0\|PARK7_MOUSE Protein deglycase DJ-1 OS = Mus musculus |
| | | | GN = Park7 PE = 1 SV = 1 |
| 0.0652 | 0.0190 | Cfl1 | P18760\|COF1_MOUSE Cofilin-1 OS = Mus musculus GN = Cfl1 PE = 1 |
| | | | SV = 3 |
| 0.0658 | 0.0191 | Fbln1 | Q08879\|FBLN1_MOUSE Fibulin-1 OS = Mus musculus GN = Fbln1 |
| | | | PE = 1 SV = 2 |
| 0.0667 | 0.0193 | Kyat3 | Q71RI9\|KAT3_MOUSE Kynurenine--oxoglutarate transaminase 3 |
| | | | OS = Mus musculus GN = Kyat3 PE = 1 SV = 1 |
| 0.0674 | 0.0194 | Cpne7 | Q0VE82\|CPNE7_MOUSE Copine-7 OS = Mus musculus GN = Cpne7 |
| | | | PE = 1 SV = 1 |
| 0.0675 | 0.0194 | Hint1 | P70349\|HINT1_MOUSE Histidine triad nucleotide-binding protein 1 |
| | | | OS = Mus musculus GN = Hint1 PE = 1 SV = 3 |
| 0.0681 | 0.0195 | Gpx1 | P11352\|GPX1_MOUSE Glutathione peroxidase 1 OS = Mus musculus |
| | | | GN = Gpx1 PE = 1 SV = 2 |
| 0.0682 | 0.0195 | Hoxa4 | P06798\|HXA4_MOUSE Homeobox protein Hox-A4 OS = Mus |
| | | | musculus GN = Hoxa4 PE = 2 SV = 4 |
| 0.0696 | 0.0198 | Lap3 | Q9CPY7\|AMPL_MOUSE Cytosol aminopeptidase OS = Mus musculus |
| | | | GN = Lap3 PE = 1 SV = 3 |
| 0.0697 | 0.0198 | Cyb5a | P56395\|CYB5_MOUSE Cytochrome b5 OS = Mus musculus |
| | | | GN = Cyb5a PE = 1 SV = 2 |
| 0.0701 | 0.0198 | Sephs2 | P97364\|SPS2_MOUSE Selenide, water dikinase 2 OS = Mus musculus |
| | | | GN = Sephs2 PE = 1 SV = 3 |
| 0.0701 | 0.0198 | Gsta3 | P30115\|GSTA3_MOUSE Glutathione S-transferase A3 OS = Mus |
| | | | musculus GN = Gsta3 PE = 1 SV = 2 |
| 0.0702 | 0.0198 | Col4a3 | Q9QZS0\|CO4A3_MOUSE Collagen alpha-3(IV) chain OS = Mus |
| | | | musculus GN = Col4a3 PE = 1 SV = 2 |
| 0.0712 | 0.0199 | Cyp2u1 | Q9CX98\|CP2U1_MOUSE Cytochrome P450 2U1 OS = Mus musculus |
| | | | GN = Cyp2u1 PE = 2 SV = 2 |
| 0.0715 | 0.0199 | Rrbp1 | Q99PL5\|RRBP1_MOUSE Ribosome-binding protein 1 OS = Mus |
| | | | musculus GN = Rrbp1 PE = 1 SV = 2 |
| 0.0716 | 0.0199 | Cpt2 | P52825\|CPT2_MOUSE Carnitine O-palmitoyltransferase 2, |
| | | | mitochondrial OS = Mus musculus GN = Cpt2 PE = 1 SV = 2 |
| 0.0716 | 0.0199 | Krt79 | Q8VED5\|K2C79_MOUSE Keratin, type II cytoskeletal 79 OS = Mus |
| | | | musculus GN = Krt79 PE = 1 SV = 2 |
| 0.0718 | 0.0199 | Txn | P10639\|THIO_MOUSE Thioredoxin OS = Mus musculus GN = Txn |
| | | | PE = 1 SV = 3 |
| 0.0720 | 0.0199 | Fabp1 | P12710\|FABPL_MOUSE Fatty acid-binding protein, liver OS = Mus |
| | | | musculus GN = Fabp1 PE = 1 SV = 2 |
| 0.0721 | 0.0199 | Idh2 | P54071\|IDHP_MOUSE Isocitrate dehydrogenase [NADP], |
| | | | mitochondrial OS = Mus musculus GN = Idh2 PE = 1 SV = 3 |
| 0.0723 | 0.0199 | Acadm | P45952\|ACADM_MOUSE Medium-chain specific acyl-CoA |
| | | | dehydrogenase, mitochondrial OS = Mus musculus GN = Acadm PE = 1 |
| | | | SV = 1 |
| 0.0724 | 0.0199 | Ces1 | Q8VCC2\|EST1_MOUSE Liver carboxylesterase 1 OS = Mus musculus |
| | | | GN = Ces1 PE = 1 SV = 1 |
| 0.0726 | 0.0199 | Akr1c13 | Q8VC28\|AK1CD_MOUSE Aldo-keto reductase family 1 member C13 |
| | | | OS = Mus musculus GN = Akr1c13 PE = 1 SV = 2 |
| 0.0726 | 0.0199 | Iigp1 | Q9QZ85\|IIGP1_MOUSE Interferon-inducible GTPase 1 OS = Mus |
| | | | musculus GN = Iigp1 PE = 1 SV = 2 |
| 0.0730 | 0.0199 | Lamb1 | P02469\|LAMB1_MOUSE Laminin subunit beta-1 OS = Mus musculus |
| | | | GN = Lamb1 PE = 1 SV = 3 |
| 0.0731 | 0.0199 | Lama4 | P97927\|LAMA4_MOUSE Laminin subunit alpha-4 OS = Mus musculus |
| | | | GN = Lama4 PE = 1 SV = 2 |
| 0.0732 | 0.0199 | Grhpr | Q91Z53\|GRHPR_MOUSE Glyoxylate reductase/hydroxypyruvate |
| | | | reductase OS = Mus musculus GN = Grhpr PE = 1 SV = 1 |
| 0.0735 | 0.0199 | Mpo | P11247\|PERM_MOUSE Myeloperoxidase OS = Mus musculus |
| | | | GN = Mpo PE = 1 SV = 2 |
| 0.0742 | 0.0200 | Cct4 | P80315\|TCPD_MOUSE T-complex protein 1 subunit delta OS = Mus |
| | | | musculus GN = Cct4 PE = 1 SV = 3 |
| 0.0745 | 0.0201 | 1 SV | Streptavidin\|P22629\|SAV_STRAV Streptavidin OS = Streptomyces |
| | | | avidinii PE = 1 SV = 1 |
| 0.0749 | 0.0201 | Lrpprc | Q6PB66\|LPPRC_MOUSE Leucine-rich PPR motif-containing protein, |
| | | | mitochondrial OS = Mus musculus GN = Lrpprc PE = 1 SV = 2 |
| 0.0752 | 0.0201 | Rpl4 | Q9D8E6\|RL4_MOUSE 60S ribosomal protein L4 OS = Mus musculus |
| | | | GN = Rpl4 PE = 1 SV = 3 |
| 0.0753 | 0.0201 | Fmo1 | P50285\|FMO1_MOUSE Dimethylaniline monooxygenase [N-oxide- |
| | | | forming] 1 OS = Mus musculus GN = Fmo1 PE = 1 SV = 1 |
| 0.0755 | 0.0201 | Marc2 | Q922Q1\|MARC2_MOUSE Mitochondrial amidoxime reducing |
| | | | component 2 OS = Mus musculus GN = Marc2 PE = 1 SV = 1 |
| 0.0762 | 0.0202 | Rpf1 | Q7TND5\|RPF1_MOUSE Ribosome production factor 1 OS = Mus |
| | | | musculus GN = Rpf1 PE = 2 SV = 2 |
| 0.0773 | 0.0205 | Pde8b | E9Q4S1\|PDE8B_MOUSE High affinity cAMP-specific and IBMX- |
| | | | insensitive 3′,5′-cyclic phosphodiesterase 8B OS = Mus musculus |
| | | | GN = Pde8b PE = 1 SV = 1 |
| 0.0781 | 0.0206 | Sec31a | Q3UPLO\|SC31A_MOUSE Protein transport protein Sec31A OS = Mus |
| | | | musculus GN = Sec31a PE = 1 SV = 2 |
| 0.0783 | 0.0206 | Cyp2d9 | P11714\|CP2D9_MOUSE Cytochrome P450 2D9 OS = Mus musculus |
| | | | GN = Cyp2d9 PE = 1 SV = 2 |
| 0.0785 | 0.0206 | Col5a2 | Q3U962\|CO5A2_MOUSE Collagen alpha-2(V) chain OS = Mus |
| | | | musculus GN = Col5a2 PE = 1 SV = 1 |
| 0.0788 | 0.0206 | Krt19 | P19001\|K1C19_MOUSE Keratin, type I cytoskeletal 19 OS = Mus |
| | | | musculus GN = Krt19 PE = 1 SV = 1 |
| 0.0789 | 0.0206 | Msn | P26041\|MOES_MOUSE Moesin OS = Mus musculus GN = Msn PE = 1 |
| | | | SV = 3 |
| 0.0793 | 0.0207 | Rdh7 | O88451\|RDH7_MOUSE Retinol dehydrogenase 7 OS = Mus musculus |
| | | | GN = Rdh7 PE = 1 SV = 1 |
| 0.0794 | 0.0207 | Cbr1 | P48758\|CBR1_MOUSE Carbonyl reductase [NADPH] 1 OS = Mus |
| | | | musculus GN = Cbr1 PE = 1 SV = 3 |
| 0.0804 | 0.0208 | Lama5 | Q61001\|LAMA5_MOUSE Laminin subunit alpha-5 OS = Mus musculus |
| | | | GN = Lama5 PE = 1 SV = 4 |
| 0.0808 | 0.0208 | Psmb3 | Q9R1P1\|PSB3_MOUSE Proteasome subunit beta type-3 OS = Mus |
| | | | musculus GN = Psmb3 PE = 1 SV = 1 |
| 0.0808 | 0.0208 | Prelp | Q9JK53\|PRELP_MOUSE Prolargin OS = Mus musculus GN = Prelp |
| | | | PE = 1 SV = 2 |
| 0.0809 | 0.0208 | D1Pas1 | P16381\|DDX3L_MOUSE Putative ATP-dependent RNA helicase PI10 |
| | | | OS = Mus musculus GN = D1Pas1 PE = 1 SV = 1 |
| 0.0810 | 0.0208 | Prdx2 | Q61171\|PRDX2_MOUSE Peroxiredoxin-2 OS = Mus musculus |
| | | | GN = Prdx2 PE = 1 SV = 3 |
| 0.0810 | 0.0208 | Ecm1 | Q61508\|ECM1_MOUSE Extracellular matrix protein 1 OS = Mus |
| | | | musculus GN = Ecm1 PE = 1 SV = 2 |
| 0.0814 | 0.0208 | Col7a1 | Q63870\|CO7A1_MOUSE Collagen alpha-1(VII) chain OS = Mus |
| | | | musculus GN = Col7a1 PE = 1 SV = 3 |
| 0.0815 | 0.0208 | Plg | P20918\|PLMN_MOUSE Plasminogen OS = Mus musculus GN = Plg |
| | | | PE = 1 SV = 3 |
| 0.0817 | 0.0208 | Rpl3 | P27659\|RL3_MOUSE 60S ribosomal protein L3 OS = Mus musculus |
| | | | GN = Rpl3 PE = 1 SV = 3 |
| 0.0828 | 0.0210 | Ncf2 | O70145\|NCF2_MOUSE Neutrophil cytosol factor 2 OS = Mus musculus |
| | | | GN = Ncf2 PE = 1 SV = 1 |
| 0.0831 | 0.0210 | Lamc1 | P02468\|LAMC1_MOUSE Laminin subunit gamma-1 OS = Mus |
| | | | musculus GN = Lamc1 PE = 1 SV = 2 |
| 0.0831 | 0.0210 | Pah | P16331\|PH4H_MOUSE Phenylalanine-4-hydroxylase OS = Mus |
| | | | musculus GN = Pah PE = 1 SV = 4 |
| 0.0839 | 0.0211 | Fbn2 | Q61555\|FBN2_MOUSE Fibrillin-2 OS = Mus musculus GN = Fbn2 PE = 1 |
| | | | SV = 2 |
| 0.0852 | 0.0213 | Lta4h | P24527\|LKHA4_MOUSE Leukotriene A-4 hydrolase OS = Mus |
| | | | musculus GN = Lta4h PE = 1 SV = 4 |
| 0.0853 | 0.0213 | Dhdh | Q9DBB8\|DHDH_MOUSE Trans-1,2-dihydrobenzene-1,2-diol |
| | | | dehydrogenase OS = Mus musculus GN = Dhdh PE = 1 SV = 1 |
| 0.0855 | 0.0213 | Col6a4 | A2AX52\|CO6A4_MOUSE Collagen alpha-4(VI) chain OS = Mus |
| | | | musculus GN = Col6a4 PE = 1 SV = 2 |
| 0.0863 | 0.0215 | Aass | Q99K67\|AASS_MOUSE Alpha-aminoadipic semialdehyde synthase, |
| | | | mitochondrial OS = Mus musculus GN = Aass PE = 1 SV = 1 |
| 0.0875 | 0.0217 | Lmna | P48678\|LMNA_MOUSE Prelamin-A/C OS = Mus musculus GN = Lmna |
| | | | PE = 1 SV = 2 |
| 0.0895 | 0.0221 | Acaa1b | Q8VCH0\|THIKB_MOUSE 3-ketoacyl-CoA thiolase B, peroxisomal |
| | | | OS = Mus musculus GN = Acaa1b PE = 1 SV = 1 |
| 0.0896 | 0.0221 | Ddx1 | Q91VR5\|DDX1_MOUSE ATP-dependent RNA helicase DDX1 |
| | | | OS = Mus musculus GN = Ddx1 PE = 1 SV = 1 |
| 0.0900 | 0.0221 | Dsp | E9Q557\|DESP_MOUSE Desmoplakin OS = Mus musculus GN = Dsp |
| | | | PE = 1 SV = 1 |
| 0.0900 | 0.0221 | Krt16 | Q9Z2K1\|K1C16_MOUSE Keratin, type I cytoskeletal 16 OS = Mus |
| | | | musculus GN = Krt16 PE = 1 SV = 3 |
| 0.0902 | 0.0221 | Rpl15 | Q9CZM2\|RL15_MOUSE 60S ribosomal protein L15 OS = Mus |
| | | | musculus GN = Rpl15 PE = 2 SV = 4 |
| 0.0903 | 0.0221 | Cmbl | Q8R1G2\|CMBL_MOUSE Carboxymethylenebutenolidase homolog |
| | | | OS = Mus musculus GN = Cmbl PE = 1 SV = 1 |
| 0.0911 | 0.0222 | Myl12b | Q3THE2\|ML12B_MOUSE Myosin regulatory light chain 12B OS = Mus |
| | | | musculus GN = Myl12b PE = 1 SV = 2 |
| 0.0923 | 0.0224 | Rab35 | Q6PHN9\|RAB35_MOUSE Ras-related protein Rab-35 OS = Mus |
| | | | musculus GN = Rab35 PE = 1 SV = 1 |
| 0.0931 | 0.0225 | Senp5 | Q6NXL6\|SENP5_MOUSE Sentrin-specific protease 5 OS = Mus |
| | | | musculus GN = Senp5 PE = 2 SV = 1 |
| 0.0947 | 0.0228 | Trim33 | Q99PP7\|TRI33_MOUSE E3 ubiquitin-protein ligase TRIM33 OS = Mus |
| | | | musculus GN = Trim33 PE = 1 SV = 2 |
| 0.0949 | 0.0228 | Mmrn2 | A6H6E2\|MMRN2_MOUSE Multimerin-2 OS = Mus musculus |
| | | | GN = Mmrn2 PE = 1 SV = 1 |
| 0.0950 | 0.0228 | Itih1 | Q61702\|ITIH1_MOUSE Inter-alpha-trypsin inhibitor heavy chain H1 |
| | | | OS = Mus musculus GN = Itih1 PE = 1 SV = 2 |
| 0.0951 | 0.0228 | Adk | P55264\|ADK_MOUSE Adenosine kinase OS = Mus musculus GN = Adk |
| | | | PE = 1 SV = 2 |
| 0.0953 | 0.0228 | Reg3g | O09049\|REG3G_MOUSE Regenerating islet-derived protein 3- |
| | | | gamma OS = Mus musculus GN = Reg3g PE = 1 SV = 1 |
| 0.0961 | 0.0229 | Cyp2c29 | Q64458\|CP2CT_MOUSE Cytochrome P450 2C29 OS = Mus musculus |
| | | | GN = Cyp2c29 PE = 1 SV = 2 |
| 0.0965 | 0.0229 | F13b | Q07968\|F13B_MOUSE Coagulation factor XIII B chain OS = Mus |
| | | | musculus GN = F13b PE = 1 SV = 2 |
| 0.0967 | 0.0229 | Acadl | P51174\|ACADL_MOUSE Long-chain specific acyl-CoA |
| | | | dehydrogenase, mitochondrial OS = Mus musculus GN = Acadl PE = 1 |
| | | | SV = 2 |
| 0.0977 | 0.0231 | Urgcp | Q5NCI0\|URGCP_MOUSE Up-regulator of cell proliferation OS = Mus |
| | | | musculus GN = Urgcp PE = 2 SV = 1 |
| 0.0978 | 0.0231 | Agmat | A2AS89\|SPEB_MOUSE Agmatinase, mitochondrial OS = Mus |
| | | | musculus GN = Agmat PE = 1 SV = 1 |
| 0.0996 | 0.0234 | Ugt2b17 | P17717\|UDB17_MOUSE UDP-glucuronosyltransferase 2B17 |
| | | | OS = Mus musculus GN = Ugt2b17 PE = 1 SV = 1 |
| 0.0998 | 0.0234 | Rps14 | P62264\|RS14_MOUSE 40S ribosomal protein S14 OS = Mus musculus |
| | | | GN = Rps14 PE = 1 SV = 3 |
| 0.1000 | 0.0234 | Stard9 | Q80TF6\|STAR9_MOUSE StAR-related lipid transfer protein 9 |
| | | | OS = Mus musculus GN = Stard9 PE = 1 SV = 2 |
| 0.1014 | 0.0237 | Lrfn1 | Q2WF71\|LRFN1_MOUSE Leucine-rich repeat and fibronectin type III |
| | | | domain-containing protein 1 OS = Mus musculus GN = Lrfn1 PE = 1 SV = 1 |
| 0.1015 | 0.0237 | Acaca | Q5SWU9\|ACACA_MOUSE Acetyl-CoA carboxylase 1 OS = Mus |
| | | | musculus GN = Acaca PE = 1 SV = 1 |
| 0.1019 | 0.0237 | Acad11 | Q80XL6\|ACD11_MOUSE Acyl-CoA dehydrogenase family member 11 |
| | | | OS = Mus musculus GN = Acad11 PE = 1 SV = 2 |
| 0.1026 | 0.0238 | Snd1 | Q78PY7\|SND1_MOUSE Staphylococcal nuclease domain-containing |
| | | | protein 1 OS = Mus musculus GN = Snd1 PE = 1 SV = 1 |
| 0.1027 | 0.0238 | Acaa1a | Q921H8\|THIKA_MOUSE 3-ketoacyl-CoA thiolase A, peroxisomal |
| | | | OS = Mus musculus GN = Acaa1a PE = 1 SV = 1 |
| 0.1057 | 0.0244 | Prodh | Q9WU79\|PROD_MOUSE Proline dehydrogenase 1, mitochondrial |
| | | | OS = Mus musculus GN = Prodh PE = 1 SV = 2 |
| 0.1065 | 0.0245 | Col6a5 | A6H584\|CO6A5_MOUSE Collagen alpha-5(VI) chain OS = Mus |
| | | | musculus GN = Col6a5 PE = 1 SV = 4 |
| 0.1088 | 0.0250 | Rpl22 | P67984\|RL22_MOUSE 60S ribosomal protein L22 OS = Mus musculus |
| | | | GN = Rpl22 PE = 1 SV = 2 |
| 0.1093 | 0.0250 | Cdkn2aip | Q8BI72\|CARF_MOUSE CDKN2A-interacting protein OS = Mus |
| | | | musculus GN = Cdkn2aip PE = 1 SV = 1 |
| 0.1095 | 0.0250 | Glyat | Q91XE0\|GLYAT_MOUSE Glycine N-acyltransferase OS = Mus |
| | | | musculus GN = Glyat PE = 1 SV = 1 |
| 0.1096 | 0.0250 | Uqcrc2 | Q9DB77\|QCR2_MOUSE Cytochrome b-c1 complex subunit 2, |
| | | | mitochondrial OS = Mus musculus GN = Uqcrc2 PE = 1 SV = 1 |
| 0.1100 | 0.0250 | Rplp1 | P47955\|RLA1_MOUSE 60S acidic ribosomal protein P1 OS = Mus |
| | | | musculus GN = Rplp1 PE = 1 SV = 1 |
| 0.1129 | 0.0256 | Lum | P51885\|LUM_MOUSE Lumican OS = Mus musculus GN = Lum PE = 1 |
| | | | SV = 2 |
| 0.1162 | 0.0262 | Dpf1 | Q9QX66\|DPF1_MOUSE Zinc finger protein neuro-d4 OS = Mus |
| | | | musculus GN = Dpf1 PE = 1 SV = 2 |
| 0.1173 | 0.0264 | Acads | Q07417\|ACADS_MOUSE Short-chain specific acyl-CoA |
| | | | dehydrogenase, mitochondrial OS = Mus musculus GN = Acads PE = 1 |
| | | | SV = 2 |
| 0.1176 | 0.0264 | Gata3 | P23772\|GATA3_MOUSE Trans-acting T-cell-specific transcription |
| | | | factor GATA-3 OS = Mus musculus GN = Gata3 PE = 1 SV = 1 |
| 0.1177 | 0.0264 | 1 SV | P01878\|IGHA_MOUSE Ig alpha chain C region OS = Mus musculus |
| | | | PE = 1 SV = 1 |
| 0.1185 | 0.0264 | Insrr | Q9WTL4\|INSRR_MOUSE Insulin receptor-related protein OS = Mus |
| | | | musculus GN = Insrr PE = 1 SV = 2 |
| 0.1186 | 0.0264 | Dyne1i1 | O88485\|DC1l1_MOUSE Cytoplasmic dynein 1 intermediate chain 1 |
| | | | OS = Mus musculus GN = Dync1i1 PE = 1 SV = 2 |
| 0.1199 | 0.0266 | Ccs | Q9WU84\|CCS_MOUSE Copper chaperone for superoxide dismutase |
| | | | OS = Mus musculus GN = Ccs PE = 1 SV = 1 |
| 0.1199 | 0.0266 | P4hb | P09103\|PDIA1_MOUSE Protein disulfide-isomerase OS = Mus |
| | | | musculus GN = P4hb PE = 1 SV = 2 |
| 0.1220 | 0.0270 | Cyb5r3 | Q9DCN2\|NB5R3_MOUSE NADH-cytochrome b5 reductase 3 |
| | | | OS = Mus musculus GN = Cyb5r3 PE = 1 SV = 3 |
| 0.1247 | 0.0275 | Dpyd | Q8CHR6\|DPYD_MOUSE Dihydropyrimidine dehydrogenase |
| | | | [NADP(+)] OS = Mus musculus GN = Dpyd PE = 1 SV = 1 |
| 0.1258 | 0.0277 | Maob | Q8BW75\|AOFB_MOUSE Amine oxidase [flavin-containing] B |
| | | | OS = Mus musculus GN = Maob PE = 1 SV = 4 |
| 0.1265 | 0.0278 | Rack1 | P68040\|RACK1_MOUSE Receptor of activated protein C kinase 1 |
| | | | OS = Mus musculus GN = Rack1 PE = 1 SV = 3 |
| 0.1273 | 0.0279 | Ppa1 | Q9D819\|IPYR_MOUSE Inorganic pyrophosphatase OS = Mus |
| | | | musculus GN = Ppa1 PE = 1 SV = 1 |
| 0.1290 | 0.0282 | Nkapl | Q5SZT7\|NKAPL_MOUSE NKAP-like protein OS = Mus musculus |
| | | | GN = Nkapl PE = 1 SV = 1 |
| 0.1293 | 0.0283 | Fah | P35505\|FAAA_MOUSE Fumarylacetoacetase OS = Mus musculus |
| | | | GN = Fah PE = 1 SV = 2 |
| 0.1298 | 0.0283 | Cdc42bpg | Q80UW5\|MRCKG_MOUSE Serine/threonine-protein kinase MRCK |
| | | | gamma OS = Mus musculus GN = Cdc42bpg PE = 1 SV = 2 |
| 0.1305 | 0.0283 | Rps16 | P14131\|RS16_MOUSE 40S ribosomal protein S16 OS = Mus musculus |
| | | | GN = Rps16 PE = 1 SV = 4 |
| 0.1306 | 0.0283 | Shmt1 | P50431\|GLYC_MOUSE Serine hydroxymethyltransferase, cytosolic |
| | | | OS = Mus musculus GN = Shmt1 PE = 1 SV = 3 |
| 0.1341 | 0.0291 | Dclk3 | Q8BWQ5\|DCLK3_MOUSE Serine/threonine-protein kinase DCLK3 |
| | | | OS = Mus musculus GN = Dclk3 PE = 2 SV = 2 |
| 0.1346 | 0.0291 | Nudt6 | Q8CH40\|NUDT6_MOUSE Nucleoside diphosphate-linked moiety X |
| | | | motif 6 OS = Mus musculus GN = Nudt6 PE = 1 SV = 1 |
| 0.1350 | 0.0291 | Cdc42 | P60766\|CDC42_MOUSE Cell division control protein 42 homolog |
| | | | OS = Mus musculus GN = Cdc42 PE = 1 SV = 2 |
| 0.1355 | 0.0292 | Hibadh | Q99L13\|3HIDH_MOUSE 3-hydroxyisobutyrate dehydrogenase, |
| | | | mitochondrial OS = Mus musculus GN = Hibadh PE = 1 SV = 1 |
| 0.1369 | 0.0294 | Rplp2 | P99027\|RLA2_MOUSE 60S acidic ribosomal protein P2 OS = Mus |
| | | | musculus GN = Rplp2 PE = 1 SV = 3 |
| 0.1382 | 0.0296 | Col4a1 | P02463\|CO4A1_MOUSE Collagen alpha-1(IV) chain OS = Mus |
| | | | musculus GN = Col4a1 PE = 1 SV = 4 |
| 0.1387 | 0.0297 | Serpina3m | Q03734\|SPA3M_MOUSE Serine protease inhibitor A3M OS = Mus |
| | | | musculus GN = Serpina3m PE = 1 SV = 2 |
| 0.1403 | 0.0299 | Saa2 | P05367\|SAA2_MOUSE Serum amyloid A-2 protein OS = Mus |
| | | | musculus GN = Saa2 PE = 1 SV = 1 |
| 0.1405 | 0.0299 | Mtch2 | Q791V5\|MTCH2_MOUSE Mitochondrial carrier homolog 2 OS = Mus |
| | | | musculus GN = Mtch2 PE = 1 SV = 1 |
| 0.1422 | 0.0302 | Pxdn | Q3UQ28\|PXDN_MOUSE Peroxidasin homolog OS = Mus musculus |
| | | | GN = Pxdn PE = 1 SV = 2 |
| 0.1436 | 0.0305 | Atp5b | P56480\|ATPB_MOUSE ATP synthase subunit beta, mitochondrial |
| | | | OS = Mus musculus GN = Atp5b PE = 1 SV = 2 |
| 0.1492 | 0.0316 | Batf3 | Q9D275\|BATF3_MOUSE Basic leucine zipper transcriptional factor |
| | | | ATF-like 3 OS = Mus musculus GN = Batf3 PE = 2 SV = 1 |
| 0.1515 | 0.0320 | Glul | P15105\|GLNA_MOUSE Glutamine synthetase OS = Mus musculus |
| | | | GN = Glul PE = 1 SV = 6 |
| 0.1570 | 0.0330 | Ugt1a1 | Q63886\|UD11_MOUSE UDP-glucuronosyltransferase 1-1 OS = Mus |
| | | | musculus GN = Ugt1a1 PE = 1 SV = 2 |
| 0.1600 | 0.0336 | Serpina1b | P22599\|A1AT2_MOUSE Alpha-1-antitrypsin 1-2 OS = Mus musculus |
| | | | GN = Serpina1b PE = 1 SV = 2 |
| 0.1618 | 0.0339 | Eef1d | P57776\|EF1D_MOUSE Elongation factor 1-delta OS = Mus musculus |
| | | | GN = Eef1d PE = 1 SV = 3 |
| 0.1630 | 0.0341 | Pgd | Q9DCDO\|6PGD_MOUSE 6-phosphogluconate dehydrogenase, |
| | | | decarboxylating OS = Mus musculus GN = Pgd PE = 1 SV = 3 |
| 0.1635 | 0.0341 | Ltbp4 | Q8K4G1\|LTBP4_MOUSE Latent-transforming growth factor beta- |
| | | | binding protein 4 OS = Mus musculus GN = Ltbp4 PE = 1 SV = 2 |
| 0.1653 | 0.0344 | Lgals9 | O08573\|LEG9_MOUSE Galectin-9 OS = Mus musculus GN = Lgals9 |
| | | | PE = 1 SV = 1 |
| 0.1668 | 0.0346 | Aldh3a2 | P47740\|AL3A2_MOUSE Fatty aldehyde dehydrogenase OS = Mus |
| | | | musculus GN = Aldh3a2 PE = 1 SV = 2 |
| 0.1669 | 0.0346 | Decr1 | Q9CQ62\|DECR_MOUSE 2,4-dienoyl-CoA reductase, mitochondrial |
| | | | OS = Mus musculus GN = Decr1 PE = 1 SV = 1 |
| 0.1678 | 0.0347 | Dbt | P53395\|ODB2_MOUSE Lipoamide acyltransferase component of |
| | | | branched-chain alpha-keto acid dehydrogenase complex, |
| | | | mitochondrial OS = Mus musculus GN = Dbt PE = 1 SV = 2 |
| 0.1697 | 0.0350 | Mccc1 | Q99MR8\|MCCA_MOUSE Methylcrotonoyl-CoA carboxylase subunit |
| | | | alpha, mitochondrial OS = Mus musculus GN = Mccc1 PE = 1 SV = 2 |
| 0.1703 | 0.0350 | Banf1 | O54962\|BAF_MOUSE Barrier-to-autointegration factor OS = Mus |
| | | | musculus GN = Banf1 PE = 1 SV = 1 |
| 0.1713 | 0.0352 | Serpina1e | Q00898\|A1AT5_MOUSE Alpha-1-antitrypsin 1-5 OS = Mus musculus |
| | | | GN = Serpina1e PE = 1 SV = 1 |
| 0.1727 | 0.0354 | Col3a1 | P08121\|CO3A1_MOUSE Collagen alpha-1(III) chain OS = Mus |
| | | | musculus GN = Col3a1 PE = 1 SV = 4 |
| 0.1755 | 0.0358 | Acta2 | P62737\|ACTA_MOUSE Actin, aortic smooth muscle OS = Mus |
| | | | musculus GN = Acta2 PE = 1 SV = 1 |
| 0.1796 | 0.0366 | Ywhab | Q9CQV8\|1433B_MOUSE 14-3-3 protein beta/alpha OS = Mus |
| | | | musculus GN = Ywhab PE = 1 SV = 3 |
| 0.1818 | 0.0369 | Slc25a20 | Q9Z2Z6\|MCAT_MOUSE Mitochondrial carnitine/acylcarnitine carrier |
| | | | protein OS = Mus musculus GN = Slc25a20 PE = 1 SV = 1 |
| 0.1834 | 0.0371 | Chd7 | A2AJK6\|CHD7_MOUSE Chromodomain-helicase-DNA-binding |
| | | | protein 7 OS = Mus musculus GN = Chd7 PE = 1 SV = 1 |
| 0.1856 | 0.0375 | Cyp2c40 | P56657\|CP240_MOUSE Cytochrome P450 2C40 OS = Mus musculus |
| | | | GN = Cyp2c40 PE = 1 SV = 2 |
| 0.1863 | 0.0376 | Rad23b | P54728\|RD23B_MOUSE UV excision repair protein RAD23 homolog |
| | | | B OS = Mus musculus GN = Rad23b PE = 1 SV = 2 |
| 0.1879 | 0.0378 | Serpinf2 | Q61247\|A2AP_MOUSE Alpha-2-antiplasmin OS = Mus musculus |
| | | | GN = Serpinf2 PE = 1 SV = 1 |
| 0.1919 | 0.0385 | Col15a1 | O35206\|COFA1_MOUSE Collagen alpha-1(XV) chain OS = Mus |
| | | | musculus GN = Col15a1 PE = 1 SV = 2 |
| 0.1941 | 0.0389 | Chat | Q03059\|CLAT_MOUSE Choline O-acetyltransferase OS = Mus |
| | | | musculus GN = Chat PE = 2 SV = 2 |
| 0.1961 | 0.0392 | F13a1 | Q8BH61\|F13A_MOUSE Coagulation factor XIII A chain OS = Mus |
| | | | musculus GN = F13a1 PE = 1 SV = 3 |
| 0.1991 | 0.0397 | Serpina1c | Q00896\|A1AT3_MOUSE Alpha-1-antitrypsin 1-3 OS = Mus musculus |
| | | | GN = Serpina1c PE = 1 SV = 2 |
| 0.2021 | 0.0402 | Cyb5b | Q9CQX2\|CYB5B_MOUSE Cytochrome b5 type B OS = Mus musculus |
| | | | GN = Cyb5b PE = 1 SV = 1 |
| 0.2026 | 0.0402 | Tfap2d | Q91ZK0\|AP2D_MOUSE Transcription factor AP-2-delta OS = Mus |
| | | | musculus GN = Tfap2d PE = 1 SV = 1 |
| 0.2028 | 0.0402 | Fbn1 | Q61554\|FBN1_MOUSE Fibrillin-1 OS = Mus musculus GN = Fbn1 PE = 1 |
| | | | SV = 2 |
| 0.2037 | 0.0403 | Nedd4 | P46935\|NEDD4_MOUSE E3 ubiquitin-protein ligase NEDD4 OS = Mus |
| | | | musculus GN = Nedd4 PE = 1 SV = 3 |
| 0.2052 | 0.0405 | Rai1 | Q61818\|RAI1_MOUSE Retinoic acid-induced protein 1 OS = Mus |
| | | | musculus GN = Rai1 PE = 1 SV = 3 |
| 0.2054 | 0.0405 | Pabpc1 | P29341\|PABP1_MOUSE Polyadenylate-binding protein 1 OS = Mus |
| | | | musculus GN = Pabpc1 PE = 1 SV = 2 |
| 0.2092 | 0.0412 | Mat1a | Q91X83\|METK1_MOUSE S-adenosylmethionine synthase isoform |
| | | | type-1 OS = Mus musculus GN = Mat1a PE = 1 SV = 1 |
| 0.2104 | 0.0414 | Col11a1 | Q61245\|COBA1_MOUSE Collagen alpha-1(XI) chain OS = Mus |
| | | | musculus GN = Col11a1 PE = 1 SV = 2 |
| 0.2133 | 0.0418 | | Cas9\|IentiCRISPR Nuclease from IentiCRISPR v2 (1aa - 1384aa) |
| | | | 1384aa |
| 0.2179 | 0.0426 | Dgcr8 | Q9EQM6\|DGCR8_MOUSE Microprocessor complex subunit DGCR8 |
| | | | OS = Mus musculus GN = Dgcr8 PE = 1 SV = 2 |
| 0.2183 | 0.0426 | Ddt | O35215\|DOPD_MOUSE D-dopachrome decarboxylase OS = Mus |
| | | | musculus GN = Ddt PE = 1 SV = 3 |
| 0.2186 | 0.0426 | Acacb | E9Q4Z2\|ACACB_MOUSE Acetyl-CoA carboxylase 2 OS = Mus |
| | | | musculus GN = Acacb PE = 1 SV = 1 |
| 0.2205 | 0.0429 | Rpl9 | P51410\|RL9_MOUSE 60S ribosomal protein L9 OS = Mus musculus |
| | | | GN = Rpl9 PE = 2 SV = 2 |
| 0.2215 | 0.0430 | Ptbp1 | P17225\|PTBP1_MOUSE Polypyrimidine tract-binding protein 1 |
| | | | OS = Mus musculus GN = Ptbp1 PE = 1 SV = 2 |
| 0.2226 | 0.0431 | Dmbt1 | Q60997\|DMBT1_MOUSE Deleted in malignant brain tumors 1 protein |
| | | | OS = Mus musculus GN = Dmbt1 PE = 1 SV = 2 |
| 0.2227 | 0.0431 | Krt7 | Q9DCV7\|K2C7_MOUSE Keratin, type II cytoskeletal 7 OS = Mus |
| | | | musculus GN = Krt7 PE = 1 SV = 1 |
| 0.2255 | 0.0436 | Col2a1 | P28481\|CO2A1_MOUSE Collagen alpha-1(II) chain OS = Mus |
| | | | musculus GN = Col2a1 PE = 1 SV = 2 |
| 0.2300 | 0.0443 | Col6a1 | Q04857\|CO6A1_MOUSE Collagen alpha-1(VI) chain OS = Mus |
| | | | musculus GN = Col6a1 PE = 1 SV = 1 |
| 0.2355 | 0.0452 | Megf6 | Q80V70\|MEGF6_MOUSE Multiple epidermal growth factor-like |
| | | | domains protein 6 OS = Mus musculus GN = Megf6 PE = 2 SV = 3 |
| 0.2421 | 0.0463 | Eef1g | Q9D8N0\|EF1G_MOUSE Elongation factor 1-gamma OS = Mus |
| | | | musculus GN = Eef1g PE = 1 SV = 3 |
| 0.2494 | 0.0476 | Tf | Q921I1\|TRFE_MOUSE Serotransferrin OS = Mus musculus GN = Tf |
| | | | PE = 1 SV = 1 |
| 0.2569 | 0.0490 | Col4a4 | Q9QZR9\|CO4A4_MOUSE Collagen alpha-4(IV) chain OS = Mus |
| | | | musculus GN = Col4a4 PE = 2 SV = 1 |
| 0.2583 | 0.0492 | Cox5b | P19536\|COX5B_MOUSE Cytochrome c oxidase subunit 5B, |
| | | | mitochondrial OS = Mus musculus GN = Cox5b PE = 1 SV = 1 |
| 0.2608 | 0.0495 | Tgfbi | P82198\|BGH3_MOUSE Transforming growth factor-beta-induced |
| | | | protein ig-h3 OS = Mus musculus GN = Tgfbi PE = 1 SV = 1 |
| 0.2610 | 0.0495 | C1qbp | O35658\|C1QBP_MOUSE Complement component 1 Q |
| | | | subcomponent-binding protein, mitochondrial OS = Mus musculus |
| | | | GN = C1qbp PE = 1 SV = 1 |
| 0.2862 | 0.0541 | S100a11 | P50543\|S10AB_MOUSE Protein S100-A11 OS = Mus musculus |
| | | | GN = S100a11 PE = 1 SV = 1 |
| 0.2883 | 0.0544 | Rps12 | P63323\|RS12_MOUSE 40S ribosomal protein S12 OS = Mus musculus |
| | | | GN = Rps12 PE = 1 SV = 2 |
| 0.2905 | 0.0547 | Gys2 | Q8VCB3\|GYS2_MOUSE Glycogen [starch] synthase, liver OS = Mus |
| | | | musculus GN = Gys2 PE = 1 SV = 2 |
| 0.2911 | 0.0547 | Rnf43 | Q5NCP0\|RNF43_MOUSE E3 ubiquitin-protein ligase RNF43 OS = Mus |
| | | | musculus GN = Rnf43 PE = 2 SV = 1 |
| 0.2969 | 0.0557 | Fam208b | Q5DTT3\|F208B_MOUSE Protein FAM208B OS = Mus musculus |
| | | | GN = Fam208b PE = 1 SV = 2 |
| 0.3022 | 0.0566 | Flg2 | Q2VIS4\|FILA2_MOUSE Filaggrin-2 OS = Mus musculus GN = Flg2 |
| | | | PE = 1 SV = 2 |
| 0.3030 | 0.0566 | Apoa1 | Q00623\|APOA1_MOUSE Apolipoprotein A-I OS = Mus musculus |
| | | | GN = Apoa1 PE = 1 SV = 2 |
| 0.3045 | 0.0568 | Pcbp1 | P60335\|PCBP1_MOUSE Poly(rC)-binding protein 1 OS = Mus |
| | | | musculus GN = Pcbp1 PE = 1 SV = 1 |
| 0.3075 | 0.0572 | Tnc | Q80YX1\|TENA_MOUSE Tenascin OS = Mus musculus GN = Tnc PE = 1 |
| | | | SV = 1 |
| 0.3112 | 0.0578 | Fbln5 | Q9WVH9\|FBLN5_MOUSE Fibulin-5 OS = Mus musculus GN = Fbln5 |
| | | | PE = 1 SV = 1 |
| 0.3181 | 0.0590 | Rabepk | Q8VCH5\|RABEK_MOUSE Rab9 effector protein with kelch motifs |
| | | | OS = Mus musculus GN = Rabepk PE = 1 SV = 2 |
| 0.3196 | 0.0592 | Ambp | Q07456\|AMBP_MOUSE Protein AMBP OS = Mus musculus GN = Ambp |
| | | | PE = 1 SV = 2 |
| 0.3229 | 0.0596 | Hpx | Q91X72\|HEMO_MOUSE Hemopexin OS = Mus musculus GN = Hpx |
| | | | PE = 1 SV = 2 |
| 0.3231 | 0.0596 | Upk1b | Q9Z2C6\|UPK1B_MOUSE Uroplakin-1b OS = Mus musculus |
| | | | GN = Upk1b PE = 2 SV = 3 |
| 0.3238 | 0.0596 | Vwf | Q8CIZ8\|VWF_MOUSE von Willebrand factor OS = Mus musculus |
| | | | GN = Vwf PE = 1 SV = 2 |
| 0.3269 | 0.0601 | Mlk4 | Q8VDG6\|M3KL4_MOUSE Mitogen-activated protein kinase kinase |
| | | | kinase MLK4 OS = Mus musculus GN = Mlk4 PE = 1 SV = 2 |
| 0.3294 | 0.0604 | Fh | P97807\|FUMH_MOUSE Fumarate hydratase, mitochondrial OS = Mus |
| | | | musculus GN = Fh PE = 1 SV = 3 |
| 0.3315 | 0.0607 | Pnma1 | Q8C1C8\|PNMA1_MOUSE Paraneoplastic antigen Ma1 homolog |
| | | | OS = Mus musculus GN = Pnma1 PE = 1 SV = 2 |
| 0.3325 | 0.0608 | Eif4g1 | Q6NZJ6\|IF4G1_MOUSE Eukaryotic translation initiation factor 4 |
| | | | gamma 1 OS = Mus musculus GN = Eif4g1 PE = 1 SV = 1 |
| 0.3372 | 0.0615 | S100a8 | P27005\|S10A8_MOUSE Protein S100-A8 OS = Mus musculus |
| | | | GN = S100a8 PE = 1 SV = 3 |
| 0.3424 | 0.0624 | Flna | Q8BTM8\|FLNA_MOUSE Filamin-A OS = Mus musculus GN = Flna |
| | | | PE = 1 SV = 5 |
| 0.3443 | 0.0626 | Adipoq | Q60994\|ADIPO_MOUSE Adiponectin OS = Mus musculus GN = Adipoq |
| | | | PE = 1 SV = 2 |
| 0.3450 | 0.0626 | Eif4h | Q9WUK2\|IF4H_MOUSE Eukaryotic translation initiation factor 4H |
| | | | OS = Mus musculus GN = Eif4h PE = 1 SV = 3 |
| 0.3463 | 0.0628 | Loxl1 | P97873\|LOXL1_MOUSE Lysyl oxidase homolog 1 OS = Mus musculus |
| | | | GN = Loxl1 PE = 2 SV = 3 |
| 0.3575 | 0.0647 | Col18a1 | P39061\|COIA1_MOUSE Collagen alpha-1(XVIII) chain OS = Mus |
| | | | musculus GN = Col18a1 PE = 1 SV = 4 |
| 0.3641 | 0.0657 | Hal | P35492\|HUTH_MOUSE Histidine ammonia-lyase OS = Mus musculus |
| | | | GN = Hal PE = 1 SV = 1 |
| 0.3648 | 0.0658 | Ltbp1 | Q8CG19\|LTBP1_MOUSE Latent-transforming growth factor beta- |
| | | | binding protein 1 OS = Mus musculus GN = Ltbp1 PE = 1 SV = 2 |
| 0.3681 | 0.0662 | Anks1b | Q8BIZ1\|ANS1B_MOUSE Ankyrin repeat and sterile alpha motif |
| | | | domain-containing protein 1B OS = Mus musculus GN = Anks1b PE = 1 |
| | | | SV = 3 |
| 0.3713 | 0.0667 | Kcnk4 | O88454\|KCNK4_MOUSE Potassium channel subfamily K member 4 |
| | | | OS = Mus musculus GN = Kcnk4 PE = 2 SV = 1 |
| 0.3767 | 0.0675 | Mup2 | P11589\|MUP2_MOUSE Major urinary protein 2 OS = Mus musculus |
| | | | GN = Mup2 PE = 1 SV = 1 |
| 0.3889 | 0.0696 | Ces1d | Q8VCT4\|CES1D_MOUSE Carboxylesterase 1D OS = Mus musculus |
| | | | GN = Ces1d PE = 1 SV = 1 |
| 0.3985 | 0.0711 | Bcl9 | Q9D219\|BCL9_MOUSE B-cell CLL/lymphoma 9 protein OS = Mus |
| | | | musculus GN = Bcl9 PE = 1 SV = 3 |
| 0.4013 | 0.0715 | Col1a2 | Q01149\|CO1A2_MOUSE Collagen alpha-2(I) chain OS = Mus |
| | | | musculus GN = Col1a2 PE = 1 SV = 2 |
| 0.4047 | 0.0719 | Fam65c | A1L3T7\|FA65C_MOUSE Protein FAM65C OS = Mus musculus |
| | | | GN = Fam65c PE = 1 SV = 1 |
| 0.4086 | 0.0725 | Aspdh | Q9DCQ2\|ASPD_MOUSE Putative L-aspartate dehydrogenase |
| | | | OS = Mus musculus GN = Aspdh PE = 1 SV = 1 |
| 0.4171 | 0.0739 | C4b | P01029\|CO4B_MOUSE Complement C4-B OS = Mus musculus |
| | | | GN = C4b PE = 1 SV = 3 |
| 0.4244 | 0.0749 | Slc25a5 | P51881\|ADT2_MOUSE ADP/ATP translocase 2 OS = Mus musculus |
| | | | GN = Slc25a5 PE = 1 SV = 3 |
| 0.4253 | 0.0749 | Calml3 | Q9D6P8\|CALL3_MOUSE Calmodulin-like protein 3 OS = Mus |
| | | | musculus GN = Calml3 PE = 2 SV = 1 |
| 0.4253 | 0.0749 | Dpysl2 | O08553\|DPYL2_MOUSE Dihydropyrimidinase-related protein 2 |
| | | | OS = Mus musculus GN = Dpysl2 PE = 1 SV = 2 |
| 0.4290 | 0.0755 | Krt35 | Q497I4\|KRT35_MOUSE Keratin, type I cuticular Ha5 OS = Mus |
| | | | musculus GN = Krt35 PE = 1 SV = 1 |
| 0.4315 | 0.0758 | 1 SV | P01837\|IGKC_MOUSE Ig kappa chain C region OS = Mus musculus |
| | | | PE = 1 SV = 1 |
| 0.4524 | 0.0793 | Sod2 | P09671\|SODM_MOUSE Superoxide dismutase [Mn], mitochondrial |
| | | | OS = Mus musculus GN = Sod2 PE = 1 SV = 3 |
| 0.4540 | 0.0794 | Sall2 | Q9QX96\|SALL2_MOUSE Sal-like protein 2 OS = Mus musculus |
| | | | GN = Sall2 PE = 1 SV = 2 |
| 0.4623 | 0.0808 | Myo16 | Q5DU14\|MYO16_MOUSE Unconventional myosin-XVI OS = Mus |
| | | | musculus GN = Myo16 PE = 1 SV = 2 |
| 0.4645 | 0.0810 | Anxa7 | Q07076\|ANXA7_MOUSE Annexin A7 OS = Mus musculus GN = Anxa7 |
| | | | PE = 1 SV = 2 |
| 0.4693 | 0.0817 | Pggt1b | Q8BUY9\|PGTB1_MOUSE Geranylgeranyl transferase type-1 subunit |
| | | | beta OS = Mus musculus GN = Pggt1b PE = 1 SV = 1 |
| 0.4811 | 0.0836 | Zc3h12a | Q5D1E7\|ZC12A_MOUSE Endoribonuclease ZC3H12A OS = Mus |
| | | | musculus GN = Zc3h12a PE = 1 SV = 2 |
| 0.4867 | 0.0844 | Vdac2 | Q60930\|VDAC2_MOUSE Voltage-dependent anion-selective channel |
| | | | protein 2 OS = Mus musculus GN = Vdac2 PE = 1 SV = 2 |
| 0.4912 | 0.0851 | Atn1 | O35126\|ATN1_MOUSE Atrophin-1 OS = Mus musculus GN = Atn1 |
| | | | PE = 1 SV = 1 |
| 0.4994 | 0.0863 | Myh11 | O08638\|MYH11_MOUSE Myosin-11 OS = Mus musculus GN = Myh11 |
| | | | PE = 1 SV = 1 |
| 0.4998 | 0.0863 | Il15ra | Q60819\|I15RA_MOUSE Interleukin-15 receptor subunit alpha |
| | | | OS = Mus musculus GN = Il15ra PE = 1 SV = 1 |
| 0.5011 | 0.0864 | Msra | Q9D6Y7\|MSRA_MOUSE Mitochondrial peptide methionine sulfoxide |
| | | | reductase OS = Mus musculus GN = Msra PE = 1 SV = 1 |
| 0.5048 | 0.0867 | Hsd11b1 | P50172\|DHI1_MOUSE Corticosteroid 11-beta-dehydrogenase |
| | | | isozyme 1 OS = Mus musculus GN = Hsd11b1 PE = 1 SV = 3 |
| 0.5144 | 0.0881 | Hdgf | P51859\|HDGF_MOUSE Hepatoma-derived growth factor OS = Mus |
| | | | musculus GN = Hdgf PE = 1 SV = 2 |
| 0.5299 | 0.0906 | LRWD1 | Q8BUI3\|LRWD1_MOUSE Leucine-rich repeat and WD repeat- |
| | | | containing protein 1 OS = Mus musculus GN = LRWD1 PE = 2 SV = 1 |
| 0.5338 | 0.0911 | Pcca | Q91ZA3\|PCCA_MOUSE Propionyl-CoA carboxylase alpha chain, |
| | | | mitochondrial OS = Mus musculus GN = Pcca PE = 1 SV = 2 |
| 0.5373 | 0.0915 | Krt71 | Q9R0H5\|K2C71_MOUSE Keratin, type II cytoskeletal 71 OS = Mus |
| | | | musculus GN = Krt71 PE = 1 SV = 1 |
| 0.5559 | 0.0945 | Dpt | Q9QZZ6\|DERM_MOUSE Dermatopontin OS = Mus musculus GN = Dpt |
| | | | PE = 1 SV = 1 |
| 0.5667 | 0.0959 | Fgb | Q8K0E8\|FIBB_MOUSE Fibrinogen beta chain OS = Mus musculus |
| | | | GN = Fgb PE = 1 SV = 1 |
| 0.5667 | 0.0959 | Apoa4 | P06728\|APOA4_MOUSE Apolipoprotein A-IV OS = Mus musculus |
| | | | GN = Apoa4 PE = 1 SV = 3 |
| 0.5815 | 0.0982 | Sfswap | Q3USH5\|SFSWA_MOUSE Splicing factor, suppressor of white-apricot |
| | | | homolog OS = Mus musculus GN = Sfswap PE = 1 SV = 2 |
| 0.5871 | 0.0989 | Efl1 | Q8C0D5\|EFL1_MOUSE Elongation factor-like GTPase 1 OS = Mus |
| | | | musculus GN = Efl1 PE = 1 SV = 1 |
| 0.5874 | 0.0989 | Itih4 | A6X935\|ITIH4_MOUSE Inter alpha-trypsin inhibitor, heavy chain 4 |
| | | | OS = Mus musculus GN = Itih4 PE = 1 SV = 2 |
| 0.5882 | 0.0989 | A1bg | Q19LI2\|A1BG_MOUSE Alpha-1B-glycoprotein OS = Mus musculus |
| | | | GN = A1bg PE = 1 SV = 1 |
| 0.5905 | 0.0990 | Tnrc6c | Q3UHC0\|TNR6C_MOUSE Trinucleotide repeat-containing gene 6C |
| | | | protein OS = Mus musculus GN = Tnrc6c PE = 1 SV = 2 |
| 0.5910 | 0.0990 | Lasp1 | Q61792\|LASP1_MOUSE LIM and SH3 domain protein 1 OS = Mus |
| | | | musculus GN = Lasp1 PE = 1 SV = 1 |
| 0.5970 | 0.0998 | Pdlim1 | O70400\|PDLI1_MOUSE PDZ and LIM domain protein 1 OS = Mus |
| | | | musculus GN = Pdlim1 PE = 1 SV = 4 |
| 0.6092 | 0.1017 | Zfhx3 | Q61329\|ZFHX3_MOUSE Zinc finger homeobox protein 3 OS = Mus |
| | | | musculus GN = Zfhx3 PE = 1 SV = 1 |
| 0.6108 | 0.1018 | Mgst1 | Q91VS7\|MGST1_MOUSE Microsomal glutathione S-transferase 1 |
| | | | OS = Mus musculus GN = Mgst1 PE = 1 SV = 3 |
| 0.6231 | 0.1037 | Col8a1 | Q00780\|CO8A1_MOUSE Collagen alpha-1(VIII) chain OS = Mus |
| | | | musculus GN = Col8a1 PE = 1 SV = 3 |
| 0.6317 | 0.1050 | Pc | Q05920\|PYC_MOUSE Pyruvate carboxylase, mitochondrial OS = Mus |
| | | | musculus GN = Pc PE = 1 SV = 1 |
| 0.6485 | 0.1076 | Prg2 | Q61878\|PRG2_MOUSE Bone marrow proteoglycan OS = Mus |
| | | | musculus GN = Prg2 PE = 1 SV = 1 |
| 0.6666 | 0.1104 | Ahsg | P29699\|FETUA_MOUSE Alpha-2-HS-glycoprotein OS = Mus musculus |
| | | | GN = Ahsg PE = 1 SV = 1 |
| 0.6700 | 0.1108 | Myh4 | Q5SX39\|MYH4_MOUSE Myosin-4 OS = Mus musculus GN = Myh4 |
| | | | PE = 2 SV = 1 |
| 0.6722 | 0.1110 | Sptan1 | P16546\|SPTN1_MOUSE Spectrin alpha chain, non-erythrocytic 1 |
| | | | OS = Mus musculus GN = Sptan1 PE = 1 SV = 4 |
| 0.6746 | 0.1112 | Serpina3k | P07759\|SPA3K_MOUSE Serine protease inhibitor A3K OS = Mus |
| | | | musculus GN = Serpina3k PE = 1 SV = 2 |
| 0.6786 | 0.1117 | Hsd17b10 | O08756\|HCD2_MOUSE 3-hydroxyacyl-CoA dehydrogenase type-2 |
| | | | OS = Mus musculus GN = Hsd17b10 PE = 1 SV = 4 |
| 0.6859 | 0.1127 | Krt4 | P07744\|K2C4_MOUSE Keratin, type II cytoskeletal 4 OS = Mus |
| | | | musculus GN = Krt4 PE = 1 SV = 2 |
| 0.6963 | 0.1142 | Postn | Q62009\|POSTN_MOUSE Periostin OS = Mus musculus GN = Postn |
| | | | PE = 1 SV = 2 |
| 0.7036 | 0.1152 | Hrg | Q9ESB3\|HRG_MOUSE Histidine-rich glycoprotein OS = Mus musculus |
| | | | GN = Hrg PE = 1 SV = 2 |
| 0.7056 | 0.1154 | Atp8a2 | P98200\|AT8A2_MOUSE Phospholipid-transporting ATPase IB |
| | | | OS = Mus musculus GN = Atp8a2 PE = 1 SV = 1 |
| 0.7099 | 0.1157 | Bgn | P28653\|PGS1_MOUSE Biglycan OS = Mus musculus GN = Bgn PE = 1 |
| | | | SV = 1 |
| 0.7101 | 0.1157 | Col14a1 | Q80X19\|COEA1_MOUSE Collagen alpha-1(XIV) chain OS = Mus |
| | | | musculus GN = Col14a1 PE = 1 SV = 2 |
| 0.7167 | 0.1166 | Polr2d | Q9D7M8\|RPB4_MOUSE DNA-directed RNA polymerase II subunit |
| | | | RPB4 OS = Mus musculus GN = Polr2d PE = 1 SV = 2 |
| 0.7202 | 0.1170 | Col1a1 | P11087\|CO1A1_MOUSE Collagen alpha-1(I) chain OS = Mus |
| | | | musculus GN = Col1a1 PE = 1 SV = 4 |
| 0.7307 | 0.1185 | Anxa1 | P10107\|ANXA1_MOUSE Annexin A1 OS = Mus musculus GN = Anxa1 |
| | | | PE = 1 SV = 2 |
| 0.7421 | 0.1200 | Ltk | P08923\|LTK_MOUSE Leukocyte tyrosine kinase receptor OS = Mus |
| | | | musculus GN = Ltk PE = 1 SV = 3 |
| 0.7509 | 0.1210 | Col16a1 | Q8BLX7\|COGA1_MOUSE Collagen alpha-1(XVI) chain OS = Mus |
| | | | musculus GN = Col16a1 PE = 1 SV = 2 |
| 0.7605 | 0.1224 | Ttn | A2ASS6\|TITIN_MOUSE Titin OS = Mus musculus GN = Ttn PE = 1 SV = 1 |
| 0.7755 | 0.1246 | Arg1 | Q61176\|ARGI1_MOUSE Arginase-1 OS = Mus musculus GN = Arg1 |
| | | | PE = 1 SV = 1 |
| 0.7919 | 0.1270 | Dcn | P28654\|PGS2_MOUSE Decorin OS = Mus musculus GN = Dcn PE = 1 |
| | | | SV = 1 |
| 0.8077 | 0.1294 | Col5a1 | O88207\|CO5A1_MOUSE Collagen alpha-1(V) chain OS = Mus |
| | | | musculus GN = Col5a1 PE = 1 SV = 2 |
| 0.8168 | 0.1306 | Krt6a | P50446\|K2C6A_MOUSE Keratin, type II cytoskeletal 6A OS = Mus |
| | | | musculus GN = Krt6a PE = 1 SV = 3 |
| 0.8216 | 0.1311 | Fga | E9PV24\|FIBA_MOUSE Fibrinogen alpha chain OS = Mus musculus |
| | | | GN = Fga PE = 1 SV = 1 |
| 0.8226 | 0.1311 | Efemp1 | Q8BPB5\|FBLN3_MOUSE EGF-containing fibulin-like extracellular |
| | | | matrix protein 1 OS = Mus musculus GN = Efemp1 PE = 1 SV = 1 |
| 0.8345 | 0.1325 | Acsl5 | Q8JZR0\|ACSL5_MOUSE Long-chain-fatty-acid--CoA ligase 5 |
| | | | OS = Mus musculus GN = Acsl5 PE = 1 SV = 1 |
| 0.8350 | 0.1325 | Col12a1 | Q60847\|COCA1_MOUSE Collagen alpha-1(XII) chain OS = Mus |
| | | | musculus GN = Col12a1 PE = 2 SV = 3 |
| 0.8351 | 0.1325 | Ces2a | Q8QZR3\|EST2A_MOUSE Pyrethroid hydrolase Ces2a OS = Mus |
| | | | musculus GN = Ces2a PE = 1 SV = 1 |
| 0.8569 | 0.1357 | Arhgap5 | P97393\|RHG05_MOUSE Rho GTPase-activating protein 5 OS = Mus |
| | | | musculus GN = Arhgap5 PE = 1 SV = 2 |
| 0.8658 | 0.1369 | Hspa5 | P20029\|GRP78_MOUSE 78 kDa glucose-regulated protein OS = Mus |
| | | | musculus GN = Hspa5 PE = 1 SV = 3 |
| 0.8702 | 0.1374 | Urah | Q9CRB3\|HIUH_MOUSE 5-hydroxyisourate hydrolase OS = Mus |
| | | | musculus GN = Urah PE = 1 SV = 1 |
| 0.8905 | 0.1404 | S100a9 | P31725\|S10A9_MOUSE Protein S100-A9 OS = Mus musculus |
| | | | GN = S100a9 PE = 1 SV = 3 |
| 0.8966 | 0.1410 | Cycs | P62897\|CYC_MOUSE Cytochrome c, somatic OS = Mus musculus |
| | | | GN = Cycs PE = 1 SV = 2 |
| 0.8970 | 0.1410 | Ptms | Q9D0J8\|PTMS_MOUSE Parathymosin OS = Mus musculus GN = Ptms |
| | | | PE = 1 SV = 3 |
| 0.9042 | 0.1419 | Rpl27a | P14115\|RL27A_MOUSE 60S ribosomal protein L27a OS = Mus |
| | | | musculus GN = Rpl27a PE = 1 SV = 5 |
| 0.9286 | 0.1453 | Aqp1 | Q02013\|AQP1_MOUSE Aquaporin-1 OS = Mus musculus GN = Aqp1 |
| | | | PE = 1 SV = 3 |
| 0.9345 | 0.1460 | Elk4 | P41158\|ELK4_MOUSE ETS domain-containing protein Elk-4 OS = Mus |
| | | | musculus GN = Elk4 PE = 2 SV = 2 |
| 0.9446 | 0.1474 | Pdia3 | P27773\|PDIA3_MOUSE Protein disulfide-isomerase A3 OS = Mus |
| | | | musculus GN = Pdia3 PE = 1 SV = 2 |
| 0.9674 | 0.1506 | Fgg | Q8VCM7\|FIBG_MOUSE Fibrinogen gamma chain OS = Mus musculus |
| | | | GN = Fgg PE = 1 SV = 1 |
| 0.9683 | 0.1506 | Ftl1 | P29391\|FRIL1_MOUSE Ferritin light chain 1 OS = Mus musculus |
| | | | GN = Ftl1 PE = 1 SV = 2 |
| 0.9950 | 0.1544 | Isoc1 | Q91V64\|ISOC1_MOUSE Isochorismatase domain-containing protein |
| | | | 1 OS = Mus musculus GN = Isoc1 PE = 1 SV = 1 |
| 0.9956 | 0.1544 | Fus | P56959\|FUS_MOUSE RNA-binding protein FUS OS = Mus musculus |
| | | | GN = Fus PE = 1 SV = 1 |
| 0.9997 | 0.1548 | C3 | P01027\|CO3_MOUSE Complement C3 OS = Mus musculus GN = C3 |
| | | | PE = 1 SV = 3 |

TABLE 3
Identified proteins in peritoneal samples

| Anova | q | Gene | |
|---|---|---|---|
| (P) | Value | symbol | Uniprot Assecion/Description |
| 0.0015 | 0.5350 | Lamc1 | P02468\|LAMC1 MOUSE Laminin subunit gamma-1 OS = Mus musculus |
| | | | GN = Lamc1 PE = 1 SV = 2 |
| 0.0031 | 0.5384 | Nid1 | P10493\|NID1_MOUSE Nidogen-1 OS = Mus musculus GN = Nid1 PE = 1 |
| | | | SV = 2 |
| 0.0075 | 0.6735 | Lama2 | Q60675\|LAMA2_MOUSE Laminin subunit alpha-2 OS = Mus musculus |
| | | | GN = Lama2 PE = 1 SV = 2 |
| 0.0089 | 0.6735 | Syne2 | Q6ZWQ0\|SYNE2_MOUSE Nesprin-2 OS = Mus musculus GN = Syne2 PE = 1 |
| | | | SV = 2 |
| 0.0146 | 0.6735 | Tpm2 | P58774\|TPM2_MOUSE Tropomyosin beta chain OS = Mus musculus |
| | | | GN = Tpm2 PE = 1 SV = 1 |
| 0.0152 | 0.6735 | Col6a2 | Q02788\|CO6A2_MOUSE Collagen alpha-2(VI) chain OS = Mus musculus |
| | | | GN = Col6a2 PE = 1 SV = 3 |
| 0.0158 | 0.6735 | Lamb2 | Q61292\|LAMB2_MOUSE Laminin subunit beta-2 OS = Mus musculus |
| | | | GN = Lamb2 PE = 1 SV = 2 |
| 0.0167 | 0.6735 | Mmp20 | P57748\|MMP20_MOUSE Matrix metalloproteinase-20 OS = Mus musculus |
| | | | GN = Mmp20 PE = 2 SV = 1 |
| 0.0172 | 0.6735 | Eno3 | P21550\|ENOB_MOUSE Beta-enolase OS = Mus musculus GN = Eno3 PE = 1 |
| | | | SV = 3 |
| 0.0193 | 0.6802 | Col6a1 | Q04857\|CO6A1_MOUSE Collagen alpha-1(VI) chain OS = Mus musculus |
| | | | GN = Col6a1 PE = 1 SV = 1 |
| 0.0218 | 0.6991 | Krt86 | P97861\|KRT86_MOUSE Keratin, type II cuticular Hb6 OS = Mus musculus |
| | | | GN = Krt86 PE = 2 SV = 2 |
| 0.0284 | 0.7678 | Col14a1 | Q80X19\|COEA1_MOUSE Collagen alpha-1(XIV) chain OS = Mus musculus |
| | | | GN = Col14a1 PE = 1 SV = 2 |
| 0.0340 | 0.7678 | Atp5b | P56480\|ATPB_MOUSE ATP synthase subunit beta, mitochondrial OS = Mus |
| | | | musculus GN = Atp5b PE = 1 SV = 2 |
| 0.0350 | 0.7678 | Art3 | Q8R2G4\|NAR3_MOUSE Ecto-ADP-ribosyltransferase 3 OS = Mus musculus |
| | | | GN = Art3 PE = 1 SV = 2 |
| 0.0372 | 0.7678 | Tinagl1 | Q99JR5\|TINAL_MOUSE Tubulointerstitial nephritis antigen-like OS = Mus |
| | | | musculus GN = Tinagl1 PE = 1 SV = 1 |
| 0.0381 | 0.7678 | Lama4 | P97927\|LAMA4_MOUSE Laminin subunit alpha-4 OS = Mus musculus |
| | | | GN = Lama4 PE = 1 SV = 2 |
| 0.0406 | 0.7678 | 1 SV | P01878\|IGHA_MOUSE Ig alpha chain C region OS = Mus musculus PE = 1 SV = 1 |
| 0.0413 | 0.7678 | Mb | P04247\|MYG_MOUSE Myoglobin OS = Mus musculus GN = Mb PE = 1 SV = 3 |
| 0.0418 | 0.7678 | Hspg2 | Q05793\|PGBM_MOUSE Basement membrane-specific heparan sulfate |
| | | | proteoglycan core protein OS = Mus musculus GN = Hspg2 PE = 1 SV = 1 |
| 0.0447 | 0.7678 | Anxa1 | P10107\|ANXA1_MOUSE Annexin A1 OS = Mus musculus GN = Anxa1 PE = 1 |
| | | | SV = 2 |
| 0.0470 | 0.7678 | Acta1 | P68134\|ACTS_MOUSE Actin, alpha skeletal muscle OS = Mus musculus |
| | | | GN = Acta1 PE = 1 SV = 1 |
| 0.0509 | 0.7678 | Cc2d1a | Q8K1A6\|C2D1A_MOUSE Coiled-coil and C2 domain-containing protein 1A |
| | | | OS = Mus musculus GN = Cc2d1a PE = 1 SV = 2 |
| 0.0515 | 0.7678 | Pkm | P52480\|KPYM_MOUSE Pyruvate kinase PKM OS = Mus musculus |
| | | | GN = Pkm PE = 1 SV = 4 |
| 0.0524 | 0.7678 | Pygm | Q9WUB3\|PYGM_MOUSE Glycogen phosphorylase, muscle form OS = Mus |
| | | | musculus GN = Pygm PE = 1 SV = 3 |
| 0.0545 | 0.7678 | Apoc1 | P34928\|APOC1_MOUSE Apolipoprotein C-I OS = Mus musculus GN = Apoc1 |
| | | | PE = 1 SV = 1 |
| 0.0582 | 0.7679 | Myh3 | P13541\|MYH3_MOUSE Myosin-3 OS = Mus musculus GN = Myh3 PE = 2 |
| | | | SV = 2 |
| 0.0609 | 0.7679 | Mdh2 | P08249\|MDHM_MOUSE Malate dehydrogenase, mitochondrial OS = Mus |
| | | | musculus GN = Mdh2 PE = 1 SV = 3 |
| 0.0611 | 0.7679 | Nid2 | O88322\|NID2_MOUSE Nidogen-2 OS = Mus musculus GN = Nid2 PE = 1 |
| | | | SV = 2 |
| 0.0687 | 0.7738 | Aldoa | P05064\|ALDOA_MOUSE Fructose-bisphosphate aldolase A OS = Mus |
| | | | musculus GN = Aldoa PE = 1 SV = 2 |
| 0.0706 | 0.7738 | Plg | P20918\|PLMN_MOUSE Plasminogen OS = Mus musculus GN = Plg PE = 1 |
| | | | SV = 3 |
| 0.0738 | 0.7738 | Apoe | P08226\|APOE_MOUSE Apolipoprotein E OS = Mus musculus GN = Apoe |
| | | | PE = 1 SV = 2 |
| 0.0738 | 0.7738 | Ca3 | P16015\|CAH3_MOUSE Carbonic anhydrase 3 OS = Mus musculus |
| | | | GN = Ca3 PE = 1 SV = 3 |
| 0.0751 | 0.7738 | Col6a6 | Q8C6K9\|CO6A6_MOUSE Collagen alpha-6(VI) chain OS = Mus musculus |
| | | | GN = Col6a6 PE = 1 SV = 2 |
| 0.0786 | 0.7738 | Tnnt3 | Q9QZ47\|TNNT3_MOUSE Troponin T, fast skeletal muscle OS = Mus |
| | | | musculus GN = Tnnt3 PE = 1 SV = 3 |
| 0.0866 | 0.7738 | Pgam2 | O70250\|PGAM2_MOUSE Phosphoglycerate mutase 2 OS = Mus musculus |
| | | | GN = Pgam2 PE = 1 SV = 3 |
| 0.0871 | 0.7738 | Srl | Q7TQ48\|SRCA_MOUSE Sarcalumenin OS = Mus musculus GN = Srl PE = 1 |
| | | | SV = 1 |
| 0.0884 | 0.7738 | Col16a1 | Q8BLX7\|COGA1_MOUSE Collagen alpha-1 (XVI) chain OS = Mus musculus |
| | | | GN = Col16a1 PE = 1 SV = 2 |
| 0.0945 | 0.7738 | Krt77 | Q6IFZ6\|K2C1B_MOUSE Keratin, type II cytoskeletal 1b OS = Mus musculus |
| | | | GN = Krt77 PE = 1 SV = 1 |
| 0.0953 | 0.7738 | Vwf | Q8CIZ8\|VWF_MOUSE von Willebrand factor OS = Mus musculus GN = Vwf |
| | | | PE = 1 SV = 2 |
| 0.0955 | 0.7738 | Rad54l2 | Q99NG0\|ARIP4_MOUSE Helicase ARIP4 OS = Mus musculus GN = Rad54l2 |
| | | | PE = 1 SV = 1 |
| 0.0985 | 0.7738 | Pvalb | P32848\|PRVA_MOUSE Parvalbumin alpha OS = Mus musculus GN = Pvalb |
| | | | PE = 1 SV = 3 |
| 0.0995 | 0.7738 | Myh7 | Q91Z83\|MYH7_MOUSE Myosin-7 OS = Mus musculus GN = Myh7 PE = 2 |
| | | | SV = 1 |
| 0.1000 | 0.7738 | Gsn | P13020\|GELS_MOUSE Gelsolin OS = Mus musculus GN = Gsn PE = 1 SV = 3 |
| 0.1005 | 0.7738 | Dcn | P28654\|PGS2_MOUSE Decorin OS = Mus musculus GN = Dcn PE = 1 SV = 1 |
| 0.1045 | 0.7738 | Lamb1 | P02469\|LAMB1_MOUSE Laminin subunit beta-1 OS = Mus musculus |
| | | | GN = Lamb1 PE = 1 SV = 3 |
| 0.1052 | 0.7738 | Col15a1 | O35206\|COFA1_MOUSE Collagen alpha-1(XV) chain OS = Mus musculus |
| | | | GN = Col15a1 PE = 1 SV = 2 |
| 0.1056 | 0.7738 | Pgm1 | Q9D0F9\|PGM1_MOUSE Phosphoglucomutase-1 OS = Mus musculus |
| | | | GN = Pgm1 PE = 1 SV = 4 |
| 0.1077 | 0.7738 | Pgk1 | P09411\|PGK1_MOUSE Phosphoglycerate kinase 1 OS = Mus musculus |
| | | | GN = Pgk1 PE = 1 SV = 4 |
| 0.1077 | 0.7738 | Fam160b2 | Q80YR2\|F16B2_MOUSE Protein FAM160B2 OS = Mus musculus |
| | | | GN = Fam160b2 PE = 1 SV = 2 |
| 0.1149 | 0.7969 | Aco2 | Q99KI0\|ACON_MOUSE Aconitate hydratase, mitochondrial OS = Mus |
| | | | musculus GN = Aco2 PE = 1 SV = 1 |
| 0.1155 | 0.7969 | Col4a2 | P08122\|CO4A2_MOUSE Collagen alpha-2(IV) chain OS = Mus musculus |
| | | | GN = Col4a2 PE = 1 SV = 4 |
| 0.1238 | 0.8384 | Myh4 | Q5SX39\|MYH4_MOUSE Myosin-4 OS = Mus musculus GN = Myh4 PE = 2 |
| | | | SV = 1 |
| 0.1283 | 0.8432 | Mpo | P11247\|PERM_MOUSE Myeloperoxidase OS = Mus musculus GN = Mpo |
| | | | PE = 1 SV = 2 |
| 0.1299 | 0.8432 | Hspa8 | P63017\|HSP7C_MOUSE Heat shock cognate 71 kDa protein OS = Mus |
| | | | musculus GN = Hspa8 PE = 1 SV = 1 |
| 0.1319 | 0.8432 | Prelp | Q9JK53\|PRELP_MOUSE Prolargin OS = Mus musculus GN = Prelp PE = 1 |
| | | | SV = 2 |
| 0.1344 | 0.8432 | Ralgapa1 | Q6GYP7\|RGPA1_MOUSE Ral GTPase-activating protein subunit alpha-1 |
| | | | OS = Mus musculus GN = Ralgapa1 PE = 1 SV = 1 |
| 0.1365 | 0.8432 | Prdx6 | O08709\|PRDX6_MOUSE Peroxiredoxin-6 OS = Mus musculus GN = Prdx6 |
| | | | PE = 1 SV = 3 |
| 0.1411 | 0.8561 | Prg2 | Q61878\|PRG2_MOUSE Bone marrow proteoglycan OS = Mus musculus |
| | | | GN = Prg2 PE = 1 SV = 1 |
| 0.1462 | 0.8713 | Tnnc2 | P20801\|TNNC2_MOUSE Troponin C, skeletal muscle OS = Mus musculus |
| | | | GN = Tnnc2 PE = 1 SV = 2 |
| 0.1518 | 0.8713 | Atp5a1 | Q03265\|ATPA_MOUSE ATP synthase subunit alpha, mitochondrial |
| | | | OS = Mus musculus GN = Atp5a1 PE = 1 SV = 1 |
| 0.1545 | 0.8713 | Vdac1 | Q60932\|VDAC1_MOUSE Voltage-dependent anion-selective channel |
| | | | protein 1 OS = Mus musculus GN = Vdac1 PE = 1 SV = 3 |
| 0.1589 | 0.8713 | Ldha | P06151\|LDHA_MOUSE L-lactate dehydrogenase A chain OS = Mus |
| | | | musculus GN = Ldha PE = 1 SV = 3 |
| 0.1593 | 0.8713 | Efl1 | Q8C0D5\|EFL1_MOUSE Elongation factor-like GTPase 1 OS = Mus |
| | | | musculus GN = Efl1 PE = 1 SV = 1 |
| 0.1617 | 0.8713 | Fga | E9PV24\|FIBA_MOUSE Fibrinogen alpha chain OS = Mus musculus |
| | | | GN = Fga PE = 1 SV = 1 |
| 0.1690 | 0.8713 | Myh8 | P13542\|MYH8_MOUSE Myosin-8 OS = Mus musculus GN = Myh8 PE = 2 |
| | | | SV = 2 |
| 0.1698 | 0.8713 | Reg3b | P35230\|REG3B_MOUSE Regenerating islet-derived protein 3-beta |
| | | | OS = Mus musculus GN = Reg3b PE = 1 SV = 1 |
| 0.1703 | 0.8713 | Fgg | Q8VCM7\|FIBG_MOUSE Fibrinogen gamma chain OS = Mus musculus |
| | | | GN = Fgg PE = 1 SV = 1 |
| 0.1724 | 0.8713 | Lum | P51885\|LUM_MOUSE Lumican OS = Mus musculus GN = Lum PE = 1 SV = 2 |
| 0.1754 | 0.8713 | Cps1 | Q8C196\|CPSM_MOUSE Carbamoyl-phosphate synthase [ammonia], |
| | | | mitochondrial OS = Mus musculus GN = Cps1 PE = 1 SV = 2 |
| 0.1755 | 0.8713 | Magi3 | Q9EQJ9\|MAGI3_MOUSE Membrane-associated guanylate kinase, WW |
| | | | and PDZ domain-containing protein 3 OS = Mus musculus GN = Magi3 PE = 1 |
| | | | SV = 2 |
| 0.1758 | 0.8713 | Pfkm | P47857\|PFKAM_MOUSE ATP-dependent 6-phosphofructokinase, muscle |
| | | | type OS = Mus musculus GN = Pfkm PE = 1 SV = 3 |
| 0.1814 | 0.8846 | Omd | O35103\|OMD_MOUSE Osteomodulin OS = Mus musculus GN = Omd PE = 2 SV = 1 |
| 0.1834 | 0.8846 | Cpe | Q00493\|CBPE_MOUSE Carboxypeptidase E OS = Mus musculus GN = Cpe |
| | | | PE = 1 SV = 2 |
| 0.1873 | 0.8909 | Tpi1 | P17751\|TPIS_MOUSE Triosephosphate isomerase OS = Mus musculus |
| | | | GN = Tpi1 PE = 1 SV = 4 |
| 0.1903 | 0.8932 | Ak1 | Q9R0Y5\|KAD1_MOUSE Adenylate kinase isoenzyme 1 OS = Mus musculus |
| | | | GN = Ak1 PE = 1 SV = 1 |
| 0.1965 | 0.8935 | Fam208b | Q5DTT3\|F208B_MOUSE Protein FAM208B OS = Mus musculus |
| | | | GN = Fam208b PE = 1 SV = 2 |
| 0.1992 | 0.8935 | Meltf | Q9R0R1\|TRFM_MOUSE Melanotransferrin OS = Mus musculus GN = Meltf |
| | | | PE = 2 SV = 1 |
| 0.2020 | 0.8935 | Krt33a | Q8K0Y2\|KT33A_MOUSE Keratin, type I cuticular Ha3-I OS = Mus musculus |
| | | | GN = Krt33a PE = 1 SV = 1 |
| 0.2033 | 0.8935 | Serpina1b | P22599\|A1AT2_MOUSE Alpha-1-antitrypsin 1-2 OS = Mus musculus |
| | | | GN = Serpina1b PE = 1 SV = 2 |
| 0.2037 | 0.8935 | Serpinf2 | Q61247\|A2AP_MOUSE Alpha-2-antiplasmin OS = Mus musculus |
| | | | GN = Serpinf2 PE = 1 SV = 1 |
| 0.2133 | 0.8935 | Col4a1 | P02463\|CO4A1_MOUSE Collagen alpha-1(IV) chain OS = Mus musculus |
| | | | GN = Col4a1 PE = 1 SV = 4 |
| 0.2206 | 0.8935 | Ttn | A2ASS6\|TITIN_MOUSE Titin OS = Mus musculus GN = Ttn PE = 1 SV = 1 |
| 0.2222 | 0.8935 | Des | P31001\|DESM_MOUSE Desmin OS = Mus musculus GN = Des PE = 1 SV = 3 |
| 0.2235 | 0.8935 | Sypl2 | O89104\|SYPL2_MOUSE Synaptophysin-like protein 2 OS = Mus musculus |
| | | | GN = Sypl2 PE = 1 SV = 1 |
| 0.2240 | 0.8935 | Hspd1 | P63038\|CH60_MOUSE 60 kDa heat shock protein, mitochondrial OS = Mus |
| | | | musculus GN = Hspd1 PE = 1 SV = 1 |
| 0.2257 | 0.8935 | Tgfbi | P82198\|BGH3_MOUSE Transforming growth factor-beta-induced protein |
| | | | ig-h3 OS = Mus musculus GN = Tgfbi PE = 1 SV = 1 |
| 0.2317 | 0.8935 | Klhdc9 | Q3USL1\|KLDC9_MOUSE Kelch domain-containing protein 9 OS = Mus |
| | | | musculus GN = Klhdc9 PE = 1 SV = 1 |
| 0.2335 | 0.8935 | Mgp | P19788\|MGP_MOUSE Matrix Gla protein OS = Mus musculus GN = Mgp |
| | | | PE = 3 SV = 1 |
| 0.2400 | 0.8935 | Hapln1 | Q9QUP5\|HPLN1_MOUSE Hyaluronan and proteoglycan link protein 1 |
| | | | OS = Mus musculus GN = Hapln1 PE = 1 SV = 1 |
| 0.2415 | 0.8935 | Akap13 | E9Q394\|AKP13_MOUSE A-kinase anchor protein 13 OS = Mus musculus |
| | | | GN = Akap13 PE = 1 SV = 1 |
| 0.2485 | 0.8935 | C3 | P01027\|CO3_MOUSE Complement C3 OS = Mus musculus GN = C3 PE = 1 |
| | | | SV = 3 |
| 0.2505 | 0.8935 | Atp2a1 | Q8R429\|AT2A1_MOUSE Sarcoplasmic/endoplasmic reticulum calcium |
| | | | ATPase 1 OS = Mus musculus GN = Atp2a1 PE = 1 SV = 1 |
| 0.2554 | 0.8935 | Bhmt | O35490\|BHMT1_MOUSE Betaine-homocysteine S-methyltransferase 1 |
| | | | OS = Mus musculus GN = Bhmt PE = 1 SV = 1 |
| 0.2569 | 0.8935 | Fbn2 | Q61555\|FBN2_MOUSE Fibrillin-2 OS = Mus musculus GN = Fbn2 PE = 1 |
| | | | SV = 2 |
| 0.2602 | 0.8935 | Kcng4 | Q80XM3\|KCNG4_MOUSE Potassium voltage-gated channel subfamily G |
| | | | member 4 OS = Mus musculus GN = Kcng4 PE = 2 SV = 1 |
| 0.2625 | 0.8935 | Mylpf | P97457\|MLRS_MOUSE Myosin regulatory light chain 2, skeletal muscle |
| | | | isoform OS = Mus musculus GN = Mylpf PE = 1 SV = 3 |
| 0.2639 | 0.8935 | Bgn | P28653\|PGS1_MOUSE Biglycan OS = Mus musculus GN = Bgn PE = 1 SV = 1 |
| 0.2648 | 0.8935 | Myot | Q9JIF9\|MYOTI_MOUSE Myotilin OS = Mus musculus GN = Myot PE = 1 SV = 1 |
| 0.2663 | 0.8935 | Rev3l | Q61493\|REV3L_MOUSE DNA polymerase zeta catalytic subunit OS = Mus |
| | | | musculus GN = Rev3l PE = 1 SV = 3 |
| 0.2708 | 0.8935 | Csf2rb | P26955\|IL3RB_MOUSE Cytokine receptor common subunit beta OS = Mus |
| | | | musculus GN = Csf2rb PE = 1 SV = 2 |
| 0.2715 | 0.8935 | Mfap4 | Q9D1H9\|MFAP4_MOUSE Microfibril-associated glycoprotein 4 OS = Mus |
| | | | musculus GN = Mfap4 PE = 1 SV = 1 |
| 0.2730 | 0.8935 | Cacna2d1 | O08532\|CA2D1_MOUSE Voltage-dependent calcium channel subunit |
| | | | alpha-2/delta-1 OS = Mus musculus GN = Cacna2d1 PE = 1 SV = 1 |
| 0.2732 | 0.8935 | Filip1l | Q6P6L0\|FIL1L_MOUSE Filamin A-interacting protein 1-like OS = Mus |
| | | | musculus GN = Filip11 PE = 1 SV = 2 |
| 0.2735 | 0.8935 | Fgb | Q8K0E8\|FIBB_MOUSE Fibrinogen beta chain OS = Mus musculus GN = Fgb |
| | | | PE = 1 SV = 1 |
| 0.2754 | 0.8935 | Myom1 | Q62234\|MYOM1_MOUSE Myomesin-1 OS = Mus musculus GN = Myom1 |
| | | | PE = 1 SV = 2 |
| 0.2782 | 0.8935 | Osbpl8 | B9EJ86\|OSBL8_MOUSE Oxysterol-binding protein-related protein 8 |
| | | | OS = Mus musculus GN = Osbpl8 PE = 1 SV = 1 |
| 0.2806 | 0.8935 | Camsap1 | A2AHC3\|CAMP1_MOUSE Calmodulin-regulated spectrin-associated |
| | | | protein 1 OS = Mus musculus GN = Camsap1 PE = 1 SV = 1 |
| 0.2807 | 0.8935 | Col9a2 | Q07643\|CO9A2_MOUSE Collagen alpha-2(IX) chain OS = Mus musculus |
| | | | GN = Col9a2 PE = 2 SV = 1 |
| 0.2938 | 0.8935 | Col5a1 | O88207\|CO5A1_MOUSE Collagen alpha-1(V) chain OS = Mus musculus |
| | | | GN = Col5a1 PE = 1 SV = 2 |
| 0.2969 | 0.8935 | Actn3 | O88990\|ACTN3_MOUSE Alpha-actinin-3 OS = Mus musculus GN = Actn3 |
| | | | PE = 2 SV = 1 |
| 0.3014 | 0.8935 | Egfbp2 | P36368\|EGFB2_MOUSE Epidermal growth factor-binding protein type B |
| | | | OS = Mus musculus GN = Egfbp2 PE = 1 SV = 1 |
| 0.3057 | 0.8935 | Tpm1 | P58771\|TPM1_MOUSE Tropomyosin alpha-1 chain OS = Mus musculus |
| | | | GN = Tpm1 PE = 1 SV = 1 |
| 0.3064 | 0.8935 | Hrg | Q9ESB3\|HRG_MOUSE Histidine-rich glycoprotein OS = Mus musculus |
| | | | GN = Hrg PE = 1 SV = 2 |
| 0.3077 | 0.8935 | Matn1 | P51942\|MATN1_MOUSE Cartilage matrix protein OS = Mus musculus |
| | | | GN = Matn1 PE = 2 SV = 2 |
| 0.3085 | 0.8935 | Fbn1 | Q61554\|FBN1_MOUSE Fibrillin-1 OS = Mus musculus GN = Fbn1 PE = 1 |
| | | | SV = 2 |
| 0.3098 | 0.8935 | Myoz1 | Q9JK37\|MYOZ1_MOUSE Myozenin-1 OS = Mus musculus GN = Myoz1 |
| | | | PE = 1 SV = 1 |
| 0.3101 | 0.8935 | Tmem207 | P86045\|TM207_MOUSE Transmembrane protein 207 OS = Mus musculus |
| | | | GN = Tmem207 PE = 2 SV = 1 |
| 0.3118 | 0.8935 | Krt19 | P19001\|K1C19_MOUSE Keratin, type I cytoskeletal 19 OS = Mus musculus |
| | | | GN = Krt19 PE = 1 SV = 1 |
| 0.3118 | 0.8935 | Ltbp4 | Q8K4G1\|LTBP4_MOUSE Latent-transforming growth factor beta-binding |
| | | | protein 4 OS = Mus musculus GN = Ltbp4 PE = 1 SV = 2 |
| 0.3128 | 0.8935 | Fhdc1 | Q3ULZ2\|FHDC1_MOUSE FH2 domain-containing protein 1 OS = Mus |
| | | | musculus GN = Fhdc1 PE = 1 SV = 3 |
| 0.3135 | 0.8935 | S100a11 | P50543\|S10AB_MOUSE Protein S100-A11 OS = Mus musculus |
| | | | GN = S100a11 PE = 1 SV = 1 |
| 0.3200 | 0.8935 | Ubb | P0CG49\|UBB_MOUSE Polyubiquitin-B OS = Mus musculus GN = Ubb PE = 2 |
| | | | SV = 1 |
| 0.3211 | 0.8935 | 1 SV | P01837\|IGKC_MOUSE Ig kappa chain C region OS = Mus musculus PE = 1 |
| | | | SV = 1 |
| 0.3219 | 0.8935 | Tpm3 | P21107\|TPM3_MOUSE Tropomyosin alpha-3 chain OS = Mus musculus |
| | | | GN = Tpm3 PE = 1 SV = 3 |
| 0.3239 | 0.8935 | Ogn | Q62000\|MIME_MOUSE Mimecan OS = Mus musculus GN = Ogn PE = 1 SV = 1 |
| 0.3242 | 0.8935 | Cdkn2aip | Q8BI72\|CARF_MOUSE CDKN2A-interacting protein OS = Mus musculus |
| | | | GN = Cdkn2aip PE = 1 SV = 1 |
| 0.3250 | 0.8935 | Ppa1 | Q9D819\|IPYR_MOUSE Inorganic pyrophosphatase OS = Mus musculus |
| | | | GN = Ppa1 PE = 1 SV = 1 |
| 0.3263 | 0.8935 | Emilin1 | Q99K41\|EMIL1_MOUSE EMILIN-1 OS = Mus musculus GN = Emilin1 PE = 1 |
| | | | SV = 1 |
| 0.3281 | 0.8935 | Serpina3m | Q03734\|SPA3M_MOUSE Serine protease inhibitor A3M OS = Mus |
| | | | musculus GN = Serpina3m PE = 1 SV = 2 |
| 0.3300 | 0.8935 | Cpa3 | P15089\|CBPA3_MOUSE Mast cell carboxypeptidase A OS = Mus musculus |
| | | | GN = Cpa3 PE = 2 SV = 1 |
| 0.3329 | 0.8938 | Dpt | Q9QZZ6\|DERM_MOUSE Dermatopontin OS = Mus musculus GN = Dpt PE = 1 |
| | | | SV = 1 |
| 0.3358 | 0.8938 | Lgals1 | P16045\|LEG1_MOUSE Galectin-1 OS = Mus musculus GN = Lgals1 PE = 1 |
| | | | SV = 3 |
| 0.3377 | 0.8938 | Tef | Q9JLC6\|TEF_MOUSE Thyrotroph embryonic factor OS = Mus musculus |
| | | | GN = Tef PE = 2 SV = 1 |
| 0.3469 | 0.8941 | Ckm | P07310\|KCRM_MOUSE Creatine kinase M-type OS = Mus musculus |
| | | | GN = Ckm PE = 1 SV = 1 |
| 0.3470 | 0.8941 | Ampd1 | Q3V1D3\|AMPD1_MOUSE AMP deaminase 1 OS = Mus musculus |
| | | | GN = Ampd1 PE = 1 SV = 2 |
| 0.3497 | 0.8941 | Ethc1 | Q9D9T8\|EFHC1_MOUSE EF-hand domain-containing protein 1 OS = Mus |
| | | | musculus GN = Efhc1 PE = 1 SV = 1 |
| 0.3498 | 0.8941 | Mmp9 | P41245\|MMP9_MOUSE Matrix metalloproteinase-9 OS = Mus musculus |
| | | | GN = Mmp9 PE = 1 SV = 2 |
| 0.3505 | 0.8941 | S100b | P50114\|S100B_MOUSE Protein S100-B OS = Mus musculus GN = S100b |
| | | | PE = 1 SV = 2 |
| 0.3556 | 0.9006 | Fasn | P19096\|FAS_MOUSE Fatty acid synthase OS = Mus musculus GN = Fasn |
| | | | PE = 1 SV = 2 |
| 0.3611 | 0.9031 | Cxcr1 | Q810W6\|CXCR1_MOUSE C-X-C chemokine receptor type 1 OS = Mus |
| | | | musculus GN = Cxcr1 PE = 1 SV = 1 |
| 0.3628 | 0.9031 | Rgn | Q64374\|RGN_MOUSE Regucalcin OS = Mus musculus GN = Rgn PE = 1 |
| | | | SV = 1 |
| 0.3696 | 0.9031 | Tuba4a | P68368\|TBA4A_MOUSE Tubulin alpha-4A chain OS = Mus musculus |
| | | | GN = Tuba4a PE = 1 SV = 1 |
| 0.3701 | 0.9031 | 1 SV | Streptavidin\|P22629\|SAV STRAV Streptavidin OS = Streptomyces avidinii |
| | | | PE = 1 SV = 1 |
| 0.3743 | 0.9031 | Plch2 | A2AP18\|PLCH2_MOUSE 1-phosphatidylinositol 4,5-bisphosphate |
| | | | phosphodiesterase eta-2 OS = Mus musculus GN = Plch2 PE = 1 SV = 2 |
| 0.3750 | 0.9031 | Hnrnpa2b1 | O88569\|ROA2_MOUSE Heterogeneous nuclear ribonucleoproteins A2/B1 |
| | | | OS = Mus musculus GN = Hnrnpa2b1 PE = 1 SV = 2 |
| 0.3759 | 0.9031 | Hpx | Q91X72\|HEMO_MOUSE Hemopexin OS = Mus musculus GN = Hpx PE = 1 |
| | | | SV = 2 |
| 0.3780 | 0.9031 | Cilp2 | D3Z7H8\|CILP2_MOUSE Cartilage intermediate layer protein 2 OS = Mus |
| | | | musculus GN = Cilp2 PE = 1 SV = 1 |
| 0.3797 | 0.9031 | Kmt2a | P55200\|KMT2A_MOUSE Histone-lysine N-methyltransferase 2A OS = Mus |
| | | | musculus GN = Kmt2a PE = 1 SV = 3 |
| 0.3828 | 0.9044 | Prdx1 | P35700\|PRDX1_MOUSE Peroxiredoxin-1 OS = Mus musculus GN = Prdx1 |
| | | | PE = 1 SV = 1 |
| 0.4018 | 0.9430 | Capza1 | P47753\|CAZA1_MOUSE F-actin-capping protein subunit alpha-1 OS = Mus |
| | | | musculus GN = Capza1 PE = 1 SV = 4 |
| 0.4083 | 0.9507 | Chrnd | P02716\|ACHD_MOUSE Acetylcholine receptor subunit delta OS = Mus |
| | | | musculus GN = Chrnd PE = 2 SV = 1 |
| 0.4157 | 0.9507 | Casq1 | O09165\|CASQ1_MOUSE Calsequestrin-1 OS = Mus musculus GN = Casq1 |
| | | | PE = 1 SV = 3 |
| 0.4262 | 0.9507 | S100a1 | P56565\|S10A1_MOUSE Protein S100-A1 OS = Mus musculus GN = S100a1 |
| | | | PE = 1 SV = 2 |
| 0.4285 | 0.9507 | Pc | Q05920\|PYC_MOUSE Pyruvate carboxylase, mitochondrial OS = Mus |
| | | | musculus GN = Pc PE = 1 SV = 1 |
| 0.4288 | 0.9507 | Apoa1 | Q00623\|APOA1_MOUSE Apolipoprotein A-I OS = Mus musculus |
| | | | GN = Apoa1 PE = 1 SV = 2 |
| 0.4314 | 0.9507 | Col10a1 | Q05306\|COAA1_MOUSE Collagen alpha-1(X) chain OS = Mus musculus |
| | | | GN = Col10a1 PE = 2 SV = 1 |
| 0.4318 | 0.9507 | Spn | P15702\|LEUK_MOUSE Leukosialin OS = Mus musculus GN = Spn PE = 1 |
| | | | SV = 1 |
| 0.4334 | 0.9507 | Actn2 | Q9JI91\|ACTN2_MOUSE Alpha-actinin-2 OS = Mus musculus GN = Actn2 |
| | | | PE = 1 SV = 2 |
| 0.4395 | 0.9507 | Npepps | Q11011\|PSA_MOUSE Puromycin-sensitive aminopeptidase OS = Mus |
| | | | musculus GN = Npepps PE = 1 SV = 2 |
| 0.4401 | 0.9507 | Apoa4 | P06728\|APOA4_MOUSE Apolipoprotein A-IV OS = Mus musculus |
| | | | GN = Apoa4 PE = 1 SV = 3 |
| 0.4423 | 0.9507 | Arhgap5 | P97393\|RHG05_MOUSE Rho GTPase-activating protein 5 OS = Mus |
| | | | musculus GN = Arhgap5 PE = 1 SV = 2 |
| 0.4506 | 0.9507 | Fnbp4 | Q6ZQ03\|FNBP4_MOUSE Formin-binding protein 4 OS = Mus musculus |
| | | | GN = Fnbp4 PE = 1 SV = 2 |
| 0.4510 | 0.9507 | Bcl9 | Q9D219\|BCL9_MOUSE B-cell CLL/lymphoma 9 protein OS = Mus musculus |
| | | | GN = Bcl9 PE = 1 SV = 3 |
| 0.4522 | 0.9507 | Serpina3k | P07759\|SPA3K_MOUSE Serine protease inhibitor A3K OS = Mus musculus |
| | | | GN = Serpina3k PE = 1 SV = 2 |
| 0.4539 | 0.9507 | Mvb12a | Q78HU3\|MB12A_MOUSE Multivesicular body subunit 12A OS = Mus |
| | | | musculus GN = Mvb12a PE = 1 SV = 1 |
| 0.4561 | 0.9507 | Sord | Q64442\|DHSO_MOUSE Sorbitol dehydrogenase OS = Mus musculus |
| | | | GN = Sord PE = 1 SV = 3 |
| 0.4595 | 0.9507 | Ecm1 | Q61508\|ECM1_MOUSE Extracellular matrix protein 1 OS = Mus musculus |
| | | | GN = Ecm1 PE = 1 SV = 2 |
| 0.4597 | 0.9507 | Ushbp1 | Q8R370\|USBP1_MOUSE Usher syndrome type-10 protein-binding protein |
| | | | 1 OS = Mus musculus GN = Ushbp1 PE = 1 SV = 2 |
| 0.4604 | 0.9507 | Insrr | Q9WTL4\|INSRR_MOUSE Insulin receptor-related protein OS = Mus |
| | | | musculus GN = Insrr PE = 1 SV = 2 |
| 0.4655 | 0.9507 | Aspn | Q99MQ4\|ASPN_MOUSE Asporin OS = Mus musculus GN = Aspn PE = 1 |
| | | | SV = 1 |
| 0.4684 | 0.9507 | Gapdh | P16858\|G3P_MOUSE Glyceraldehyde-3-phosphate dehydrogenase |
| | | | OS = Mus musculus GN = Gapdh PE = 1 SV = 2 |
| 0.4708 | 0.9507 | 1 SV | P01631\|KV2A7_MOUSE Ig kappa chain V-II region 26-10 OS = Mus |
| | | | musculus PE = 1 SV = 1 |
| 0.4750 | 0.9507 | Alb | P07724\|ALBU_MOUSE Serum albumin OS = Mus musculus GN = Alb PE = 1 |
| | | | SV = 3 |
| 0.4809 | 0.9507 | Plec | Q9QXS1\|PLEC_MOUSE Plectin OS = Mus musculus GN = Plec PE = 1 SV = 3 |
| 0.4861 | 0.9507 | Tprn | A2AI08\|TPRN_MOUSE Taperin OS = Mus musculus GN = Tprn PE = 1 SV = 1 |
| 0.4901 | 0.9507 | Krt10 | P02535\|K1C10_MOUSE Keratin, type I cytoskeletal 10 OS = Mus musculus |
| | | | GN = Krt10 PE = 1 SV = 3 |
| 0.4925 | 0.9507 | Eef1a1 | P10126\|EF1A1_MOUSE Elongation factor 1-alpha 1 OS = Mus musculus |
| | | | GN = Eef1a1 PE = 1 SV = 3 |
| 0.4994 | 0.9507 | Comp | Q9ROG6\|COMP_MOUSE Cartilage oligomeric matrix protein OS = Mus |
| | | | musculus GN = Comp PE = 1 SV = 2 |
| 0.4995 | 0.9507 | Clu | Q06890\|CLUS_MOUSE Clusterin OS = Mus musculus GN = Clu PE = 1 SV = 1 |
| 0.5034 | 0.9507 | Lama5 | Q61001\|LAMA5_MOUSE Laminin subunit alpha-5 OS = Mus musculus |
| | | | GN = Lama5 PE = 1 SV = 4 |
| 0.5047 | 0.9507 | Ppip5k1 | A2ARP1\|VIP1_MOUSE Inositol hexakisphosphate and diphosphoinositol- |
| | | | pentakisphosphate kinase 1 OS = Mus musculus GN = Ppip5k1 PE = 1 SV = 1 |
| 0.5083 | 0.9507 | Col5a2 | Q3U962\|CO5A2_MOUSE Collagen alpha-2(V) chain OS = Mus musculus |
| | | | GN = Col5a2 PE = 1 SV = 1 |
| 0.5121 | 0.9507 | Dsp | E9Q557\|DESP_MOUSE Desmoplakin OS = Mus musculus GN = Dsp PE = 1 SV = 1 |
| 0.5179 | 0.9507 | Postn | Q62009\|POSTN_MOUSE Periostin OS = Mus musculus GN = Postn PE = 1 |
| | | | SV = 2 |
| 0.5225 | 0.9507 | Ppfia3 | P60469\|LIPA3_MOUSE Liprin-alpha-3 OS = Mus musculus GN = Ppfia3 |
| | | | PE = 1 SV = 2 |
| 0.5237 | 0.9507 | Actc1 | P68033\|ACTC_MOUSE Actin, alpha cardiac muscle 1 OS = Mus musculus |
| | | | GN = Actc1 PE = 1 SV = 1 |
| 0.5242 | 0.9507 | Tf | Q92111\|TRFE_MOUSE Serotransferrin OS = Mus musculus GN = Tf PE = 1 |
| | | | SV = 1 |
| 0.5246 | 0.9507 | Wipf1 | Q8K117\|WIPF1_MOUSE WAS/WASL-interacting protein family member 1 |
| | | | OS = Mus musculus GN = Wipf1 PE = 1 SV = 1 |
| 0.5267 | 0.9507 | Itih4 | A6X935\|ITIH4_MOUSE Inter alpha-trypsin inhibitor, heavy chain 4 OS = Mus |
| | | | musculus GN = ltih4 PE = 1 SV = 2 |
| 0.5301 | 0.9507 | Col11a1 | Q61245\|COBA1_MOUSE Collagen alpha-1(XI) chain OS = Mus musculus |
| | | | GN = Col11a1 PE = 1 SV = 2 |
| 0.5301 | 0.9507 | Sepp1 | P70274\|SEPP1_MOUSE Selenoprotein P OS = Mus musculus GN = Sepp1 |
| | | | PE = 1 SV = 3 |
| 0.5319 | 0.9507 | Cycs | P62897\|CYC_MOUSE Cytochrome c, somatic OS = Mus musculus |
| | | | GN = Cycs PE = 1 SV = 2 |
| 0.5331 | 0.9507 | Asb3 | Q9WV72\|ASB3_MOUSE Ankyrin repeat and SOCS box protein 3 OS = Mus |
| | | | musculus GN = Asb3 PE = 1 SV = 2 |
| 0.5361 | 0.9507 | Tgm2 | P21981\|TGM2_MOUSE Protein-glutamine gamma-glutamyltransferase 2 |
| | | | OS = Mus musculus GN = Tgm2 PE = 1 SV = 4 |
| 0.5363 | 0.9507 | Serpina1d | Q00897\|A1AT4_MOUSE Alpha-1-antitrypsin 1-4 OS = Mus musculus |
| | | | GN = Serpina1d PE = 1 SV = 1 |
| 0.5374 | 0.9507 | Fn1 | P11276\|FINC_MOUSE Fibronectin OS = Mus musculus GN = Fn1 PE = 1 |
| | | | SV = 4 |
| 0.5381 | 0.9507 | Map2k4 | P47809\|MP2K4_MOUSE Dual specificity mitogen-activated protein kinase |
| | | | kinase 4 OS = Mus musculus GN = Map2k4 PE = 1 SV = 2 |
| 0.5411 | 0.9507 | Abcc3 | B2RX12\|MRP3_MOUSE Canalicular multispecific organic anion transporter |
| | | | 2 OS = Mus musculus GN = Abcc3 PE = 1 SV = 1 |
| 0.5423 | 0.9507 | Krtap3-1 | A2A591\|KRA31_MOUSE Keratin-associated protein 3-1 OS = Mus |
| | | | musculus GN = Krtap3-1 PE = 3 SV = 1 |
| 0.5458 | 0.9507 | Tmem184c | Q3TPR7\|T184C_MOUSE Transmembrane protein 184C OS = Mus |
| | | | musculus GN = Tmem184c PE = 2 SV = 1 |
| 0.5459 | 0.9507 | Vim | P20152\|VIME_MOUSE Vimentin OS = Mus musculus GN = Vim PE = 1 SV = 3 |
| 0.5477 | 0.9507 | Fbln5 | Q9WVH9\|FBLN5_MOUSE Fibulin-5 OS = Mus musculus GN = Fbln5 PE = 1 |
| | | | SV = 1 |
| 0.5482 | 0.9507 | Cma1 | P21844\|CMA1_MOUSE Chymase OS = Mus musculus GN = Cma1 PE = 1 |
| | | | SV = 2 |
| 0.5534 | 0.9515 | Slc25a37 | Q920G8\|MFRN1_MOUSE Mitoferrin-1 OS = Mus musculus GN = Slc25a37 |
| | | | PE = 1 SV = 1 |
| 0.5541 | 0.9515 | Myo3b | Q1EG27\|MYO3B_MOUSE Myosin-IIIb OS = Mus musculus GN = Myo3b |
| | | | PE = 1 SV = 2 |
| 0.5613 | 0.9591 | Med1 | Q925J9\|MED1_MOUSE Mediator of RNA polymerase II transcription |
| | | | subunit 1 OS = Mus musculus GN = Med1 PE = 1 SV = 2 |
| 0.5740 | 0.9641 | Col2a1 | P28481\|CO2A1_MOUSE Collagen alpha-1 (II) chain OS = Mus musculus |
| | | | GN = Col2a1 PE = 1 SV = 2 |
| 0.5762 | 0.9641 | Amy2 | P00688\|AMYP_MOUSE Pancreatic alpha-amylase OS = Mus musculus |
| | | | GN = Amy2 PE = 1 SV = 2 |
| 0.5796 | 0.9641 | Ube3b | Q9ES34\|UBE3B_MOUSE Ubiquitin-protein ligase E3B OS = Mus musculus |
| | | | GN = Ube3b PE = 1 SV = 3 |
| 0.5834 | 0.9641 | Apobec2 | Q9WV35\|ABEC2_MOUSE C->U-editing enzyme APOBEC-2 OS = Mus |
| | | | musculus GN = Apobec2 PE = 1 SV = 1 |
| 0.5859 | 0.9641 | Tnni2 | P13412\|TNNI2_MOUSE Troponin I, fast skeletal muscle OS = Mus |
| | | | musculus GN = Tnni2 PE = 2 SV = 2 |
| 0.5880 | 0.9641 | Eno1 | P17182\|ENOA_MOUSE Alpha-enolase OS = Mus musculus GN = Eno1 |
| | | | PE = 1 SV = 3 |
| 0.5901 | 0.9641 | Lef1 | P27782\|LEF1_MOUSE Lymphoid enhancer-binding factor 1 OS = Mus |
| | | | musculus GN = Lef1 PE = 1 SV = 1 |
| 0.5919 | 0.9641 | Ryr1 | E9PZQ0\|RYR1_MOUSE Ryanodine receptor 1 OS = Mus musculus |
| | | | GN = Ryr1 PE = 1 SV = 1 |
| 0.5956 | 0.9641 | Loxl1 | P97873\|LOXL1_MOUSE Lysyl oxidase homolog 1 OS = Mus musculus |
| | | | GN = Loxl1 PE = 2 SV = 3 |
| 0.5968 | 0.9641 | Tuba1b | P05213\|TBA1B_MOUSE Tubulin alpha-1B chain OS = Mus musculus |
| | | | GN = Tuba1b PE = 1 SV = 2 |
| 0.5982 | 0.9641 | Bsg | P18572\|BASI_MOUSE Basigin OS = Mus musculus GN = Bsg PE = 1 SV = 2 |
| 0.5991 | 0.9641 | Nup98 | Q6PFD9\|NUP98_MOUSE Nuclear pore complex protein Nup98-Nup96 |
| | | | OS = Mus musculus GN = Nup98 PE = 1 SV = 2 |
| 0.6021 | 0.9641 | Ass1 | P16460\|ASSY_MOUSE Argininosuccinate synthase OS = Mus musculus |
| | | | GN = Ass1 PE = 1 SV = 1 |
| 0.6040 | 0.9641 | Acan | Q61282\|PGCA_MOUSE Aggrecan core protein OS = Mus musculus |
| | | | GN = Acan PE = 1 SV = 2 |
| 0.6053 | 0.9641 | Sgf29 | Q9DA08\|SGF29_MOUSE SAGA-associated factor 29 OS = Mus musculus |
| | | | GN = Sgf29 PE = 2 SV = 1 |
| 0.6114 | 0.9674 | Rps15 | P62843\|RS15_MOUSE 40S ribosomal protein S15 OS = Mus musculus |
| | | | GN = Rps15 PE = 1 SV = 2 |
| 0.6151 | 0.9674 | Krt42 | Q6IFX2\|K1C42_MOUSE Keratin, type I cytoskeletal 42 OS = Mus musculus |
| | | | GN = Krt42 PE = 1 SV = 1 |
| 0.6164 | 0.9674 | Col1a2 | Q01149\|CO1A2_MOUSE Collagen alpha-2(I) chain OS = Mus musculus |
| | | | GN = Col1a2 PE = 1 SV = 2 |
| 0.6184 | 0.9674 | Aldh2 | P47738\|ALDH2_MOUSE Aldehyde dehydrogenase, mitochondrial OS = Mus |
| | | | musculus GN = Aldh2 PE = 1 SV = 1 |
| 0.6251 | 0.9695 | Dock8 | Q8C147\|DOCK8_MOUSE Dedicator of cytokinesis protein 8 OS = Mus |
| | | | musculus GN = Dock8 PE = 1 SV = 4 |
| 0.6278 | 0.9695 | Cys1 | Q8R4T1\|CYS1_MOUSE Cystin-1 OS = Mus musculus GN = Cys1 PE = 1 |
| | | | SV = 1 |
| 0.6286 | 0.9695 | Obscn | A2AAJ9\|OBSCN_MOUSE Obscurin OS = Mus musculus GN = Obscn PE = 1 |
| | | | SV = 2 |
| 0.6313 | 0.9695 | Fbxo48 | Q8CAT8\|FBX48_MOUSE F-box only protein 48 OS = Mus musculus |
| | | | GN = Fbxo48 PE = 2 SV = 1 |
| 0.6345 | 0.9695 | Col9a1 | Q05722\|CO9A1_MOUSE Collagen alpha-1(IX) chain OS = Mus musculus |
| | | | GN = Col9a1 PE = 2 SV = 2 |
| 0.6382 | 0.9695 | Kiaa1109 | A2AAE1\|K1109_MOUSE Uncharacterized protein KIAA1109 OS = Mus |
| | | | musculus GN = Kiaa1109 PE = 1 SV = 4 |
| 0.6390 | 0.9695 | Wdfy3 | Q6VNB8\|WDFY3_MOUSE WD repeat and FYVE domain-containing |
| | | | protein 3 OS = Mus musculus GN = Wdfy3 PE = 1 SV = 1 |
| 0.6485 | 0.9723 | Taco1 | Q8K0Z7\|TACO1_MOUSE Translational activator of cytochrome c oxidase 1 |
| | | | OS = Mus musculus GN = Taco1 PE = 1 SV = 1 |
| 0.6518 | 0.9723 | Lyz1 | P17897\|LYZ1_MOUSE Lysozyme C-1 OS = Mus musculus GN = Lyz1 PE = 1 |
| | | | SV = 1 |
| 0.6520 | 0.9723 | GMCL1P1 | Q99N64\|GMCLL_MOUSE Putative germ cell-less protein-like 1-like |
| | | | OS = Mus musculus GN = GMCL1P1 PE = 2 SV = 2 |
| 0.6552 | 0.9723 | Krtap15-1 | Q9QZU5\|KR151_MOUSE Keratin-associated protein 15-1 OS = Mus |
| | | | musculus GN = Krtap15-1 PE = 2 SV = 1 |
| 0.6553 | 0.9723 | Hsp90ab1 | P11499\|HS90B_MOUSE Heat shock protein HSP 90-beta OS = Mus |
| | | | musculus GN = Hsp90ab1 PE = 1 SV = 3 |
| 0.6574 | 0.9723 | Krt6a | P50446\|K2C6A_MOUSE Keratin, type II cytoskeletal 6A OS = Mus |
| | | | musculus GN = Krt6a PE = 1 SV = 3 |
| 0.6623 | 0.9754 | Hbb-b1 | P02088\|HBB1_MOUSE Hemoglobin subunit beta-1 OS = Mus musculus |
| | | | GN = Hbb-b1 PE = 1 SV = 2 |
| 0.6658 | 0.9766 | Ddx25 | Q9QY15\|DDX25_MOUSE ATP-dependent RNA helicase DDX25 OS = Mus |
| | | | musculus GN = Ddx25 PE = 1 SV = 2 |
| 0.6748 | 0.9767 | Foxe3 | Q9QY14\|FOXE3_MOUSE Forkhead box protein E3 OS = Mus musculus |
| | | | GN = Foxe3 PE = 1 SV = 1 |
| 0.6766 | 0.9767 | Mnt | O08789\|MNT_MOUSE Max-binding protein MNT OS = Mus musculus |
| | | | GN = Mnt PE = 2 SV = 2 |
| 0.6767 | 0.9767 | Ppia | P17742\|PPIA_MOUSE Peptidyl-prolyl cis-trans isomerase A OS = Mus |
| | | | musculus GN = Ppia PE = 1 SV = 2 |
| 0.6784 | 0.9767 | Gabrb3 | P63080\|GBRB3_MOUSE Gamma-aminobutyric acid receptor subunit beta- |
| | | | 3 OS = Mus musculus GN = Gabrb3 PE = 2 SV = 1 |
| 0.6798 | 0.9767 | Spp1 | P10923\|OSTP_MOUSE Osteopontin OS = Mus musculus GN = Spp1 PE = 1 SV = 1 |
| 0.6899 | 0.9858 | Pou2f3 | P31362\|PO2F3_MOUSE POU domain, class 2, transcription factor 3 |
| | | | OS = Mus musculus GN = Pou2f3 PE = 2 SV = 2 |
| 0.6917 | 0.9858 | Eef1a2 | P62631\|EF1A2_MOUSE Elongation factor 1-alpha 2 OS = Mus musculus |
| | | | GN = Eef1a2 PE = 1 SV = 1 |
| 0.6977 | 0.9864 | Dopey2 | Q3UHQ6\|DOP2_MOUSE Protein dopey-2 OS = Mus musculus GN = Dopey2 |
| | | | PE = 1 SV = 3 |
| 0.6989 | 0.9864 | Pcca | Q91ZA3\|PCCA_MOUSE Propionyl-CoA carboxylase alpha chain, |
| | | | mitochondrial OS = Mus musculus GN = Pcca PE = 1 SV = 2 |
| 0.7031 | 0.9864 | Fmod | P50608\|FMOD_MOUSE Fibromodulin OS = Mus musculus GN = Fmod PE = 2 |
| | | | SV = 1 |
| 0.7061 | 0.9864 | Pank1 | Q8K4K6\|PANK1_MOUSE Pantothenate kinase 1 OS = Mus musculus |
| | | | GN = Pank1 PE = 1 SV = 1 |
| 0.7062 | 0.9864 | Met | P16056\|MET_MOUSE Hepatocyte growth factor receptor OS = Mus |
| | | | musculus GN = Met PE = 1 SV = 1 |
| 0.7102 | 0.9880 | Prdx2 | Q61171\|PRDX2_MOUSE Peroxiredoxin-2 OS = Mus musculus GN = Prdx2 |
| | | | PE = 1 SV = 3 |
| 0.7139 | 0.9880 | Hba | P01942\|HBA_MOUSE Hemoglobin subunit alpha OS = Mus musculus |
| | | | GN = Hba PE = 1 SV = 2 |
| 0.7168 | 0.9880 | Chad | O55226\|CHAD_MOUSE Chondroadherin OS = Mus musculus GN = Chad |
| | | | PE = 2 SV = 1 |
| 0.7194 | 0.9880 | Tmem45a | Q60774\|TM45A_MOUSE Transmembrane protein 45A OS = Mus musculus |
| | | | GN = Tmem45a PE = 2 SV = 1 |
| 0.7226 | 0.9880 | F13a1 | Q8BH61\|F13A_MOUSE Coagulation factor XIII A chain OS = Mus musculus |
| | | | GN = F13a1 PE = 1 SV = 3 |
| 0.7249 | 0.9880 | Krt16 | Q9Z2K1\|K1C16_MOUSE Keratin, type I cytoskeletal 16 OS = Mus musculus |
| | | | GN = Krt16 PE = 1 SV = 3 |
| 0.7292 | 0.9880 | Irf4 | Q64287\|IRF4_MOUSE Interferon regulatory factor 4 OS = Mus musculus |
| | | | GN = Irf4 PE = 1 SV = 1 |
| 0.7306 | 0.9880 | Cdh13 | Q9WTR5\|CAD13_MOUSE Cadherin-13 OS = Mus musculus GN = Cdh13 |
| | | | PE = 1 SV = 2 |
| 0.7326 | 0.9880 | Aldob | Q91Y97\|ALDOB_MOUSE Fructose-bisphosphate aldolase B OS = Mus |
| | | | musculus GN = Aldob PE = 1 SV = 3 |
| 0.7387 | 0.9884 | Klhl41 | A2AUC9\|KLH41_MOUSE Kelch-like protein 41 OS = Mus musculus |
| | | | GN = Klhl41 PE = 1 SV = 1 |
| 0.7422 | 0.9884 | Dgcr2 | P98154\|IDD_MOUSE Integral membrane protein DGCR2/IDD OS = Mus |
| | | | musculus GN = Dgcr2 PE = 2 SV = 1 |
| 0.7431 | 0.9884 | Ogdh | Q60597\|ODO1_MOUSE 2-oxoglutarate dehydrogenase, mitochondrial |
| | | | OS = Mus musculus GN = Ogdh PE = 1 SV = 3 |
| 0.7441 | 0.9884 | Hmcn2 | A2AJ76\|HMCN2_MOUSE Hemicentin-2 OS = Mus musculus GN = Hmcn2 |
| | | | PE = 1 SV = 1 |
| 0.7473 | 0.9890 | Peli3 | Q8BXR6\|PELI3_MOUSE E3 ubiquitin-protein ligase pellino homolog 3 |
| | | | OS = Mus musculus GN = Peli3 PE = 2 SV = 2 |
| 0.7521 | 0.9915 | Sparc | P07214\|SPRC_MOUSE SPARC OS = Mus musculus GN = Sparc PE = 1 |
| | | | SV = 1 |
| 0.7572 | 0.9936 | Ywhab | Q9CQV8\|1433B_MOUSE 14-3-3 protein beta/alpha OS = Mus musculus |
| | | | GN = Ywhab PE = 1 SV = 3 |
| 0.7593 | 0.9936 | Thbs4 | Q9Z1T2\|TSP4_MOUSE Thrombospondin-4 OS = Mus musculus GN = Thbs4 |
| | | | PE = 1 SV = 1 |
| 0.7651 | 0.9968 | Hist1h2bc | Q6ZWY9\|H2B1C_MOUSE Histone H2B type 1-C/E/G OS = Mus musculus |
| | | | GN = Hist1h2bc PE = 1 SV = 3 |
| 0.7774 | 0.9968 | Hist1h4a | P62806\|H4_MOUSE Histone H4 OS = Mus musculus GN = Hist1h4a PE = 1 |
| | | | SV = 2 |
| 0.7798 | 0.9968 | Matn3 | O35701\|MATN3_MOUSE Matrilin-3 OS = Mus musculus GN = Matn3 PE = 1 |
| | | | SV = 2 |
| 0.7818 | 0.9968 | S100a9 | P31725\|S10A9_MOUSE Protein S100-A9 OS = Mus musculus GN = S100a9 |
| | | | PE = 1 SV = 3 |
| 0.7839 | 0.9968 | Adipoq | Q60994\|ADIPO_MOUSE Adiponectin OS = Mus musculus GN = Adipoq |
| | | | PE = 1 SV = 2 |
| 0.7947 | 0.9968 | Col1a1 | P11087\|CO1A1_MOUSE Collagen alpha-1(I) chain OS = Mus musculus |
| | | | GN = Col1a1 PE = 1 SV = 4 |
| 0.7949 | 0.9968 | Asap2 | Q7SIG6\|ASAP2_MOUSE Arf-GAP with SH3 domain, ANK repeat and PH |
| | | | domain-containing protein 2 OS = Mus musculus GN = Asap2 PE = 1 SV = 3 |
| 0.7973 | 0.9968 | 1 SV | Q8R1Y2\|CP045_MOUSE Uncharacterized protein C16orf45 homolog |
| | | | OS = Mus musculus PE = 1 SV = 1 |
| 0.8023 | 0.9968 | Krt14 | Q61781\|K1C14_MOUSE Keratin, type I cytoskeletal 14 OS = Mus musculus |
| | | | GN = Krt14 PE = 1 SV = 2 |
| 0.8087 | 0.9968 | Krt75 | Q8BGZ7\|K2C75_MOUSE Keratin, type II cytoskeletal 75 OS = Mus |
| | | | musculus GN = Krt75 PE = 1 SV = 1 |
| 0.8117 | 0.9968 | Anxa6 | P14824\|ANXA6_MOUSE Annexin A6 OS = Mus musculus GN = Anxa6 PE = 1 |
| | | | SV = 3 |
| 0.8119 | 0.9968 | Tcap | O70548\|TELT_MOUSE Telethonin OS = Mus musculus GN = Tcap PE = 1 SV = 1 |
| 0.8162 | 0.9968 | Cpb2 | Q9JHH6\|CBPB2_MOUSE Carboxypeptidase B2 OS = Mus musculus |
| | | | GN = Cpb2 PE = 1 SV = 1 |
| 0.8185 | 0.9968 | Atf4 | Q06507\|ATF4_MOUSE Cyclic AMP-dependent transcription factor ATF-4 |
| | | | OS = Mus musculus GN = Atf4 PE = 1 SV = 2 |
| 0.8230 | 0.9968 | Ldb3 | Q9JKS4\|LDB3_MOUSE LIM domain-binding protein 3 OS = Mus musculus |
| | | | GN = Ldb3 PE = 1 SV = 1 |
| 0.8239 | 0.9968 | Calml3 | Q9D6P8\|CALL3_MOUSE Calmodulin-like protein 3 OS = Mus musculus |
| | | | GN = Calml3 PE = 2 SV = 1 |
| 0.8263 | 0.9968 | Ighg1 | P01868\|IGHG1_MOUSE Ig gamma-1 chain C region secreted form |
| | | | OS = Mus musculus GN = Ighg1 PE = 1 SV = 1 |
| 0.8299 | 0.9968 | Trim33 | Q99PP7\|TRI33_MOUSE E3 ubiquitin-protein ligase TRIM33 OS = Mus |
| | | | musculus GN = Trim33 PE = 1 SV = 2 |
| 0.8320 | 0.9968 | Chat | Q03059\|CLAT_MOUSE Choline O-acetyltransferase OS = Mus musculus |
| | | | GN = Chat PE = 2 SV = 2 |
| 0.8337 | 0.9968 | Acaa1b | Q8VCH0\|THIKB_MOUSE 3-ketoacyl-CoA thiolase B, peroxisomal OS = Mus |
| | | | musculus GN = Acaa1b PE = 1 SV = 1 |
| 0.8363 | 0.9968 | Brd3 | Q8K2F0\|BRD3_MOUSE Bromodomain-containing protein 3 OS = Mus |
| | | | musculus GN = Brd3 PE = 1 SV = 2 |
| 0.8404 | 0.9968 | Tubb4b | P68372\|TBB4B_MOUSE Tubulin beta-4B chain OS = Mus musculus |
| | | | GN = Tubb4b PE = 1 SV = 1 |
| 0.8409 | 0.9968 | Hoxa4 | P06798\|HXA4_MOUSE Homeobox protein Hox-A4 OS = Mus musculus |
| | | | GN = Hoxa4 PE = 2 SV = 4 |
| 0.8463 | 0.9968 | Myl1 | P05977\|MYL1_MOUSE Myosin light chain 1/3, skeletal muscle isoform |
| | | | OS = Mus musculus GN = Myl1 PE = 1 SV = 2 |
| 0.8468 | 0.9968 | Spata6l | B2RV46\|SPA6L_MOUSE Spermatogenesis associated 6-like protein |
| | | | OS = Mus musculus GN = Spata6l PE = 1 SV = 1 |
| 0.8477 | 0.9968 | S100a8 | P27005\|S10A8_MOUSE Protein S100-A8 OS = Mus musculus GN = S100a8 |
| | | | PE = 1 SV = 3 |
| 0.8502 | 0.9968 | Riok3 | Q9DBU3\|RIOK3_MOUSE Serine/threonine-protein kinase RIO3 OS = Mus |
| | | | musculus GN = Riok3 PE = 1 SV = 3 |
| 0.8569 | 0.9968 | Alms1 | Q8K4EO\|ALMS1_MOUSE Alstrom syndrome protein 1 homolog OS = Mus |
| | | | musculus GN = Alms1 PE = 1 SV = 2 |
| 0.8603 | 0.9968 | Jup | Q02257\|PLAK_MOUSE Junction plakoglobin OS = Mus musculus GN = Jup |
| | | | PE = 1 SV = 3 |
| 0.8609 | 0.9968 | Krt35 | Q497I4\|KRT35_MOUSE Keratin, type I cuticular Ha5 OS = Mus musculus |
| | | | GN = Krt35 PE = 1 SV = 1 |
| 0.8614 | 0.9968 | Isoc2a | P85094\|ISC2A_MOUSE Isochorismatase domain-containing protein 2A |
| | | | OS = Mus musculus GN = lsoc2a PE = 1 SV = 1 |
| 0.8631 | 0.9968 | Myh1 | Q5SX40\|MYH1_MOUSE Myosin-1 OS = Mus musculus GN = Myh1 PE = 1 SV = 1 |
| 0.8703 | 0.9968 | Krt76 | Q3UV17\|K22O_MOUSE Keratin, type II cytoskeletal 2 oral OS = Mus |
| | | | musculus GN = Krt76 PE = 1 SV = 1 |
| 0.8706 | 0.9968 | Grem1 | O70326\|GREM1_MOUSE Gremlin-1 OS = Mus musculus GN = Grem1 PE = 2 |
| | | | SV = 1 |
| 0.8752 | 0.9968 | Gnmt | Q9QXF8\|GNMT_MOUSE Glycine N-methyltransferase OS = Mus musculus |
| | | | GN = Gnmt PE = 1 SV = 3 |
| 0.8818 | 0.9968 | Elavl2 | Q60899\|ELAV2_MOUSE ELAV-like protein 2 OS = Mus musculus |
| | | | GN = Elavl2 PE = 2 SV = 1 |
| 0.8821 | 0.9968 | Hcls1 | P49710\|HCLS1_MOUSE Hematopoietic lineage cell-specific protein |
| | | | OS = Mus musculus GN = Hcls1 PE = 1 SV = 2 |
| 0.8828 | 0.9968 | Krt34 | Q9D646\|KRT34_MOUSE Keratin, type I cuticular Ha4 OS = Mus musculus |
| | | | GN = Krt34 PE = 2 SV = 1 |
| | | | P35486\|ODPA_MOUSE Pyruvate dehydrogenase E1 component subunit |
| 0.8939 | 0.9968 | Pdha1 | alpha, somatic form, mitochondrial OS = Mus musculus GN = Pdha1 PE = 1 |
| | | | SV = 1 |
| 0.8978 | 0.9968 | Krt17 | Q9QWL7\|K1C17_MOUSE Keratin, type I cytoskeletal 17 OS = Mus |
| | | | musculus GN = Krt17 PE = 1 SV = 3 |
| 0.9006 | 0.9968 | Hivep2 | Q3UHF7\|ZEP2_MOUSE Transcription factor HIVEP2 OS = Mus musculus |
| | | | GN = Hivep2 PE = 1 SV = 1 |
| 0.9008 | 0.9968 | Mrps2 | Q924T2\|RT02_MOUSE 28S ribosomal protein S2, mitochondrial OS = Mus |
| | | | musculus GN = Mrps2 PE = 1 SV = 1 |
| 0.9042 | 0.9968 | Ica1 | P97411\|ICA69_MOUSE Islet cell autoantigen 1 OS = Mus musculus |
| | | | GN = lca1 PE = 1 SV = 3 |
| 0.9060 | 0.9968 | Col11a2 | Q64739\|COBA2_MOUSE Collagen alpha-2(XI) chain OS = Mus musculus |
| | | | GN = Col11a2 PE = 2 SV = 3 |
| 0.9131 | 0.9968 | H2afz | P0C0S6\|H2AZ_MOUSE Histone H2A.Z OS = Mus musculus GN = H2afz |
| | | | PE = 1 SV = 2 |
| 0.9158 | 0.9968 | Flnc | Q8VHX6\|FLNC_MOUSE Filamin-C OS = Mus musculus GN = Flnc PE = 1 |
| | | | SV = 3 |
| 0.9186 | 0.9968 | Mybpc2 | Q5XKEO\|MYPC2_MOUSE Myosin-binding protein C, fast-type OS = Mus |
| | | | musculus GN = Mybpc2 PE = 1 SV = 1 |
| 0.9245 | 0.9968 | Mtco2 | P00405\|COX2_MOUSE Cytochrome c oxidase subunit 2 OS = Mus |
| | | | musculus GN = Mtco2 PE = 1 SV = 1 |
| 0.9248 | 0.9968 | Vtn | P29788\|VTNC_MOUSE Vitronectin OS = Mus musculus GN = Vtn PE = 1 |
| | | | SV = 2 |
| 0.9264 | 0.9968 | Tmprss6 | Q9DBIO\|TMPS6_MOUSE Transmembrane protease serine 6 OS = Mus |
| | | | musculus GN = Tmprss6 PE = 1 SV = 4 |
| 0.9271 | 0.9968 | Rtn1 | Q8KOTO\|RTN1_MOUSE Reticulon-1 OS = Mus musculus GN = Rtn1 PE = 1 |
| | | | SV = 1 |
| 0.9285 | 0.9968 | Krt8 | P11679\|K2C8_MOUSE Keratin, type II cytoskeletal 8 OS = Mus musculus |
| | | | GN = Krt8 PE = 1 SV = 4 |
| 0.9296 | 0.9968 | Col3a1 | P08121\|CO3A1_MOUSE Collagen alpha-1 (III) chain OS = Mus musculus |
| | | | GN = Col3a1 PE = 1 SV = 4 |
| 0.9302 | 0.9968 | Col12a1 | Q60847\|COCA1_MOUSE Collagen alpha-1 (XII) chain OS = Mus musculus |
| | | | GN = Col12a1 PE = 2 SV = 3 |
| 0.9343 | 0.9968 | Mknk2 | Q8CDBO\|MKNK2_MOUSE MAP kinase-interacting serine/threonine-protein |
| | | | kinase 2 OS = Mus musculus GN = Mknk2 PE = 1 SV = 3 |
| 0.9358 | 0.9968 | Anxa5 | P48036\|ANXA5_MOUSE Annexin A5 OS = Mus musculus GN = Anxa5 PE = 1 |
| | | | SV = 1 |
| 0.9387 | 0.9968 | Timmdc1 | Q8BUY5\|TIDC1_MOUSE Complex I assembly factor TIMMDC1, |
| | | | mitochondrial OS = Mus musculus GN = Timmdc1 PE = 1 SV = 1 |
| 0.9407 | 0.9968 | Acat1 | Q8QZT1\|THIL_MOUSE Acetyl-CoA acetyltransferase, mitochondrial |
| | | | OS = Mus musculus GN = Acat1 PE = 1 SV = 1 |
| 0.9418 | 0.9968 | Eif3a | P23116\|EIF3A_MOUSE Eukaryotic translation initiation factor 3 subunit A |
| | | | OS = Mus musculus GN = Eif3a PE = 1 SV = 5 |
| 0.9522 | 0.9968 | Bmp2k | Q91Z96\|BMP2K_MOUSE BMP-2-inducible protein kinase OS = Mus |
| | | | musculus GN = Bmp2k PE = 1 SV = 1 |
| 0.9533 | 0.9968 | Krt5 | Q922U2\|K2C5_MOUSE Keratin, type II cytoskeletal 5 OS = Mus musculus |
| | | | GN = Krt5 PE = 1 SV = 1 |
| 0.9537 | 0.9968 | Ccdc18 | Q640L5\|CCD18_MOUSE Coiled-coil domain-containing protein 18 |
| | | | OS = Mus musculus GN = Ccdc18 PE = 1 SV = 1 |
| 0.9581 | 0.9968 | Gsk3a | Q2NL51\|GSK3A_MOUSE Glycogen synthase kinase-3 alpha OS = Mus |
| | | | musculus GN = Gsk3a PE = 1 SV = 2 |
| | | | E9Q4S1\|PDE8B_MOUSE High affinity cAMP-specific and IBMX-insensitive |
| 0.9617 | 0.9968 | Pde8b | 3′,5′-cyclic phosphodiesterase 8B OS = Mus musculus GN = Pde8b PE = 1 |
| | | | SV = 1 |
| 0.9632 | 0.9968 | Krt2 | Q3TTY5\|K22E_MOUSE Keratin, type II cytoskeletal 2 epidermal OS = Mus |
| | | | musculus GN = Krt2 PE = 1 SV = 1 |
| 0.9716 | 0.9968 | Actb | P60710\|ACTB_MOUSE Actin, cytoplasmic 1 OS = Mus musculus GN = Actb |
| | | | PE = 1 SV = 1 |
| 0.9753 | 0.9968 | Krt1 | P04104\|K2C1_MOUSE Keratin, type II cytoskeletal 1 OS = Mus musculus |
| | | | GN = Krt1 PE = 1 SV = 4 |
| 0.9774 | 0.9968 | Krt72 | Q6IME9\|K2C72_MOUSE Keratin, type II cytoskeletal 72 OS = Mus musculus |
| | | | GN = Krt72 PE = 3 SV = 1 |
| 0.9777 | 0.9968 | Cd22 | P35329\|CD22_MOUSE B-cell receptor CD22 OS = Mus musculus GN = Cd22 |
| | | | PE = 1 SV = 1 |
| 0.9781 | 0.9968 | Krt79 | Q8VED5\|K2C79_MOUSE Keratin, type II cytoskeletal 79 OS = Mus |
| | | | musculus GN = Krt79 PE = 1 SV = 2 |
| 0.9810 | 0.9968 | Krt24 | A1L317\|K1C24_MOUSE Keratin, type I cytoskeletal 24 OS = Mus musculus |
| | | | GN = Krt24 PE = 2 SV = 2 |
| 0.9823 | 0.9968 | Krtap19-5 | O08632\|KR195_MOUSE Keratin-associated protein 19-5 OS = Mus |
| | | | musculus GN = Krtap19-5 PE = 2 SV = 1 |
| 0.9834 | 0.9968 | Anxa2 | P07356\|ANXA2_MOUSE Annexin A2 OS = Mus musculus GN = Anxa2 PE = 1 |
| | | | SV = 2 |
| 0.9863 | 0.9968 | Serpinc1 | P32261\|ANT3_MOUSE Antithrombin-Ill OS = Mus musculus GN = Serpinc1 |
| | | | PE = 1 SV = 1 |
| 0.9888 | 0.9968 | 9913 GN | P02769\|ALBU_BOVIN Serum albumin OS = Bos taurus OX = 9913 GN = ALB |
| | | | PE = 1 SV = 4 |
| 0.9892 | 0.9968 | Bcl2l14 | Q9CPT0\|B2L14_MOUSE Apoptosis facilitator Bcl-2-like protein 14 OS = Mus |
| | | | musculus GN = Bcl2l14 PE = 1 SV = 1 |
| 0.9903 | 0.9968 | Banp | Q8VBU8\|BANP_MOUSE Protein BANP OS = Mus musculus GN = Banp |
| | | | PE = 1 SV = 1 |
| 0.9928 | 0.9968 | Med19 | Q8C1S0\|MED19_MOUSE Mediator of RNA polymerase II transcription |
| | | | subunit 19 OS = Mus musculus GN = Med19 PE = 1 SV = 1 |
| 0.9928 | 0.9968 | Arg1 | Q61176\|ARGI1_MOUSE Arginase-1 OS = Mus musculus GN = Arg1 PE = 1 |
| | | | SV = 1 |
| 0.9930 | 0.9968 | Zmym3 | Q9JLM4\|ZMYM3_MOUSE Zinc finger MYM-type protein 3 OS = Mus |
| | | | musculus GN = Zmym3 PE = 1 SV = 1 |
| 0.9939 | 0.9968 | Upf1 | Q9EPU0\|RENT1_MOUSE Regulator of nonsense transcripts 1 OS = Mus |
| | | | musculus GN = Upf1 PE = 1 SV = 2 |
| 0.9940 | 0.9968 | Slc25a4 | P48962\|ADT1_MOUSE ADP/ATP translocase 1 OS = Mus musculus |
| | | | GN = Slc25a4 PE = 1 SV = 4 |
| 0.9969 | 0.9969 | Ndrg2 | Q9QYG0\|NDRG2_MOUSE Protein NDRG2 OS = Mus musculus GN = Ndrg2 |
| | | | PE = 1 SV = 1 |

TABLE 4
Identified proteins in cecal samples

| Anova | q | Gene | |
|---|---|---|---|
| (P) | Value | symbol | Uniprot Assecion/Description |
| 0.8723 | 0.3908 | Lamb1 | P02469\|LAMB1_MOUSE Laminin subunit beta-1 OS = Mus musculus |
| | | | GN = Lamb1 PE = 1 SV = 3 |
| 0.6294 | 0.3158 | Ubac1 | Q8VDI7\|UBAC1_MOUSE Ubiquitin-associated domain-containing protein 1 |
| | | | OS = Mus musculus GN = Ubac1 PE = 1 SV = 2 |
| 0.6729 | 0.3342 | Col4a2 | P08122\|CO4A2_MOUSE Collagen alpha-2(IV) chain OS = Mus musculus |
| | | | GN = Col4a2 PE = 1 SV = 4 |
| 0.7067 | 0.3457 | Serpina1c | Q00896\|A1AT3_MOUSE Alpha-1-antitrypsin 1-3 OS = Mus musculus |
| | | | GN = Serpina1c PE = 1 SV = 2 |
| 0.7015 | 0.3457 | Col19a1 | QOVF58\|COJA1_MOUSE Collagen alpha-1(XIX) chain OS = Mus musculus |
| | | | GN = Col19a1 PE = 2 SV = 2 |
| 0.9834 | 0.4212 | Tln1 | P26039\|TLN1_MOUSE Talin-1 OS = Mus musculus GN = Tln1 PE = 1 SV = 2 |
| 0.7963 | 0.3680 | Lamb2 | Q61292\|LAMB2_MOUSE Laminin subunit beta-2 OS = Mus musculus |
| | | | GN = Lamb2 PE = 1 SV = 2 |
| 0.7805 | 0.3636 | Col8a1 | Q00780\|C08A1_MOUSE Collagen alpha-1(VIII) chain OS = Mus musculus |
| | | | GN = Col8a1 PE = 1 SV = 3 |
| 0.7820 | 0.3636 | Cdc37 | Q61081\|CDC37_MOUSE Hsp90 co-chaperone Cdc37 OS = Mus musculus |
| | | | GN = Cdc37 PE = 1 SV = 1 |
| 0.7836 | 0.3636 | Clu | Q06890\|CLUS_MOUSE Clusterin OS = Mus musculus GN = Clu PE = 1 SV = 1 |
| 0.5665 | 0.2931 | Col4a1 | P02463\|CO4A1_MOUSE Collagen alpha-1 (IV) chain OS = Mus musculus |
| | | | GN = Col4a1 PE = 1 SV = 4 |
| 0.7773 | 0.3636 | Krt31 | Q61765\|K1H1_MOUSE Keratin, type I cuticular Ha1 OS = Mus musculus |
| | | | GN = Krt31 PE = 1 SV = 2 |
| 0.7059 | 0.3457 | Postn | Q62009\|POSTN_MOUSE Periostin OS = Mus musculus GN = Postn PE = 1 |
| | | | SV = 2 |
| 0.7311 | 0.3518 | Lama5 | Q61001\|LAMA5_MOUSE Laminin subunit alpha-5 OS = Mus musculus |
| | | | GN = Lama5 PE = 1 SV = 4 |
| 0.5336 | 0.2787 | Epg5 | Q80TA9\|EPG5_MOUSE Ectopic P granules protein 5 homolog OS = Mus |
| | | | musculus GN = Epg5 PE = 1 SV = 2 |
| 0.4417 | 0.2448 | Ino80d | Q66JY2\|IN80D_MOUSE INO80 complex subunit D OS = Mus musculus |
| | | | GN = Ino80d PE = 2 SV = 3 |
| 0.6745 | 0.3342 | Pcca | Q91ZA3\|PCCA_MOUSE Propionyl-CoA carboxylase alpha chain, |
| | | | mitochondrial OS = Mus musculus GN = Pcca PE = 1 SV = 2 |
| 0.5047 | 0.2707 | Lamc1 | P02468\|LAMC1_MOUSE Laminin subunit gamma-1 OS = Mus musculus |
| | | | GN = Lamc1 PE = 1 SV = 2 |
| 0.5284 | 0.2787 | Golga4 | Q91VW5\|GOGA4_MOUSE Golgin subfamily A member 4 OS = Mus |
| | | | musculus GN = Golga4 PE = 1 SV = 2 |
| 0.7500 | 0.3566 | Nid2 | O88322\|NID2_MOUSE Nidogen-2 OS = Mus musculus GN = Nid2 PE = 1 |
| | | | SV = 2 |
| 0.5009 | 0.2699 | Ptrf | O54724\|PTRF_MOUSE Polymerase I and transcript release factor |
| | | | OS = Mus musculus GN = Ptrf PE = 1 SV = 1 |
| 0.8486 | 0.3846 | Lum | P51885\|LUM_MOUSE Lumican OS = Mus musculus GN = Lum PE = 1 SV = 2 |
| 0.6672 | 0.3334 | Hrg | Q9ESB3\|HRG_MOUSE Histidine-rich glycoprotein OS = Mus musculus |
| | | | GN = Hrg PE = 1 SV = 2 |
| 0.4724 | 0.2581 | Vtn | P29788\|VTNC_MOUSE Vitronectin OS = Mus musculus GN = Vtn PE = 1 |
| | | | SV = 2 |
| 0.3921 | 0.2269 | Hspg2 | Q05793\|PGBM_MOUSE Basement membrane-specific heparan sulfate |
| | | | proteoglycan core protein OS = Mus musculus GN = Hspg2 PE = 1 SV = 1 |
| 0.5363 | 0.2787 | Krt6a | P50446\|K2C6A_MOUSE Keratin, type II cytoskeletal 6A OS = Mus |
| | | | musculus GN = Krt6a PE = 1 SV = 3 |
| 0.9820 | 0.4212 | C1qbp | O35658\|C1QBP_MOUSE Complement component 1 Q subcomponent- |
| | | | binding protein, mitochondrial OS = Mus musculus GN = C1qbp PE = 1 SV = 1 |
| 0.9966 | 0.4253 | Banf1 | O54962\|BAF_MOUSE Barrier-to-autointegration factor OS = Mus musculus |
| | | | GN = Banf1 PE = 1 SV = 1 |
| 0.3826 | 0.2226 | Trerf1 | Q8BXJ2\|TREF1_MOUSE Transcriptional-regulating factor 1 OS = Mus |
| | | | musculus GN = Trerf1 PE = 1 SV = 1 |
| 0.4505 | 0.2485 | Col16a1 | Q8BLX7\|COGA1_MOUSE Collagen alpha-1(XVI) chain OS = Mus musculus |
| | | | GN = Col16a1 PE = 1 SV = 2 |
| 0.7593 | 0.3594 | Hnrnpa3 | Q8BG05\|ROA3_MOUSE Heterogeneous nuclear ribonucleoprotein A3 |
| | | | OS = Mus musculus GN = Hnrnpa3 PE = 1 SV = 1 |
| 0.1145 | 0.1425 | Lama4 | P97927\|LAMA4_MOUSE Laminin subunit alpha-4 OS = Mus musculus |
| | | | GN = Lama4 PE = 1 SV = 2 |
| 0.4268 | 0.2411 | Col4a4 | Q9QZR9\|CO4A4_MOUSE Collagen alpha-4(IV) chain OS = Mus musculus |
| | | | GN = Col4a4 PE = 2 SV = 1 |
| 0.4204 | 0.2386 | Tgfbi | P82198\|BGH3_MOUSE Transforming growth factor-beta-induced protein |
| | | | ig-h3 OS = Mus musculus GN = Tgfbi PE = 1 SV = 1 |
| 0.0548 | 0.1425 | Myh11 | O08638\|MYH11_MOUSE Myosin-11 OS = Mus musculus GN = Myh11 PE = 1 |
| | | | SV = 1 |
| 0.1855 | 0.1549 | 1 SV | P01878\|IGHA_MOUSE Ig alpha chain C region OS = Mus musculus PE = 1 |
| | | | SV = 1 |
| 0.7423 | 0.3557 | Serpinf2 | Q61247\|A2AP_MOUSE Alpha-2-antiplasmin OS = Mus musculus |
| | | | GN = Serpinf2 PE = 1 SV = 1 |
| 0.2847 | 0.1819 | Col15a1 | O35206\|COFA1_MOUSE Collagen alpha-1 (XV) chain OS = Mus musculus |
| | | | GN = Col15a1 PE = 1 SV = 2 |
| 0.5073 | 0.2708 | Acta2 | P62737\|ACTA_MOUSE Actin, aortic smooth muscle OS = Mus musculus |
| | | | GN = Acta2 PE = 1 SV = 1 |
| 0.2241 | 0.1622 | Gp2 | Q9D733\|GP2_MOUSE Pancreatic secretory granule membrane major |
| | | | glycoprotein GP2 OS = Mus musculus GN = Gp2 PE = 1 SV = 3 |
| 0.5990 | 0.3019 | Dpt | Q9QZZ6\|DERM_MOUSE Dermatopontin OS = Mus musculus GN = Dpt |
| | | | PE = 1 SV = 1 |
| 0.7176 | 0.3481 | Cep295nl | Q497N6\|C295L_MOUSE CEP295 N-terminal-like protein OS = Mus |
| | | | musculus GN = Cep295nl PE = 1 SV = 1 |
| 0.8454 | 0.3846 | Insrr | Q9WTL4\|INSRR_MOUSE Insulin receptor-related protein OS = Mus |
| | | | musculus GN = lnsrr PE = 1 SV = 2 |
| 0.3395 | 0.2057 | Fn1 | P11276\|FINC_MOUSE Fibronectin OS = Mus musculus GN = Fn1 PE = 1 |
| | | | SV = 4 |
| 0.3616 | 0.2135 | Tgm2 | P21981\|TGM2_MOUSE Protein-glutamine gamma-glutamyltransferase 2 |
| | | | OS = Mus musculus GN = Tgm2 PE = 1 SV = 4 |
| 0.0818 | 0.1425 | Tpm1 | P58771\|TPM1_MOUSE Tropomyosin alpha-1 chain OS = Mus musculus |
| | | | GN = Tpm1 PE = 1 SV = 1 |
| 0.2019 | 0.1573 | Als2 | Q920R0\|ALS2_MOUSE Alsin OS = Mus musculus GN = Als2 PE = 1 SV = 3 |
| 0.2257 | 0.1622 | Anxa6 | P14824\|ANXA6_MOUSE Annexin A6 OS = Mus musculus GN = Anxa6 PE = 1 |
| | | | SV = 3 |
| 0.0129 | 0.1289 | Myl6 | Q60605\|MYL6_MOUSE Myosin light polypeptide 6 OS = Mus musculus |
| | | | GN = Myl6 PE = 1 SV = 3 |
| 0.2331 | 0.1642 | Plg | P20918\|PLMN_MOUSE Plasminogen OS = Mus musculus GN = Plg PE = 1 |
| | | | SV = 3 |
| 0.0968 | 0.1425 | Lama2 | Q60675\|LAMA2_MOUSE Laminin subunit alpha-2 OS = Mus musculus |
| | | | GN = Lama2 PE = 1 SV = 2 |
| 0.0477 | 0.1425 | Pigr | O70570\|PIGR_MOUSE Polymeric immunoglobulin receptor OS = Mus |
| | | | musculus GN = Pigr PE = 1 SV = 1 |
| 0.2166 | 0.1603 | Selp | Q01102\|LYAM3_MOUSE P-selectin OS = Mus musculus GN = Selp PE = 1 |
| | | | SV = 1 |
| 0.1205 | 0.1425 | Serpina1d | Q00897\|A1AT4_MOUSE Alpha-1-antitrypsin 1-4 OS = Mus musculus |
| | | | GN = Serpina1d PE = 1 SV = 1 |
| 0.0438 | 0.1425 | Muc2 | Q80Z19\|MUC2_MOUSE Mucin-2 (Fragments) OS = Mus musculus |
| | | | GN = Muc2 PE = 1 SV = 2 |
| 0.3395 | 0.2057 | Nid1 | P10493\|NID1_MOUSE Nidogen-1 OS = Mus musculus GN = Nid1 PE = 1 |
| | | | SV = 2 |
| 0.1500 | 0.1440 | Ecm1 | Q61508\|ECM1_MOUSE Extracellular matrix protein 1 OS = Mus musculus |
| | | | GN = Ecm1 PE = 1 SV = 2 |
| 0.0175 | 0.1425 | Tpm2 | P58774\|TPM2_MOUSE Tropomyosin beta chain OS = Mus musculus |
| | | | GN = Tpm2 PE = 1 SV = 1 |
| 0.1566 | 0.1442 | Col3a1 | P08121\|CO3A1_MOUSE Collagen alpha-1(III) chain OS = Mus musculus |
| | | | GN = Col3a1 PE = 1 SV = 4 |
| 0.1840 | 0.1549 | Runx1 | Q03347\|RUNX1_MOUSE Runt-related transcription factor 1 OS = Mus |
| | | | musculus GN = Runx1 PE = 1 SV = 1 |
| 0.1547 | 0.1442 | Fgb | Q8K0E8\|FIBB_MOUSE Fibrinogen beta chain OS = Mus musculus GN = Fgb |
| | | | PE = 1 SV = 1 |
| 0.0897 | 0.1425 | Flna | Q8BTM8\|FLNA_MOUSE Filamin-A OS = Mus musculus GN = Flna PE = 1 |
| | | | SV = 5 |
| 0.4317 | 0.2427 | Pc | Q05920\|PYC_MOUSE Pyruvate carboxylase, mitochondrial OS = Mus |
| | | | musculus GN = Pc PE = 1 SV = 1 |
| 0.1170 | 0.1425 | Fga | E9PV24\|FIBA_MOUSE Fibrinogen alpha chain OS = Mus musculus |
| | | | GN = Fga PE = 1 SV = 1 |
| 0.2825 | 0.1819 | Ltbp1 | Q8CG19\|LTBP1_MOUSE Latent-transforming growth factor beta-binding |
| | | | protein 1 OS = Mus musculus GN = Ltbp1 PE = 1 SV = 2 |
| 0.3414 | 0.2057 | Col6a4 | A2AX52\|CO6A4_MOUSE Collagen alpha-4(VI) chain OS = Mus musculus |
| | | | GN = Col6a4 PE = 1 SV = 2 |
| 0.0919 | 0.1425 | Col5a1 | O88207\|CO5A1_MOUSE Collagen alpha-1(V) chain OS = Mus musculus |
| | | | GN = Col5a1 PE = 1 SV = 2 |
| 0.0264 | 0.1425 | Jchain | P01592\|IGJ_MOUSE Immunoglobulin J chain OS = Mus musculus |
| | | | GN = Jchain PE = 1 SV = 4 |
| 0.0463 | 0.1425 | Synm | Q70IV5\|SYNEM_MOUSE Synemin OS = Mus musculus GN = Synm PE = 1 |
| | | | SV = 2 |
| 0.0754 | 0.1425 | Serpina3k | P07759\|SPA3K_MOUSE Serine protease inhibitor A3K OS = Mus musculus |
| | | | GN = Serpina3k PE = 1 SV = 2 |
| 0.9108 | 0.4019 | Tubb5 | P99024\|TBB5_MOUSE Tubulin beta-5 chain OS = Mus musculus |
| | | | GN = Tubb5 PE = 1 SV = 1 |
| 0.1091 | 0.1425 | Fgg | Q8VCM7\|FIBG_MOUSE Fibrinogen gamma chain OS = Mus musculus |
| | | | GN = Fgg PE = 1 SV = 1 |
| 0.2156 | 0.1603 | Usp18 | Q9WTV6\|UBP18_MOUSE Ubl carboxyl-terminal hydrolase 18 OS = Mus |
| | | | musculus GN = Usp18 PE = 1 SV = 2 |
| 0.1400 | 0.1440 | Serpinc1 | P32261\|ANT3_MOUSE Antithrombin-III OS = Mus musculus GN = Serpinc1 |
| | | | PE = 1 SV = 1 |
| 0.1961 | 0.1573 | Flnc | Q8VHX6\|FLNC_MOUSE Filamin-C OS = Mus musculus GN = Flnc PE = 1 |
| | | | SV = 3 |
| 0.0951 | 0.1425 | Dscaml1 | Q4VA61\|DSCL1_MOUSE Down syndrome cell adhesion molecule-like |
| | | | protein 1 homolog OS = Mus musculus GN = Dscaml1 PE = 1 SV = 2 |
| 0.0824 | 0.1425 | Phkb | Q7TSH2\|KPBB_MOUSE Phosphorylase b kinase regulatory subunit beta |
| | | | OS = Mus musculus GN = Phkb PE = 1 SV = 1 |
| 0.1088 | 0.1425 | Npnt | Q91V88\|NPNT_MOUSE Nephronectin OS = Mus musculus GN = Npnt PE = 1 |
| | | | SV = 1 |
| 0.0626 | 0.1425 | Col5a2 | Q3U962\|CO5A2_MOUSE Collagen alpha-2(V) chain OS = Mus musculus |
| | | | GN = Col5a2 PE = 1 SV = 1 |
| 0.3392 | 0.2057 | Ahsg | P29699\|FETUA_MOUSE Alpha-2-HS-glycoprotein OS = Mus musculus |
| | | | GN = Ahsg PE = 1 SV = 1 |
| 0.9613 | 0.4178 | Krt16 | Q9Z2K1\|K1C16_MOUSE Keratin, type I cytoskeletal 16 OS = Mus musculus |
| | | | GN = Krt16 PE = 1 SV = 3 |
| 0.1269 | 0.1425 | Itln1b | Q80ZA0\|ITL1B_MOUSE Intelectin-1 b OS = Mus musculus GN = Itln1b PE = 1 |
| | | | SV = 1 |
| 0.2508 | 0.1705 | Apoa4 | P06728\|APOA4_MOUSE Apolipoprotein A-IV OS = Mus musculus |
| | | | GN = Apoa4 PE = 1 SV = 3 |
| 0.4770 | 0.2589 | Mfap5 | Q9QZJ6\|MFAP5_MOUSE Microfibrillar-associated protein 5 OS = Mus |
| | | | musculus GN = Mfap5 PE = 1 SV = 1 |
| 0.0374 | 0.1425 | Smtn | Q921U8\|SMTN_MOUSE Smoothelin OS = Mus musculus GN = Smtn PE = 1 |
| | | | SV = 2 |
| 0.1304 | 0.1425 | Col6a2 | Q02788\|CO6A2_MOUSE Collagen alpha-2(VI) chain OS = Mus musculus |
| | | | GN = Col6a2 PE = 1 SV = 3 |
| 0.0238 | 0.1425 | 1 SV | P01786\|HVM17_MOUSE Ig heavy chain V region MOPC 47A OS = Mus |
| | | | musculus PE = 1 SV = 1 |
| 0.0621 | 0.1425 | Fbln2 | P37889\|FBLN2_MOUSE Fibulin-2 OS = Mus musculus GN = Fbln2 PE = 1 |
| | | | SV = 2 |
| 0.0327 | 0.1425 | Synpo2 | Q91YE8\|SYNP2_MOUSE Synaptopodin-2 OS = Mus musculus GN = Synpo2 |
| | | | PE = 1 SV = 2 |
| 0.2282 | 0.1622 | Fbn2 | Q61555\|FBN2_MOUSE Fibrillin-2 OS = Mus musculus GN = Fbn2 PE = 1 |
| | | | SV = 2 |
| 0.0802 | 0.1425 | Col1a1 | P11087\|CO1A1_MOUSE Collagen alpha-1(I) chain OS = Mus musculus |
| | | | GN = Col1a1 PE = 1 SV = 4 |
| 0.0733 | 0.1425 | Fbn1 | Q61554\|FBN1_MOUSE Fibrillin-1 OS = Mus musculus GN = Fbn1 PE = 1 |
| | | | SV = 2 |
| 0.1510 | 0.1440 | Iglc2 | P01844\|LAC2_MOUSE Ig lambda-2 chain C region OS = Mus musculus |
| | | | GN = lglc2 PE = 1 SV = 1 |
| 0.0578 | 0.1425 | Col6a1 | Q04857\|CO6A1_MOUSE Collagen alpha-1 (VI) chain OS = Mus musculus |
| | | | GN = Col6a1 PE = 1 SV = 1 |
| 0.2221 | 0.1622 | Ltbp4 | Q8K4G1\|LTBP4_MOUSE Latent-transforming growth factor beta-binding |
| | | | protein 4 OS = Mus musculus GN = Ltbp4 PE = 1 SV = 2 |
| 0.0598 | 0.1425 | Col1a2 | Q01149\|CO1A2_MOUSE Collagen alpha-2(l) chain OS = Mus musculus |
| | | | GN = Col1a2 PE = 1 SV = 2 |
| 0.0001 | 0.0055 | Dmbt1 | Q60997\|DMBT1_MOUSE Deleted in malignant brain tumors 1 protein |
| | | | OS = Mus musculus GN = Dmbt1 PE = 1 SV = 2 |
| 0.1426 | 0.1440 | Palld | Q9ET54\|PALLD_MOUSE Palladin OS = Mus musculus GN = Palld PE = 1 |
| | | | SV = 2 |
| 0.0374 | 0.1425 | Prelp | Q9JK53\|PRELP_MOUSE Prolargin OS = Mus musculus GN = Prelp PE = 1 |
| | | | SV = 2 |
| 0.4143 | 0.2363 | Col6a5 | A6H584\|CO6A5_MOUSE Collagen alpha-5(VI) chain OS = Mus musculus |
| | | | GN = Col6a5 PE = 1 SV = 4 |
| 0.0431 | 0.1425 | Col18a1 | P39061\|COIA1_MOUSE Collagen alpha-1 (XVIII) chain OS = Mus musculus |
| | | | GN = Col18a1 PE = 1 SV = 4 |
| 0.0125 | 0.1289 | Reg3g | O09049\|REG3G_MOUSE Regenerating islet-derived protein 3-gamma |
| | | | OS = Mus musculus GN = Reg3g PE = 1 SV = 1 |
| 0.0012 | 0.0280 | Reg3b | P35230\|REG3B_MOUSE Regenerating islet-derived protein 3-beta |
| | | | OS = Mus musculus GN = Reg3b PE = 1 SV = 1 |
| 0.1100 | 0.1425 | Loxl1 | P97873\|LOXL1_MOUSE Lysyl oxidase homolog 1 OS = Mus musculus |
| | | | GN = Loxl1 PE = 2 SV = 3 |
| 0.1527 | 0.1440 | Pou3f3 | P31361\|PO3F3_MOUSE POU domain, class 3, transcription factor 3 |
| | | | OS = Mus musculus GN = Pou3f3 PE = 2 SV = 2 |
| 0.1047 | 0.1425 | Col2a1 | P28481 \|CO2A1_MOUSE Collagen alpha-1(II) chain OS = Mus musculus |
| | | | GN = Col2a1 PE = 1 SV = 2 |
| 0.1449 | 0.1440 | Hap1 | O35668\|HAP1_MOUSE Huntingtin-associated protein 1 OS = Mus musculus |
| | | | GN = Hap1 PE = 1 SV = 1 |
| 0.9797 | 0.4212 | Smchd1 | Q6P5D8\|SMHD1_MOUSE Structural maintenance of chromosomes flexible |
| | | | hinge domain-containing protein 1 OS = Mus musculus GN = Smchd1 PE = 1 |
| | | | SV = 2 |
| 0.2756 | 0.1805 | Trpv6 | Q91WD2\|TRPV6_MOUSE Transient receptor potential cation channel |
| | | | subfamily V member 6 OS = Mus musculus GN = Trpv6 PE = 1 SV = 2 |
| 0.1510 | 0.1440 | Svs4 | P18419\|SVS4_MOUSE Seminal vesicle secretory protein 4 OS = Mus |
| | | | musculus GN = Svs4 PE = 1 SV = 2 |
| 0.0885 | 0.1425 | Adamts14 | Q80T21\|ATL4_MOUSE ADAMTS-like protein 4 OS = Mus musculus |
| | | | GN = Adamtsl4 PE = 2 SV = 1 |
| 0.0036 | 0.0707 | Exd2 | Q8VEG4\|EXD2_MOUSE Exonuclease 3′-5′ domain-containing protein 2 |
| | | | OS = Mus musculus GN = Exd2 PE = 1 SV = 2 |
| 0.0001 | 0.0055 | Clca1 | Q9D7Z6\|CLCA1_MOUSE Calcium-activated chloride channel regulator 1 |
| | | | OS = Mus musculus GN = Clca1 PE = 1 SV = 2 |
| 0.0744 | 0.1425 | Ace | P09470\|ACE_MOUSE Angiotensin-converting enzyme OS = Mus musculus |
| | | | GN = Ace PE = 1 SV = 3 |
| 0.3744 | 0.2189 | Krt85 | Q9Z2T6\|KRT85_MOUSE Keratin, type II cuticular Hb5 OS = Mus musculus |
| | | | GN = Krt85 PE = 1 SV = 2 |
| 0.9439 | 0.4118 | Hspb1 | P14602\|HSPB1_MOUSE Heat shock protein beta-1 OS = Mus musculus |
| | | | GN = Hspb1 PE = 1 SV = 3 |
| 0.9342 | 0.4106 | Des | P31001\|DESM_MOUSE Desmin OS = Mus musculus GN = Des PE = 1 SV = 3 |
| 0.8585 | 0.3867 | Hspd1 | P63038\|CH60_MOUSE 60 kDa heat shock protein, mitochondrial OS = Mus |
| | | | musculus GN = Hspd1 PE = 1 SV = 1 |
| 0.8998 | 0.4000 | Ckmt1 | P30275\|KCRU_MOUSE Creatine kinase U-type, mitochondrial OS = Mus |
| | | | musculus GN = Ckmt1 PE = 1 SV = 1 |
| 0.2045 | 0.1573 | Ivl | P48997\|INVO_MOUSE Involucrin OS = Mus musculus GN = lvl PE = 1 SV = 1 |
| 0.8031 | 0.3683 | Zg16 | Q8KOC5\|ZG16_MOUSE Zymogen granule membrane protein 16 OS = Mus |
| | | | musculus GN = Zg16 PE = 1 SV = 1 |
| 0.8864 | 0.3956 | Tinagl1 | Q99JR5\|TINAL_MOUSE Tubulointerstitial nephritis antigen-like OS = Mus |
| | | | musculus GN = Tinagl1 PE = 1 SV = 1 |
| 0.5910 | 0.3005 | Abca3 | Q8R420\|ABCA3_MOUSE ATP-binding cassette sub-family A member 3 |
| | | | OS = Mus musculus GN = Abca3 PE = 1 SV = 3 |
| 0.5169 | 0.2747 | Got2 | P05202\|AATM_MOUSE Aspartate aminotransferase, mitochondrial |
| | | | OS = Mus musculus GN = Got2 PE = 1 SV = 1 |
| 0.7239 | 0.3497 | Emilin1 | Q99K41\|EMIL1_MOUSE EMILIN-1 OS = Mus musculus GN = Emilin1 PE = 1 |
| | | | SV = 1 |
| 0.3518 | 0.2099 | Fndc7 | A2AED3\|FNDC7_MOUSE Fibronectin type III domain-containing protein 7 |
| | | | OS = Mus musculus GN = Fndc7 PE = 2 SV = 1 |
| 0.3542 | 0.2102 | 1 SV | P01837\|IGKC_MOUSE Ig kappa chain C region OS = Mus musculus PE = 1 |
| | | | SV = 1 |
| 0.7832 | 0.3636 | Hnrnpa2b1 | O88569\|ROA2_MOUSE Heterogeneous nuclear ribonucleoproteins A2/B1 |
| | | | OS = Mus musculus GN = Hnrnpa2b1 PE = 1 SV = 2 |
| 0.8294 | 0.3788 | Slc25a4 | P48962\|ADT1_MOUSE ADP/ATP translocase 1 OS = Mus musculus |
| | | | GN = Slc25a4 PE = 1 SV = 4 |
| 0.7998 | 0.3682 | Actn1 | Q7TPR4\|ACTN1_MOUSE Alpha-actinin-1 OS = Mus musculus GN = Actn1 |
| | | | PE = 1 SV = 1 |
| 0.4718 | 0.2581 | Myl9 | Q9CQ19\|MYL9_MOUSE Myosin regulatory light polypeptide 9 OS = Mus |
| | | | musculus GN = Myl9 PE = 1 SV = 3 |
| 0.4341 | 0.2429 | Cnn1 | Q08091\|CNN1_MOUSE Calponin-1 OS = Mus musculus GN = Cnn1 PE = 1 |
| | | | SV = 1 |
| 0.3435 | 0.2059 | Tubb4b | P68372\|TBB4B_MOUSE Tubulin beta-4B chain OS = Mus musculus |
| | | | GN = Tubb4b PE = 1 SV = 1 |
| 0.3157 | 0.1985 | Ptma | P26350\|PTMA_MOUSE Prothymosin alpha OS = Mus musculus GN = Ptma |
| | | | PE = 1 SV = 2 |
| 0.2287 | 0.1622 | Vim | P20152\|VIME_MOUSE Vimentin OS = Mus musculus GN = Vim PE = 1 SV = 3 |
| 0.8599 | 0.3867 | Ca1 | P13634\|CAH1_MOUSE Carbonic anhydrase 1 OS = Mus musculus |
| | | | GN = Ca1 PE = 1 SV = 4 |
| 0.0565 | 0.1425 | Epx | P49290\|PERE_MOUSE Eosinophil peroxidase OS = Mus musculus |
| | | | GN = Epx PE = 1 SV = 2 |
| 0.4782 | 0.2589 | Col12a1 | Q60847\|COCA1_MOUSE Collagen alpha-1(XII) chain OS = Mus musculus |
| | | | GN = Col12a1 PE = 2 SV = 3 |
| 0.7503 | 0.3566 | Prg2 | Q61878\|PRG2_MOUSE Bone marrow proteoglycan OS = Mus musculus |
| | | | GN = Prg2 PE = 1 SV = 1 |
| 0.1486 | 0.1440 | Tagln | P37804\|TAGL_MOUSE Transgelin OS = Mus musculus GN = Tagln PE = 1 |
| | | | SV = 3 |
| 0.2438 | 0.1697 | 1 SV | P01675\|KV6A1_MOUSE Ig kappa chain V-VI region XRPC 44 OS = Mus |
| | | | musculus PE = 1 SV = 1 |
| 0.3949 | 0.2275 | S100a8 | P27005\|S10A8_MOUSE Protein S100-A8 OS = Mus musculus GN = S100a8 |
| | | | PE = 1 SV = 3 |
| 0.1072 | 0.1425 | Alb | P07724\|ALBU_MOUSE Serum albumin OS = Mus musculus GN = Alb PE = 1 |
| | | | SV = 3 |
| 0.5343 | 0.2787 | Tuba1b | P05213\|TBA1B_MOUSE Tubulin alpha-1B chain OS = Mus musculus |
| | | | GN = Tuba1b PE = 1 SV = 2 |
| 0.2764 | 0.1805 | 9913 GN | P02769\|ALBU_BOVIN Serum albumin OS = Bos taurus OX = 9913 GN = ALB |
| | | | PE = 1 SV = 4 |
| 0.1980 | 0.1573 | Hnrnpk | P61979\|HNRPK_MOUSE Heterogeneous nuclear ribonucleoprotein K |
| | | | OS = Mus musculus GN = Hnrnpk PE = 1 SV = 1 |
| 0.1474 | 0.1440 | Colec12 | Q8K4Q8\|COL12_MOUSE Collectin-12 OS = Mus musculus GN = Colec12 |
| | | | PE = 1 SV = 1 |
| 0.5837 | 0.2980 | 1 SV | Streptavidin\|P22629\|SAV_STRAV Streptavidin OS = Streptomyces avidinii |
| | | | PE = 1 SV = 1 |
| 0.9768 | 0.4212 | D1Pas1 | P16381\|DDX3L_MOUSE Putative ATP-dependent RNA helicase PI10 |
| | | | OS = Mus musculus GN = D1Pas1 PE = 1 SV = 1 |
| 0.0337 | 0.1425 | Pglyrp1 | O88593\|PGRP1_MOUSE Peptidoglycan recognition protein 1 OS = Mus |
| | | | musculus GN = Pglyrp1 PE = 1 SV = 1 |
| 0.3384 | 0.2057 | Ezr | P26040\|EZRI_MOUSE Ezrin OS = Mus musculus GN = Ezr PE = 1 SV = 3 |
| 0.1205 | 0.1425 | 1 SV | P01635\|KV5A3_MOUSE Ig kappa chain V-V region K2 (Fragment) |
| | | | OS = Mus musculus PE = 1 SV = 1 |
| 0.0224 | 0.1425 | S100a9 | P31725\|S10A9_MOUSE Protein S100-A9 OS = Mus musculus GN = S100a9 |
| | | | PE = 1 SV = 3 |
| 0.2973 | 0.1879 | Tpm3 | P21107\|TPM3_MOUSE Tropomyosin alpha-3 chain OS = Mus musculus |
| | | | GN = Tpm3 PE = 1 SV = 3 |
| 0.0668 | 0.1425 | Nlrp3 | Q8R4B8\|NLRP3_MOUSE NACHT, LRR and PYD domains-containing |
| | | | protein 3 OS = Mus musculus GN = Nlrp3 PE = 1 SV = 1 |
| 0.0699 | 0.1425 | Cdsn | Q7TPC1\|CDSN_MOUSE Corneodesmosin OS = Mus musculus GN = Cdsn |
| | | | PE = 2 SV = 2 |
| 0.3370 | 0.2057 | Psmb7 | P70195\|PSB7_MOUSE Proteasome subunit beta type-7 OS = Mus |
| | | | musculus GN = Psmb7 PE = 1 SV = 1 |
| 0.0113 | 0.1289 | Hist2h2aa1 | Q6GSS7\|H2A2A_MOUSE Histone H2A type 2-A OS = Mus musculus |
| | | | GN = Hist2h2aa1 PE = 1 SV = 3 |
| 0.7097 | 0.3457 | Mbl2 | P41317\|MBL2_MOUSE Mannose-binding protein C OS = Mus musculus |
| | | | GN = Mbl2 PE = 1 SV = 2 |
| 0.1891 | 0.1557 | Efemp1 | Q8BPB5\|FBLN3_MOUSE EGF-containing fibulin-like extracellular matrix |
| | | | protein 1 OS = Mus musculus GN = Efemp1 PE = 1 SV = 1 |
| 0.1878 | 0.1557 | Krt5 | Q922U2\|K2C5_MOUSE Keratin, type II cytoskeletal 5 OS = Mus musculus |
| | | | GN = Krt5 PE = 1 SV = 1 |
| 0.1844 | 0.1549 | Diras2 | Q5PR73\|DIRA2_MOUSE GTP-binding protein Di-Ras2 OS = Mus musculus |
| | | | GN = Diras2 PE = 1 SV = 1 |
| 0.2143 | 0.1603 | Krt79 | Q8VED5\|K2C79_MOUSE Keratin, type II cytoskeletal 79 OS = Mus |
| | | | musculus GN = Krt79 PE = 1 SV = 2 |
| 0.1778 | 0.1517 | Vcl | Q64727\|VINC_MOUSE Vinculin OS = Mus musculus GN = Vcl PE = 1 SV = 4 |
| 0.1463 | 0.1440 | Lyz1 | P17897\|LYZ1_MOUSE Lysozyme C-1 OS = Mus musculus GN = Lyz1 PE = 1 |
| | | | SV = 1 |
| 0.1365 | 0.1425 | Krt42 | Q6IFX2\|K1C42_MOUSE Keratin, type I cytoskeletal 42 OS = Mus musculus |
| | | | GN = Krt42 PE = 1 SV = 1 |
| 0.0541 | 0.1425 | Itih4 | A6X935\|ITIH4_MOUSE Inter alpha-trypsin inhibitor, heavy chain 4 OS = Mus |
| | | | musculus GN = ltih4 PE = 1 SV = 2 |
| 0.1360 | 0.1425 | S100a11 | P50543\|S10AB_MOUSE Protein S100-A11 OS = Mus musculus |
| | | | GN = S100a11 PE = 1 SV = 1 |
| 0.2346 | 0.1642 | Krt1 | P04104\|K2C1_MOUSE Keratin, type II cytoskeletal 1 OS = Mus musculus |
| | | | GN = Krt1 PE = 1 SV = 4 |
| 0.3207 | 0.2005 | Krt77 | Q6IFZ6\|K2C1B_MOUSE Keratin, type II cytoskeletal 1b OS = Mus musculus |
| | | | GN = Krt77 PE = 1 SV = 1 |
| 0.5334 | 0.2787 | Krt14 | Q61781\|K1C14_MOUSE Keratin, type I cytoskeletal 14 OS = Mus musculus |
| | | | GN = Krt14 PE = 1 SV = 2 |
| 0.1615 | 0.1448 | Krt76 | Q3UV17\|K22O_MOUSE Keratin, type II cytoskeletal 2 oral OS = Mus |
| | | | musculus GN = Krt76 PE = 1 SV = 1 |
| 0.1949 | 0.1573 | Arg1 | Q61176\|ARGI1_MOUSE Arginase-1 OS = Mus musculus GN = Arg1 PE = 1 |
| | | | SV = 1 |
| 0.1069 | 0.1425 | Cyfip2 | Q5SQX6\|CYFP2_MOUSE Cytoplasmic FMR1-interacting protein 2 |
| | | | OS = Mus musculus GN = Cyfip2 PE = 1 SV = 2 |
| 0.5742 | 0.2945 | Pkd1l3 | Q2EG98\|PK1L3_MOUSE Polycystic kidney disease protein 1 -like 3 |
| | | | OS = Mus musculus GN = Pkd1l3 PE = 1 SV = 2 |
| 0.2750 | 0.1805 | Calml3 | Q9D6P8\|CALL3_MOUSE Calmodulin-like protein 3 OS = Mus musculus |
| | | | GN = Calml3 PE = 2 SV = 1 |
| 0.0990 | 0.1425 | Nup214 | Q80U93\|NU214_MOUSE Nuclear pore complex protein Nup214 OS = Mus |
| | | | musculus GN = Nup214 PE = 1 SV = 2 |
| 0.0850 | 0.1425 | Atp5a1 | Q03265\|ATPA_MOUSE ATP synthase subunit alpha, mitochondrial |
| | | | OS = Mus musculus GN = Atp5a1 PE = 1 SV = 1 |
| 0.0251 | 0.1425 | Hist1h2bc | Q6ZWY9\|H2B1C_MOUSE Histone H2B type 1-C/E/G OS = Mus musculus |
| | | | GN = Hist1h2bc PE = 1 SV = 3 |
| 0.1236 | 0.1425 | Prdx1 | P35700\|PRDX1_MOUSE Peroxiredoxin-1 OS = Mus musculus GN = Prdx1 |
| | | | PE = 1 SV = 1 |
| 0.0880 | 0.1425 | Krt2 | Q3TTY5\|K22E_MOUSE Keratin, type II cytoskeletal 2 epidermal OS = Mus |
| | | | musculus GN = Krt2 PE = 1 SV = 1 |
| 0.1647 | 0.1448 | Ppia | P17742\|PPIA_MOUSE Peptidyl-prolyl cis-trans isomerase A OS = Mus |
| | | | musculus GN = Ppia PE = 1 SV = 2 |
| 0.1570 | 0.1442 | Ykt6 | Q9CQW1\|YKT6_MOUSE Synaptobrevin homolog YKT6 OS = Mus |
| | | | musculus GN = Ykt6 PE = 1 SV = 1 |
| 0.0050 | 0.0836 | Hist1h1c | P15864\|H12_MOUSE Histone H1.2 OS = Mus musculus GN = Hist1h1c |
| | | | PE = 1 SV = 2 |
| 0.2000 | 0.1573 | Nes | Q6P5H2\|NEST_MOUSE Nestin OS = Mus musculus GN = Nes PE = 1 SV = 1 |
| 0.2010 | 0.1573 | Gapdh | P16858\|G3P_MOUSE Glyceraldehyde-3-phosphate dehydrogenase |
| | | | OS = Mus musculus GN = Gapdh PE = 1 SV = 2 |
| 0.1423 | 0.1440 | Krt17 | Q9QWL7\|K1C17_MOUSE Keratin, type I cytoskeletal 17 OS = Mus |
| | | | musculus GN = Krt17 PE = 1 SV = 3 |
| 0.1292 | 0.1425 | Dsp | E9Q557\|DESP_MOUSE Desmoplakin OS = Mus musculus GN = Dsp PE = 1 |
| | | | SV = 1 |
| 0.0931 | 0.1425 | Spag5 | Q7TME2\|SPAG5_MOUSE Sperm-associated antigen 5 OS = Mus musculus |
| | | | GN = Spag5 PE = 1 SV = 1 |
| 0.1008 | 0.1425 | Pde8b | E9Q4S1\|PDE8B_MOUSE High affinity cAMP-specific and IBMX-insensitive |
| | | | 3′,5′-cyclic phosphodiesterase 8B OS = Mus musculus GN = Pde8b PE = 1 |
| | | | SV = 1 |
| 0.5985 | 0.3019 | Lipc | P27656\|LIPC_MOUSE Hepatic triacylglycerol lipase OS = Mus musculus |
| | | | GN = Lipc PE = 2 SV = 2 |
| 0.1062 | 0.1425 | Krt75 | Q8BGZ7\|K2C75_MOUSE Keratin, type II cytoskeletal 75 OS = Mus |
| | | | musculus GN = Krt75 PE = 1 SV = 1 |
| 0.0896 | 0.1425 | Txn | P10639\|THIO_MOUSE Thioredoxin OS = Mus musculus GN = Txn PE = 1 |
| | | | SV = 3 |
| 0.0363 | 0.1425 | S100a6 | P14069\|S10A6_MOUSE Protein S100-A6 OS = Mus musculus GN = S100a6 |
| | | | PE = 1 SV = 3 |
| 0.1519 | 0.1440 | Jup | Q02257\|PLAK_MOUSE Junction plakoglobin OS = Mus musculus GN = Jup |
| | | | PE = 1 SV = 3 |
| 0.0615 | 0.1425 | Actb | P60710\|ACTB_MOUSE Actin, cytoplasmic 1 OS = Mus musculus GN = Actb |
| | | | PE = 1 SV = 1 |
| 0.1293 | 0.1425 | Krt10 | P02535\|K1C10_MOUSE Keratin, type I cytoskeletal 10 OS = Mus musculus |
| | | | GN = Krt10 PE = 1 SV = 3 |
| 0.1104 | 0.1425 | Mpo | P11247\|PERM_MOUSE Myeloperoxidase OS = Mus musculus GN = Mpo |
| | | | PE = 1 SV = 2 |
| 0.0391 | 0.1425 | H3f3c | P02301\|H3C_MOUSE Histone H3.3C OS = Mus musculus GN = H3f3c PE = 3 |
| | | | SV = 3 |
| 0.1760 | 0.1513 | Krt71 | Q9ROH5\|K2C71_MOUSE Keratin, type II cytoskeletal 71 OS = Mus |
| | | | musculus GN = Krt71 PE = 1 SV = 1 |
| 0.0523 | 0.1425 | Dsg1b | Q7TSF1\|DSG1B_MOUSE Desmoglein-1-beta OS = Mus musculus |
| | | | GN = Dsg1b PE = 1 SV = 1 |
| 0.0277 | 0.1425 | Hspa2 | P17156\|HSP72_MOUSE Heat shock-related 70 kDa protein 2 OS = Mus |
| | | | musculus GN = Hspa2 PE = 1 SV = 2 |
| 0.0064 | 0.0932 | Apoa1 | Q00623\|APOA1_MOUSE Apolipoprotein A-I OS = Mus musculus |
| | | | GN = Apoa1 PE = 1 SV = 2 |
| 0.0626 | 0.1425 | Pkm | P52480\|KPYM_MOUSE Pyruvate kinase PKM OS = Mus musculus |
| | | | GN = Pkm PE = 1 SV = 4 |
| 0.0213 | 0.1425 | 1 SV | P01631\|KV2A7_MOUSE Ig kappa chain V-II region 26-10 OS = Mus |
| | | | musculus PE = 1 SV = 1 |
| 0.0001 | 0.0055 | Hba | P01942\|HBA_MOUSE Hemoglobin subunit alpha OS = Mus musculus |
| | | | GN = Hba PE = 1 SV = 2 |
| 0.0913 | 0.1425 | Plec | Q9QXS1\|PLEC_MOUSE Plectin OS = Mus musculus GN = Plec PE = 1 SV = 3 |
| 0.0012 | 0.0280 | Hbb-b1 | P02088\|HBB1_MOUSE Hemoglobin subunit beta-1 OS = Mus musculus |
| | | | GN = Hbb-b1 PE = 1 SV = 2 |
| 0.4362 | 0.2429 | Eef1a1 | P10126\|EF1A1_MOUSE Elongation factor 1-alpha 1 OS = Mus musculus |
| | | | GN = Eef1a1 PE = 1 SV = 3 |
| 0.1117 | 0.1425 | Anxa2 | P07356\|ANXA2_MOUSE Annexin A2 OS = Mus musculus GN = Anxa2 PE = 1 |
| | | | SV = 2 |
| 0.1038 | 0.1425 | Tmprss13 | Q5U405\|TMPSD_MOUSE Transmembrane protease serine 13 OS = Mus |
| | | | musculus GN = Tmprss13 PE = 2 SV = 2 |
| 0.1361 | 0.1425 | Anxa1 | P10107\|ANXA1_MOUSE Annexin A1 OS = Mus musculus GN = Anxa1 PE = 1 |
| | | | SV = 2 |
| 0.9414 | 0.4118 | Krt25 | Q8VCW2\|K1C25_MOUSE Keratin, type I cytoskeletal 25 OS = Mus |
| | | | musculus GN = Krt25 PE = 1 SV = 1 |
| 0.0605 | 0.1425 | Tnc | Q80YX1\|TENA_MOUSE Tenascin OS = Mus musculus GN = Tnc PE = 1 |
| | | | SV = 1 |
| 0.1705 | 0.1477 | Rgs8 | Q8BXT1\|RGS8_MOUSE Regulator of G-protein signaling 8 OS = Mus |
| | | | musculus GN = Rgs8 PE = 1 SV = 1 |
| 0.1924 | 0.1573 | Ubb | P0CG49\|UBB_MOUSE Polyubiquitin-B OS = Mus musculus GN = Ubb PE = 2 |
| | | | SV = 1 |
| 0.1581 | 0.1442 | Psmb4 | P99026\|PSB4_MOUSE Proteasome subunit beta type-4 OS = Mus |
| | | | musculus GN = Psmb4 PE = 1 SV = 1 |
| 0.1591 | 0.1442 | Krt80 | Q0VBK2\|K2C80_MOUSE Keratin, type II cytoskeletal 80 OS = Mus |
| | | | musculus GN = Krt80 PE = 1 SV = 1 |
| 0.1703 | 0.1477 | Pkp1 | P97350\|PKP1_MOUSE Plakophilin-1 OS = Mus musculus GN = Pkp1 PE = 1 |
| | | | SV = 1 |
| 0.0132 | 0.1289 | Cfl1 | P18760\|COF1_MOUSE Cofilin-1 OS = Mus musculus GN = Cfl1 PE = 1 SV = 3 |
| 0.2254 | 0.1622 | Myh9 | Q8VDD5\|MYH9_MOUSE Myosin-9 OS = Mus musculus GN = Myh9 PE = 1 |
| | | | SV = 4 |
| 0.2076 | 0.1586 | Tgm1 | Q9JLF6\|TGM1_MOUSE Protein-glutamine gamma-glutamyltransferase K |
| | | | OS = Mus musculus GN = Tgm1 PE = 1 SV = 2 |
| 0.2600 | 0.1737 | Ywhaz | P63101\|1433Z_MOUSE 14-3-3 protein zeta/delta OS = Mus musculus |
| | | | GN = Ywhaz PE = 1 SV = 1 |
| 0.0556 | 0.1425 | Hbb-b2 | P02089\|HBB2_MOUSE Hemoglobin subunit beta-2 OS = Mus musculus |
| | | | GN = Hbb-b2 PE = 1 SV = 2 |
| 0.2751 | 0.1805 | Poli | Q6R3M4\|POLI_MOUSE DNA polymerase iota OS = Mus musculus GN = Poli |
| | | | PE = 1 SV = 1 |
| 0.0683 | 0.1425 | Hsp90ab1 | P11499\|HS90B_MOUSE Heat shock protein HSP 90-beta OS = Mus |
| | | | musculus GN = Hsp90ab1 PE = 1 SV = 3 |
| 0.1253 | 0.1425 | Eef2 | P58252\|EF2_MOUSE Elongation factor 2 OS = Mus musculus GN = Eef2 |
| | | | PE = 1 SV = 2 |
| 0.0462 | 0.1425 | Brap | Q99MP8\|BRAP_MOUSE BRCA1-associated protein OS = Mus musculus |
| | | | GN = Brap PE = 1 SV = 1 |
| 0.0360 | 0.1425 | Krt24 | A1L317\|K1C24_MOUSE Keratin, type I cytoskeletal 24 OS = Mus musculus |
| | | | GN = Krt24 PE = 2 SV = 2 |
| 0.0923 | 0.1425 | Lmna | P48678\|LMNA_MOUSE Prelamin-A/C OS = Mus musculus GN = Lmna PE = 1 |
| | | | SV = 2 |
| 0.1201 | 0.1425 | Prdx2 | Q61171\|PRDX2_MOUSE Peroxiredoxin-2 OS = Mus musculus GN = Prdx2 |
| | | | PE = 1 SV = 3 |
| 0.2158 | 0.1603 | Srcin1 | Q9QWI6\|SRCN1_MOUSE SRC kinase signaling inhibitor 1 OS = Mus |
| | | | musculus GN = Srcin1 PE = 1 SV = 2 |
| 0.4094 | 0.2347 | Rps3 | P62908\|RS3_MOUSE 40S ribosomal protein S3 OS = Mus musculus |
| | | | GN = Rps3 PE = 1 SV = 1 |
| 0.1334 | 0.1425 | Nccrp1 | G3X9C2\|FBX50_MOUSE F-box only protein 50 OS = Mus musculus |
| | | | GN = Nccrp1 PE = 1 SV = 2 |
| 0.0156 | 0.1403 | Mylk | Q6PDN3\|MYLK_MOUSE Myosin light chain kinase, smooth muscle |
| | | | OS = Mus musculus GN = Mylk PE = 1 SV = 3 |
| 0.0473 | 0.1425 | Tgm3 | Q08189\|TGM3_MOUSE Protein-glutamine gamma-glutamyltransferase E |
| | | | OS = Mus musculus GN = Tgm3 PE = 1 SV = 2 |
| 0.5738 | 0.2945 | Krt23 | Q99PS0\|K1C23_MOUSE Keratin, type I cytoskeletal 23 OS = Mus musculus |
| | | | GN = Krt23 PE = 1 SV = 1 |
| 0.1639 | 0.1448 | Cpa4 | Q6P8K8\|CBPA4_MOUSE Carboxypeptidase A4 OS = Mus musculus |
| | | | GN = Cpa4 PE = 2 SV = 2 |
| 0.1049 | 0.1425 | Psmb2 | Q9R1P3\|PSB2_MOUSE Proteasome subunit beta type-2 OS = Mus |
| | | | musculus GN = Psmb2 PE = 1 SV = 1 |
| 0.1283 | 0.1425 | Eno1 | P17182\|ENOA_MOUSE Alpha-enolase OS = Mus musculus GN = Eno1 |
| | | | PE = 1 SV = 3 |
| 0.0536 | 0.1425 | Pgk1 | P09411\|PGK1_MOUSE Phosphoglycerate kinase 1 OS = Mus musculus |
| | | | GN = Pgk1 PE = 1 SV = 4 |
| 0.2476 | 0.1703 | Blmh | Q8R016\|BLMH_MOUSE Bleomycin hydrolase OS = Mus musculus |
| | | | GN = Blmh PE = 1 SV = 1 |
| 0.1641 | 0.1448 | Eif6 | O55135\|IF6_MOUSE Eukaryotic translation initiation factor 6 OS = Mus |
| | | | musculus GN = Eif6 PE = 1 SV = 2 |
| 0.1965 | 0.1573 | Aldh9a1 | Q9JLJ2\|AL9A1_MOUSE 4-trimethylaminobutyraldehyde dehydrogenase |
| | | | OS = Mus musculus GN = Aldh9a1 PE = 1 SV = 1 |
| 0.0884 | 0.1425 | Krt19 | P19001\|K1C19_MOUSE Keratin, type I cytoskeletal 19 OS = Mus musculus |
| | | | GN = Krt19 PE = 1 SV = 1 |
| 0.1157 | 0.1425 | Fabp5 | Q05816\|FABP5_MOUSE Fatty acid-binding protein, epidermal OS = Mus |
| | | | musculus GN = Fabp5 PE = 1 SV = 3 |
| 0.2457 | 0.1700 | Krt35 | Q497I4\|KRT35_MOUSE Keratin, type I cuticular Ha5 OS = Mus musculus |
| | | | GN = Krt35 PE = 1 SV = 1 |
| 0.2836 | 0.1819 | Psmb5 | O55234\|PSB5_MOUSE Proteasome subunit beta type-5 OS = Mus |
| | | | musculus GN = Psmb5 PE = 1 SV = 3 |
| 0.2044 | 0.1573 | Serpina1e | Q00898\|A1AT5_MOUSE Alpha-1-antitrypsin 1-5 OS = Mus musculus |
| | | | GN = Serpina1e PE = 1 SV = 1 |
| 0.3233 | 0.2011 | Vdac2 | Q60930\|VDAC2_MOUSE Voltage-dependent anion-selective channel |
| | | | protein 2 OS = Mus musculus GN = Vdac2 PE = 1 SV = 2 |
| 0.2289 | 0.1622 | Hspa5 | P20029\|GRP78_MOUSE 78 kDa glucose-regulated protein OS = Mus |
| | | | musculus GN = Hspa5 PE = 1 SV = 3 |
| 0.2121 | 0.1603 | Gsdma3 | Q5Y4Y6\|GSDA3_MOUSE Gasdermin-A3 OS = Mus musculus GN = Gsdma3 |
| | | | PE = 1 SV = 1 |
| 0.0720 | 0.1425 | Aldoa | P05064\|ALDOA_MOUSE Fructose-bisphosphate aldolase A OS = Mus |
| | | | musculus GN = Aldoa PE = 1 SV = 2 |
| 0.1013 | 0.1425 | Tpi1 | P17751\|TPIS_MOUSE Triosephosphate isomerase OS = Mus musculus |
| | | | GN = Tpi1 PE = 1 SV = 4 |
| 0.2842 | 0.1819 | Hal | P35492\|HUTH_MOUSE Histidine ammonia-lyase OS = Mus musculus |
| | | | GN = Hal PE = 1 SV = 1 |
| 0.0667 | 0.1425 | Krt73 | Q6NXH9\|K2C73_MOUSE Keratin, type II cytoskeletal 73 OS = Mus |
| | | | musculus GN = Krt73 PE = 1 SV = 1 |
| 0.2497 | 0.1705 | Set | Q9EQU5\|SET_MOUSE Protein SET OS = Mus musculus GN = Set PE = 1 |
| | | | SV = 1 |
| 0.1177 | 0.1425 | Krt13 | P08730\|K1C13_MOUSE Keratin, type I cytoskeletal 13 OS = Mus musculus |
| | | | GN = Krt13 PE = 1 SV = 2 |
| 0.1327 | 0.1425 | Eif4a1 | P60843\|IF4A1_MOUSE Eukaryotic initiation factor 4A-I OS = Mus musculus |
| | | | GN = Eif4a1 PE = 1 SV = 1 |
| 0.2865 | 0.1821 | Pof1b | Q8K4L4\|POF1 B_MOUSE Protein POF1B OS = Mus musculus GN = Pof1 b |
| | | | PE = 2 SV = 3 |
| 0.1333 | 0.1425 | Krt12 | Q64291\|K1C12_MOUSE Keratin, type I cytoskeletal 12 OS = Mus musculus |
| | | | GN = Krt12 PE = 1 SV = 2 |
| 0.0957 | 0.1425 | Psma1 | Q9R1P4\|PSA1_MOUSE Proteasome subunit alpha type-1 OS = Mus |
| | | | musculus GN = Psma1 PE = 1 SV = 1 |
| 0.9054 | 0.401 | Ankrd17 | Q99NHO\|ANR17_MOUSE Ankyrin repeat domain-containing protein 17 |
| | | | OS = Mus musculus GN = Ankrd17 PE = 1 SV = 2 |
| 0.0749 | 0.1425 | Capn1 | O35350\|CAN1_MOUSE Calpain-1 catalytic subunit OS = Mus musculus |
| | | | GN = Capn1 PE = 1 SV = 1 |
| 0.0806 | 0.1425 | Ccdc6 | D3YZP9\|CCDC6_MOUSE Coiled-coil domain-containing protein 6 OS = Mus |
| | | | musculus GN = Ccdc6 PE = 1 SV = 1 |
| 0.0742 | 0.1425 | Psma3 | O70435\|PSA3_MOUSE Proteasome subunit alpha type-3 OS = Mus |
| | | | musculus GN = Psma3 PE = 1 SV = 3 |
| 0.0579 | 0.1425 | Krt4 | P07744\|K2C4_MOUSE Keratin, type II cytoskeletal 4 OS = Mus musculus |
| | | | GN = Krt4 PE = 1 SV = 2 |
| 0.0625 | 0.1425 | Psma6 | Q9QUM9\|PSA6_MOUSE Proteasome subunit alpha type-6 OS = Mus |
| | | | musculus GN = Psma6 PE = 1 SV = 1 |
| 0.1028 | 0.1425 | Psma7 | Q9Z2UO\|PSA7_MOUSE Proteasome subunit alpha type-7 OS = Mus |
| | | | musculus GN = Psma7 PE = 1 SV = 1 |
| 0.2583 | 0.1737 | Ide | Q9JHR7\|IDE_MOUSE Insulin-degrading enzyme OS = Mus musculus |
| | | | GN = Ide PE = 1 SV = 1 |
| 0.0399 | 0.1425 | Ahcy | P50247\|SAHH_MOUSE Adenosylhomocysteinase OS = Mus musculus |
| | | | GN = Ahcy PE = 1 SV = 3 |
| 0.1355 | 0.1425 | Mast1 | Q9R1L5\|MAST1_MOUSE Microtubule-associated serine/threonine-protein |
| | | | kinase 1 OS = Mus musculus GN = Mast1 PE = 1 SV = 3 |
| 0.2597 | 0.1737 | Rpl22 | P67984\|RL22_MOUSE 60S ribosomal protein L22 OS = Mus musculus |
| | | | GN = Rpl22 PE = 1 SV = 2 |
| 0.3739 | 0.2189 | Vdac1 | Q60932\|VDAC1_MOUSE Voltage-dependent anion-selective channel |
| | | | protein 1 OS = Mus musculus GN = Vdac1 PE = 1 SV = 3 |

### Example 8: NHS-Ester Directed Targeting of Molecules into Wound Areas

An essential step in the phenomenon of ECM movement is crosslinking of moved material in in wound areas. Primary amines of proteins and peptides of distinct protein classes are covalently linked. Since the NHS esters also mark primary amines, the Inventors asked ourselves whether the restructuring in wound areas has led to an increase in free amine groups and whether the Inventors can visualize these via intraperitoneal application of NSH-Esters.

### Methods

### Animals

All mouse lines were obtained (C57BL/6J, B6.129P2-Lyz2tm1(cre)lfo/J (Lyz2Cre), B6; 129S6-Gt(ROSA)26Sor^{tm14(CAG-tdTomato)Hze}/J (Ai14)) from Jackson Laboratories or Charles River and bred and maintained at Helmholtz Animal Facility in accordance to the EU directive 2010/63. Animals were housed in individual ventilated cages (IVC) and animal housing rooms were maintained at constant temperature and humidity with a 12-h light cycle. Animals were supplied with water and chow ad libitum. All animal experiments were reviewed and approved by the Government of Upper Bavaria and registered under the project number ROB-55.2-2532.Vet_02-19-133 or ROB-2532.Vet_02-19-148 and conducted under strict governmental and international guidelines. This study is compliant with all relevant ethical regulations regarding animal research.

### Murine Models

Mice received 30 minutes before surgery a preemptive subcutaneous injection with Metamizole (200 mg/kg bw). Anesthesia was supplied by an intraperitoneal injection of a Medetomidin (500 pg/kg), Midazolam (5 mg/kg) and Fentanyl (50 pg/kg) cocktail, hereafter referred to as MMF. Monitoring anesthetic depth was assessed by toe reflex. Eyes were covered with Bepanthen-cream to avoid dehydration, and the abdomen was shaved and disinfected with betadine and sterile phosphate buffered saline (PBS). Animals were kept on their backs on a heating plate at 39° C. A midline laparotomy (1-1.5 cm) was performed through the skin and peritoneum. Four hooks, positioned around the incision and fixed to a retractor and magnetic base plate, allowed for clear access to the abdominal cavity and liver.

Labelling of liver surfaces was performed. Local damage to the liver surface was induced via electroporation tweezers by applying electric voltage: 30V, pulse: 50 ms, interval: 1 second, cycles: 8. Before closure of the incision, Buprenorphine (0.1 mg/kg) was pipetted in the abdomen to allow for initial post-surgical analgesia. For long-term analgesia, Metamizole (Novalgin, 200 mg/kg) was provided through daily injections. The peritoneum and skin were closed with two separate 4-0 silk sutures (Ethicon). Upon closure of the incision, mice were woken up by antagonizing Medetomidin and Midazolam through a subcutaneous cocktail injection of Atipamezol (1 mg/kg) and Flumazenil (0.25 mg/kg). Mice were allowed to recover on a heating pad, after which they were single housed. Mice where sacrificed after indicated time point and liver tissue was obtained. In the peritoneal model, surgical procedure was as described above, but the peritoneal areas were marked.

Inhibitors were injected 2 hours before surgery with a concentration of 10 μM of the corresponding small molecules dissolved in sterile PBS i.p.

### Labelling of ECM Components

Succinimidyl esters (NHS-esters; Thermo Fisher) were diluted in DMSO to 25 mg/ml and stored at −80° C. For local matrix staining labelling solution was generated by mixing NHS-ester 1:1 with 100 mM pH 9.0 sodium bicarbonate buffer. For global abdominal labelling, 20 μl of NHS-labelling solution were mixed with 100 μl sterile PBS and injected i.p.

### Tissue Preparation

Upon organ excision, organs were fixed overnight at 4° C. in 2% formaldehyde. The next day, fixed tissues were washed three times in Dulbecco's phosphate buffered saline (DPBS, GIBCO, #14190-094), and depending on the purpose, either embedded, frozen in optimal cutting temperature compound (Sakura, #4583) and stored at −20° C., or stored at 4° C. in PBS containing 0.2% gelatin (Sigma Aldrich, #G1393), 0.5% Triton X-100 (Sigma Aldrich, #X100) and 0.01% Thimerosal (Sigma Aldrich, #T8784) (PBS-GT). Fixed tissues were embedded in optimal cutting temperature (OCT) and cut with a Microm HM 525 (Thermo Scientific). In short, sections were fixed in ice-cold acetone for 5 min at −20° C., and then washed with PBS. Sections were then blocked for non-specific binding with 10% serum in PBS for 60 minutes at room temperature, and then incubated with primary antibody in blocking solution O/N at 4° C. The next day, following washing, sections were incubated in PBS with fluorescent secondary antibody, for 120 min at RT. Finally, sections were washed and incubated with Hoechst 33342 nucleic acid stain (Invitrogen, #H1399), washed in ddH₂O, mounted with Fluoromount-G® (Southern Biotech, #0100-01), and stored at 4° C. in the dark. Primary antibodies: rabbit-anti-collagen I (1:150, Rockland), rabbit-anti-Cytokeratin (1:100, Sigma Aldrich), rabbit-anti-Ki67 (1:100, Abcam), rabbit-anti-Fibronectin (1:100, Abcam), rabbit-anti-HSP70 (1:100, Elabscience), rabbit-anti-HSPG2 (1:100, Elabscience), rabbit-anti-Keratin9 (1:100, Elabscience), rabbit-anti-Ki67 (1:100, Abcam), rabbit-anti-cleaved Caspase 3 (1:100, Abcam), rabbit-anti-Laminin (1:100, Abcam), rabbit-anti-HSP70 (1:100, Elabscience), hamster-anti-PDPNα (1:100, Abcam), rat-anti-LY6G(Sca1) (1:100, Abcam), rabbit-anti-MMP23(1:100, Elabscience), rabbit-anti-Vitronectin (1:100, Elabscience) and rabbit-anti-WT1 (1:100, Abcam). Alexa Fluor 488-, Alexa Fluor 568- or Alexa Fluor 647-conjugated antibodies (1:500, Life technologies) against suitable species were used as secondary antibodies. H&E stainings where performed according to MMM.

### Microscopy

Histological sections were imaged using a using a M205 FCA Stereomicroscope (Leica). 2D, 3D and 4D data was processed with Imaris 9.1.0 (Bitplane) and ImageJ (1.52i). Contrast and brightness were adjusted for better visibility.

### Proteinbiochemistry

Tissues were snap frozen and grinded using a tissue lyser (Quiagen). Pulverised tissues were resuspended in lysis buffer (20 mM Tris-HCl pH 7.5, 1% Triton X-100, 2% SDS, 100 mM NaCl, 1 mM sodium orthovanandate, 9.5 mM sodium fluoride, 10 mM sodium pyruvate, 10 mM beta-glycerophosphate, and supplemented with protease inhibitors (complete protease inhibitor cocktail, Pierce) and kept 10 min on ice. Samples were sonicated and spinned down for 5 minutes with 10.000g. Supernatants were stored at −80° C. Protein concentration was determined via BCA-Assay according to manufactures protocol (Pierce).

Protein pulldown was as follows. Lysates were diluted with a pulldown buffer (20 mM Tris-HCl pH 7.5, 1% Triton X-100, 100 mM NaCl and supplemented with protease and phosphatase inhibitors) and incubated overnight with dynabeads (Thermo Fisher) according to manufacturer's instructions at 4° C. on a rotator. The next day, the samples were each diluted twice with Wash Buffer 1 (20 mM Tris-HCl pH 7.5, 1% Triton X-100, 2% SDS, 100 mM NaCl and supplemented with protease and phosphatase inhibitors) and then with Wash Buffer 2 (20 mM Tris-HCl pH 7).5, 0.5% Triton X-100, 100 mM NaCl and supplemented with protease and phosphatase inhibitors) and finally washed twice with Wash Buffer 3 (20 mM Tris-HCl pH 7.5 and 100 mM NaCl). Beads were then resuspended in Elution Buffer (20 mM Tris-HCl pH 7.5, 100 mM NaCl and 50 mM DTT) and incubated for 30 minutes in Elution Buffer at 37° C. Finally, the samples were boiled for 5 minutes at 98° C. and the supernatants were stored at −80° C. Fluorescence intensities of lysates were measured were measured in a Fluostar optima fluorometer (BMGlabtech).

### Mass Spectrometry

Tissues were marked locally with an EZ-LINK-NHS 100:1 FITC-NHS mixture. After 24 hours the organs were removed, tissue pieces of the original marking were separated from moved matrix fraction and snap frozen. Tissue lysis was performed as described above. Samples were digested using a modified FASP procedure²⁵. After reduction and alkylation using DTT and IAA, the proteins were centrifuged on Microcon® centrifugal filters (Sartorius Vivacon 500 30 kDa), washed thrice with 8 M urea in 0.1 M Tris/HCl pH 8.5 and twice with 50 mM ammoniumbicarbonate. The proteins on filter were digested for 2 hours at room temperature using 0.5 pg Lys-C (Wako Chemicals, Neuss, Germany) and for 16 hours at 37° C. using 1 pg trypsin (Promega, Mannheim, Germany). Peptides were collected by centrifugation (10 min at 14000 g), acidified with 0.5% TFA and stored at −20° C. until measurements. The digested peptides were loaded automatically to a HPLC system (Thermo Fisher Scientific) equipped with a nano trap column (100 μm ID×2 cm, Acclaim PepMAP 100 C18, 5 μm, 100 Å/size, LC Packings, Thermo Fisher Scientific, Bremen, Germany) in 95% buffer A (2% ACN, 0.1% formic acid (FA) in HPLC-grade water) and 5% buffer B (98% ACN, 0.1% FA in HPLC-grade water) at 30 μl/min. After 5 min, the peptides were eluted and separated on the analytical column (nanoEase MZ HSS T3 Column, 100 Å, 1.8 μm, 75 μm×250 mm, Waters) at 250 nl/min flow rate in a 105 minutes non-linear acetonitrile gradient from 3 to 40% in 0.1% formic acid. The eluting peptides were analyzed online in a Q Exactive HF mass spectrometer (Thermo Fisher Scientific, Bremen, Germany) coupled to the HPLC system with a nano spray ion source and operated in the data-dependent mode. MS spectra were recorded at a resolution of 60,000 and after each MS1 cycle, the 10 most abundant peptide ions were selected for fragmentation. Raw spectra were imported into Progenesis QIsoftware (version 4.1, Nonlinear Dynamics, Waters). After feature alignment and normalization, spectra were exported as Mascot Generic files and searched against the SwissProt mouse database (16,872 sequences) with Mascot (Matrix Science, version 2.6.2) with the following search parameters: 10 ppm peptide mass tolerance and 0.02 Da fragment mass tolerance, two missed cleavages allowed, carbamidomethylation was set as fixed modification, camthiopropanoyl, methionine and proline oxidation were allowed as variable modifications. A Mascot-integrated decoy database search calculated an average false discovery of <1% when searches were performed with a mascot percolator score cut-off of 13 and an appropriate significance threshold p.

Peptide assignments were re-imported into the Progenesis QI software and the abundances of all unique peptides allocated to each protein were summed up. The resulting normalized abundances of the individual proteins were used for calculation of protein ratios and p-values (ANOVA) between sample groups using a nested design. Gene ontology analysis was performed using EnrichR webtool^{26,27}.

### Results

Electroporation of murine livers with a tweezer electrode leads to local damage of the dorsal and ventral side. These round sides of electroporation could be visualized with an NHS-Rhodamine ester by intra peritoneal injection (FIGS. 38a and b).

The discovery the Inventors have made here has many potential implications. The data show that there is an accumulation of primary amines in abdominal wound areas. These can be labelled via NHS-linked reaction. This would allow abdominal wounds to be marked by a simple intra peritoneal injection. By using an NHS ester coupled to deeper wavelength reporters, this would open a new dimension of wound visualization in the clinics. In addition to image wounds, effector molecules, like drugs, could also be coupled to NHS esters to target wound areas with a global injection.

### Example 9: Matrix Motions Elements as Biomarker for Fibrotic Pathologies

Fibrotic processes take place over long periods of time and are usually identified too late. To date, there are no meaningful biomarkers for early stage fibrotic processes.

ECM movement takes place at rapid kinetics. Therefore, the Inventors asked ourselves the question whether parts of the mobilized elements are transferred into the circulating blood stream and whether the Inventors can detect these fluid elements in the blood. These fluid elements can provide information about the stage of a fibrotic process. Since the Inventors have observed that the fluid fractions are organ-specific, the protocol could even provide organ-specific biomarkers.

### Methods

### Animals

All mouse lines were obtained (C57BL/6J, B6.129P2-Lyz2tm1(cre)lfo/J (Lyz2Cre), B6; 129S6-Gt(ROSA)26Sor^{tm14(CAG-tdTomato)Hze}/J (Ai14)) from Jackson Laboratories or Charles River and bred and maintained at Helmholtz Animal Facility in accordance to the EU directive 2010/63. Animals were housed in individual ventilated cages (IVC) and animal housing rooms were maintained at constant temperature and humidity with a 12-h light cycle. Animals were supplied with water and chow ad libitum. All animal experiments were reviewed and approved by the Government of Upper Bavaria and registered under the project number ROB-55.2-2532.Vet_02-19-133 or ROB-2532.Vet_02-19-148 and conducted under strict governmental and international guidelines. This study is compliant with all relevant ethical regulations regarding animal research.

### Murine Models

Mice received 30 minutes before surgery a preemptive subcutaneous injection with Metamizole (200 mg/kg bw). Anesthesia was supplied by an intraperitoneal injection of a Medetomidin (500 pg/kg), Midazolam (5 mg/kg) and Fentanyl (50 pg/kg) cocktail, hereafter referred to as MMF. Monitoring anesthetic depth was assessed by toe reflex. Eyes were covered with Bepanthen-cream to avoid dehydration, and the abdomen was shaved and disinfected with betadine and sterile phosphate buffered saline (PBS). Animals were kept on their backs on a heating plate at 39° C. A midline laparotomy (1-1.5 cm) was performed through the skin and peritoneum. Four hooks, positioned around the incision and fixed to a retractor and magnetic base plate, allowed for clear access to the abdominal cavity and liver.

Labelling of liver surfaces was performed. Local damage to the liver surface was induced via electroporation tweezers by applying electric voltage: 30V, pulse: 50 ms, interval: 1 second, cycles: 8. Before closure of the incision, Buprenorphine (0.1 mg/kg) was pipetted in the abdomen to allow for initial post-surgical analgesia. For long-term analgesia, Metamizole (Novalgin, 200 mg/kg) was provided through daily injections. The peritoneum and skin were closed with two separate 4-0 silk sutures (Ethicon). Upon closure of the incision, mice were woken up by antagonizing Medetomidin and Midazolam through a subcutaneous cocktail injection of Atipamezol (1 mg/kg) and Flumazenil (0.25 mg/kg). Mice were allowed to recover on a heating pad, after which they were single housed. Mice where sacrificed after indicated time point and liver tissue was obtained. In the peritoneal model, surgical procedure was as described above, but the peritoneal areas were marked.

Inhibitors were injected 2 hours before surgery with a concentration of 10 μM of the corresponding small molecules dissolved in sterile PBS i.p.

### Labelling of ECM Components

Succinimidyl esters (NHS-esters; Thermo Fisher) were diluted in DMSO to 25 mg/ml and stored at −80° C. For local matrix staining labelling solution was generated by mixing NHS-ester 1:1 with 100 mM pH 9.0 sodium bicarbonate buffer. For global abdominal labelling, 20 μl of NHS-labelling solution were mixed with 100 μl sterile PBS and injected i.p.

### Tissue Preparation

Upon organ excision, organs were fixed overnight at 4° C. in 2% formaldehyde. The next day, fixed tissues were washed three times in Dulbecco's phosphate buffered saline (DPBS, GIBCO, #14190-094), and depending on the purpose, either embedded, frozen in optimal cutting temperature compound (Sakura, #4583) and stored at −20° C., or stored at 4° C. in PBS containing 0.2% gelatin (Sigma Aldrich, #G1393), 0.5% Triton X-100 (Sigma Aldrich, #X100) and 0.01% Thimerosal (Sigma Aldrich, #T8784) (PBS-GT). Fixed tissues were embedded in optimal cutting temperature (OCT) and cut with a Microm HM 525 (Thermo Scientific). In short, sections were fixed in ice-cold acetone for 5 min at −20° C., and then washed with PBS. Sections were then blocked for non-specific binding with 10% serum in PBS for 60 minutes at room temperature, and then incubated with primary antibody in blocking solution O/N at 4° C. The next day, following washing, sections were incubated in PBS with fluorescent secondary antibody, for 120 min at RT. Finally, sections were washed and incubated with Hoechst 33342 nucleic acid stain (Invitrogen, #H1399), washed in ddH₂O, mounted with Fluoromount-G® (Southern Biotech, #0100-01), and stored at 4° C. in the dark. Primary antibodies: rabbit-anti-collagen I (1:150, Rockland), rabbit-anti-Cytokeratin (1:100, Sigma Aldrich), rabbit-anti-Ki67 (1:100, Abcam), rabbit-anti-Fibronectin (1:100, Abcam), rabbit-anti-HSP70 (1:100, Elabscience), rabbit-anti-HSPG2 (1:100, Elabscience), rabbit-anti-Keratin9 (1:100, Elabscience), rabbit-anti-Ki67 (1:100, Abcam), rabbit-anti-cleaved Caspase 3 (1:100, Abcam), rabbit-anti-Laminin (1:100, Abcam), rabbit-anti-HSP70 (1:100, Elabscience), hamster-anti-PDPNα (1:100, Abcam), rat-anti-LY6G(Sca1) (1:100, Abcam), rabbit-anti-MMP23(1:100, Elabscience), rabbit-anti-Vitronectin (1:100, Elabscience) and rabbit-anti-WT1 (1:100, Abcam). Alexa Fluor 488-, Alexa Fluor 568- or Alexa Fluor 647-conjugated antibodies (1:500, Life technologies) against suitable species were used as secondary antibodies. H&E stainings where performed according to MMM.

### Microscopy

Histological sections were imaged using a using a M205 FCA Stereomicroscope (Leica). 2D, 3D and 4D data was processed with Imaris 9.1.0 (Bitplane) and ImageJ (1.52i). Contrast and brightness were adjusted for better visibility.

### Proteinbiochemistry

Tissues were snap frozen and grinded using a tissue lyser (Quiagen). Pulverised tissues were resuspended in lysis buffer (20 mM Tris-HCl pH 7.5, 1% Triton X-100, 2% SDS, 100 mM NaCl, 1 mM sodium orthovanandate, 9.5 mM sodium fluoride, 10 mM sodium pyruvate, 10 mM beta-glycerophosphate, and supplemented with protease inhibitors (complete protease inhibitor cocktail, Pierce) and kept 10 min on ice. Samples were sonicated and spinned down for 5 minutes with 10.000g. Supernatants were stored at −80° C. Protein concentration was determined via BCA-Assay according to manufactures protocol (Pierce).

Protein pulldown was as follows. Lysates were diluted with a pulldown buffer (20 mM Tris-HCl pH 7.5, 1% Triton X-100, 100 mM NaCl and supplemented with protease and phosphatase inhibitors) and incubated overnight with dynabeads (Thermo Fisher) according to manufacturer's instructions at 4° C. on a rotator. The next day, the samples were each diluted twice with Wash Buffer 1 (20 mM Tris-HCl pH 7.5, 1% Triton X-100, 2% SDS, 100 mM NaCl and supplemented with protease and phosphatase inhibitors) and then with Wash Buffer 2 (20 mM Tris-HCl pH 7).5, 0.5% Triton X-100, 100 mM NaCl and supplemented with protease and phosphatase inhibitors) and finally washed twice with Wash Buffer 3 (20 mM Tris-HCl pH 7.5 and 100 mM NaCl). Beads were then resuspended in Elution Buffer (20 mM Tris-HCl pH 7.5, 100 mM NaCl and 50 mM DTT) and incubated for 30 minutes in Elution Buffer at 37° C. Finally, the samples were boiled for 5 minutes at 98° C. and the supernatants were stored at −80° C. Fluorescence intensities of lysates were measured were measured in a Fluostar optima fluorometer (BMGlabtech).

### Mass Spectrometry

Tissues were marked locally with an EZ-LINK-NHS 100:1 FITC-NHS mixture. After 24 hours the organs were removed, tissue pieces of the original marking were separated from moved matrix fraction and snap frozen. Tissue lysis was performed as described above. Samples were digested using a modified FASP procedure²⁵. After reduction and alkylation using DTT and IAA, the proteins were centrifuged on Microcon® centrifugal filters (Sartorius Vivacon 500 30 kDa), washed thrice with 8 M urea in 0.1 M Tris/HCl pH 8.5 and twice with 50 mM ammoniumbicarbonate. The proteins on filter were digested for 2 hours at room temperature using 0.5 pg Lys-C (Wako Chemicals, Neuss, Germany) and for 16 hours at 37° C. using 1 pg trypsin (Promega, Mannheim, Germany). Peptides were collected by centrifugation (10 min at 14000 g), acidified with 0.5% TFA and stored at −20° C. until measurements. The digested peptides were loaded automatically to a HPLC system (Thermo Fisher Scientific) equipped with a nano trap column (100 μm ID×2 cm, Acclaim PepMAP 100 C18, 5 μm, 100 Å/size, LC Packings, Thermo Fisher Scientific, Bremen, Germany) in 95% buffer A (2% ACN, 0.1% formic acid (FA) in HPLC-grade water) and 5% buffer B (98% ACN, 0.1% FA in HPLC-grade water) at 30 μl/min. After 5 min, the peptides were eluted and separated on the analytical column (nanoEase MZ HSS T3 Column, 100 Å, 1.8 μm, 75 μm×250 mm, Waters) at 250 nl/min flow rate in a 105 minutes non-linear acetonitrile gradient from 3 to 40% in 0.1% formic acid. The eluting peptides were analyzed online in a Q Exactive HF mass spectrometer (Thermo Fisher Scientific, Bremen, Germany) coupled to the HPLC system with a nano spray ion source and operated in the data-dependent mode. MS spectra were recorded at a resolution of 60,000 and after each MS1 cycle, the 10 most abundant peptide ions were selected for fragmentation. Raw spectra were imported into Progenesis QIsoftware (version 4.1, Nonlinear Dynamics, Waters). After feature alignment and normalization, spectra were exported as Mascot Generic files and searched against the SwissProt mouse database (16,872 sequences) with Mascot (Matrix Science, version 2.6.2) with the following search parameters: 10 ppm peptide mass tolerance and 0.02 Da fragment mass tolerance, two missed cleavages allowed, carbamidomethylation was set as fixed modification, camthiopropanoyl, methionine and proline oxidation were allowed as variable modifications. A Mascot-integrated decoy database search calculated an average false discovery of <1% when searches were performed with a mascot percolator score cut-off of 13 and an appropriate significance threshold p.

Peptide assignments were re-imported into the Progenesis QI software and the abundances of all unique peptides allocated to each protein were summed up. The resulting normalized abundances of the individual proteins were used for calculation of protein ratios and p-values (ANOVA) between sample groups using a nested design. Gene ontology analysis was performed using EnrichR webtool^{26,27}.

### Results

Pulmonary fibrosis is a disease that is usually fatal for humans, with no treatment or biomarker options. Therefore, the Inventors tested the biomarker hypothesis in a murine pulmonary fibrosis model. After application of bleomycin, fibrotic plaques develop within the lung in the course of 2 weeks. First, the Inventors checked whether there is mobilization of fluid matrix elements in the model. The Inventors therefore followed 2 setups (FIG. 39a). To check for surface activation of the lungs the Inventors injected intra pleural NHS-FITC and for protein purification in other animals NHS-EZ-LINK. After 2 weeks the Inventors could observe massive recruitment of pleural basal lamina elements into the inner lung (FIG. 39b). Mass spectroscopic analysis of lung tissue and blood revealed proteins significantly enriched in bleomycin treated animals (FIGS. 39c and d).

Bleomycin-induced pulmonary fibrosis has different degrees of severity depending on the animal. Robust biomarkers should therefore show different abundancies depending on the severity of pulmonary fibrosis. First mass spectrometric analyses of lung tissue found varying amounts of proteins in the lungs of bleomycin versus control animals. This indicates that the primarily labelled proteins undergo changes due to the stimulus. Proteins such as fibrinogen are known to form net-like structures. It could be that fibrinogen is covalently bound to the primary labelled proteins. In fact, the Inventors were also able to identify proteins of varying abundance of the initially labelled lung matrix in the blood of the animals. In summary, the Inventors show here that fluid elements enter the blood stream during mobilization of the lung matrix during fibrotic events. These elements can be detected and could serve as biomarkers for fibrotic events.

### Example 10: Matrix Motion Pathway Analysis

Since the ECM movement is a global phenomenon, the Inventors wanted to find out which signaling pathways and mediators play a role in Matrix currents. Here the Matrix studied currents in livers and peritoneas. Here the Inventors investigated matrix currents in livers and peritoneas.

### Methods

### Animals

All mouse lines were obtained (C57BL/6J, B6.129P2-Lyz2tm1(cre)lfo/J (Lyz2Cre), B6; 129S6-Gt(ROSA)26Sor^{tm14(CAG-tdTomato)Hze}/J (Ai14)) from Jackson Laboratories or Charles River and bred and maintained at Helmholtz Animal Facility in accordance to the EU directive 2010/63. Animals were housed in individual ventilated cages (IVC) and animal housing rooms were maintained at constant temperature and humidity with a 12-h light cycle. Animals were supplied with water and chow ad libitum. All animal experiments were reviewed and approved by the Government of Upper Bavaria and registered under the project number ROB-55.2-2532.Vet_02-19-133 or ROB-2532.Vet_02-19-148 and conducted under strict governmental and international guidelines. This study is compliant with all relevant ethical regulations regarding animal research.

### Murine Models

Mice received 30 minutes before surgery a preemptive subcutaneous injection with Metamizole (200 mg/kg bw). Anesthesia was supplied by an intraperitoneal injection of a Medetomidin (500 μg/kg), Midazolam (5 mg/kg) and Fentanyl (50 μg/kg) cocktail, hereafter referred to as MMF. Monitoring anesthetic depth was assessed by toe reflex. Eyes were covered with Bepanthen-cream to avoid dehydration, and the abdomen was shaved and disinfected with betadine and sterile phosphate buffered saline (PBS). Animals were kept on their backs on a heating plate at 39° C. A midline laparotomy (1-1.5 cm) was performed through the skin and peritoneum. Four hooks, positioned around the incision and fixed to a retractor and magnetic base plate, allowed for clear access to the abdominal cavity and liver.

Labelling of liver surfaces was performed. Local damage to the liver surface was induced via electroporation tweezers by applying electric voltage: 30V, pulse: 50 ms, interval: 1 second, cycles: 8. Before closure of the incision, Buprenorphine (0.1 mg/kg) was pipetted in the abdomen to allow for initial post-surgical analgesia. For long-term analgesia, Metamizole (Novalgin, 200 mg/kg) was provided through daily injections. The peritoneum and skin were closed with two separate 4-0 silk sutures (Ethicon). Upon closure of the incision, mice were woken up by antagonizing Medetomidin and Midazolam through a subcutaneous cocktail injection of Atipamezol (1 mg/kg) and Flumazenil (0.25 mg/kg). Mice were allowed to recover on a heating pad, after which they were single housed. Mice where sacrificed after indicated time point and liver tissue was obtained. In the peritoneal model, surgical procedure was as described above, but the peritoneal areas were marked.

Inhibitors were injected 2 hours before surgery with a concentration of 10 μM of the corresponding small molecules dissolved in sterile PBS i.p.

### Labelling of ECM Components

Succinimidyl esters (NHS-esters; Thermo Fisher) were diluted in DMSO to 25 mg/ml and stored at −80° C. For local matrix staining labelling solution was generated by mixing NHS-ester 1:1 with 100 mM pH 9.0 sodium bicarbonate buffer. For global abdominal labelling, 20 μl of NHS-labelling solution were mixed with 100 μl sterile PBS and injected i.p.

### Tissue Preparation

Upon organ excision, organs were fixed overnight at 4° C. in 2% formaldehyde. The next day, fixed tissues were washed three times in Dulbecco's phosphate buffered saline (DPBS, GIBCO, #14190-094), and depending on the purpose, either embedded, frozen in optimal cutting temperature compound (Sakura, #4583) and stored at −20° C., or stored at 4° C. in PBS containing 0.2% gelatin (Sigma Aldrich, #G1393), 0.5% Triton X-100 (Sigma Aldrich, #X100) and 0.01% Thimerosal (Sigma Aldrich, #T8784) (PBS-GT). Fixed tissues were embedded in optimal cutting temperature (OCT) and cut with a Microm HM 525 (Thermo Scientific). In short, sections were fixed in ice-cold acetone for 5 min at −20° C., and then washed with PBS. Sections were then blocked for non-specific binding with 10% serum in PBS for 60 minutes at room temperature, and then incubated with primary antibody in blocking solution O/N at 4° C. The next day, following washing, sections were incubated in PBS with fluorescent secondary antibody, for 120 min at RT. Finally, sections were washed and incubated with Hoechst 33342 nucleic acid stain (Invitrogen, #H1399), washed in ddH₂O, mounted with Fluoromount-G® (Southern Biotech, #0100-01), and stored at 4° C. in the dark. Primary antibodies: rabbit-anti-collagen I (1:150, Rockland), rabbit-anti-Cytokeratin (1:100, Sigma Aldrich), rabbit-anti-Ki67 (1:100, Abcam), rabbit-anti-Fibronectin (1:100, Abcam), rabbit-anti-HSP70 (1:100, Elabscience), rabbit-anti-HSPG2 (1:100, Elabscience), rabbit-anti-Keratin9 (1:100, Elabscience), rabbit-anti-Ki67 (1:100, Abcam), rabbit-anti-cleaved Caspase 3 (1:100, Abcam), rabbit-anti-Laminin (1:100, Abcam), rabbit-anti-HSP70 (1:100, Elabscience), hamster-anti-PDPNα (1:100, Abcam), rat-anti-LY6G(Sca1) (1:100, Abcam), rabbit-anti-MMP23(1:100, Elabscience), rabbit-anti-Vitronectin (1:100, Elabscience) and rabbit-anti-WT1 (1:100, Abcam). Alexa Fluor 488-, Alexa Fluor 568- or Alexa Fluor 647-conjugated antibodies (1:500, Life technologies) against suitable species were used as secondary antibodies. H&E stainings where performed according to MMM.

### Microscopy

Histological sections were imaged using a using a M205 FCA Stereomicroscope (Leica). 2D, 3D and 4D data was processed with Imaris 9.1.0 (Bitplane) and ImageJ (1.52i). Contrast and brightness were adjusted for better visibility.

### Proteinbiochemistry

Tissues were snap frozen and grinded using a tissue lyser (Quiagen). Pulverised tissues were resuspended in lysis buffer (20 mM Tris-HCl pH 7.5, 1% Triton X-100, 2% SDS, 100 mM NaCl, 1 mM sodium orthovanandate, 9.5 mM sodium fluoride, 10 mM sodium pyruvate, 10 mM beta-glycerophosphate, and supplemented with protease inhibitors (complete protease inhibitor cocktail, Pierce) and kept 10 min on ice. Samples were sonicated and spinned down for 5 minutes with 10.000g. Supernatants were stored at −80° C. Protein concentration was determined via BCA-Assay according to manufactures protocol (Pierce).

Protein pulldown was as follows. Lysates were diluted with a pulldown buffer (20 mM Tris-HCl pH 7.5, 1% Triton X-100, 100 mM NaCl and supplemented with protease and phosphatase inhibitors) and incubated overnight with dynabeads (Thermo Fisher) according to manufacturer's instructions at 4° C. on a rotator. The next day, the samples were each diluted twice with Wash Buffer 1 (20 mM Tris-HCl pH 7.5, 1% Triton X-100, 2% SDS, 100 mM NaCl and supplemented with protease and phosphatase inhibitors) and then with Wash Buffer 2 (20 mM Tris-HCl pH 7).5, 0.5% Triton X-100, 100 mM NaCl and supplemented with protease and phosphatase inhibitors) and finally washed twice with Wash Buffer 3 (20 mM Tris-HCl pH 7.5 and 100 mM NaCl). Beads were then resuspended in Elution Buffer (20 mM Tris-HCl pH 7.5, 100 mM NaCl and 50 mM DTT) and incubated for 30 minutes in Elution Buffer at 37° C. Finally, the samples were boiled for 5 minutes at 98° C. and the supernatants were stored at −80° C. Fluorescence intensities of lysates were measured were measured in a Fluostar optima fluorometer (BMGlabtech).

### Mass Spectrometry

Tissues were marked locally with an EZ-LINK-NHS 100:1 FITC-NHS mixture. After 24 hours the organs were removed, tissue pieces of the original marking were separated from moved matrix fraction and snap frozen. Tissue lysis was performed as described above. Samples were digested using a modified FASP procedure. After reduction and alkylation using DTT and IAA, the proteins were centrifuged on Microcon® centrifugal filters (Sartorius Vivacon 500 30 kDa), washed thrice with 8 M urea in 0.1 M Tris/HCl pH 8.5 and twice with 50 mM ammoniumbicarbonate. The proteins on filter were digested for 2 hours at room temperature using 0.5 μg Lys-C (Wako Chemicals, Neuss, Germany) and for 16 hours at 37° C. using 1 μg trypsin (Promega, Mannheim, Germany). Peptides were collected by centrifugation (10 min at 14000 g), acidified with 0.5% TFA and stored at −20° C. until measurements. The digested peptides were loaded automatically to a HPLC system (Thermo Fisher Scientific) equipped with a nano trap column (100 μm ID×2 cm, Acclaim PepMAP 100 C18, 5 μm, 100 Å/size, LC Packings, Thermo Fisher Scientific, Bremen, Germany) in 95% buffer A (2% ACN, 0.1% formic acid (FA) in HPLC-grade water) and 5% buffer B (98% ACN, 0.1% FA in HPLC-grade water) at 30 μl/min. After 5 min, the peptides were eluted and separated on the analytical column (nanoEase MZ HSS T3 Column, 100 Å, 1.8 μm, 75 μm×250 mm, Waters) at 250 nl/min flow rate in a 105 minutes non-linear acetonitrile gradient from 3 to 40% in 0.1% formic acid. The eluting peptides were analyzed online in a Q Exactive HF mass spectrometer (Thermo Fisher Scientific, Bremen, Germany) coupled to the HPLC system with a nano spray ion source and operated in the data-dependent mode. MS spectra were recorded at a resolution of 60,000 and after each MS1 cycle, the 10 most abundant peptide ions were selected for fragmentation. Raw spectra were imported into Progenesis QIsoftware (version 4.1, Nonlinear Dynamics, Waters). After feature alignment and normalization, spectra were exported as Mascot Generic files and searched against the SwissProt mouse database (16,872 sequences) with Mascot (Matrix Science, version 2.6.2) with the following search parameters: 10 ppm peptide mass tolerance and 0.02 Da fragment mass tolerance, two missed cleavages allowed, carbamidomethylation was set as fixed modification, camthiopropanoyl, methionine and proline oxidation were allowed as variable modifications. A Mascot-integrated decoy database search calculated an average false discovery of <1% when searches were performed with a mascot percolator score cut-off of 13 and an appropriate significance threshold p.

Peptide assignments were re-imported into the Progenesis QI software and the abundances of all unique peptides allocated to each protein were summed up. The resulting normalized abundances of the individual proteins were used for calculation of protein ratios and p-values (ANOVA) between sample groups using a nested design. Gene ontology analysis was performed using EnrichR webtool^{26,27}.

### Results

To analyze the ECM movement the Inventors chose the electroporation model for livers and in the case of peritoneas the laparotomy section as local injury (FIG. 40a and methods). The investigations showed that the inhibition of lysyl oxidases and elastases resulted in increased matrix movement. The inhibition of motor proteins showed inhibitory effects on matrix currents only in peritoneas. Heat shock factor inhibition blocked matrix currents in both organs. Among the protease inhibitors, the broad-spectrum MMP inhibitor GM6001 proved to be the most potent.

Using the inventors signaling pathway analysis, they identified multiple molecules that inhibited or amplified matrix flows. Interestingly, some effector molecules like Blebbistatin and ciliobrevin effected matrix currents of only one organ. Since the composition of the fluid matrix differs from organ to organ, organ-specific modulators of the matrix currents could be applied after identification of appropriate biomarkers.

In summary, they show a new method to attach molecules to wounds, new potential markers for pulmonary fibrosis and signaling pathways to modulate matrix movements.

### Example 11: Fluid Matrix Reservoirs Irrigate Lungs to Cause Fibrosis

Lung disease leads to organ failure from inflammation, connective tissue matrix accretion and fibrotic scarring. Although the mechanism of fibrosis is poorly understood, it is thought to occur through de novo synthesis and deposition of extra cellular matrix by fibroblasts. Here the Inventors discover that lungs are actually sheathed by a ready-made adventitial reservoir of fluid-like matrix and that injury triggers a massive flow of this reservoir into mouse lungs, culminating in fibrotic scars. Furthermore, these adventitial reservoirs of fluid matrix also exist and flow in ex vivo human diseased lung samples. Using mass spectrometric analysis of mouse and human lungs, the Inventors uncover that fluid matrix irrigation liberates basement membranes, elastic and collagenous fibers and crosslinking enzymes, decreasing lung surface elasticity while stiffening the lungs.

Addressing the mechanism in mice, the Inventors demonstrate that immune cells trigger fluid matrix irrigation and this effect is exacerbated by immune cells from patients with lung disease. By uncovering how inflammation liberates matrix reservoirs the findings reveal a new phenomenon that fundamentally changes the view of fibrotic processes. This study thus creates new therapeutic and diagnostic avenues to treat a variety of incurable, and hard to diagnose, lung diseases.

Methods

Patient derived PFA and ST Tissue

All tissues (PFA, ST) used in this study were obtained with properly informed consent of patients. All experimental procedures were performed in accordance with the Research Ethics Boards (REB 1000055059) at The Hospital for Sick Children (Toronto, Canada). Primary tumor cultures used in this study are from patients that have not undergone radiotherapy or chemotherapy prior to surgical resection.

### Patient Derived PFA and ST Cell Cultures

All samples used in this study were obtained with properly informed consent of patients. All experimental procedures were performed in accordance with the Research Ethics Boards at The Hospital for Sick Children (Toronto, Canada). Patient derived PFA-ependymoma cell lines (MDT-PFA1, MDT-PFA2, MDT-PFA3, MDT-PFA4, MDT-PFA5, MDT-PFA7 MDT-PFA8, MDT-PFA9, MDT-PFA13, MDT-PFA15) and supratentorial ependymoma cell lines (MDT-ST1, MDT-ST4) were established in this study. GBM and DIPG, K27M cell cultures were obtained from Dr. Peter Dirks (The Hospital for sick Children, Canada) and Dr. Nada Jabado (McGil University, Canada) respectively. All cell cultures were confirmed to match original tumors by STR fingerprinting, where tumor tissues were available. The following PFA and ST cell cultures were derived from male patients: MDT-PFA1, MDT-PFA2, MDT-PFA3, MDT-PFA5, MDT-PFA7, MDT-PFA8, MDT-PFA9, MDT-PFA13, MDT-PFA15, MDT-ST4. The following PFA and ST cell cultures were derived from female patients: MDT-PFA4, MDT-ST1. Human fetal neural stem cells, fNSC (HF7450, HF6562) and immortalized normal human astrocytes (iNHA) were obtained from Dr. Peter Dirks (The Hospital for sick Children, Canada) and Dr. Nada Jabado (McGil University, Canada) respectively.

### Mouse Housing and Husbandry

All mouse breeding and procedures were performed as approved by The Centre for Phenogenomics (Toronto). Pairs of C57BL/6J mice were obtained from The Jackson laboratory for mouse breeding. Embryos of mated C57BL/6J female mice were dissected to collect hindbrain tissue from E10, E12, E14, E16 and E18 gestational time points. Hindbrain of C57BL/6J pups was dissected to collect tissue from P0, P5, P7 and P14 postnatal time points.

### In Vivo Matrix Fate Tracing

The inventors generated a labelling solution by mixing 5 μl NHS-ester (25 mg/ml) with 5 μl of 100 mM pH 9.0 sodium bicarbonate buffer, combining with 40 μl PBS to a total volume of 50 μl. The labelling solution was applied intra pleural under isoflurane anaesthesia by using a 30G cannula.

### Bleomycin Induced Pneumonia Model

The oropharyngeal administration of bleomycin for the induction of pulmonary fibrosis was carried out in an antagonistic anesthesia in C57BL/6J mice of both sexes (6-8 weeks age). After the toe-pinch reflex was absent, the mouse was placed on the incisors of the upper jaw and thus kept in an upright position. The tongue was carefully fixed and held to the side with tweezers while the nose of the animal is covered with tweezers. By keeping the nose closed, the mouse was forced to breathe through the mouth. With the help of a pipette, bleomycin was dissolved in a dosage of 2 units/kg KGW in 80 μl PBS carefully into the throat. As soon as the animal has inhaled the solution, it was tansferred to a Hot plate (duration approx. 30 to 60 seconds). After antagonization animals were housed for 14 days. Nintedanib was added 1 hour before bleomycin installation and every other day intra peritoneal 10 μM.

### Ex Vivo Culture of Lung Biopsies

C57BL/6J male mice (6-8 weeks age) were used to study the movement of the lung matrix. After the organ withdrawal 4 mm biopsy punches of murine lungs were generated. To obtain ectopic labeling of matrix, the inventors generated a labelling solution by mixing NHS-ester 1:1 with 100 mM pH 9.0 sodium bicarbonate buffer. Sterile Whatman filter paper (Sigma Aldrich) biopsy punches where soaked in NHS-labelling solution, and locally placed on the lung biopsy surface. After one minute, the labelling punch was removed. Mouse lung biopsies were cocultured in the RPMI medium (10% FBS with 1% Pen/Strep and 0.1% AmB) consist of different sub types of immune cells (0.1×106 cells/biopsy) isolated from the healthy and idiopathic pulmonary fibrosis (IPF) donors. Mouse lung biopsies with immune cells were then cultured in the ex vivo condition and provided with 5% CO2 at 37° C.

After 48 hours, mouse lung biopsies were fixed with the 4% formalin and incubated for overnight at 40 C followed by PBS wash. Human lung tissues where obtained, labelled, and cultivated for 24 hours as described above.

### Tissue Preparation Histology

Upon organ excision, organs were fixed overnight at 4° C. in 2% formaldehyde. The next day, fixed tissues were washed three times in Dulbecco's phosphate buffered saline (DPBS, GIBCO, #14190-094), and depending on the purpose, either embedded, frozen in optimal cutting temperature compound (Sakura, #4583) and stored at −20° C., or stored at 4° C. in PBS containing 0.2% gelatin (Sigma Aldrich, #G1393), 0.5% Triton X-100 (Sigma Aldrich, #X100) and 0.01% Thimerosal (Sigma Aldrich, #T8784) (PBS-GT). Fixed tissues were embedded in optimal cutting temperature (OCT) and cut with a Microm HM 525 (Thermo Scientific). In short, sections were fixed in ice-cold acetone for 5 min at −20° C., and then washed with PBS. Sections were then blocked for non-specific binding with 10% serum in PBS for 60 minutes at room temperature, and then incubated with primary antibody in blocking solution O/N at 4° C. The next day, following washing, sections were incubated in PBS with fluorescent secondary antibody, for 120 min at RT. Finally, sections were washed and incubated with Hoechst 33342 nucleic acid stain (Invitrogen, #H1399), washed in ddH2O, mounted with Fluoromount-G® (Southern Biotech, #0100-01), and stored at 4° C. in the dark.

### 3D Multiphoton Imaging

For multi-photon imaging, samples were embedded in a 4% NuSieve GTG agarose solution (Lonza, #50080). Imaging was performed using a 25× water-dipping objective (HC IRAPO L 25×/1.00W) coupled to a tunable pulsed laser (Spectra Physics, Insight DS+). Multi-photon excited images were recorded with external, non-descanned hybrid photo detectors (HyDs). Following band pass (BP) filters were used for detection: HC 405/150 BP for Second Harmonic Generation (SHG) and a ET 525/50 BP for green channel Tiles were merged using Leica Application suite X (v3.3.0, Leica) with smooth overlap blending and data were visualized with Imaris software (v9.1.3, Bitplane).

### 3D Lightsheet Imaging

Whole-mount samples were stained and cleared with a modified 3DISCO protocol (Ertürk et al., 2012). Samples were dehydrated in an ascending tetrahydrofuran (Sigma Aldrich, #186562) series (50%, 70%, 3×100%; 60 minutes each), and subsequently cleared in dichloromethane (Sigma Aldrich, #270997) for 30 min and eventually immersed in benzyl ether (Sigma Aldrich, #108014). Cleared samples were imaged whilst submerged in benzyl-ether with a light-sheet fluorescence microscope (LaVision BioTec). Whilst submerged in benzyl-ether, specimens were illuminated on two sides by a planar light-sheet using a white-light laser (SuperK Extreme EXW-9; NKT Photonics). Optical sections were recorded by moving the specimen chamber vertically at 5-mm steps through the laser light-sheet. Three-dimensional reconstructions were obtained using Imaris imaging software (v9.1.3, Bitplane).

### Histology and Murine Ex Vivo Imaging

Histological sections were imaged under a M205 FCA Stereomicroscope (Leica) and ZEISS AxioImager Z2m (Carl Zeiss). Murine biopsy punches were imaged under a M205 FCA Stereomicroscope (Leica). Data was processed with Imaris 9.1.3 (Bitplane) and ImageJ (1.52i). Contrast and brightness were adjusted for better visibility. Thundering was performed with fluoromount and standard parameter settings for histology cuts.

### Immune Cell Isolation

Human whole peripheral blood from healthy control and interstitial lung disease patients was collected in EDTA—tubes and processed within two hours of. PBMCs were isolated using density gradient centrifugation (Stemcell, Catalog #07851). The PMBC layer and additionally the white cells directly above the red blood cells (RBC) were collected for isolation of the different immune cell subsets.

### Monocyte and Lymphocyte Isolation

The PBMCs layer was split in half and underwent autoMACS® (Miltenyi Biotec, Catalog #130-092-545) bead isolations for the different cellular subtypes.

a) One half was used for monocyte isolation following the protocol provided by the Pan monocyte isolation kit (Miltenyi Biotec, Catalog #130-096-537).

b) The other half was used to isolate lymphocytes. (i) First isolation was performed with CD19 microbeads (Miltenyi Biotec, Catalog #130-050-301) and the positive fraction corresponding to B cells was collected. (ii) Then, using the negative fraction, T cells were isolated according to the protocol provided with the human Pan T isolation kit (Miltenyi Biotec, Catalog #130-096-535).

### Granulocyte Isolation

In parallel with the isolations above, the white cell pellet of the RBCs was used to isolate the different granulocyte subtypes. Initially, the cells were resuspended in PBS and centrifuged at 300g for 15 minutes. In the next step, red blood cell lysis was performed on the pellet using the TQ-Prep Workstation (Beckam Coulter, Catalog #6605429). The lysed pellet was washed with PBS and centrifuged at 300g for 10 minutes, to procure all granulocytic cells.

To be able to separate the different granulocyte subtypes, the inventors proceeded with CD16 microbeads (Miltenyi Biotec, Catalog #130-045-701) following the protocol for magnetic isolation using autoMACS®. The resulting negative fraction corresponded to granulocytes and the positive fraction a mixed population of neutrophils and basophils. The quality of the different cell types was determined by flow cytometry.

### Protein Biochemistry

Tissues were snap frozen and grinded using a tissue lyser (Quiagen). Pulverised tissues were resuspended in lysis buffer (20 mM Tris-HCl pH 7.5, 1% Triton X-100, 2% SDS, 100 mM NaCl, 1 mM sodium orthovanandate, 9.5 mM sodium fluoride, 10 mM sodium pyruvate, 10 mM beta-glycerophosphate), and supplemented with protease inhibitors (complete protease inhibitor cocktail, Pierce) and kept 10 min on ice. Samples were sonicated and spun down for 5 minutes at 10,000g. Supernatants were stored at −80° C. Protein concentration was determined via BCA-Assay according to manufactures protocol (Pierce).

Protein pulldown was as follows. Lysates were diluted with a pulldown buffer (20 mM Tris-HCl pH 7.5, 1% Triton X-100, 100 mM NaCl and supplemented with protease and phosphatase inhibitors) and incubated overnight with Dynabeads at 4° C. on a rotator according to the manufacturer's instructions. The next day, the samples were each diluted twice with Wash Buffer 1 (20 mM Tris-HCl pH 7.5, 1% Triton X-100, 2% SDS, 100 mM NaCl, supplemented with protease and phosphatase inhibitors) and then with Wash Buffer 2 (20 mM Tris-HCl pH 7.5, 0.5% Triton X-100, 100 mM NaCl, supplemented with protease and phosphatase inhibitors) and finally washed twice with Wash Buffer 3 (20 mM Tris-HCl pH 7.5 and 100 mM NaCl). Beads were then resuspended in Elution Buffer (20 mM Tris-HCl pH 7.5, 100 mM NaCl and 50 mM DTT) and incubated for 30 minutes at 37° C. Finally, the samples were boiled for 5 minutes at 98° C. and the supernatants were stored at −80° C.

### Mass Spectrometry

Tissue lysis was performed as described above. Samples were digested using a modified FASP procedure as described by Wiśniewski et al., 2009. After reduction and alkylation using DTT and IAA, the proteins were centrifuged on Microcon® centrifugal filters (Sartorius Vivacon 500 30 kDa), washed thrice with 8 M urea in 0.1 M Tris/HCl pH 8.5 and twice with 50 mM ammonium bicarbonate. The proteins on filters were digested for 2 hours at room temperature using 0.5 μg Lys-C (Wako Chemicals) and for 16 hours at 37° C. with 1 μg trypsin (Promega). Peptides were collected by centrifugation (10 min at 14000 g), acidified with 0.5% TFA and stored at −20° C. until measurements. The digested peptides were loaded automatically on a HPLC system (Thermo Fisher Scientific) equipped with a nano trap column (100 μm ID×2 cm, Acclaim PepMAP 100 C18, 5 μm, 100 Å/size, LC Packings, Thermo Fisher Scientific) in 95% buffer A (2% ACN, 0.1% formic acid (FA) in HPLC-grade water) and 5% buffer B (98% ACN, 0.1% FA in HPLC-grade water) flowing at 30 μl/min. After 5 min, the peptides were eluted and separated on the analytical column (nanoEase MZ HSS T3 Column, 100 Å, 1.8 μm, 75 μm×250 mm, Waters) for 105 minutes at 250 nl/min flow rate in a 3 to 40% non-linear acetonitrile gradient in 0.1% formic acid. The eluting peptides were analyzed online in a Q Exactive HF mass spectrometer (Thermo Fisher Scientific) coupled to the HPLC system with a nano spray ion source, operated in the data-dependent mode. MS spectra were recorded at a resolution of 60,000 and after each MS1 cycle, the 10 most abundant peptide ions were selected for fragmentation. Raw spectra were imported with Progenesis QI software (version 4.1, Nonlinear Dynamics, Waters). After feature alignment and normalization, spectra were exported as Mascot Generic files and searched against the SwissProt mouse database (16,872 sequences) with Mascot (Matrix Science, version 2.6.2) with the following search parameters: 10 ppm peptide mass tolerance and 0.02 Da fragment mass tolerance, two missed cleavages allowed, carbamidomethylation was set as fixed modification, camthiopropanoyl, methionine and proline oxidation were allowed as variable modifications. A Mascot-integrated decoy database search calculated an average false discovery of <5% when searches were performed with a mascot percolator score cut-off of 13 and a significance threshold p-value.

Peptide assignments were re-imported into the Progenesis QI software and the abundances of all unique peptides allocated to each protein were summed. The resulting normalized abundances of the individual proteins were used for calculation of protein ratios and p-values (ANOVA) between sample groups using a nested design. Extracellular elements were identified through a database search against a matrisomal database (Shao et al., 2020). Gene ontology analysis was performed using EnrichR webtool (Chen et al., 2013; Kuleshov et al., 2016).

Results

A Pleuro-Vascular Axis Irrigates Injured Lungs with Pre-Existing Fluid Matrix

Damaged and inflamed lungs rebuild with a complex mixture of tissue and matrix, the provenance of which has remained obscure. The Inventors recently demonstrated in skin that loose connective tissue (matrix) serves a source for dermal scars (Correa-Gallegos et al., 2019). The Inventors therefore set out to test the possibility that preexisting matrix translocates in, another injured tissue namely, lung. For this, the Inventors locally tagged and fate-mapped the matrix lining the lungs (pleura) of live mice with N-hydroxysuccinimide ester fluorescein isothiocyanate (FIG. 41A, 41B).

Foci of labeled pleura matrix clearly coincided with the second harmonic signal, indicating the extracellular collagenous fibers were correctly labeled. The second harmonic signal also revealed a rigid intertwined framework of large mature collagen fibers in lung pleura. The fibers formed a web across the lung surface with large gaps with no signal. Gaps and distances between adjacent mature fibers were filled with a matrix of minute fibrils and multi-fibril aggregates in an immature arrangement (FIG. 41B). The immature matrix of the pleural surfaces is organized in volumes of fibers and protein-rich clouds or mists that surround and enwrap the rigid connective tissue frames of the lung surface. These protein-rich clouds adhere to rigid frames through filaments that interconnect them with the rigid frames of woven collagen fibers. These findings indicate lung surfaces are composed of two distinct sub-structures, a rigid mature collagenous frame and a surrounding protein-rich immature connective tissue matrix.

To test the mobility of the immature matrix in response to disease in mice, the Inventors instilled Bleomycin in trachea (FIG. 41D). The Inventors chose Bleomycin because it induces a robust pneumonia that obstructs the bronchioles, leading to extensive pulmonary scarring and respiratory failure. It thus allowed us to study the provenance of matrix during two key steps in lung disease: inflammation and fibrosis. Strikingly, Bleomycin induced extensive inward movement of pre-labeled matrix from pleural surfaces to the center of the lungs. This mobility of pleural matrix was progressive. In the first days it continuously irrigated the outermost airways (bronchioles) and their surrounding interstitial space with matrix deposits that effectively encapsulated the alveoli and bronchioles. Over subsequent days, fluid matrix moved even further inwards, accumulating around the bronchi and vascular adventitia, generating thickened layers and accretions of matrix that surrounded the major blood vessels and bronchi. These movements thickened the adventitia, media and intima compartments of the lung's major vessels (FIG. 41E). Over the course of 2 weeks from injury, fluid matrix accumulated along the entire bronchial tree and had completely interpenetrated the lung interstitium, forming large regions with dense scars, in which myofibroblast foci emerged (FIGS. 41F and G). The Inventors also found that the fine fibrillar structures of lung surfaces underwent changes in response to Bleomycin treatment with pockets of reduced fluorescence intensity, indicating loss of protein from the surface (FIG. 45). Indeed, this reduction of fluorescence intensity from surfaces of diseased lungs was associated with loss of fine fibrillar volumes and with changes in matrix organization, as compared to healthy pleura (FIG. 41H and FIGS. 45A and 1B). The Inventors remarked whirl-like fiber structures, which were reduced in fiber content in diseased lungs.

Next, the Inventors sought to define the protein constituents of the fluid matrix from pleural reservoirs by mass-spectrometry. Briefly, the Inventors injected a Biotin-conjugated EZ-link sulfo-N-Hydroxysuccinimide ester into the pleural space, tagging pools of matrix reservoirs on pleural lung surfaces as the Inventors did before. The Inventors followed up by subjecting mice to Bleomycin-induced injury (FIG. 46A). Two weeks post-injury, the Inventors collected diseased lungs, and purified labeled matrix via Streptavidin pull-down followed by mass spectrometric proteomics of all tagged peptides. Principle component analysis revealed that the fluid matrix resembles fluid scar tissue (FIG. 46D), with 73% of all identified extra cellular proteins being constituents of collagenous fibers (FIGS. 46B and C). This was confirmed with immunolabeling diseased lungs. Overall, these data uncover an inward axis of fluid matrix movement, from pleural surfaces into deep lung adventitial and bronchial spaces. They further indicate that lung surfaces harbor large reservoirs of fluid matrix that irrigate the entire lung over days, effectively laying down the connective tissue that forms fibrotic scars.

### Immunity Orchestrates Matrix Homing

Lung fibrosis implicates a wide variety of immune cells although any causality and or mechanisms remain to be established. Motivated by this, the Inventors set out to investigate the influence of distinct immune cell populations on matrix invasion in lungs. The Inventors purified populations of lymphocytes (B and T cells), monocytes, and granulocytes (neutrophils, eosinophils, basophils) from healthy human volunteers. Lung explant fluid matrix reservoirs were labeled on pleural surfaces with dye ester as before, and they were individually cultivated with subsets of immune cells obtained from healthy human volunteers (FIG. 42A). There was a consistent decline in lung surface fluorescence intensity in lung explants with fluorescently labeled surfaces when they were cultivated with immune cells. Fluorescence remained constant in immune-deficient samples. This indicates that immune cells caused matrix to be lost from injured lung surfaces. Fluorescence intensity values dramatically halved within 48 hours of adding immune cells to the lung explants. Granulocytes, neutrophils and monocytes were the most potent inducers of matrix loss from surfaces (FIG. 42B). As above in 42B, this loss of protein and fiber from lung surfaces was accompanied by vigorous inward movement of labeled matrix, which irrigated the alveolar and interstitial spaces of lung biopsies (FIG. 42C).

As immune cells from healthy volunteers triggered matrix invasion into the lungs, the Inventors next closely analyzed the impact of lung disease patient immune cells on matrix movement. For this the Inventors isolated immune cells directly from idiopathic pulmonary fibrosis patients and added them to the lung explant cultures. All types of patient immune cells augmented vigorous movements of matrix from pleural surfaces. This led to decreased surface fluorescence intensity, from 49 to 20, that was accompanied by a high inward invasion index of fluid matrix from 60 mm to 110 mm. Fluorescence histologic images of lung explants revealed that monocytes and lymphocytes from idiopathic pulmonary fibrosis patients induced the most significant invasion of pleural matrix into the alveolar and interstitial space, which remarkably resembled the initial findings in Bleomycin-treated animals.

These findings strongly suggest that both monocytes and lymphocytes play key roles in liberating fluid matrix from pleural surfaces and irrigating the lungs. Moreover, the Inventors can deduce that immune cells from diseased patients are ‘primed’ for this task, much more then in healthy individuals.

### Diseased Human Lungs Undergo Vigorous Matrix Movements

To study if human diseased lungs undergo matrix movements in the same way as in the mouse model, the Inventors adapted the labeling technique to human lung samples from diseased patients (FIG. 43A). Although human lungs had much more elaborate patterns of elastic fibers than mouse, the same two types of connective tissue organizations were visible. Rigid and static frames of thick mature collagenous fibers were bathed in volumes of protein-rich fluid-like immature matrix.

Labelled human pleural reservoirs, underwent dramatic inward movement of fluid matrix within 24 hours. Importantly the Inventors were able to detect matrix currents into the interior of injured human lung tissue (FIG. 43A).

Two-photon images of labeled diseased lung biopsies showed protein-rich fluid and fibers completely irrigated the interstitial spaces surrounding the bronchioles and blood vessels of injured lungs, down to the major bronchus. Here, the Inventors observed multiple accretion layers of connective tissue fibers laid down and intermingled within the tunica adventitia, media and intima, generating a thickened bronchial wall and rim replete with new fibers, as the Inventors initially found in Bleomycin-treated mice (FIG. 43B).

To summarize, the Inventors observed the same protein-rich matrix movements in human diseased lungs that the Inventors observed above in a mouse model of lung disease. Furthermore these movements were also induced in healthy lung samples from mouse after cultivating with immune cells from diseased patients. Thus, immune cells trigger irrigation of protein-rich matrix from reservoirs on pleural surfaces. These data therefore establish a functional link between inflammation and downstream fibrosis.

To study the composition of this protein-rich fluid matrix in human lungs, the Inventors tagged diseased biopsy pleura, and incubated them for 24 hours. The Inventors then separated pleural and interstitial tissues for protein extraction, followed by mass spectrometry proteomics (FIG. 43D). The proteomic inventory revealed a protein-rich fluid matrix of 1,346 different protein types that invaded inwards, accumulating within lung interstitial spaces and surrounding lung bronchioles. A survey of proteins against a database consisting of extracellular components revealed that most (˜76%) of the human pleural fluid matrix fraction consisted of collagenous fibers (FIG. 43E). Principle component analysis indicated that this protein-rich soup was similar in composition to atrophic scars and to abnormal stiffened vascular connective tissue matrix (FIG. 43F). Fluid matrix also consisted of numerous ground-substance proteins, such as glycoproteins, proteoglycans and extracellular matrix-affiliated proteins. In agreement with the multi-photon imaging, the Inventors detected abundant fibrillar collagenous fibers such as Collagen type I and III, and their covalent crosslinking enzymes such as Lysyl oxidase (Lox) and Transglutaminases (Tgm): both involved in connective tissue remodeling and maturation and with the activation of fibroblasts into pathologic myofibroblasts (FIG. 43G). The Inventors also found proteins involved in basement membrane formation and stability such as Collagen type IV, VI, including elastic fiber complexes such as elastins and fibrillins: needed to maintain organ pliancy and elasticity, all of which were removed from the pleural surfaces. Overall, the Inventors identified a protein inventory that contributes to tissue rigidity in multiple ways.

By analyzing the relative fractions of proteins remaining on lung surfaces versus those removed, the Inventors found that individual proteins had vastly distinct translocation profiles (FIG. 46A). For example, elastic fibers and fibrillary collagens had a high translocation index of ˜1.2, whereas basement membrane components were extremely slow, with a poor translocation index of ˜0.8 (FIG. 46B). Out of the list of cross-linking enzymes, the non-specific transglutaminase 2 (TGM2) had a higher translocation index than any other cross-linking enzyme or isoform, indicating that unspecific cross-linking is predominant in fibrotic plaques. Apolipoprotein (LPA) also stood out as having an extremely high translocation index, consistent with LPA serving as an early fibrosis marker. These widely varying translocation profiles indicate that protein liberation from pleural surfaces is dynamic, and that the protein soup that bathes injured lungs changes its composition depending on rate of movement of individual proteins.

### Nintedanib Inhibits Lung Fibrosis by Blocking Connective Tissue Irrigation

Having discovered that immune cells trigger matrix translocations, the Inventors went on to study if an anti-fibrotic anti-inflammatory drug inhibits matrix motions in animals. The pan-tyrosine kinase inhibitor Nintedanib is currently one of only two anti-fibrotic drugs on the market that have been approved for pulmonary fibrosis, as it has anti-fibrosis and anti-inflammatory activities that impede disease progression. To check a possible effect of Nintedanib on matrix reservoirs, the Inventors performed a global kinase enrichment assay across the entire fluid matrix proteome. Indeed, the Inventors were encouraged to find that the fluid matrix proteome was highly enriched in tyrosine kinase and therefore significantly affected by its activity (FIG. 44A).

To directly answer if Nintedanib's anti-fibrotic actions are mediated through its effects on fluid matrix movements, the Inventors labeled fluid matrix reservoirs on lung surfaces as before, induced lung inflammation and fibrosis with Bleomycin, and followed these mice with daily injections of Nintedanib (FIG. 44B). Whereas Bleomycin triggered massive inward translocations of fluid matrix, Nintedanib injections completely abrogated these matrix movements (FIGS. 44C and 44D). Nintedanib-injected animals maintained the same structures and fluorescence intensity of matrix on lung surfaces, reminiscent of control healthy lungs. Moreover, histologic analysis showed that matrix reservoirs refrained from translocating inwards and irrigating the lungs. Both lung alveoli and major blood vessels lacked accretions of labeled fibers. In the absence of matrix movements, lung interstitial fibroblasts were refrained from activating into pathologic YAP/TAZ-positive myofibroblasts. As a result lung structure appeared much healthier, with very minimal signs of fibrotic developments along lung airways and bronchioles (FIG. 44D). This indicates that Nintedanib exerts anti-fibrotic effects by inhibiting matrix translocation from reservoirs, thereby uncoupling inflammation from the accretion of new connective tissue in the lungs.

In sum, the Inventors reveal here the connection between inflammation and downstream fibrogenesis. The Inventors demonstrate that inflammation mobilizes protein-rich fluid matrix from pleural reservoirs to irrigate lungs with scar tissue, and that Nintedanib acts by inhibiting fluid matrix irrigation, thereby improving disease progression. Matrix irrigation is likely a general principle of organ injury and disease with potential clinical ramifications to many human fibrotic conditions.

### REFERENCES

1. Marshall, C. D., Hu, M. S., Leavitt, T., Barnes, L. A., Lorenz, H. P. & Longaker, M. T. Cutaneous scarring: basic science, current treatments, and future directions. Advances in wound care 7 (2), 29-45 (2018).
2. Hinz, B. Myofibroblasts. Exp Eye Res 142, 56-70 (2016).
3. Driskell, R. R., Lichtenberger, B. M., Hoste, E., Kretzschmar, K., Simons, B. D., Charalambous, M., Ferron, S. R., Herault, Y., Pavlovic, G., Ferguson-Smith, A. C., & Watt, F. M. Distinct fibroblast lineages determine dermal architecture in skin development and repair. Nature 504 (7479), 277-281 (2013).
4. Greenhalgh, S. N., Conroy, K. P., & Henderson, N., C. Healing scars: targeting pericytes to treat fibrosis. QJM 108 (1), 3-7 (2015).
5. Plikus, M. V., Guerrero-Juarez, C. F., Ito, M., Li, Y. R., Dedhia, P. H., Zheng, Y., Shao, M., Gay, D. L., Ramos, R., His, T. C., Oh, J. W., Wang, X., Ramirez, A., Konopelski, S. E., Elzein, A., Wang, A., Supapannachart, R. J., Lee, H. L., Lim, C. H., Nace, A., Guo, A., Treffeisen, E., Andl, T., Ramirez, R. N., Murad, R., Offermanns, S., Metzger, D., Chambon, P., Widgerow, A. D., Tuan, T. L., Mortazavi, A., Gupta, R. K., Hamilton, B. A., Millar, S. E., Seale, P., Pear, W. S., Lazar, M. A., & Cotsarelis, G. Regeneration of fat cells from myofibroblasts during wound healing. Science 355 (6326), 748-752 (2017).
6. Shook, B. A., Wasko, R. R., Rivera-Gonzalez, G. C., Salazar-Gatzimas, E., López-Giráldez, F., Dash, B. C., Munoz-Rojas, A. R., Aultman, K. D., Zwick, R. K., Lei, V., Arbiser, J. L., Miller-Jensen, K., Clark, D. A., Hsia, H. C., & Horsley, V. Myofibroblast proliferation and heterogeneity are supported by macrophages during skin repair. Science 362 (6417), eaar2971 (2018).
7. Mori, L., Bellini, A., Stacey, M. A., Schmidt, M., & Mattoli, S. Fibrocytes contribute to the myofibroblast population in wounded skin and originate from the bone marrow. Exp Cell Res 304 (1), 81-90 (2005).
8. Finnerty, C. C., Jeschke, M. G., Branski, L. K., Barret, J. P., Dziewwulski, P. & Herndon, D. N. Hypertrophic scarring: the greatest unmet challenge after burn injury. The Lancet 388, 1437-1436 (2016).
9. Morton, L. M. & Phillips, T. J. Wound healing and treating wounds: differential diagnosis and evaluation of chronic wounds. J Am Acad Dermatol 74 (4), 589-605 (2016).
10. Do, N. N. & Eming, S. A. Skin fibrosis: models and mechanisms. Curr Res Transl Med 64 (4), 185-193 (2016).
11. Sen, C. K., Gordillo, G. M., Roy, S., Kirsner, R., Lambert, L., Hunt, T. K., Gottrup, F., Gurtner, G. C. & Longaker, M. T. Human skin wounds: a major and snowballing threat to public health and the economy. Wound Repair Regen 17 (6), 763-71 (2009).
12. Rinkevich, Y., Walmsley, G. G., Hu, M. S., Maan, Z. N., Newman, A. M., Drukker, M., Januszyk, M., Krampitz, G. W., Gurtner, G. C., Lorenz, H. P., Weissman, I. L. & Longaker, M. T. Skin fibrosis. Identification and isolation of a dermal lineage with intrinsic fibrogenic potential. Science 348 (6232), aaa2151 (2015).
13. Jiang, D., Correa-Gallegos, D., Christ, S., Stefanska, A., Liu, J., Ramesh, P., Rajendran, V., De Santis, M. M., Wagner, D. E. & Rinkevich, Y. Two succeeding fibroblastic lineages drive dermal development and the transition from regeneration to scarring. Nat Cell Biol 20 (4), 422-431 (2018).
14. Adstrum, S., Hedley, G., Schleip, R., Stecco, C., Yucesoy, C. A. Defining the fascial system. J Bodyw Mov Ther 21 (1), 173-177 (2017).
15. Stecco, C. & Schleip, R. A fascia and the fascial system. J Bodyw Mov Ther 20 (1), 139-140 (2016).
16. Dunkin, C. S., Pleat, J. M., Gillespie, P. H., Tyler, M. P., Roberts, A. H. & McGrouther, D. A. Scarring occurs at a critical depth of skin injury: precise measurement in a graduated dermal scratch in human volunteers. Plast Reconstr Surg 119 (6), 1722-34 (2007).
17. Koehler, R., H., Begos, D., Berger, D., Carey, S., LeBlanc, K., Park, A., Ramshaw, B., Smoot, R., & Voeller, G. Minimal adhesions to ePTFE mesh after laparoscopic ventral incisional hernia repair: reoperative findings in 65 cases. JSLS 7 (4), 335-40 (2003).
18. Zamir, E. A., Rongish, B. J. & Little, C. D. The ECM moves during primitive streak formation—computation of ECM versus cellular motion. PLoS Biol 6 (10), e247 (2008).
19. Szabó, A., Rupp, P. A., Rongish, B. J., Little, C. D. & Czirók, A. Extracellular matrix fluctuations during early embryogenesis. Phys Biol 8 (4), 045006 (2011).
20. Aleksandrova, A., Czirók, A., Szabó, A., Filla, M. B., Hossain, M. J., Whelan. P. F., Lansford, R. & Rongish, B. J. Convective tissue movements play a major role in avian endocardial morphogenesis. Dev Biol 363 (2), 348-61 (2012).
21. Loganathan, R., Rongish, B. J., Smith, C. M., Filla, M. B., Czirok, A., Bénazéraf, B. & Little, C. D. Extracellular matrix motion and early morphogenesis. Development 143 (12), 2056-65 (2016).
22. Miron-Mendoza, M., Koppaka, V., Zhou, C. & Petroll, W. M. Techniques for assessing 3-D cell-matrix mechanical interactions in vitro and in vivo. Exp Cell Res 319 (16), 2470-80 (2013).
23. Sakar, M., S., Eyckmans, J., Pieters, R., Eberli, D., Nelson, B., J., & Chen, C., S., Cellular forces and matrix assembly coordinate fibrous tissue repair. Nat Commun 16 (7). 11036, (2016).
24. Abu-Hijleh, M., F., Roshier, A., L., Al-Shboul, Q., Dharap, A., S., & Harris, P., F. The membranous layer of superficial fascia: evidence for its widespread distribution in the body. Surg Radiol Anat 28 (6). 606-19 (2006).
25. Avelar, J. Regional distribution and behavior of the subcutaneous tissue concerning selection and indication for liposuction. Aesthetic Plast Surg 13 (3). 155-65 (1989).
26. Lockwood, T., E. Superficial fascial system (SFS) of the trunk and extremities: a new concept. Plast Reconstr Surg 87 (6). 1009-18 (1991).
27. Rippa, A., L., Kalabusheva, E., P., & Vorotelyak, E., A. Regeneration of dermis: scarring and cells involved. Cells 8 (6). 607 (2019).

1. A method for identifying modulators of extracellular matrix (ECM) movement towards a site requiring deposition of ECM, comprising
(a) contacting extracellular matrix of organ tissue obtainable by biopsy from a mammalian subject with a label;
(b) contacting said labelled extracellular matrix of organ tissue with a compound of interest;
(c) determining whether said compound of interest modulates ECM movement towards said site requiring deposition of ECM in comparison to labelled extracellular matrix of organ tissue obtainable by biopsy from a mammalian subject which is not contacted with said compound of interest,
wherein modulation of ECM movement towards said site requiring deposition of ECM is indicative for said compound of interest to be a modulator of said ECM movement.

2. The method of claim 1, wherein modulation is inhibition or promotion.

3. The method of any one of the preceding claims, wherein said organ tissue comprises fascia matrix, serosa and/or adventitia.

4. The method of any one of the preceding claims, wherein fascia matrix, serosa and/or adventitia comprises macrophages, neutrophils, mesothelial cells and/or fibroblasts.

5. The method of any one of the preceding claims, wherein the label is a dye or tag.

6. The method of any one of the preceding claims, wherein the organ tissue is from skin, kidney, lung, heart, liver, bone, peritoneum, intestine, diaphragm or pleura.

7. A method for identifying a biomarker associated with extracellular matrix (ECM) movement towards a site requiring deposition of ECM, comprising:
(a) contacting extracellular matrix of organ tissue obtainable by biopsy from a mammalian subject with a label;
(b) isolating proteins from said labelled ECM which move towards said site requiring deposition of ECM;
(c) determining at least a partial amino acid sequence of said proteins, thereby identifying said proteins as a biomarker associated with ECM movement.

8. A compound for use in a method for the modulation of extracellular matrix (ECM) movement towards a site requiring deposition of ECM, preferably in the treatment of a condition involving ECM deposition.

9. The compound for the use of claim 8, wherein ECM movement is mediated by fascia matrix.

10. The compound for the use of claim 8 or 9, wherein fascia matrix, serosa and/or adventitia comprises macrophages, neutrophils, mesothelial cells, and/or fibroblasts.

11. The compound for the use of any one of claims 8 to 10, wherein the site requiring deposition of ECM is a wound.

12. The compound for the use of any one of claims 8 to 11, wherein modulation is inhibition.

13. The compound for the use of any one of claims 8 to 12, wherein the condition involving ECM deposition is excessive deposition of ECM.

14. The compound for the use of any one of claims 8 to 11, wherein modulation is promotion.

15. The compound for the use of any one of claims 8 to 11 and 14, wherein the condition involving ECM deposition is insufficient deposition of ECM.
